(19) United States
(12) Patent Application Publication
(10) Pub. No.: US 2025/0059213 A1
(43) Pub. Date: Feb. 20, 2025

(54) LONP1 INHIBITORS, USES AND METHODS

(71) Applicant: Pretzel Therapeutics, Inc., Waltham, MA (US)

(72) Inventors: Jeremy Green, Waltham, MA (US); Paul Charifson, Carrabassett Valley, ME (US); Gabriel Martinez Botella, Wayland, MA (US); Andrew Griffin, L'Île Bizard, Quebec (CA)

(73) Assignee: Pretzel Therapeutics, Inc., Waltham, MA (US)

(21) Appl. No.: 18/716,894

(22) PCT Filed: Dec. 6, 2022
(86) PCT No.: PCT/US2022/051985

§ 371 Date: Jun. 5, 2024

Related U.S. Application Data
(60) Provisional application No. 63/286,441, filed on Dec. 6, 2021.
(60) Provisional application No. 63/290,841, filed on Dec. 17, 2021.
(60) Provisional application No. 63/319,596, filed on Mar. 14, 2022.
(60) Provisional application No. 63/358,741, filed on Jul. 6, 2022.

(51) Int. Cl. C07F 5/02; A61K 31/69; A61K 45/06

(52) U.S. Cl. CPC C07F 5/025; A61K 31/69; A61K 45/06

(57) ABSTRACT

Disclosed are compounds according to Formula (1) and Formula (2) which inhibit LONP1, and pharmaceutical compositions comprising compounds of the disclosure. Compounds and pharmaceutical compositions of the disclosure may be useful for the treatment of diseases and disorders associated with LONP1, including oncologic diseases and disorders, such as cancer, and diseases and disorders related to mitochondrial dysfunction, such as neurodegenerative disorders, metabolic disorders, and diseases associated with the aging process. The disclosure also relates to methods of using such compounds and compositions for the treatment of such diseases and disorders.

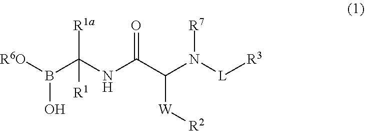

### FIELD OF THE INVENTION

The present invention relates to novel LONP1 inhibitors, their pharmaceutically acceptable salts, and pharmaceutical compositions thereof. The present invention also relates to methods of using such compounds and compositions, including to inhibit LONP1 and to treat oncologic diseases and disorders, such as cancer, and various diseases and disorders related to mitochondrial dysfunction, such as neurodegenerative disorders, metabolic disorders, and diseases associated with the aging process.

### BACKGROUND OF THE INVENTION

The mitochondrial Lon serine protease, LONP1, is an enzyme that is a member of the AAA+ superfamily of proteases (i.e., ATP-dependent proteases (ATPases) associated with diverse cellular activities). Widely conserved across eukaryotic species, human LONP1 is a 959-amino acid protein that consists of three domains: the N-terminal domain involved in substrate binding, the AAA+ (ATPase) domain, and the C-terminal domain (named the P-domain) involved in proteolytic activity. The ATPase and protease domains are the most well-conserved across species, while the N-terminal domain is the most variable.

LONP1 performs at least four different functions: proteolysis of damaged and oxidized proteins of the mitochondrial matrix; chaperone activity, namely the correct folding of proteins imported into the mitochondria; regulation of mitochondrial protein levels, including mitochondrial transcription factor A (TFAM); and binding to mitochondrial DNA (“mtDNA”) and RNA. As for the proteolytic activity of LONP1, like all the other proteases in the AAA+ family, it binds its substrate, unfolds it using the ATPase domain, and then digests it from the N or C-terminus. Its chaperone activity, mediated by the ATP-binding domain and the N-terminal domain, is crucial for mitochondrial homeostasis, as it is involved in the assembly of mitochondrial membrane complexes.

LONP1 has multiple, natural substrates, one of which is the mtDNA binding and packaging protein TFAM. TFAM has a crucial role in transcription initiation and mtDNA replication. Inhibition of LONP1 reportedly leads to increased levels of the TFAM protein, which in turn may lead to higher levels of mtDNA.

TFAM and mtDNA have a mutual dependence for stability, whereby TFAM binds mtDNA and protects it from degradation, but when not bound to mtDNA, TFAM is rapidly degraded. LONP1 has been shown to regulate mtDNA copy number in Drosophila melanogaster by cleaving TFAM. In human cells with severe mtDNA deficits, depletion of LONP1 can increase levels of TFAM and upregulate mtDNA content.

Another natural substrate of LONP1 is POLγA, the catalytic subunit of DNA polymerase γ (POLγ). POLγ is the main protein responsible for mitochondrial DNA (mtDNA) replication. The accessory POLγB subunit acts to stabilize POLγA and to prevent LONP1-dependent degradation. Disease causing mutations such as A467T weaken interactions between POLγA and POLγB, which in turn makes POLγA susceptible to degradation by LONP1.

LONP1 is also required during embryogenesis. A homozygous deletion of the LONP1 gene in a mouse causes embryonic lethality. In line with this observation, mutations that change LONP1 activity during embryogenesis can cause a congenital syndrome known as CODAS, characterized by Cerebral, Ocular, Dental, Auricular and Skeletal anomalies. Further supporting a role during embryogenesis, defective mitochondrial protease LONP1 has also been linked to a classical, congenital mitochondrial disease. The mutant (Tyr565His) protein displayed higher ATPase activity, but reduced protease activity. See Peter, B. et. al., “Defective Mitochondrial Protease LonP1 Can Cause Classical Mitochondrial Disease,” Hum. Mol. Genet., 27, 10, 1743-1750 (2018).

Additionally, LONP1 has a central role in the regulation of mitochondrial function, impacting bioenergetics in various cells and often causing disease (see Gibellini L. et. al., “LonP1 Differently Modulates Mitochondrial Function and Bioenergetics of Primary Versus Metastatic Colon Cancer Cells,” Front. Oncol. 8, 254 (2018). LONP1 upregulation is a characteristic shared by various types of cancer cells. Higher expression of LONP1 is correlated with tumor progression and aggressiveness. For instance, LONP1 overproduction is functionally linked to colorectal cancer cells by inducing the epithelial mesenchymal transition, an early step in the formation of metastases (see id). Furthermore, LONP1 is a regulator of mitochondrial proteostasis, which is required for maintaining the respiratory chain and degrading misfolded, oxidatively damaged or unassembled proteins. As such, inhibition of LONP1 is believed to be a mechanism by which various oncogenic diseases, such as cancers may be treated.

Similarly, multiple myeloma is an exceedingly prevalent and incurable cancer in the elderly (see Maneix, L. et al., “The Mitochondrial Protease LonP1 Promotes Proteasome Inhibitor Resistance in Multiple Myeloma,” Cancers 13, 843, 14-19 (2021)). Proteasome inhibitors are a common treatment for myeloma, but for unknown reasons, over time, a resistance to treatment develops. Compounds that inhibit LONP1 may provide a means to more thoroughly understand the molecular mechanisms that lead to such drug resistance in the treatment of multiple myeloma (see id).

While aspects of LONP1 biochemistry are known, its full physiological role in mitochondrial gene expression and homeostasis, as well as its underlying impact in the etiology of various disease states, remains unclear. LONP1 inhibitors will provide insight into, for example, the relationship between LONP1, mtDNA copy number, and human diseases. Pharmacological inhibition of LONP1 is one means by which to gain a further understanding of the role of this protease in cell physiology and the development of disease. LONP1 inhibitors have been reported, for example, in Kingsley, L. J. et al., J. Med. Chem. 64, 8, 4857-4869 (2021). In view of the numerous and varied roles of LONP1, there is a need for additional, potent, and specific inhibitors of LONP1.

### SUMMARY OF THE INVENTION

Provided are compounds, pharmaceutically acceptable salts of the compounds, pharmaceutical compositions comprising the compounds or their salts, methods of using the compounds, salts of the compounds, or pharmaceutical compositions of the compounds or their salts, and therapeutic uses of the compounds, or pharmaceutical compositions of the compounds or their salts, for treating diseases related to oncologic diseases and disorders, such as cancer, and/or various diseases and disorders related to mitochondrial dysfunction, such as neurodegenerative disorders, metabolic disorders, and diseases associated with the aging process. The compounds and their pharmaceutically acceptable salts are particularly useful as inhibitors of LONP1.

In one aspect, there is provided a compound, or a pharmaceutically acceptable salt thereof, represented by Formula 1:

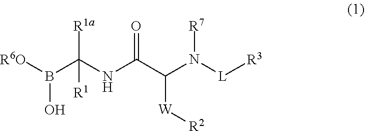

or a pharmaceutically acceptable salt, solvate, stereoisomer or mixture of stereoisomers, tautomer, isotopic form, pharmaceutically active metabolite thereof, or combinations thereof, wherein:
R¹ is selected from the group consisting of: deuterium, C1-C4 alkyl, C1-C4 alkoxyl, C₁-C₄ oxoalkyl, C1-C5 alkyl-alkoxyl, wherein each alkyl, oxoalkyl or alkoxyl is optionally substituted with C3-C6 cycloalkyl, phenyl, phenoxy, or a 5- or 6-membered heteroaryl, wherein said phenyl, phenoxy, or heteroaryl are each optionally substituted with one or more substituent selected from deuterium, halogen, hydroxyl, CN, CO2H, CO2R¹², CONR¹²R¹³, NR¹²R¹³, SR¹², SO2NR¹²R¹³, C1-C4 alkyl, C1-C4 haloalkyl, C1-C4 alkoxy, phenyl, or a 5- or 6-membered heteroaryl;
R^{1a} is selected from hydrogen, deuterium or C1-C2 alkyl;
W is C1-C4 alkylene, optionally substituted with one or more of deuterium, halogen, hydroxyl, oxo, CN, methyl or ethyl;
R² is selected from the group consisting of: hydrogen, deuterium, R⁴, —OR⁴, —C(O)R⁴, —S—R⁴, —SO—R⁴, —SO₂—R⁴, —SO₂—NR⁵R¹¹,
L is C(O), C(O)O, C(O)NR⁸, S(O)₂, or a bond;
R³ is C₁-C₄ alkyl optionally substituted with one or more substituents each independently selected from the group consisting of deuterium, halogen, cyano, hydroxyl, C₁-C₄ alkoxyl, C₁-C₄ alkylthiol or phenyl; or
R³ is saturated or unsaturated cycloalkyl or saturated or unsaturated heterocycloalkyl having one or more heteroatoms selected from N, O and S, wherein the cycloalkyl or heterocycloalkyl is optionally substituted with one or more substituents selected from deuterium, halogen, cyano, hydroxyl, oxo, C1-C₄ alkoxyl, or C₁-C₄ alkyl that is optionally substituted with one to three substituents selected from deuterium, halogen, cyano, hydroxyl, C₁-C₄ alkylthiol or C₁-C₄ alkoxyl; or
R³ is aryl or heteroaryl having one or more heteroatoms selected from N, O and S, wherein aryl or heteroaryl is optionally substituted with one or more substituents selected from deuterium, halogen, cyano, hydroxyl, OR, CO2H, CO2R¹², CONR¹²R¹³, NR¹²R¹³, SR¹², SO2NR¹²R¹³, C₁-C₄ alkoxyl, or C₁-C₄ alkyl that is optionally substituted with one to three substituents selected from deuterium, halogen, cyano, hydroxyl, or C₁-C₄ alkoxyl;
R⁴ is hydrogen, deuterium, C₁-C₄ alkyl or C₁-C₄ alkoxyl optionally substituted with one or more substituents each independently selected from the group consisting of deuterium, halogen, cyano, hydroxyl, C₁-C₄ alkoxyl, C₁-C₄ alkylthiol, C₁-C₅ alkyl-alkoxyl or phenyl; or
R⁴ is saturated or unsaturated cycloalkyl or saturated or unsaturated heterocycloalkyl having one or more heteroatoms selected from N, O and S, wherein the cycloalkyl or heterocycloalkyl is optionally substituted with one or more substituents selected from deuterium, halogen, cyano, hydroxyl, C₁-C₄ alkoxyl, or C₁-C₄ alkyl that is optionally substituted with one to three substituents selected from deuterium, halogen, cyano, hydroxyl, or C₁-C₄ alkoxyl, C₁-C₄ alkylthiol and C₁-C₅ alkyl-alkoxyl; or
R⁴ is aryl or heteroaryl having one or more heteroatoms selected from N, O and S, wherein aryl, or heteroaryl is optionally substituted with one or more substituents selected from deuterium, halogen, cyano, hydroxyl, C₁-C₄ alkoxyl, or C₁-C₄ alkyl that is optionally substituted with one to three substituents selected from deuterium, halogen, cyano, hydroxyl, or C₁-C₄ alkoxyl, C₁-C₄ alkylthiol and C₁-C₅ alkyl-alkoxyl; or
R⁴ is NR⁹R¹⁰;
R⁵ and R¹¹ are each independently selected from hydrogen, deuterium or C1-C5 alkyl optionally substituted with one to three halogen; or
R⁵ and R¹¹ together with the N to which they are attached form a 5 or 6 membered saturated or unsaturated heterocyclic ring optionally having one or more additional heteroatoms selected from N, O and S, wherein the 5 or 6 membered heterocyclic ring is optionally substituted with one or more substituent selected from deuterium, halogen, hydroxyl, CN, C1-C4 alkyl, C1-C4 haloalkyl or C1-C4 alkoxy;
R⁶ is hydrogen, or R⁶ and R¹, together with the boron atom to which —OR⁶ is attached form a 5-membered heteroalkyl ring.
R⁷ is selected from hydrogen, deuterium, or C₁-C₄ alkyl optionally substituted with one or more substituents each independently selected from the group consisting of deuterium, halogen, hydroxyl, cyano, methoxyl, methylthiol and phenyl;
R⁸ is hydrogen or C₁-C₄ alkyl optionally substituted with one or more of deuterium, halogen, hydroxyl and phenyl, wherein phenyl is optionally substituted with one or more substituent selected from deuterium, halogen, hydroxyl and C₁-C₂ alkyl;
R⁹ and R¹⁰ are each independently selected from hydrogen, deuterium or C₁-C₆ alkyl that is optionally substituted with one to three substituents selected from deuterium, halogen, cyano, or C₁-C₄ alkoxyl; or
R⁹ and R¹⁰ together with the N to which they are attached form a 5 or 6 membered saturated or unsaturated heterocyclic ring optionally having one or more additional heteroatoms selected from N, O and S, wherein the 5 or 6 membered heterocyclic ring is optionally substituted with one or more substituent selected from deuterium, halogen, hydroxyl, CN, C1-C4 alkyl, C1-C4 haloalkyl or C1-C4 alkoxy; and
R¹² and R¹³ are each independently selected from hydrogen, deuterium, C1-C4 alkyl; C1-C4 haloalkyl, C1-C5 alkyl-alkoxyl, C3-C7 cycloalkyl, or R¹² and R¹³ together with the N to which they are attached form 3 to 7 membered heterocyclic ring optionally having one or more additional heteroatoms selected from N, O and S, wherein the C3-C7 cycloalkyl or 3 to 7 membered heterocyclic ring is optionally substituted with one or more substituent selected from deuterium, halogen, hydroxyl, oxo, CN, C1-C4 alkyl, C1-C4 haloalkyl or C1-C4 alkoxyl.
R² may particularly be selected from the group consisting of: R⁴, —OR⁴, —C(O)R⁴, —S—R⁴, —SO—R⁴, —SO₂—R⁴, —SO₂—NR⁵R¹¹.

In another aspect, there is provided a compound, or a pharmaceutically acceptable salt thereof, represented by Formula 2:

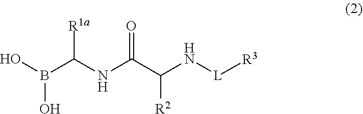

wherein:
R¹ is C₁-C₄ alkyl, C₁-C₅ alkoxy, C₁-C₄ alkyl alkoxy, each optionally substituted with phenyl or heteroaryl;
R² is (C₁-C₄ alkyl)-C(O)R⁴, (C₁-C₄ alkyl)-OR⁴, (C₁-C₄ alkyl)-R⁴, (C₁-C₄ alkyl)-SO₂—NR⁵R⁶, (C₁-C₄ alkyl)-SO₂—R⁴, (C₁-C₄ alkyl)-SO—R⁴;
L is C(O), C(O)O, C(O)NR⁵, S(O)₂, or a bond;
R³ is C₁-C₄ alkyl optionally substituted with one or more substituents each independently selected from the groups consisting of fluoro, chloro, cyano, or methoxyl, cycloalkyl, heterocyclyl having one or more heteroatoms, aryl, cycloaryl, or heteroaryl having one or more heteroatoms, any of which is optionally substituted with one or more fluoro, chloro, cyano, methoxyl, or C₁-C₄ alkyl that is optionally substituted with one to three fluoro, chloro, cyano, or methoxyl;
R⁴ is C₁-C₄ alkyl, an amino optionally substituted with one or more C₁-C₄ alkyl, an optionally substituted 5- or 6-membered heterocyclic ring having one or more heteroatoms, an optionally substituted 5- or 6-membered heteroaromatic ring having one or more heteroatoms, or an optionally substituted 5- or 6-membered aromatic ring; and
R⁵ and R⁶ are each independently hydrogen, or C₁-C₄ alkyl optionally substituted with one to three fluoro.

Another aspect of the disclosure is directed to pharmaceutical compositions comprising a compound of the disclosure (that is, compounds of Formula I) or a pharmaceutically acceptable salt thereof and a pharmaceutically acceptable excipient.

Other aspects of the disclosure are directed to methods of treating a disease or disorder, such as a disease or disorder characterized by mitochondrial dysfunction, such methods comprising administering to a subject in need thereof a therapeutically effective amount of a compound of the disclosure, a pharmaceutically acceptable salt thereof, or a composition comprising such as compound.

In various aspects and embodiments of the methods and therapeutic uses disclosed herein, the disease is selected from Alper's syndrome (Alpers-Huttenlocher syndrome), ataxia neuropathy syndrome (ANS), Mitochondrial DNA Depletion Syndrome (MDDS), Leigh Syndrome (Leigh Disease), Leber's Hereditary Optic Neuropathy (LHON), chronic progressive external ophthalmoplegia (CPEO), myoclonic epilepsy myopathy sensory ataxia (MEMSA), MELAS (Mitochondrial Encephalopathy, Lactic Acidosis, and Stroke-like episodes) syndrome, MERRF (myoclonus epilepsy with ragged-red fibers) syndrome, mitochondrial neurogastrointestinal encephalomyopathy (MNGIE), neuropathy, ataxia, and retinitis pigmentosa (NARP), Kearn's-Sayre Syndrome (KSS), and Pearson's Syndrome. In some aspects and embodiments the disease or disorder is selected from Alzheimer's disease, Parkinson's disease, obesity, diabetes, non-alcoholic steatohepatitis (NASH), and related metabolic syndromes such as non-alcoholic fatty liver disease (NAFLD).

Other aspects of the disclosure are directed to compounds or (pharmaceutical) compositions comprising compounds of the disclosure for use in methods for treating a disease or disorder, such as a disease or disorder characterized by mitochondrial dysfunction. These therapeutic uses may comprise administering to a subject in need thereof a therapeutically effective amount of a compound of the disclosure, a pharmaceutically acceptable salt thereof, or a composition comprising such a compound. Suitable diseases or disorders are those described above and herein below.

In some embodiments, the disease to be treated with a compound or composition of the disclosure is associated with mtDNA mutations or deletions, for example: m.3243A>G, m.11778G>A, m.14484T>C, m.3460G>A, m.8344A>G, m.3271T>C, m.3251A>G, m.8356T>C, m.4274T>C, m.14709T>C, m.12320A>G, m.4269A>G, m.12258C>A, m.1606G>A, m.10010T>C, m.7445A>G and m.1555A>G (see https://mitomap.org/MITOMAP).

Additional aspects and embodiments of the disclosure relate to methods of treating cancers and compounds or compositions for use in such methods: for example, those identified in Wong, K. S. et al. “Recent Advances in Targeting Human Mitochondrial AAA+ Proteases to Develop Novel Cancer Therapeutics,” Advances in Experimental Medicine and Biology, 1158, 119-142 (2019), wherein the use or method comprising using a compound or composition of the disclosure or its pharmaceutically acceptable salt.

Further aspects and embodiments of the disclosure relate to methods of treating cancer, neurodegenerative disorders, metabolic disorders, and diseases associated with the aging process; and compounds and compositions of the disclosure for use in such methods.

Within the scope of this disclosure it is expressly intended that the various aspects, embodiments, examples and alternatives set out in the preceding paragraphs, in the claims and/or in the following description, and in particular the individual features thereof, may be taken independently or in any combination. That is, all embodiments and/or features of any aspect or embodiment can be combined in any way and/or combination, unless such features are incompatible. More particularly, it is specifically intended that any embodiment of any aspect may form an embodiment of any other aspect, and all such combinations are encompassed within the scope of the disclosure. The applicant reserves the right to change any originally filed claim or file any new claim accordingly, including the right to amend any originally filed claim to depend from and/or incorporate any feature of any other claim although not originally claimed in that manner.

### DETAILED DESCRIPTION OF THE INVENTION

Described herein are compounds and compositions (e.g. organic molecules, research tools, pharmaceutical formulations and therapeutics); uses for the compounds and compositions of the disclosure (in vitro and in vivo); as well as corresponding methods, whether diagnostic, therapeutic or for research applications. The chemical synthesis and biological testing of the compounds of the disclosure are also described. Beneficially, the compounds, compositions, uses and methods have utility in research towards and/or the treatment of diseases or disorders in animals, such as humans. Diseases or disorders which may benefit from LONP1 modulation include mitochondrial diseases, cancer and/or oncologic disease.

However, the compounds of the disclosure may also or alternatively be useful as lead molecules for the selection, screening and development of further derivatives that may have one or more improved beneficial drug property, as desired.

The disclosure also encompasses salts, solvates and functional derivatives of the compounds described herein. These compounds may be useful in the treatment of diseases or disorders characterized by mitochondrial disfunction; particularly those which may benefit from LONP1 inhibition.

Inhibitors of LONP1 are useful in compositions and methods suitable for treating many disorders, such as disorders characterized by mitochondrial dysfunction, including cancer. In some embodiments, the disease is selected from the group consisting of adrenal gland cancer, anal cancer, adenocarcinoma, angiosarcoma, bile duct cancer, bladder cancer, blastic plasmacytoid dendritic cell neoplasm, bone cancer, brain cancer, breast cancer, bronchogenic carcinoma, central nervous system (CNS) cancer, cervical cancer, cholangiocarcinoma, chondrosarcoma, colon cancer, choriocarcinoma, colorectal cancer, cancer of connective tissue, esophageal cancer, embryonal carcinoma, fibrosarcoma, gall bladder cancer, gastric cancer, glioblastomas, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), liposarcoma, liver cancer, lung cancer, lymphoid cancers (e.g., Hodgkin's and non-Hodgkin's lymphomas), melanoma, Merkel cell carcinoma, mesothelioma, multiple myeloma, muscular cancer, myxosarcoma, neuroblastomas, non-small cell lung cancer, ocular cancer, oral/digestive tract cancer, osteogenic sarcoma, ovarian cancer, papillary carcinoma, pancreatic cancer, polycythemia vera, prostate cancer, rhabdomyosarcoma, renal cancer, retinal cancer, skin cancer, small cell lung carcinoma, stomach cancer, testicular cancer, throat cancer, thyroid cancer, uterine cancer, vaginal cancer, and vulvar cancer. In some embodiments, the disease is selected from the group consisting of Alzheimer's disease and Parkinson's disease. In some embodiments, the disease is selected from the group consisting of obesity, diabetes, non-alcoholic steatohepatitis (NASH), and related metabolic syndromes such as non-alcoholic fatty liver disease (NAFLD). In some embodiments, the disease is related to aging or a mitochondrial disorder.

Provided herein are compounds of Formula I, pharmaceutically acceptable salts thereof, and pharmaceutical compositions comprising such compounds or their salts that are useful in treating a condition or disease characterized by mitochondrial dysfunction.

### Definitions

Unless defined otherwise, all technical and scientific terms used herein have the same meaning as commonly understood by one of ordinary skill in the art (e.g. in organic, physical or theoretical chemistry; biochemistry and molecular biology).

Unless otherwise indicated, the practice of the present invention employs conventional techniques in chemistry and chemical methods, biochemistry, molecular biology, pharmaceutical formulation, and delivery and treatment regimens for patients, which are within the capabilities of a person of ordinary skill in the art. Such techniques are also described in the literature cited herein. All documents cited in this disclosure are herein incorporated by reference in their entirety.

Prior to setting forth the detailed description of the inventions, a number of definitions are provided that will assist in the understanding of the disclosure.

In accordance with this disclosure, the terms ‘molecule’ or ‘molecules’ are used interchangeably with the terms ‘compound’ or ‘compounds’, and sometimes the term ‘chemical structure’. The term ‘drug’ is typically used in the context of a pharmaceutical, pharmaceutical composition, medicament or the like, which has a known or predicted physiological or in vitro activity of medical significance; but such characteristics and qualities are not excluded in a molecule or compound of the disclosure. The term ‘drug’ is therefore used interchangeably with the alternative terms and phrases ‘therapeutic (agent)’, ‘pharmaceutical (agent)’, and ‘active (agent)’. Therapeutics according to the disclosure also encompass compositions and pharmaceutical formulations comprising the compounds of the disclosure.

Prodrugs and solvates of the compounds of the disclosure are also encompassed within the scope of the disclosure. The term ‘prodrug’ means a compound (e.g. a drug precursor) that is transformed in vivo to yield a compound of the disclosure or a pharmaceutically acceptable salt, solvate or ester of the compound. The transformation may occur by various mechanisms (e.g. by metabolic or chemical processes), such as by hydrolysis of a hydrolysable bond, e.g. in blood (see Higuchi & Stella (1987), “Pro-drugs as Novel Delivery Systems”, vol. 14 of the A.C.S. Symposium Series; (1987), “Bioreversible Carriers in Drug Design”, Roche, ed., American Pharmaceutical Association and Pergamon Press). The compositions and medicaments of the disclosure therefore may comprise prodrugs of the compounds of the disclosure. In some aspects and embodiments the compounds of the disclosure may be themselves prodrugs which may be metabolised in vivo to give the therapeutically effective compound.

The scope of this disclosure also includes various deuterated forms of the compounds of any of Formula I (inc. corresponding subgeneric formulas defined herein), respectively, or a pharmaceutically acceptable salt and/or a corresponding tautomer form thereof (including subgeneric formulas, as defined above). Each available hydrogen atom attached to a carbon atom may be independently replaced with a deuterium atom. A person of ordinary skill in the art will know how to synthesize deuterated forms of the compounds of Formula I disclosed herein (including subgeneric formulas, as defined above) or a pharmaceutically acceptable salt and/or a corresponding tautomer form thereof (including subgeneric formulas, as defined above) of the present disclosure. For example, deuterated materials, such as alkyl groups may be prepared by conventional techniques (see for example: methyl-d₃-amine available from Aldrich Chemical Co., Milwaukee, WI, Cat. No. 489,689-2).

The disclosure also includes isotopically-labelled compounds which are identical to those recited in Formula I disclosed herein (inc. corresponding subgeneric formulas defined herein), respectively, or a pharmaceutically acceptable salt and/or a corresponding tautomer form thereof (including subgeneric formulas, as defined above), but for the fact that one or more atoms are replaced by an atom having an atomic mass or mass number different from the atomic mass or mass number most commonly found in nature. Examples of isotopes that can be incorporated into compounds of the disclosure include isotopes of hydrogen, carbon, nitrogen, oxygen, fluorine, iodine and chlorine such as 2 H, 3 H, 11 C, 14 C, 18 F, 123 I or 125 I. Compounds of the present disclosure and pharmaceutically acceptable salts of said compounds that contain the aforementioned isotopes and/or other isotopes of other atoms are within the scope of the present disclosure. Isotopically labelled compounds of the present disclosure, for example those into which radioactive isotopes such as 3 H or 14 C have been incorporated, are useful in drug and/or substrate tissue distribution assays. Tritiated, i.e. 3 H, and carbon-14, i.e. 14 C, isotopes are particularly preferred for their ease of preparation and detectability. 11 C and 18 F isotopes are particularly useful in PET (positron emission tomography).

In the context of the present disclosure, the terms ‘individual’, ‘subject’, or ‘patient’ are used interchangeably to indicate an animal that may be suffering from a medical (pathological) condition and may be responsive to a compound/molecule, pharmaceutical drug, medical treatment or therapeutic treatment regimen of the disclosure. The animal is suitably a mammal, such as a human, cow, sheep, pig, dog, cat, bat, mouse or rat. In particular, the subject may be a human.

The term ‘alkyl’ refers to a monovalent, optionally substituted, saturated aliphatic hydrocarbon radical. Any number of carbon atoms may be present, but typically the number of carbon atoms in the alkyl group may be from 1 to about 20, from 1 to about 12, from 1 to about 6 or from 1 to about 4. Usefully, the number of carbon atoms is indicated, for example, a C1-C12 alkyl (or C₁-C₁₂ alkyl) refers to any alkyl group containing 1 to 12 carbon atoms in the chain. An alkyl group may be a straight chain (i.e. linear), branched chain, or cyclic. ‘Lower alkyl’ refers to an alkyl of 1 to 6 carbon atoms in the chain, and may have from 1 to 4 carbon atoms, or 1 to 2 carbon atoms. Thus, representative examples of lower alkyl radicals include methyl, ethyl, n-propyl, n-butyl, n-pentyl, n-hexyl, isopropyl, isobutyl, isopentyl, amyl (C₅H₁₁), sec-butyl, tert-butyl, sec-amyl, tert-pentyl, 2-ethylbutyl, 2,3-dimethylbutyl, and the like. ‘Higher alkyl’ refers to alkyls of 7 carbons and above, including n-heptyl, n-octyl, n-nonyl, n-decyl, n-dodecyl, n-tetradecyl, n-hexadecyl, n-octadecyl, n-eicosyl, and the like, along with branched variations thereof. A linear carbon chain of say 4 to 6 carbons would refer to the chain length not including any carbons residing on a branch, whereas in a branched chain it would refer to the total number. Optional substituents for alkyl and other groups are described below.

The term ‘alkoxy’ or ‘alkoxyl’ as used herein refers to a monovalent radical of the formula RO—, where R is any alkyl, alkenyl or alkynyl as defined herein. Alkoxy groups may be optionally substituted by any of the optional substituents described herein. ‘Lower alkoxy’ has the formula RO—, where the R group is a lower alkyl, alkenyl or alkynyl. Representative alkoxy radicals include methoxyl, ethoxyl, n-propoxy, n-butoxyl, n-pentyloxyl, n-hexyloxyl, isopropoxyl, isobutoxyl, isopentyloxyl, amyloxyl, sec-butoxyl, tert-butoxyl, tert-pentyloxyl, and the like. Preferred alkoxyl groups are methoxyl and ethoxyl.

The term ‘cycloalkyl’ as used herein refers to a cyclized alkyl ring having the indicated number of carbon atoms in a specified range. Thus, for example, “C₃-C₆ cycloalkyl” encompasses each of cyclopropyl, cyclobutyl, cyclopentyl, and cyclohexyl.

The term ‘aryl’ as used herein refers to a substituted or unsubstituted aromatic carbocyclic radical containing from 5 to about 15 carbon atoms (“C₆-C₁₅ aryl”); and preferably 6 to 12 carbon atoms (“C₆-C₁₂ aryl”). An aryl group may have only one individual carbon ring, or may comprise one or more fused rings in which at least one ring is aromatic in nature. A ‘phenyl’ is a radical formed by removal of a hydrogen atom from a benzene ring, and may be substituted or unsubstituted. A ‘phenoxy’ group, therefore, is a radical of the formula RO—, wherein R is a phenyl radical. ‘Benzyl’ is a radical of the formula R—CH₂—, wherein R is phenyl, and ‘benzyloxyl’ is a radical of the formula RO—, wherein R is benzyl. The point of attachment to the base molecule on such fused aryl ring systems may be a C atom of the aromatic portion or a C or a N atom of the non-aromatic portion of the ring system. Non-limiting examples of aryl radicals include, phenyl, naphthyl, anthracenyl, benzyl, biphenyl, furanyl, pyridinyl, indanyl, anthraquinolyl, tetrahydronaphthyl, a benzoic acid radical, a furan-2-carboxylic acid radical, and the like.

The term ‘cycloaryl’ herein refers to a polycyclic group wherein an aryl group is fused to a 5- or 6-membered aliphatic ring. For example, C₆-C₁₂ cycloaryl means a C₆-C₁₂ aryl fused to a 5- or 6-membered aliphatic ring.

The term ‘heteroaryl’ as used herein refers to (i) a 5- or 6-membered ring having the characteristics of aromaticity containing at least one heteroatom selected from N, O and S, wherein each N is optionally in the form of an oxide, and (ii) a 9- or 10-membered bicyclic fused ring system, wherein the fused ring system of (ii) contains at least one heteroatom independently selected from N, O and S, wherein each ring in the fused ring system contains zero, one or more than one heteroatom, at least one ring is aromatic, each N is optionally in the form of an oxide, and each S in a ring which is not aromatic is optionally S(O) or S(O)₂. Typically, heteroaryl groups contain 5 to 14 ring atoms (‘5-14 membered heteroaryl’), and preferably 5 to 12 ring atoms (‘5-12 membered heteroaryl’). Heteroaryl rings are attached to the base molecule via a ring atom of the heteroaromatic ring, such that aromaticity is maintained. Suitable 5- and 6-membered heteroaromatic rings include, for example, pyridyl, 3-fluroropyridyl, 4-fluoropyridyl, 3-methoxypyridyl, 4-methoxypyridyl, pyrrolyl, pyrazinyl, pyrimidinyl, pyridazinyl, triazinyl, thienyl, furanyl, imidazolyl, pyrazolyl, triazolyl (i.e., 1, 2, 3-triazolyl or 1, 2, 4-triazolyl), tetrazolyl, oxazolyl, isooxazolyl, oxadiazolyl (i.e., the 1, 2, 3—, 1, 2, 4-, 1, 2, 5-(furazanyl), or 1, 3, 4-isomer), oxatriazolyl, thiazolyl, isothiazolyl, and thiadiazolyl. Suitable 9- and 10-membered heterobicyclic, fused ring systems include, for example, benzofuranyl, indolyl, indazolyl, naphthyridinyl, isobenzofuranyl, benzisoxazolyl, benzoxazolyl, benzothiazolyl, chromenyl, quinolinyl, isoquinolinyl, benzopiperidinyl, benzofuranyl, imidazo[1, 2-a]pyridinyl, benzotriazolyl, indazolyl, indolinyl, and isoindolinyl.

The term ‘heteroaryloxy’ or ‘heteroaryloxyl’ as used herein refers to an —O-heteroaryl group.

The terms ‘heterocycle’ or ‘heterocyclic’ group or ‘heterocyclyl’ as used herein refer to a monovalent radical of from about 4-to about 15-ring atoms, and preferably 3—, 4-, 5-, 6-, 7-, 8-, 9- or 10-ring members. Generally, the heterocyclic group contains one, two or three heteroatoms, selected independently from nitrogen, oxygen and sulphur. A preferred heteroatom is N. A heterocyclic group may have only one individual ring or may comprise one or more fused rings in which at least one ring contains a heteroatom. It may be fully saturated or partially saturated and may be substituted or unsubstituted as in the case or aryl and heteroaryl groups. Representative examples of unsaturated 5-membered heterocycles with only one heteroatom include 2- or 3-pyrrolyl, 2- or 3-furanyl, and 2- or 3-thiophenyl. Corresponding partially saturated or fully saturated radicals include 3-pyrrolin-2-yl, 2- or 3-pyrrolindinyl, 2- or 3-tetrahydrofuranyl, and 2- or 3-tetrahydrothiophenyl. Representative unsaturated 5-membered heterocyclic radicals having two heteroatoms include imidazolyl, oxazolyl, thiazolyl, pyrazolyl, and the like. The corresponding fully saturated and partially saturated radicals are also included. Representative examples of unsaturated 6-membered heterocycles with only one heteroatom include 2—, 3-, or 4-pyridinyl, 2H-pyranyl, and 4H-pryanyl. Corresponding partially saturated or fully saturated radicals include 2—, 3-, or 4-piperidinyl, 2—, 3-, or 4-tetrahydropyranyl and the like. Representative unsaturated 6-membered heterocyclic radicals having two heteroatoms include 3- or 4-pyridazinyl, 2—, 4-, or 5-pyrimidinyl, 2-pyrazinyl, morpholino, and the like. The corresponding fully saturated and partially saturated radicals are also included, e.g. 2-piperazine. The heterocyclic radical is bonded through an available carbon atom or heteroatom in the heterocyclic ring directly to the entity or through a linker such as an alkylene such as methylene or ethylene.

The term ‘substituted’ means that one or more hydrogen atoms (attached to a carbon or heteroatom) is replaced with a selection from the indicated group of substituents, provided that the designated atom's normal valency under the existing circumstances is not exceeded. The group may be optionally substituted with particular substituents at positions that do not significantly interfere with the preparation of compounds falling within the scope of this disclosure and on the understanding that the substitution(s) does not significantly adversely affect the biological activity or structural stability of the compound. Combinations of substituents are permissible only if such combinations result in stable compounds. By ‘stable compound’ or ‘stable structure’, it is meant a compound that is sufficiently robust to survive isolation to a useful degree of purity from a reaction mixture and/or formulation into an efficacious therapeutic agent. The term ‘optionally substituted’ or ‘optional substituents’ as used herein means that the groups in question are either unsubstituted or substituted with one or more of the substituents specified. When the groups in question are substituted with more than one substituent, the substituents may be the same or different. Furthermore, the terms ‘independently’, ‘independently are’, and ‘independently selected from’ mean that the substituents in question may be the same or different.

The term ‘deuterium’ as used herein refers to an isotope of hydrogen that has one proton and one neutron in its nucleus and that has twice the mass of ordinary hydrogen. Deuterium herein is represented by the symbol ‘D’. The term ‘deuterated’ by itself or used to modify a compound or group as used herein refers to the presence of at least one deuterium atom attached to carbon. For example, the term ‘deuterated compound’ refers to a compound which contains one or more carbon-bound deuterium(s). In a deuterated compound of the present disclosure, when a particular position is designated as having deuterium, it is understood that the abundance of deuterium at that position is substantially greater than the natural abundance of deuterium, which is about 0.015%. The term ‘undeuterated’ or ‘non-deuterated’ as used herein refers to the ratio of deuterium atoms of which is not more than the natural isotopic deuterium content, which is about 0.015%; in other words, all hydrogens are present at their natural isotopic percentages. Unless otherwise stated, when a position is designated specifically as ‘H’ or ‘hydrogen’, the position is understood to have hydrogen at its natural abundance isotopic composition.

The term ‘isotopic enrichment factor’ as used herein refers to the ratio between the isotope abundance and the natural abundance of a specified isotope.

The term ‘isotopologue’ as used herein refers to a species in which the chemical structure differs from a specific compound of the disclosure only in the isotopic composition thereof.

The term ‘substantially free of other stereoisomers’ as used herein means less than 10% of other stereoisomers, preferably less than 5% of other stereoisomers, more preferably less than 2% of other stereoisomers and most preferably less than 1% of other stereoisomers are present.

The term ‘pharmaceutically acceptable salt’ as used herein refers to a salt that is not biologically or otherwise undesirable (e.g., not toxic or otherwise harmful). A salt of a compound of the disclosure is formed between an acid and a basic group of the compound, or a base and an acidic group of the compound. For example, when the compounds of the disclosure contain at least one basic group (i.e., groups that can be protonated), the disclosure includes the compounds in the form of their acid addition salts with organic or inorganic acids such as, for example, but not limited to salts with hydrogen chloride, hydrogen bromide, phosphoric acid, sulfuric acid, nitric acid, benzenesulfonic acid, acetic acid, citric acid, glutamic acid, lactic acid, and methanesulfonic acid. When compounds of the disclosure contain one or more acidic groups (e.g., a carboxylic acid), the disclosure includes the pharmaceutically acceptable salts of the compounds formed with but not limited to alkali metal salts, alkaline earth metal salts or ammonium salts. Examples of such salts include, but are not limited to, sodium salts, potassium salts, calcium salts, magnesium salts or salts with ammonia or organic amines such as, for example, ethylamine, ethanolamine, triethanolamine or amino acids. Additional examples of such salts can be, found in Stahl, P. H. et al. Pharmaceutical Salts: Properties, Selection, and Use, 2nd Revised Edition, Wiley, 2011.

The terms ‘treatment’, ‘treating’ and ‘treat’ as used herein, include their generally accepted meanings, i.e., the management and care of a patient for the purpose of preventing, reducing the risk in incurring or developing a given condition or disease, prohibiting, restraining, alleviating, ameliorating, slowing, stopping, delaying, or reversing the progression or severity, and holding in check existing characteristics of a disease, disorder, or pathological condition, including the alleviation or relief of symptoms or complications, or the cure or elimination of the disease, disorder, or condition.

The term ‘therapeutically effective amount’ as used herein refers to that amount of compound of the disclosure that will elicit the biological or medical response of a tissue, system, animal, or human that is being sought by a researcher, veterinarian, medical doctor or other. As will be recognized by a person of ordinary skill in the art, a therapeutically effective amount of the compounds of the disclosure will vary and will depend on the disease treated, the severity of the disease, the route of administration, and the gender, age, and general health condition of the subject to whom the compound is being administered. The therapeutically effective amount may be administered as a single dose once a day, or as split doses administered multiple (e.g., two, three or four) times a day. The therapeutically effective amount may also be administered through continuous dosing, such as through infusion or with an implant.

Unless defined otherwise, ‘room temperature’ is intended to mean a temperature of from about 18 to 28° C., typically between about 18 and 25° C., and more typically between about 18 and 22° C. As used herein, the phrase ‘room temperature’ may be shortened to ‘rt’ or ‘RT’.

### Compounds:

In first aspects and embodiments, the present disclosure is directed to a compound, or a pharmaceutically acceptable salt thereof, represented by Formula 1:

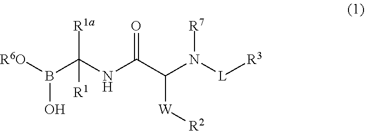

or a pharmaceutically acceptable salt, solvate, stereoisomer or mixture of stereoisomers, tautomer, isotopic form, pharmaceutically active metabolite thereof, or combinations thereof, wherein:
R¹ is selected from the group consisting of: deuterium, C1-C4 alkyl, C1-C4 alkoxyl, C₁-C₄ oxoalkyl, C1-C5 alkyl-alkoxyl, wherein each alkyl, oxoalkyl or alkoxyl is optionally substituted with C3-C6 cycloalkyl, phenyl, phenoxy, or a 5- or 6-membered heteroaryl, wherein said phenyl, phenoxy, or heteroaryl are each optionally substituted with one or more substituent selected from deuterium, halogen, hydroxyl, CN, CO2H, CO2R¹², CONR¹²R¹³, NR¹²R¹³, SR¹², SO2NR¹²R¹³, C1-C4 alkyl, C1-C4 haloalkyl, C1-C4 alkoxy, phenyl, or a 5- or 6-membered heteroaryl;
R^{1a} is selected from hydrogen, deuterium or C1-C2 alkyl;
W is C1-C4 alkylene, optionally substituted with one or more of deuterium, halogen, hydroxyl, oxo, CN, methyl or ethyl;
R² is selected from the group consisting of: hydrogen, deuterium, R⁴, —OR⁴, —C(O)R⁴, —S—R⁴, —SO—R⁴, —SO₂—R⁴, —SO₂—NR⁵R¹¹,
L is C(O), C(O)O, C(O)NR⁸, S(O)₂, or a bond;
R³ is C₁-C₄ alkyl optionally substituted with one or more substituents each independently selected from the group consisting of deuterium, halogen, cyano, hydroxyl, C₁-C₄ alkoxyl, C₁-C₄ alkylthiol or phenyl; or
R³ is saturated or unsaturated cycloalkyl or saturated or unsaturated heterocycloalkyl having one or more heteroatoms selected from N, O and S, wherein the cycloalkyl or heterocycloalkyl is optionally substituted with one or more substituents selected from deuterium, halogen, cyano, hydroxyl, oxo, C₁-C₄ alkoxyl, or C₁-C₄ alkyl that is optionally substituted with one to three substituents selected from deuterium, halogen, cyano, hydroxyl, C₁-C₄ alkylthiol or C₁-C₄ alkoxyl; or
R³ is aryl or heteroaryl having one or more heteroatoms selected from N, O and S, wherein aryl or heteroaryl is optionally substituted with one or more substituents selected from deuterium, halogen, cyano, hydroxyl, OR, CO2H, CO2R¹², CONR¹²R¹³, NR¹²R¹³, SR¹², SO2NR¹²R¹³, C₁-C₄ alkoxyl, or C₁-C₄ alkyl that is optionally substituted with one to three substituents selected from deuterium, halogen, cyano, hydroxyl, or C₁-C₄ alkoxyl;
R⁴ is hydrogen, deuterium, C₁-C₄ alkyl or C₁-C₄ alkoxyl optionally substituted with one or more substituents each independently selected from the group consisting of deuterium, halogen, cyano, hydroxyl, C₁-C₄ alkoxyl, C₁-C₄ alkylthiol, C₁-C₅ alkyl-alkoxyl or phenyl; or
R⁴ is saturated or unsaturated cycloalkyl or saturated or unsaturated heterocycloalkyl having one or more heteroatoms selected from N, O and S, wherein the cycloalkyl or heterocycloalkyl is optionally substituted with one or more substituents selected from deuterium, halogen, cyano, hydroxyl, C₁-C₄ alkoxyl, or C₁-C₄ alkyl that is optionally substituted with one to three substituents selected from deuterium, halogen, cyano, hydroxyl, or C₁-C₄ alkoxyl, C₁-C₄ alkylthiol and C₁-C₅ alkyl-alkoxyl; or
R⁴ is aryl or heteroaryl having one or more heteroatoms selected from N, O and S, wherein aryl, or heteroaryl is optionally substituted with one or more substituents selected from deuterium, halogen, cyano, hydroxyl, C₁-C₄ alkoxyl, or C₁-C₄ alkyl that is optionally substituted with one to three substituents selected from deuterium, halogen, cyano, hydroxyl, or C₁-C₄ alkoxyl, C₁-C₄ alkylthiol and C₁-C₅ alkyl-alkoxyl; or
R⁴ is NR⁹R¹⁰;
R⁵ and R¹¹ are each independently selected from hydrogen, deuterium or C1-C5 alkyl optionally substituted with one to three halogen; or
R⁵ and R¹¹ together with the N to which they are attached form a 5 or 6 membered saturated or unsaturated heterocyclic ring optionally having one or more additional heteroatoms selected from N, O and S, wherein the 5 or 6 membered heterocyclic ring is optionally substituted with one or more substituent selected from deuterium, halogen, hydroxyl, CN, C1-C4 alkyl, C1-C4 haloalkyl or C1-C4 alkoxy;
R⁶ is hydrogen, or R⁶ and R¹, together with the boron atom to which —OR⁶ is attached form a 5-membered heteroalkyl ring.
R⁷ is selected from hydrogen, deuterium, or C₁-C₄ alkyl optionally substituted with one or more substituents each independently selected from the group consisting of deuterium, halogen, hydroxyl, cyano, methoxyl, methylthiol and phenyl;
R⁸ is hydrogen or C₁-C₄ alkyl optionally substituted with one or more of deuterium, halogen, hydroxyl and phenyl, wherein phenyl is optionally substituted with one or more substituent selected from deuterium, halogen, hydroxyl and C₁-C₂ alkyl;
R⁹ and R¹⁰ are each independently selected from hydrogen, deuterium or C₁-C₆ alkyl that is optionally substituted with one to three substituents selected from deuterium, halogen, cyano, or C₁-C₄ alkoxyl; or
R⁹ and R¹⁰ together with the N to which they are attached form a 5 or 6 membered saturated or unsaturated heterocyclic ring optionally having one or more additional heteroatoms selected from N, O and S, wherein the 5 or 6 membered heterocyclic ring is optionally substituted with one or more substituent selected from deuterium, halogen, hydroxyl, CN, C1-C4 alkyl, C1-C4 haloalkyl or C1-C4 alkoxy; and
R¹² and R¹³ are each independently selected from hydrogen, deuterium, C1-C4 alkyl; C1-C4 haloalkyl, C1-C5 alkyl-alkoxyl, C3-C7 cycloalkyl, or R¹² and R¹³ together with the N to which they are attached form 3 to 7 membered heterocyclic ring optionally having one or more additional heteroatoms selected from N, O and S, wherein the C3-C7 cycloalkyl or 3 to 7 membered heterocyclic ring is optionally substituted with one or more substituent selected from deuterium, halogen, hydroxyl, oxo, CN, C1-C4 alkyl, C1-C4 haloalkyl or C1-C4 alkoxyl.

In any embodiments of this first aspect, R² may particularly be selected from the group consisting of: R⁴, —OR⁴, —C(O)R⁴, —S—R⁴, —SO—R⁴, —SO₂—R⁴ or —SO₂—NR⁵R¹¹. In embodiments, R² is selected from R⁴, OR⁴, C(O)R⁴, SO—R⁴, SO₂—R⁴ or SO₂—NR⁵R¹¹

In any embodiments of this first aspect, R¹ may be selected from the group consisting of: C1-C4 alkyl, C1-C5 alkoxyl, C1-C5 alkyl-alkoxyl, each optionally substituted with C3-C6 cycloalkyl, phenyl, phenoxyl, or a 5- or 6-membered heteroaryl, wherein said phenyl, phenoxyl, or heteroaryl are each optionally substituted with one or more substituent selected from deuterium, halogen, hydroxyl, CN, C1-C4 alkyl, C1-C4 haloalkyl, C1-C4 alkoxy, phenyl, or a 5- or 6-membered heteroaryl.

In embodiments, R¹ is selected from C1-C4 alkyl, C1-C4 alkoxyl, C1-C4 alkyl-alkoxyl, each optionally substituted with phenyl or a 5- or 6-membered heteroaryl.

In embodiments, R¹ is deuterium. In embodiments, R¹ is C₁-C₄ alkyl. In embodiments, R¹ is C₁-C₄ oxoalkyl. In embodiments, R¹ is C₁-C₅ alkoxyl. In embodiments, R¹ is C₁-C₅ alkyl-alkoxyl. In any such embodiments, each alkyl, oxoalkyl or alkoxyl may be optionally substituted. In particular, the substituents may be selected from C3-C6 cycloalkyl, phenyl, phenoxy, or a 5- or 6-membered heteroaryl. In such embodiments, said phenyl, phenoxy, or heteroaryl may each be substituted with one or more substituent selected from deuterium, halogen, hydroxyl, CN, C₁-C₄ alkyl, C₁-C₄ haloalkyl or C₁-C₄ alkoxyl, phenyl, or a 5- or 6-membered heteroaryl.

In embodiments, R¹ is CO2H. In embodiments, R¹ is CO2R¹². In embodiments, R¹ is CONR¹²R¹³. In embodiments, R¹ is NR¹²R¹³. In embodiments, R¹ is SR¹². In embodiments, R¹ is SO2NR¹²R¹³.

In embodiments, R^{1a} is hydrogen. In embodiments, R^{1a} is deuterium. In embodiments, R^{1a} is C1-C2 alkyl. In embodiments, R^{1a} is hydrogen or methyl.

In embodiments, W is C1-C4 alkylene. In embodiments, W is substituted C₁-C₄ alkylene. In embodiments, suitable substituents may be selected from one or more (for example, 1, 2 or 3) substituent selected from any one or more of deuterium, halogen, hydroxyl, oxo, CN, methyl or ethyl. In embodiments, W is C1-C4 alkylene optionally substituted with one or more of hydroxyl or oxo. In embodiments, W is methylene. In embodiments, W is ethylene. In embodiments, W is propylene. In embodiments, W is butylene. Said propylene or butylene may be straight or branched.

In embodiments, R² is hydrogen. In embodiments, R² is deuterium.

In embodiments, R² is R⁴. In embodiments, R² is —OR⁴. In embodiments, R² is —C(O)R⁴. In embodiments, R² is —S—R⁴. In embodiments, R² is —SO—R⁴. In embodiments, R² is —SO₂—R⁴. In embodiments, R² is —SO₂—NR⁵R¹¹.

In embodiments, R⁵ and R¹¹ are each independently selected from hydrogen, deuterium or C1-C5 alkyl optionally substituted with one to three halogen. In other embodiments, R⁵ and R¹¹ together with the N to which they are attached form a 5 or 6 membered saturated or unsaturated heterocyclic ring optionally having one or more additional heteroatoms selected from N, O and S, wherein the 5 or 6 membered heterocyclic ring is optionally substituted with one or more substituent selected from deuterium, halogen, hydroxyl, CN, C1-C4 alkyl, C1-C4 haloalkyl or C1-C4 alkoxyl.

In embodiments, R⁵ and R¹¹ are each independently selected from hydrogen, or C₁-C₄ alkyl optionally substituted with one to three fluoro.

In embodiments, L is C(O). In embodiments, L is C(O)O. In embodiments, L is C(O)NR⁸. In embodiments, L is S(O)₂. In embodiments, L is a bond.

In embodiments, R³ is C₁-C₄ alkyl. In such embodiments, C₁-C₄ alkyl may be substituted with one or more substituents. Said substituents may be selected independently from one or more of the group consisting of deuterium, halogen, cyano, hydroxyl, C₁-C₄ alkoxyl, C₁-C₄ alkylthiol or phenyl.

In other embodiments, R³ is saturated or unsaturated cycloalkyl. In other embodiments, R³ is saturated or unsaturated heterocycloalkyl having one or more heteroatoms selected from N, O and S. In any such embodiments, the cycloalkyl or heterocycloalkyl may be substituted; for example, with one or more substituents selected independently from one or more of deuterium, halogen, cyano, hydroxyl, oxo, C₁-C₄ alkoxyl, or C₁-C₄ alkyl. In such embodiments, C₁-C₄ alkoxyl, and C₁-C₄ alkyl may be optionally substituted with one to three substituents; for example, selected independently from one or more of deuterium, halogen, cyano, hydroxyl, I, C₁-C₄ alkylthiol or C₁-C₄ alkoxyl.

In other embodiments, R³ is aryl. In other embodiments, R³ is heteroaryl having one or more heteroatoms selected from N, O and S. In any such embodiments, the aryl or heteroaryl may be substituted with one or more substituents; for example, said substituents may be selected independently from one or more of deuterium, halogen, cyano, hydroxyl, C₁-C₄ alkoxyl, or C₁-C₄ alkyl. In such embodiments, C₁-C₄ alkoxyl, and C₁-C₄ alkyl may be optionally substituted with one to three substituents; for example, selected independently from one or more of deuterium, halogen, cyano, hydroxyl, I, C₁-C₄ alkylthiol or C₁-C₄ alkoxyl.

In embodiments, R³ is CO2H. In embodiments, R³ is CO2R¹². In embodiments, R³ is CONR¹²R¹³. In embodiments, R³ is NR¹²R¹³. In embodiments, R³ is SR¹². In embodiments, R³ is SO2NR¹²R¹³.

In embodiments, R¹² and R¹³ are each independently selected from hydrogen, deuterium, C1-C4 alkyl; C1-C4 haloalkyl or C1-C5 alkyl-alkoxyl. In embodiments, R¹² and R¹³ are each independently selected from hydrogen, deuterium, C1-C4 alkyl; C1-C4 haloalkyl, C1-C5 alkyl-alkoxyl or C3-C7 cycloalkyl. In embodiments, R¹² and R¹³ are each independently selected from hydrogen, deuterium, C1-C2 alkyl; C1-C2 haloalkyl, C1-C2 alkyl-alkoxyl or C3-C5 cycloalkyl. In embodiments, R¹² and R¹³ together with the N to which they are attached form 3 to 7 membered heterocyclic ring optionally having one or more additional heteroatoms selected from N, O and S. In any such embodiments, the C3-C7 cycloalkyl, C₃-C₅ cycloalkyl or 3 to 7 membered heterocyclic ring is optionally substituted with one or more substituent selected from deuterium, halogen, hydroxyl, oxo, CN, C1-C4 alkyl, C1-C4 haloalkyl or C1-C4 alkoxyl. In embodiments, the substituents may be selected from one, two or three of deuterium, F, Cl, hydroxyl, oxo, CN, C1-C2 alkyl, C1-C2 haloalkyl or C1-C2 alkoxyl.

In embodiments, R⁴ is C₁-C₄ alkyl, an amino optionally substituted with one or more C₁-C₄ alkyl, an optionally substituted 5- or 6-membered heterocyclic ring having one or more heteroatoms, an optionally substituted 5- or 6-membered heteroaromatic ring having one or more heteroatoms, or an optionally substituted 5- or 6-membered aromatic ring.

In embodiments, R⁴ is hydrogen. In embodiments, R⁴ is deuterium. In embodiments, R⁴ is C₁-C₄ alkyl. In embodiments, R⁴ is C₁-C₄ alkoxyl. In such embodiments, C₁-C₄ alkyl and C₁-C₄ alkoxyl may be substituted with one or more substituents. Said substituents may be independently selected, for example, from the group consisting of deuterium, halogen, cyano, hydroxyl, C₁-C₄ alkoxyl, C₁-C₄ alkylthiol, C₁-C₅ alkyl-alkoxyl or phenyl.

In other embodiments, R⁴ is saturated or unsaturated cycloalkyl. In other embodiments, R⁴ is saturated or unsaturated heterocycloalkyl having one or more heteroatoms selected from N, O and S. In any such embodiments, the cycloalkyl or heterocycloalkyl group may be substituted with one or more substituents. Such substituents may be independently selected from one or more of deuterium, halogen, cyano, hydroxyl, C₁-C₄ alkoxyl, or C₁-C₄ alkyl. Said C₁-C₄ alkoxyl, or C₁-C₄ alkyl may also be substituted with one to three substituents; for example, independently selected from one or more of deuterium, halogen, cyano, hydroxyl, or C₁-C₄ alkoxyl, C₁-C₄ alkylthiol and C₁-C₅ alkyl-alkoxyl.

In other embodiments, R⁴ is aryl. In other embodiments, R⁴ is heteroaryl having one or more heteroatoms selected from N, O and S. In any such embodiments, the aryl, or heteroaryl may be substituted with one or more substituents; for example, independently selected from one or more of deuterium, halogen, cyano, hydroxyl, C₁-C₄ alkoxyl, or C₁-C₄ alkyl. In such embodiments, C₁-C₄ alkoxyl, or C₁-C₄ alkyl may also be substituted with one to three substituents; for example, independently selected from one or more of deuterium, halogen, cyano, hydroxyl, or C₁-C₄ alkoxyl, C₁-C₄ alkylthiol and C₁-C₅ alkyl-alkoxyl.

In other embodiments, R⁴ is NR⁹R¹⁰.

In embodiments, R⁹ is selected from hydrogen, deuterium or C₁-C₆ alkyl. In embodiments, R¹⁰ is selected from hydrogen, deuterium or C₁-C₆ alkyl. In such embodiments, C₁-C₆ alkyl may be substituted with one to three substituents selected from deuterium, halogen, cyano, or C₁-C₄ alkoxyl.

In embodiments, R⁹ and R¹⁰ together with the N to which they are attached may form a 5 or 6 membered saturated or unsaturated heterocyclic ring. The heterocyclic ring may have one or more additional heteroatom selected from N, O and S. In embodiments, the 5 or 6 membered heterocyclic ring may be substituted with one or more substituent selected from deuterium, halogen, hydroxyl, CN, C1-C4 alkyl, C1-C4 haloalkyl or C1-C4 alkoxyl.

In embodiments, R⁹ and R¹⁰ are each independently selected from hydrogen or C₁-C₄ alkyl that is optionally substituted with one to three substituents selected from halogen, cyano, or C₁-C₂ alkoxyl. In embodiments, R⁹ and R¹⁰ are each independently selected from hydrogen or C₁-C₂ alkyl that is optionally substituted with one to three substituents selected from fluoro or chloro. In embodiments, R⁹ and R¹⁰ are each methyl.

In embodiments, R⁶ is hydrogen. In other embodiments, R⁶ and R¹, together with the boron atom to which —OR⁶ is attached form a 5-membered heteroalkyl ring.

In embodiments, R⁷ is selected from hydrogen, F, Cl, hydroxyl, methyl, ethyl, CF₃, OMe, phenyl-(CH₂)— or phenyl-(CH₂)₂—.

In embodiments, R⁷ is hydrogen. In embodiments, R⁷ is deuterium. In embodiments, R⁷ is C₁-C₄ alkyl optionally substituted with one or more substituents. The substituents may be selected independently from the group consisting of deuterium, halogen, hydroxyl, cyano, methoxyl, methylthiol and phenyl.

In embodiments, R⁸ is selected from hydrogen, or C₁-C₄ alkyl optionally substituted with one to three fluoro.

In embodiments, R⁸ is hydrogen. In other embodiments, R⁸ is C₁-C₄ alkyl, C₁-C₄ alkyl may be substituted with one or more of deuterium, halogen, hydroxyl and phenyl. In embodiments, phenyl may be optionally substituted with one or more substituent selected from deuterium, halogen, hydroxyl and C₁-C₂ alkyl.

In second aspects and embodiments, the present disclosure is directed to a compound, or a pharmaceutically acceptable salt thereof, represented by Formula 2:

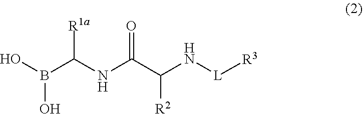

wherein:
R¹ is C₁-C₄ alkyl, C₁-C₅ alkoxy, C₁-C₄ alkyl alkoxy, each optionally substituted with phenyl or heteroaryl;
R² is (C₁-C₄ alkyl)-C(O)R⁴, (C₁-C₄ alkyl)-OR⁴, (C₁-C₄ alkyl)-R⁴, (C₁-C₄ alkyl)-SO₂—NR⁵R⁶, (C₁-C₄ alkyl)-SO₂—R⁴, (C₁-C₄ alkyl)-SO—R⁴;
L is C(O), C(O)O, C(O)NR⁵, S(O)₂, or a bond;
R³ is C₁-C₄ alkyl optionally substituted with one or more substituents each independently selected from the groups consisting of fluoro, chloro, cyano, or methoxyl, cycloalkyl, heterocyclyl having one or more heteroatoms, aryl, cycloaryl, or heteroaryl having one or more heteroatoms, any of which is optionally substituted with one or more fluoro, chloro, cyano, methoxyl, or C₁-C₄ alkyl that is optionally substituted with one to three fluoro, chloro, cyano, or methoxyl;
R⁴ is C₁-C₄ alkyl, an amino optionally substituted with one or more C₁-C₄ alkyl, an optionally substituted 5- or 6-membered heterocyclic ring having one or more heteroatoms, an optionally substituted 5- or 6-membered heteroaromatic ring having one or more heteroatoms, or an optionally substituted 5- or 6-membered aromatic ring; and
R⁵ and R⁶ are each independently hydrogen, or C₁-C₄ alkyl optionally substituted with one to three fluoro.

The following exemplary embodiments are intended to form part of the first and/or the second aspects disclosed herein, and are intended to be combined in any suitable combination, unless otherwise stated.

In certain embodiments, R¹ is methyl, ethyl, n-propyl, i-propyl, n-butyl, tert-butyl, or n-butyl, each optionally substituted with a phenyl ring. In certain embodiment, R¹ is methyl. In certain embodiment, R¹ is methyl substituted with a phenyl ring. In certain embodiments, R¹ is ethyl. In certain embodiments, R¹ is ethyl substituted with a phenyl ring. In certain embodiments, R¹ is n-propyl. In certain embodiments, R¹ is n-propyl substituted with a phenyl ring. In certain embodiments, R¹ is tert-butyl. In certain embodiments, R¹ is n-butyl. In certain embodiments, R¹ is methoxymethyl optionally substituted with a phenyl ring.

In certain embodiments, R² is CH₂—R⁴, CH₂—CH₂—R⁴, CH₂—OR⁴, CH₂CH₂—OR⁴, CH₂—C(O)R⁴, CH₂—CH₂—C(O)R⁴, CH₂—SO₂NR⁵R⁶, CH₂SO₂R⁵, CH₂CH₂SO₂R⁵, CH₂S(O)R⁵. In certain embodiments, R² is CH₂—R⁴. In certain embodiments, R² is CH₂—CH₂—R⁴. In certain embodiments, R² is CH₂—OR⁴. In certain embodiments, R² is CH₂CH₂—OR⁴. In certain embodiments, R² is CH₂—C(O)R⁴. In certain embodiments, R² is CH₂—CH₂—C(O)R⁴. In certain embodiments, R² is CH₂—SO₂NR⁵R⁶. In certain embodiments, R² is CH₂SO₂R⁵. In certain embodiments, R² is CH₂S(O)R⁵.

In certain embodiments, L is a bond, C(O), C(O)O, C(O)NR⁵, or SO₂. In certain embodiments, L is a bond. In certain embodiments, L is C(O). In certain embodiments, L is C(O)O. In certain embodiments, L is C(O)NR⁵. In certain embodiments, L is SO₂.

In certain embodiments, R³ is C₁-C₄ alkyl optionally substituted with one or more substituents each independently selected from the groups consisting of fluoro, chloro, cyano, or methoxyl, cycloalkyl, heterocyclyl, aryl, cycloaryl, or heteroaryl, any of which is optionally substituted one or more fluoro, chloro, cyano, methoxyl, or C₁-C₄ alkyl that is optionally substituted with one to three fluoro, chloro, cyano, or methoxyl. In certain embodiments, R³ is C₁-C₄ alkyl optionally substituted with one or more substituents each independently selected from the groups consisting of fluoro, chloro, cyano, or methoxyl. In certain embodiments, R³ is cycloalkyl which is optionally substituted one or more fluoro, chloro, cyano, methoxyl, or C₁-C₄ alkyl that is optionally substituted with one to three fluoro, chloro, cyano, or methoxyl. In certain embodiments, R³ is heterocyclyl which is optionally substituted one or more fluoro, chloro, cyano, methoxyl, or C₁-C₄ alkyl that is optionally substituted with one to three fluoro, chloro, cyano, or methoxyl. In certain embodiments, R³ is aryl which is optionally substituted one or more fluoro, chloro, cyano, methoxyl, or C₁-C₄ alkyl that is optionally substituted with one to three fluoro, chloro, cyano, or methoxyl. In certain embodiments, R³ is cycloaryl which is optionally substituted one or more fluoro, chloro, cyano, methoxyl, or C₁-C₄ alkyl that is optionally substituted with one to three fluoro, chloro, cyano, or methoxyl. In certain embodiments, R³ is heteroaryl which is optionally substituted one or more fluoro, chloro, cyano, methoxyl, or C₁-C₄ alkyl that is optionally substituted with one to three fluoro, chloro, cyano, or methoxyl.

In certain embodiments, R³ is methyl, tert-butyl, trifluoromethyl, phenyl optionally substituted one or more fluoro, chloro, cyano, methoxyl, or C₁-C₄ alkyl that is optionally substituted with one to three fluoro, chloro, cyano, or methoxyl; pyridinyl optionally substituted one or more fluoro, chloro, cyano, methoxyl, or C₁-C₄ alkyl that is optionally substituted with one to three fluoro, chloro, cyano, or methoxyl; piperidinyl optionally substituted one or more fluoro, chloro, cyano, methoxyl, or C₁-C₄ alkyl that is optionally substituted with one to three fluoro, chloro, cyano, or methoxyl; pyrrolidinyl optionally substituted one or more fluoro, chloro, cyano, methoxyl, or C₁-C₄ alkyl that is optionally substituted with one to three fluoro, chloro, cyano, or methoxyl; imidazolyl optionally substituted one or more fluoro, chloro, cyano, methoxyl, or C₁-C₄ alkyl that is optionally substituted with one to three fluoro, chloro, cyano, or methoxyl; pyrazolyl optionally substituted one or more fluoro, chloro, cyano, methoxyl, or C₁-C₄ alkyl that is optionally substituted with one to three fluoro, chloro, cyano, or methoxyl; thiazolyl optionally substituted one or more fluoro, chloro, cyano, methoxyl, or C₁-C₄ alkyl that is optionally substituted with one to three fluoro, chloro, cyano, or methoxyl; pyrazinyl optionally substituted one or more fluoro, chloro, cyano, methoxyl, or C₁-C₄ alkyl that is optionally substituted with one to three fluoro, chloro, cyano, or methoxyl; oxazolyl optionally substituted one or more fluoro, chloro, cyano, methoxyl, or C₁-C₄ alkyl that is optionally substituted with one to three fluoro, chloro, cyano, or methoxyl; or morpholinyl optionally substituted one or more fluoro, chloro, cyano, methoxyl, or C₁-C₄ alkyl that is optionally substituted with one to three fluoro, chloro, cyano, or methoxyl.

In certain embodiments, R³ is methyl. In certain embodiments, R³ is tert-butyl. In certain embodiments, R³ is trifluoromethyl.

In certain embodiments, R³ is phenyl optionally substituted one or more fluoro, chloro, cyano, methoxyl, or C₁-C₄ alkyl that is optionally substituted with one to three fluoro, chloro, cyano, or methoxyl. In certain embodiments, R³ is pyridinyl optionally substituted one or more fluoro, chloro, cyano, methoxyl, or C₁-C₄ alkyl that is optionally substituted with one to three fluoro, chloro, cyano, or methoxyl. In certain embodiments, R³ is piperidinyl optionally substituted one or more fluoro, chloro, cyano, methoxyl, or C₁-C₄ alkyl that is optionally substituted with one to three fluoro, chloro, cyano, or methoxyl. In certain embodiments, R³ is pyrrolidinyl optionally substituted one or more fluoro, chloro, cyano, methoxyl, or C₁-C₄ alkyl that is optionally substituted with one to three fluoro, chloro, cyano, or methoxyl. In certain embodiments, R³ is imidazolyl optionally substituted one or more fluoro, chloro, cyano, methoxyl, or C₁-C₄ alkyl that is optionally substituted with one to three fluoro, chloro, cyano, or methoxyl. In certain embodiments, R³ is pyrazolyl optionally substituted one or more fluoro, chloro, cyano, methoxyl, or C₁-C₄ alkyl that is optionally substituted with one to three fluoro, chloro, cyano, or methoxyl. In certain embodiments, R³ is thiazolyl optionally substituted one or more fluoro, chloro, cyano, methoxyl, or C₁-C₄ alkyl that is optionally substituted with one to three fluoro, chloro, cyano, or methoxyl. In certain embodiments, R³ is pyrazinyl optionally substituted one or more fluoro, chloro, cyano, methoxyl, or C₁-C₄ alkyl that is optionally substituted with one to three fluoro, chloro, cyano, or methoxyl. In certain embodiments, R³ is oxazolyl optionally substituted one or more fluoro, chloro, cyano, methoxyl, or C₁-C₄ alkyl that is optionally substituted with one to three fluoro, chloro, cyano, or methoxyl. In certain embodiments, R³ is morpholinyl optionally substituted one or more fluoro, chloro, cyano, methoxyl, or C₁-C₄ alkyl that is optionally substituted with one to three fluoro, chloro, cyano, or methoxyl.

In certain embodiments, R⁴ is CH₃, NH₂, NH(CH₂CH₃), N(CH₃)₂, phenyl, morpholinyl, piperidinyl, pyrrolidinyl, pyrazolyl, pyridinyl, imidazolyl, oxadiazolyl optionally substituted with CH₃, or thiazolyl. In certain embodiments, R⁴ is CH₃. In certain embodiments, R⁴ is NH₂. In certain embodiments, R⁴ is NH(CH₂CH₃). In certain embodiments, R⁴ is N(CH₃)₂. In certain embodiments, R⁴ is phenyl. In certain embodiments, R⁴ is morpholinyl. In certain embodiments, R⁴ is piperidinyl. In certain embodiments, R⁴ is pyrrolidinyl. In certain embodiments, R⁴ is pyrazolyl. In certain embodiments, R⁴ is pyridinyl. In certain embodiments, R⁴ is imidazolyl. In certain embodiments, R⁴ is oxadiazolyl optionally substituted with CH₃. In certain embodiments, R⁴ is thiazolyl.

In certain embodiments, R⁵ and R⁶ are each independently hydrogen, methyl, ethyl, tert-butyl, or trifluoromethyl. In certain embodiments, R⁵ is hydrogen. In certain embodiments, R⁵ is methyl. In certain embodiments, R⁵ is ethyl. In certain embodiments, R⁵ is tert-butyl. In certain embodiments, R⁵ is trifluoromethyl.

In certain embodiments, R⁶ is hydrogen. In certain embodiments, R⁶ is methyl. In certain embodiments, R⁶ is ethyl. In certain embodiments, R⁶ is tert-butyl. In certain embodiments, R⁶ is trifluoromethyl.

In another embodiment, the present disclosure is directed to a compound, or pharmaceutically acceptable salt thereof, represented by any one of the following structures:

TABLE 1
Chemical structures of compounds according to the disclosure. Any of
these compounds may also exist in the racemic forms, particularly the
oxaborolane isomer derivative, and such isomers are explicitly intended to
be encompassed within the scope of this disclosure.

| | |
|---|---|
| 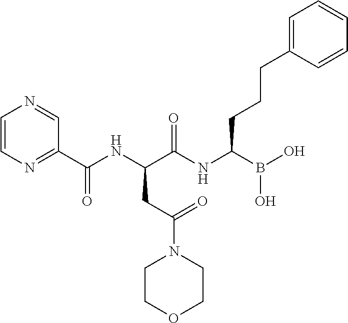 [SMILES: O=C(N[C@H](CC(=O)N1CCOCC1)C(=O)N[C@@H](CCCc1ccccc1)B(O)O)c1cnccn1] | Structure 1 |
| 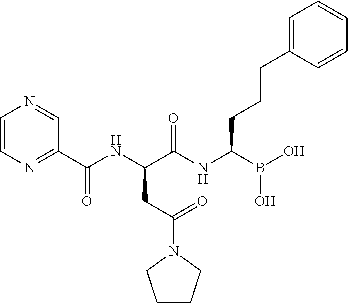 [SMILES: O=C(N[C@H](CC(=O)N1CCCC1)C(=O)N[C@@H](CCCc1ccccc1)B(O)O)c1cnccn1] | Structure 2 |
| 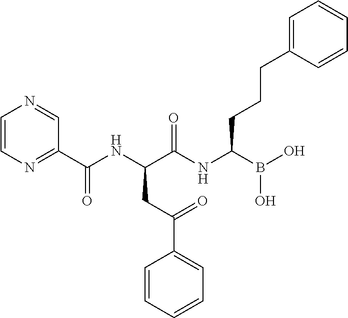 [SMILES: O=C(C[C@@H](NC(=O)c1cnccn1)C(=O)N[C@@H](CCCc1ccccc1)B(O)O)c1ccccc1] | Structure 3 |
| 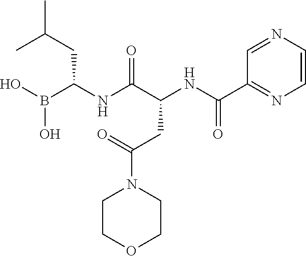 [SMILES: CC(C)C[C@H](NC(=O)[C@@H](CC(=O)N1CCOCC1)NC(=O)c1cnccn1)B(O)O] | Structure 4 |
| 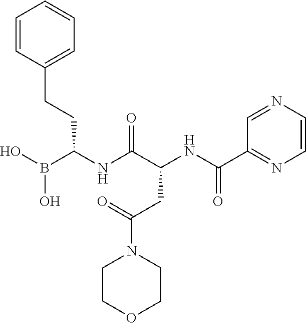 [SMILES: O=C(N[C@H](CC(=O)N1CCOCC1)C(=O)N[C@@H](CCc1ccccc1)B(O)O)c1cnccn1] | Structure 5 |
| 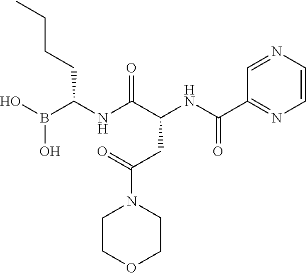 [SMILES: CCCC[C@H](NC(=O)[C@@H](CC(=O)N1CCOCC1)NC(=O)c1cnccn1)B(O)O] | Structure 6 |
| 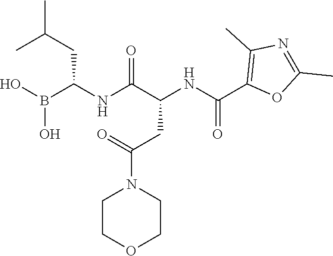 [SMILES: Cc1nc(C)c(C(=O)N[C@H](CC(=O)N2CCOCC2)C(=O)N[C@@H](CC(C)C)B(O)O)o1] | Structure 7 |
| 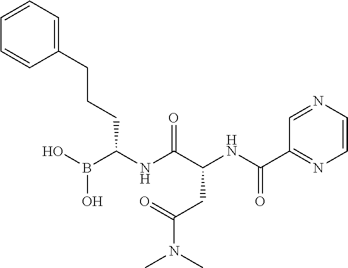 [SMILES: CN(C)C(=O)C[C@@H](NC(=O)c1cnccn1)C(=O)N[C@@H](CCCc1ccccc1)B(O)O] | Structure 8 |
| 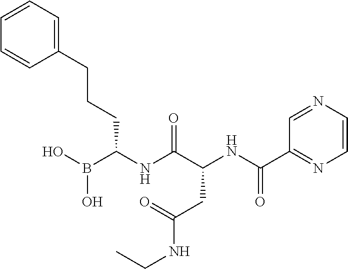 [SMILES: CCNC(=O)C[C@@H](NC(=O)c1cnccn1)C(=O)N[C@@H](CCCc1ccccc1)B(O)O] | Structure 9 |
| 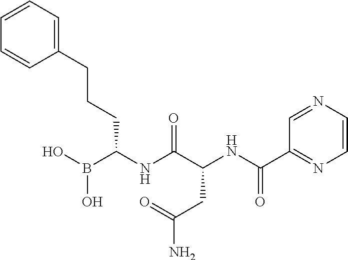 [SMILES: NC(=O)C[C@@H](NC(=O)c1cnccn1)C(=O)N[C@@H](CCCc1ccccc1)B(O)O] | Structure 10 |
| 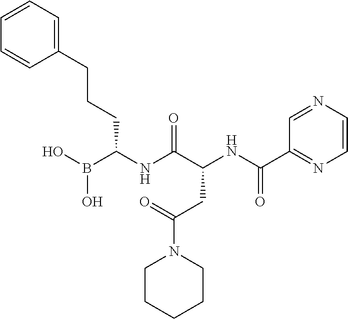 [SMILES: O=C(N[C@H](CC(=O)N1CCCCC1)C(=O)N[C@@H](CCCc1ccccc1)B(O)O)c1cnccn1] | Structure 11 |
| 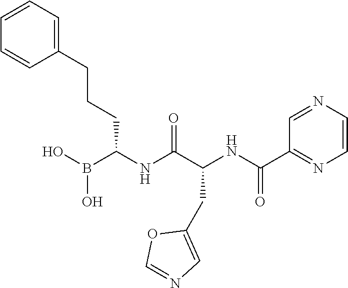 [SMILES: O=C(N[C@H](Cc1cnco1)C(=O)N[C@@H](CCCc1ccccc1)B(O)O)c1cnccn1] | Structure 12 |
| 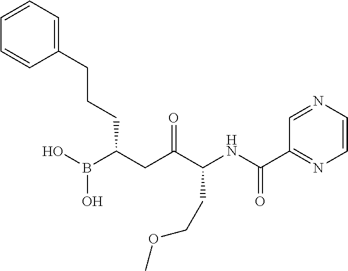 [SMILES: COCC[C@@H](NC(=O)c1cnccn1)C(=O)C[C@@H](CCCc1ccccc1)B(O)O] | Structure 13 |
| 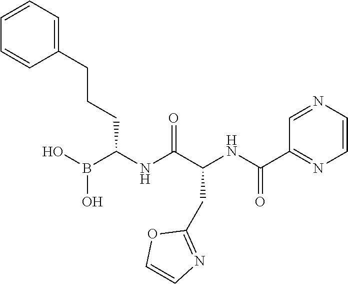 [SMILES: O=C(N[C@H](Cc1ncco1)C(=O)N[C@@H](CCCc1ccccc1)B(O)O)c1cnccn1] | Structure 14 |
| 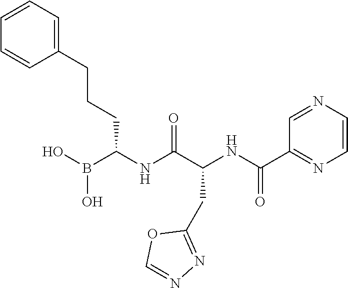 [SMILES: O=C(N[C@H](Cc1nnco1)C(=O)N[C@@H](CCCc1ccccc1)B(O)O)c1cnccn1] | Structure 15 |
| 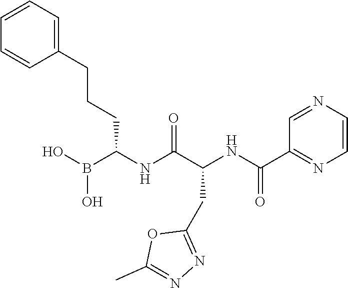 [SMILES: Cc1nnc(C[C@@H](NC(=O)c2cnccn2)C(=O)N[C@@H](CCCc2ccccc2)B(O)O)o1] | Structure 16 |
| 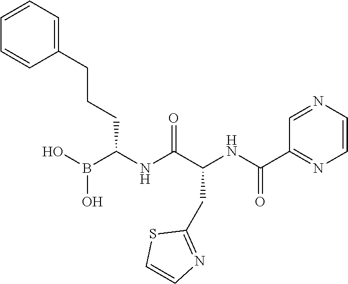 [SMILES: O=C(N[C@H](Cc1nccs1)C(=O)N[C@@H](CCCc1ccccc1)B(O)O)c1cnccn1] | Structure 17 |
| 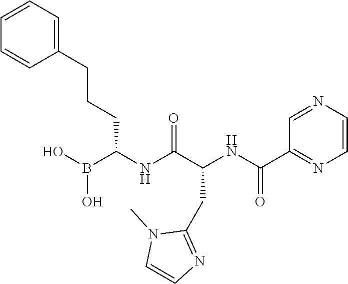 [SMILES: Cn1ccnc1C[C@@H](NC(=O)c1cnccn1)C(=O)N[C@@H](CCCc1ccccc1)B(O)O] | Structure 18 |
| 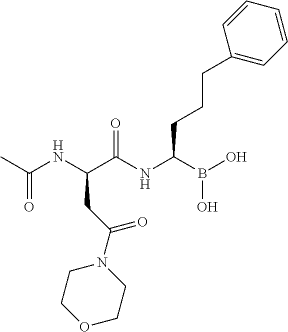 [SMILES: CC(=O)N[C@H](CC(=O)N1CCOCC1)C(=O)N[C@@H](CCCc1ccccc1)B(O)O] | Structure 19 |
| 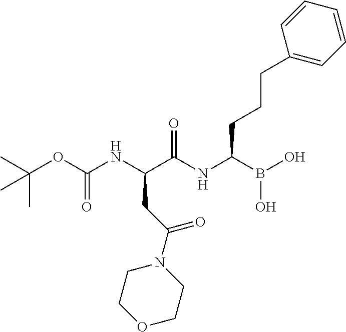 [SMILES: CC(C)(C)OC(=O)N[C@H](CC(=O)N1CCOCC1)C(=O)N[C@@H](CCCc1ccccc1)B(O)O] | Structure 20 |
| 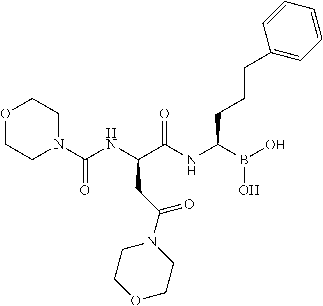 [SMILES: O=C(N[C@@H](CCCc1ccccc1)B(O)O)[C@@H](CC(=O)N1CCOCC1)NC(=O)N1CCOCC1] | Structure 21 |
| 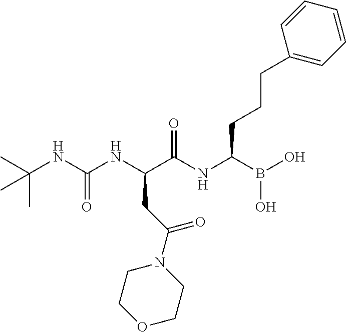 [SMILES: CC(C)(C)NC(=O)N[C@H](CC(=O)N1CCOCC1)C(=O)N[C@@H](CCCc1ccccc1)B(O)O] | Structure 22 |
| 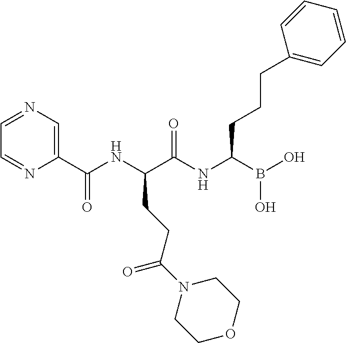 [SMILES: O=C(N[C@H](CCC(=O)N1CCOCC1)C(=O)N[C@@H](CCCc1ccccc1)B(O)O)c1cnccn1] | Structure 23 |
| 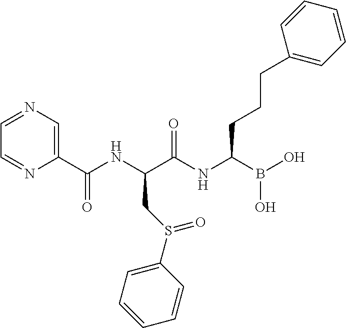 [SMILES: O=C(N[C@H](CS(=O)c1ccccc1)C(=O)N[C@@H](CCCc1ccccc1)B(O)O)c1cnccn1] | Structure 24 |
| 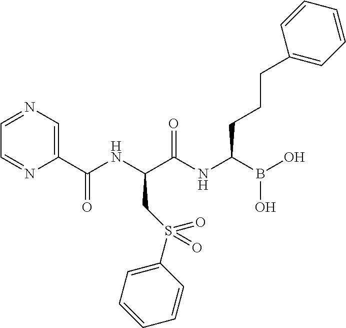 [SMILES: O=C(N[C@H](CS(=O)(=O)c1ccccc1)C(=O)N[C@@H](CCCc1ccccc1)B(O)O)c1cnccn1] | Structure 25 |
| 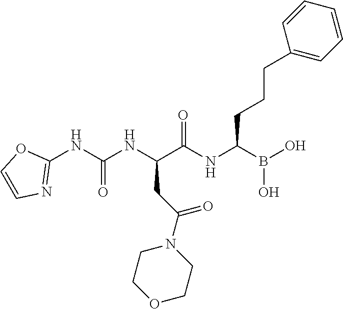 [SMILES: O=C(Nc1ncco1)N[C@H](CC(=O)N1CCOCC1)C(=O)N[C@@H](CCCc1ccccc1)B(O)O] | Structure 26 |
| 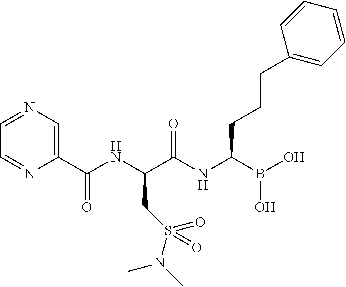 [SMILES: CN(C)S(=O)(=O)C[C@@H](NC(=O)c1cnccn1)C(=O)N[C@@H](CCCc1ccccc1)B(O)O] | Structure 27 |
| 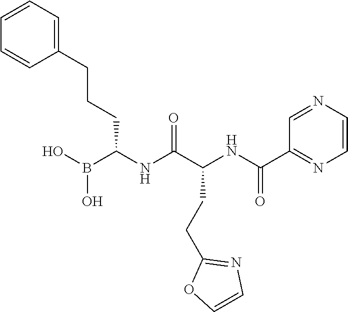 [SMILES: O=C(N[C@H](CCc1ncco1)C(=O)N[C@@H](CCCc1ccccc1)B(O)O)c1cnccn1] | Structure 28 |
| 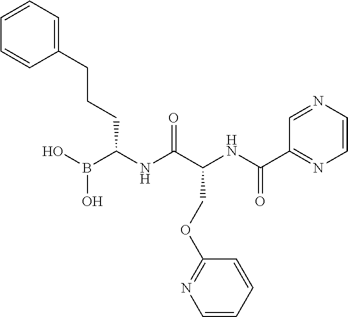 [SMILES: O=C(N[C@H](COc1ccccn1)C(=O)N[C@@H](CCCc1ccccc1)B(O)O)c1cnccn1] | Structure 29 |
| 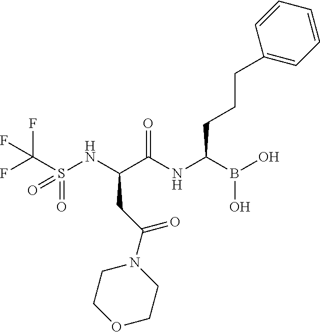 [SMILES: O=C(N[C@@H](CCCc1ccccc1)B(O)O)[C@@H](CC(=O)N1CCOCC1)NS(=O)(=O)C(F)(F)F] | Structure 30 |
| 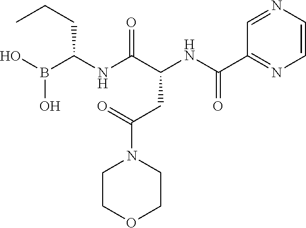 [SMILES: CCC[C@H](NC(=O)[C@@H](CC(=O)N1CCOCC1)NC(=O)c1cnccn1)B(O)O] | Structure 31 |
| 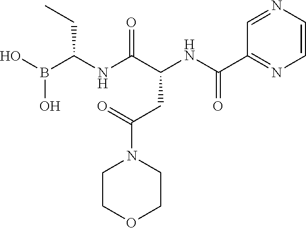 [SMILES: CC[C@H](NC(=O)[C@@H](CC(=O)N1CCOCC1)NC(=O)c1cnccn1)B(O)O] | Structure 32 |
| 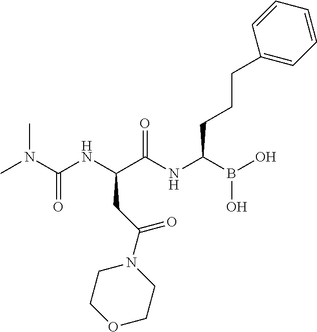 [SMILES: CN(C)C(=O)N[C@H](CC(=O)N1CCOCC1)C(=O)N[C@@H](CCCc1ccccc1)B(O)O] | Structure 33 |
| 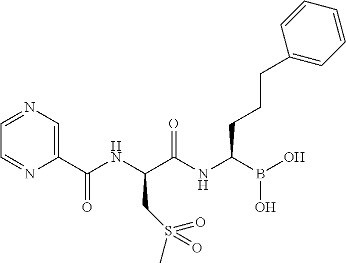 [SMILES: CS(=O)(=O)C[C@@H](NC(=O)c1cnccn1)C(=O)N[C@@H](CCCc1ccccc1)B(O)O] | Structure 34 |
| 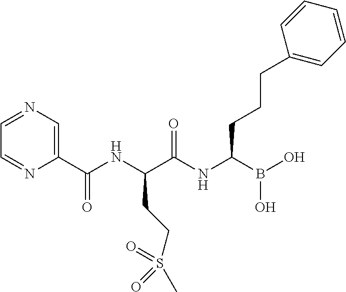 [SMILES: CS(=O)(=O)CC[C@@H](NC(=O)c1cnccn1)C(=O)N[C@@H](CCCc1ccccc1)B(O)O] | Structure 35 |
| 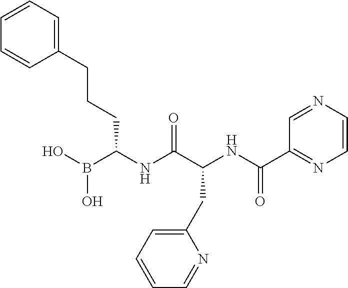 [SMILES: O=C(N[C@H](Cc1ccccn1)C(=O)N[C@@H](CCCc1ccccc1)B(O)O)c1cnccn1] | Structure 36 |
| 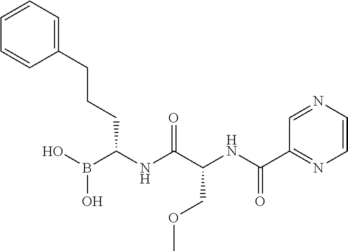 [SMILES: COC[C@@H](NC(=O)c1cnccn1)C(=O)N[C@@H](CCCc1ccccc1)B(O)O] | Structure 37 |
| 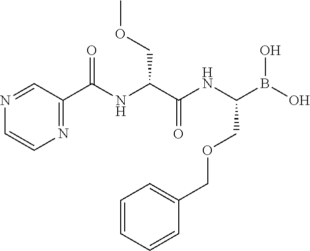 [SMILES: COC[C@@H](NC(=O)c1cnccn1)C(=O)N[C@@H](COCc1ccccc1)B(O)O] | Structure 38 |
| 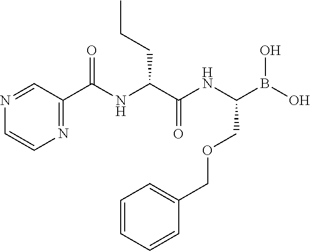 [SMILES: CCC[C@@H](NC(=O)c1cnccn1)C(=O)N[C@@H](COCc1ccccc1)B(O)O] | Structure 39 |
| 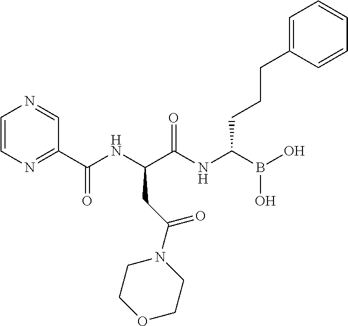 [SMILES: O=C(N[C@H](CC(=O)N1CCOCC1)C(=O)N[C@H](CCCc1ccccc1)B(O)O)c1cnccn1] | Structure 40 |
| 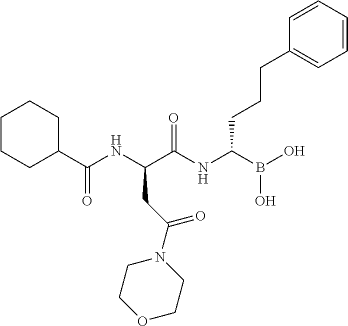 [SMILES: O=C(N[C@H](CC(=O)N1CCOCC1)C(=O)N[C@H](CCCc1ccccc1)B(O)O)C1CCCCC1] | Structure 41 |
| 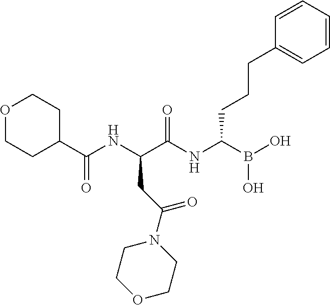 [SMILES: O=C(N[C@H](CC(=O)N1CCOCC1)C(=O)N[C@H](CCCc1ccccc1)B(O)O)C1CCOCC1] | Structure 42 |
| 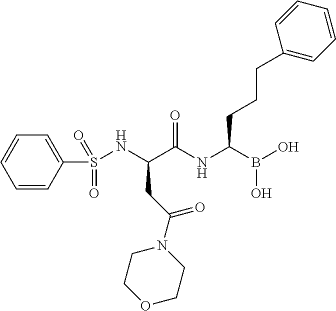 [SMILES: O=C(N[C@@H](CCCc1ccccc1)B(O)O)[C@@H](CC(=O)N1CCOCC1)NS(=O)(=O)c1ccccc1] | Structure 43 |
| 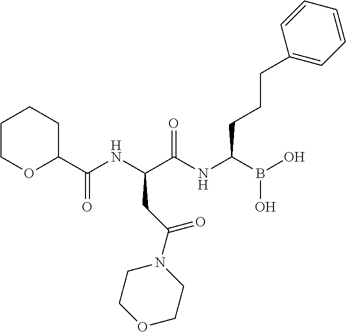 [SMILES: O=C(N[C@H](CC(=O)N1CCOCC1)C(=O)N[C@@H](CCCc1ccccc1)B(O)O)C1CCCCO1] | Structure 44 |
| 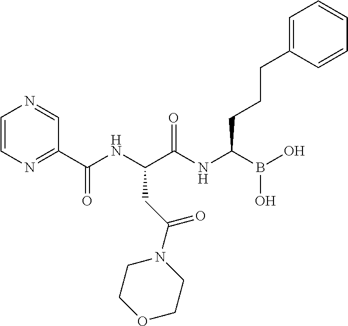 [SMILES: O=C(N[C@@H](CC(=O)N1CCOCC1)C(=O)N[C@@H](CCCc1ccccc1)B(O)O)c1cnccn1] | Structure 45 |
| 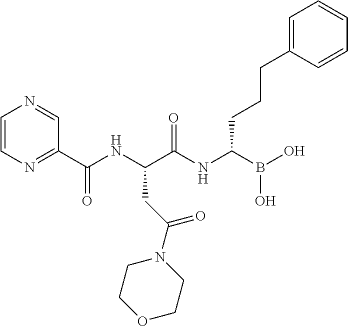 [SMILES: O=C(N[C@@H](CC(=O)N1CCOCC1)C(=O)N[C@H](CCCc1ccccc1)B(O)O)c1cnccn1] | Structure 46 |
| 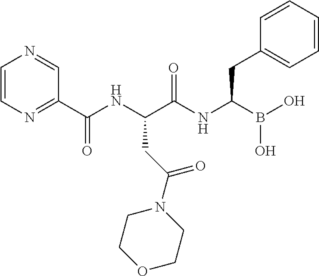 [SMILES: O=C(N[C@@H](CC(=O)N1CCOCC1)C(=O)N[C@@H](Cc1ccccc1)B(O)O)c1cnccn1] | Structure 47 |
| 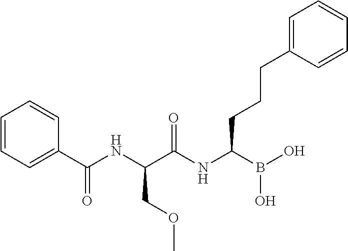 [SMILES: COC[C@@H](NC(=O)c1ccccc1)C(=O)N[C@@H](CCCc1ccccc1)B(O)O] | Structure 48 |
| 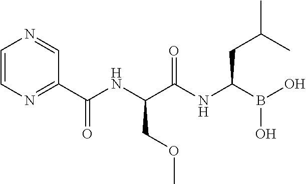 [SMILES: COC[C@@H](NC(=O)c1cnccn1)C(=O)N[C@@H](CC(C)C)B(O)O] | Structure 49 |
| 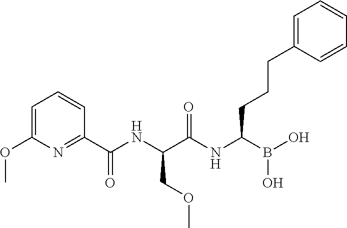 [SMILES: COC[C@@H](NC(=O)c1cccc(OC)n1)C(=O)N[C@@H](CCCc1ccccc1)B(O)O] | Structure 50 |
| 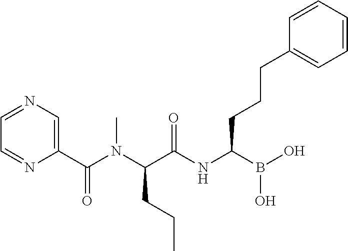 [SMILES: CCC[C@H](C(=O)N[C@@H](CCCc1ccccc1)B(O)O)N(C)C(=O)c1cnccn1] | Structure 51 |
| 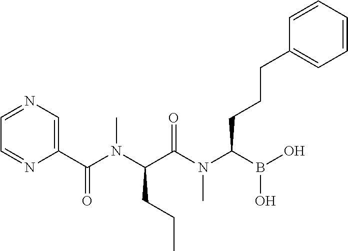 [SMILES: CCC[C@H](C(=O)N(C)[C@@H](CCCc1ccccc1)B(O)O)N(C)C(=O)c1cnccn1] | Structure 52 |

In certain embodiments, the compound is ((R)-1-((R)-4-morpholino-4-oxo-2-(pyrazine-2-carboxamido)butanamido)-4-phenylbutyl)boronic acid (Compound 1) or a pharmaceutically acceptable salt thereof.

In certain embodiments, the compound is ((R)-1-((R)-4-oxo-2-(pyrazine-2-carboxamido)-4-(pyrrolidin-1-yl)butanamido)-4-phenylbutyl)boronic acid (Compound 2) or a pharmaceutically acceptable salt thereof.

In certain embodiments, the compound is ((1R)-1-(4-oxo-4-phenyl-2-(pyrazine-2-carboxamido)butanamido)-4-phenylbutyl)boronic acid (Compound 3) or a pharmaceutically acceptable salt thereof.

In certain embodiments, the compound is ((R)-1-((R)-4-oxo-4-phenyl-2-(pyrazine-2-carboxamido)butanamido)-4-phenylbutyl)boronic acid (Compound 3a) or a pharmaceutically acceptable salt thereof.

In certain embodiments, the compound is ((R)-1-((S)-4-oxo-4-phenyl-2-(pyrazine-2-carboxamido)butanamido)-4-phenylbutyl)boronic acid Compound 3b) or a pharmaceutically acceptable salt thereof.

In certain embodiments, the compound is ((R)-3-methyl-1-((R)-4-morpholino-4-oxo-2-(pyrazine-2-carboxamido)butanamido)butyl)boronic acid (Compound 4) or a pharmaceutically acceptable salt thereof.

In certain embodiments, the compound is ((R)-1-((R)-4-morpholino-4-oxo-2-(pyrazine-2-carboxamido)butanamido)-3-phenylpropyl)boronic acid (Compound 5) or a pharmaceutically acceptable salt thereof.

In certain embodiments, the compound is ((R)-1-((R)-4-morpholino-4-oxo-2-(pyrazine-2-carboxamido)butanamido)pentyl)boronic acid (Compound 6) or a pharmaceutically acceptable salt thereof.

In certain embodiments, the compound is ((R)-1-((R)-2-(2,4-dimethyloxazole-5-carboxamido)-4-morpholino-4-oxobutanamido)-3-methylbutyl)boronic acid (Compound 7) or a pharmaceutically acceptable salt thereof.

In certain embodiments, the compound is ((R)-1-((R)-4-(dimethylamino)-4-oxo-2-(pyrazine-2-carboxamido)butanamido)-4-phenylbutyl)boronic acid (Compound 8) or a pharmaceutically acceptable salt thereof.

In certain embodiments, the compound is ((R)-1-((R)-4-(ethylamino)-4-oxo-2-(pyrazine-2-carboxamido)butanamido)-4-phenylbutyl)boronic acid (Compound 9) or a pharmaceutically acceptable salt thereof.

In certain embodiments, the compound is ((R)-1-((R)-4-amino-4-oxo-2-(pyrazine-2-carboxamido)butanamido)-4-phenylbutyl)boronic acid (Compound 10) or a pharmaceutically acceptable salt thereof.

In certain embodiments, the compound is ((R)-1-((R)-4-oxo-4-(piperidin-1-yl)-2-(pyrazine-2-carboxamido)butanamido)-4-phenylbutyl)boronic acid (Compound 11) or a pharmaceutically acceptable salt thereof.

In certain embodiments, the compound is ((R)-1-((R)-3-(oxazol-5-yl)-2-(pyrazine-2-carboxamido)propanamido)-4-phenylbutyl)boronic acid (Compound 12) or a pharmaceutically acceptable salt thereof.

In certain embodiments, the compound is ((R)-1-((R)-4-methoxy-2-(pyrazine-2-carboxamido)butanamido)-4-phenylbutyl)boronic acid (Compound 13) or a pharmaceutically acceptable salt thereof.

In certain embodiments, the compound is ((R)-1-((R)-3-(oxazol-2-yl)-2-(pyrazine-2-carboxamido)propanamido)-4-phenylbutyl)boronic acid (Compound 14) or a pharmaceutically acceptable salt thereof.

In certain embodiments, the compound is ((R)-1-((R)-3-(1,3,4-oxadiazol-2-yl)-2-(pyrazine-2-carboxamido)propanamido)-4-phenylbutyl)boronic acid (Compound 15) or a pharmaceutically acceptable salt thereof.

In certain embodiments, the compound is ((R)-1-((R)-3-(5-methyl-1,3,4-oxadiazol-2-yl)-2-(pyrazine-2-carboxamido)propanamido)-4-phenylbutyl)boronic acid (Compound 16) or a pharmaceutically acceptable salt thereof.

In certain embodiments, the compound is ((R)-4-phenyl-1-((R)-2-(pyrazine-2-carboxamido)-3-(thiazol-2-yl)propanamido)butyl)boronic acid (Compound 17) or a pharmaceutically acceptable salt thereof.

In certain embodiments, the compound is ((R)-1-((R)-3-(1-methyl-1H-imidazol-2-yl)-2-(pyrazine-2-carboxamido)propanamido)-4-phenylbutyl)boronic acid (Compound 18) or a pharmaceutically acceptable salt thereof.

In certain embodiments, the compound is ((R)-1-((R)-2-acetamido-4-morpholino-4-oxobutanamido)-4-phenylbutyl)boronic acid (Compound 19) or a pharmaceutically acceptable salt thereof.

In certain embodiments, the compound is ((R)-1-((R)-2-((tert-butoxycarbonyl)amino)-4-morpholino-4-oxobutanamido)-4-phenylbutyl)boronic acid (Compound 20) or a pharmaceutically acceptable salt thereof.

In certain embodiments, the compound is ((R)-1-((R)-2-(morpholine-4-carboxamido)-4-morpholino-4-oxobutanamido)-4-phenylbutyl)boronic acid (Compound 21) or a pharmaceutically acceptable salt thereof.

In certain embodiments, the compound is ((R)-1-((R)-2-(3-(tert-butyl) ureido)-4-morpholino-4-oxobutanamido)-4-phenylbutyl)boronic acid (Compound 22) or a pharmaceutically acceptable salt thereof.

In certain embodiments, the compound is ((R)-1-((R)-5-morpholino-5-oxo-2-(pyrazine-2-carboxamido) pentanamido)-4-phenylbutyl)boronic acid (Compound 23) or a pharmaceutically acceptable salt thereof.

In certain embodiments, the compound is ((1R)-4-phenyl-1-((2S)-3-(phenylsulfinyl)-2-(pyrazine-2-carboxamido)propanamido)butyl)boronic acid (Compound 24) or a pharmaceutically acceptable salt thereof.

In certain embodiments, the compound is ((R)-4-phenyl-1-((S)-3-(phenylsulfonyl)-2-(pyrazine-2-carboxamido)propanamido)butyl)boronic acid (Compound 25) or a pharmaceutically acceptable salt thereof.

In certain embodiments, the compound is ((R)-1-((R)-4-morpholino-2-(oxazol-2-ylamino)-4-oxobutanamido)-4-phenylbutyl)boronic acid (Compound 26) or a pharmaceutically acceptable salt thereof.

In certain embodiments, the compound is ((R)-1-((S)-3-(N,N-dimethylsulfamoyl)-2-(pyrazine-2-carboxamido)propanamido)-4-phenylbutyl)boronic acid (Compound 27) or a pharmaceutically acceptable salt thereof.

In certain embodiments, the compound is ((R)-1-((R)-4-(oxazol-2-yl)-2-(pyrazine-2-carboxamido)butanamido)-4-phenylbutyl)boronic acid (Compound 28) or a pharmaceutically acceptable salt thereof.

In certain embodiments, the compound is ((R)-4-phenyl-1-((R)-2-(pyrazine-2-carboxamido)-3-(pyridin-2-yloxy)propanamido)butyl)boronic acid (Compound 29) or a pharmaceutically acceptable salt thereof.

In certain embodiments, the compound is ((R)-1-((R)-4-morpholino-4-oxo-2-(trifluoromethylsulfonamido)butanamido)-4-phenylbutyl)boronic acid (Compound 30) or a pharmaceutically acceptable salt thereof.

In certain embodiments, the compound is ((R)-1-((R)-4-morpholino-4-oxo-2-(pyrazine-2-carboxamido)butanamido)butyl)boronic acid (Compound 31) or a pharmaceutically acceptable salt thereof.

In certain embodiments, the compound is ((R)-1-((R)-4-morpholino-4-oxo-2-(pyrazine-2-carboxamido)butanamido)propyl)boronic acid (Compound 32) or a pharmaceutically acceptable salt thereof.

In certain embodiments, the compound is ((R)-1-((R)-2-(3,3-dimethylureido)-4-morpholino-4-oxobutanamido)-4-phenylbutyl)boronic acid (Compound 33) or a pharmaceutically acceptable salt thereof.

In certain embodiments, the compound is ((R)-1-((S)-3-(methylsulfonyl)-2-(pyrazine-2-carboxamido)propanamido)-4-phenylbutyl)boronic acid (Compound 34) or a pharmaceutically acceptable salt thereof.

In certain embodiments, the compound is ((R)-1-((R)-4-(methylsulfonyl)-2-(pyrazine-2-carboxamido)butanamido)-4-phenylbutyl)boronic acid (Compound 35) or a pharmaceutically acceptable salt thereof.

In certain embodiments, the compound is ((R)-4-phenyl-1-((R)-2-(pyrazine-2-carboxamido)-3-(pyridin-2-yl)propanamido)butyl)boronic acid (Compound 36) or a pharmaceutically acceptable salt thereof.

In certain embodiments, the compound is ((R)-4-((R)-1-((R)-3-methoxy-2-(pyrazine-2-carboxamido)propanamido)-4-phenylbutyl)boronic acid (Compound 37) or a pharmaceutically acceptable salt thereof.

In certain embodiments, the compound is ((R)-2-(benzyloxy)-1-((R)-3-methoxy-2-(pyrazine-2-carboxamido)propanamido)ethyl)boronic acid (Compound 38) or a pharmaceutically acceptable salt thereof.

In certain embodiments, the compound is ((R)-2-(benzyloxy)-1-((R)-2-(pyrazine-2-carboxamido) pentanamido)ethyl)boronic acid (Compound 39) or a pharmaceutically acceptable salt thereof.

In certain embodiments, the compound is ((S)-1-((R)-4-morpholino-4-oxo-2-(pyrazine-2-carboxamido)butanamido)-4-phenylbutyl)boronic acid (Compound 40) or a pharmaceutically acceptable salt thereof.

In certain embodiments, the compound is ((R)-1-((R)-2-(cyclohexanecarboxamido)-4-morpholino-4-oxobutanamido)-4-phenylbutyl)boronic acid (Compound 41) or a pharmaceutically acceptable salt thereof.

In certain embodiments, the compound is ((R)-1-((R)-4-morpholino-4-oxo-2-(tetrahydro-2H-pyran-4-carboxamido)butanamido)-4-phenylbutyl)boronic acid (Compound 42) or a pharmaceutically acceptable salt thereof.

In certain embodiments, the compound is ((R)-1-((R)-4-morpholino-4-oxo-2-(phenylsulfonamido)butanamido)-4-phenylbutyl)boronic acid (Compound 43) or a pharmaceutically acceptable salt thereof.

In certain embodiments, the compound is ((1R)-1-((2R)-4-morpholino-4-oxo-2-(tetrahydro-2H-pyran-2-carboxamido)butanamido)-4-phenylbutyl)boronic acid (Compound 44) or a pharmaceutically acceptable salt thereof.

In certain embodiments, the compound is ((R)-1-((S)-4-morpholino-4-oxo-2-(pyrazine-2-carboxamido)butanamido)-4-phenylbutyl)boronic acid (Compound 45) or a pharmaceutically acceptable salt thereof.

In certain embodiments, the compound is ((S)-1-((S)-4-morpholino-4-oxo-2-(pyrazine-2-carboxamido)butanamido)-4-phenylbutyl)boronic acid (Compound 46) or a pharmaceutically acceptable salt thereof.

In certain embodiments, the compound is ((R)-1-((R)-4-morpholino-4-oxo-2-(pyrazine-2-carboxamido)butanamido)-2-phenylethyl)boronic acid (Compound 47) or a pharmaceutically acceptable salt thereof.

In certain embodiments, the compound is ((R)-1-((R)-2-benzamido-3-methoxypropanamido)-4-phenylbutyl)boronic acid (Compound 48) or a pharmaceutically acceptable salt thereof.

In certain embodiments, the compound is ((R)-1-((R)-3-methoxy-2-(pyrazine-2-carboxamido)propanamido)-3-methylbutyl)boronic acid (Compound 49) or a pharmaceutically acceptable salt thereof.

In certain embodiments, the compound is ((R)-1-((R)-3-methoxy-2-(6-methoxypicolinamido)propanamido)-4-phenylbutyl)boronic acid (Compound 50) or a pharmaceutically acceptable salt thereof.

In certain embodiments, the compound is ((R)-1-((R)-2-(N-methylpyrazine-2-carboxamido) pentanamido)-4-phenylbutyl)boronic acid (Compound 51) or a pharmaceutically acceptable salt thereof.

In certain embodiments, the compound is ((R)-1-((R)—N-methyl-2-(N-methylpyrazine-2-carboxamido) pentanamido)-4-phenylbutyl)boronic acid (Compound 52) or a pharmaceutically acceptable salt thereof.

The compounds of the present disclosure may contain asymmetric carbon atoms (sometimes as the result of a deuterium atom) and thereby can exist as either individual stereoisomers or mixtures of enantiomers or mixtures of diastereomers. Accordingly, a compound of the present disclosure may exist as either a racemic mixture, a mixture of diastereomers, or as individual stereoisomers that are substantially free of other stereoisomers. Synthetic, separation, or purification methods to be used to obtain an enantiomer of a given compound are known in the art and are applicable for obtaining the compounds identified herein.

Unless otherwise indicated, when a disclosed compound is named or depicted by a structure without specifying the stereochemistry and has one or more chiral centers, it is understood to represent all possible stereoisomers of the compound. In other words, chiral centers that lack solid wedged or hashed wedged bonds indicate a mixture of stereoisomers.

Certain compounds of the present disclosure may be able to exist as tautomers. All tautomeric forms of these compounds, whether isolated individually or in mixtures, are within the scope of the present disclosure. For example, in instances where an —OH substituent is permitted on a heteroaromatic ring and keto-enol tautomerism is possible, it is understood that the substituent might in fact be present, in whole or in part, in the oxo (═O) form.

Compounds of the present disclosure may exist in amorphous form and/or one or more crystalline forms. As such, all amorphous and crystalline forms and mixtures thereof of the compounds of the disclosure are intended to be included within the scope of the present disclosure. In addition, some of the compounds of the present disclosure may form solvates with water (i.e., a hydrate) or common organic solvents. Such solvates and hydrates, particularly the pharmaceutically acceptable solvates and hydrates, of the compounds of this disclosure are likewise encompassed within the scope of the compounds of the disclosure and the pharmaceutically acceptable salts thereof, along with un-solvated and anhydrous forms of such compounds.

In one embodiment, deuterium isotope content at the deuterium substituted position is greater than the natural isotopic deuterium content (0.015%), more preferably greater than 50%, more preferably greater than 60%, more preferably greater than 75%, more preferably greater than 90%, more preferably greater than 95%, more preferably greater than 97%, more preferably greater than 99%. It will be understood that some variation of natural isotopic abundance may occur in any compound depending upon the source of the reagents used in the synthesis. Thus, a preparation of undeuterated compounds may inherently contain small amounts of deuterated isotopologues, such amounts being insignificant as compared to the degree of stable isotopic substitution of the deuterated compounds of the disclosure (see e.g., Gannes, L. Z. et al., Comp. Biochem. Physiol. Mol. Integr. Physiol, 119, 725 (1998)). Replacement of hydrogen with deuterium may affect the activity, toxicity, and pharmacokinetics (e.g., absorption, distribution, metabolism, and excretion (“ADME”)) of some drugs. For instance, such replacement may alter the chemical stability and biochemical reactivity of a compound through kinetic isotope effects. Because of the increased mass of deuterium relative to hydrogen, epimerization at stereogenic carbons may be slowed down when hydrogen is replaced with deuterium (see Pirali, T. et al, J. Med. Chem. 62, 5276-97 (2019)). Additionally, the presence of deuterium may affect how a molecule interacts with enzymes, thereby impacting enzyme kinetics. While in certain cases the increased mass of deuterium as compared to hydrogen can stabilize a compound and thereby improve activity, toxicity, or half-life, such impact is not predictable. In other instances, deuteration may have little to no impact on these properties, or may affect them in an undesirable manner. Whether and/or how such replacement will impact drug properties can only be determined if the drug is synthesized, evaluated, and compared to its non-deuterated counterpart (see Fukuto, J. M., et al., J. Med. Chem. 34, 2871-76 (1991)). Because some drugs have multiple sites of metabolism or more than one active sites for binding to a target, it is unpredictable as to which sites may benefit by deuterium replacement or to what extent isotope enrichment is necessary to produce a beneficial effect.

Further aspects and embodiments of the present disclosure are directed to compounds of Formula I or their pharmaceutically acceptable salts wherein one or more hydrogen is substituted with a deuterium atom.

A further aspect of the present disclosure provides a pharmaceutical composition comprising one or more compound of the first aspect of the disclosure or a pharmaceutically acceptable salt, solvate, stereoisomer or mixture of stereoisomers, tautomer, isotopic form, or pharmaceutically active metabolite thereof, or combinations thereof and one or more pharmaceutically acceptable excipient or carrier.

Further embodiments of the disclosure are methods of treating a disease characterized by mitochondrial dysfunction, such methods comprising administering to a subject in need thereof a therapeutically effective amount of a compound of the disclosure, or a pharmaceutically acceptable salt thereof. In some embodiments, the disease is selected from the group consisting of Alzheimer's disease, Parkinson's disease, obesity, diabetes, non-alcoholic steatohepatitis (NASH), and related metabolic syndromes such as non-alcoholic fatty liver disease (NAFLD), Alper's syndrome (Alpers-Huttenlocher syndrome), ataxia neuropathy syndrome (ANS), Mitochondrial DNA Depletion Syndrome (MDDS), Leigh Syndrome (Leigh Disease), Leber's Hereditary Optic Neuropathy (LHON), chronic progressive external ophthalmoplegia (CPEO), myoclonic epilepsy myopathy sensory ataxia (MEMSA), MELAS (Mitochondrial Encephalopathy, Lactic Acidosis, and Stroke-like episodes) syndrome, MERRF (myoclonus epilepsy with ragged-red fibers) syndrome, mitochondrial neurogastrointestinal encephalomyopathy (MNGIE), neuropathy, ataxia, and retinitis pigmentosa (NARP), Kearn's-Sayre Syndrome (KSS), and Pearson's Syndrome.

In some embodiments, the disease to be treated with a compounds of the disclosure or pharmaceutically acceptable salts thereof is associated with mtDNA mutations or deletions, for example m.3243A>G, m.11778G>A, m.14484T>C, m.3460G>A, m.8344A>G, m.3271T>C, m.3251A>G, m.8356T>C, m.4274T>C, m.14709T>C, m.12320A>G, m.4269A>G, m.12258C>A, m.1606G>A, m.10010T>C, m.7445A>G, and m.1555A>G (see https://mitomap.org/MITOMAP).

Additional embodiments of the disclosure relate to methods of treating cancers, such as those identified in Wong, K. S. et al. “Recent Advances in Targeting Human Mitochondrial AAA+ Proteases to Develop Novel Cancer Therapeutics,” Advances in Experimental Medicine and Biology, 1158, 119-142 (2019), using a compound of the disclosure or its pharmaceutically acceptable salt.

Further aspects and embodiments are directed to one or more compounds or pharmaceutical compositions of any aspect of the disclosure for use in medicine. In particular, the compounds and pharmaceutical compositions may be for use in the treatment of a disorder or a disease characterized by a mitochondrial dysfunction as defined elsewhere herein. In embodiments, the use comprises administering to a subject in need thereof a therapeutically effective amount of a compound of the disclosure, or a pharmaceutically acceptable salt thereof, according to any aspect.

For the treatment of cancer, the compounds described herein may be administered in combination with a chemotherapeutic agent. Therapeutically effective amounts of the additional chemotherapeutic agent(s) are well known to those skilled in the art. However, it is well within the attending physician to determine the amount of other chemotherapeutic agent(s) to be delivered.

Examples of these chemotherapeutic agents include, but are not limited to, Abitrexate (Methotrexate Injection), Abraxane (Paclitaxel Injection), Actemra (Tocilizumab), Adcetris (Brentuximab Vedotin Injection), Adriamycin (Doxorubicin), Adrucil Injection (5-FU (fluorouracil)), Afinitor (Everolimus), Afinitor Disperz (Everolimus), Aldara (Imiquimod), Alimta (PEMET EXED), Alkeran Injection (Melphalan Injection), Alkeran Tablets (Melphalan), Aredia (Pamidronate), Arimidex (Anastrozole), Aromasin (Exemestane), Arranon (Nelarabine), Arzerra (Ofatumumab Injection), Avastin (Bevacizumab), Avelumab, Bexxar (Tositumomab), BiCNU (Carmustine), Blenoxane (Bleomycin), Blincyto (Blinatumomab), Bosulif (Bosutinib), Busulfex Injection (Busulfan Injection), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Clolar (Clofarabine Injection), Cometriq (Cabozantinib), Cosmegen (Dactinomycin), CytosarU (Cytarabine), Cytoxan (Cytoxan), Cytoxan Injection (Cyclophosphamide Injection), Cyramza (Ramucirumab), Dacogen (Decitabine), Darzalex (Daratumumab), DaunoXome (Daunorubicin Lipid Complex Injection), Decadron (Dexamethasone), DepoCyt (Cytarabine Lipid Complex Injection), Dexamethasone Intensol (Dexamethasone), Dexpak Taperpak (Dexamethasone), Docefrez (Docetaxel), Doxil (Doxorubicin Lipid Complex Injection), Droxia (Hydroxyurea), DTIC (Decarbazine), Durvalumab, Eligard (Leuprolide), Ellence (Ellence (epirubicin)), Eloxatin (Eloxatin (oxaliplatin)), Elspar (Asparaginase), Emcyt (Estramustine), Empliciti (Elotuzumab), Enhertu (fam-trastuzumab deruxtecan-nxki), Erbitux (Cetuximab), Erivedge (Vismodegib), Erwinaze (Asparaginase Erwinia chrysanthemi), Ethyol (Amifostine), Etopophos (Etoposide Injection), Eulexin (Flutamide), Fareston (Toremifene), Faslodex (Fulvestrant), Femara (Letrozole), Firmagon (Degarelix Injection), Fludara (Fludarabine), Folex (Methotrexate Injection), Folotyn (Pralatrexate Injection), FUDR (FUDR (floxuridine)), Gazyva (Obinutuzumab), Gemzar (Gemcitabine), Gilotrif (Afatinib), Gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Iclusig (Ponatinib), Idamycin PFS (Idarubicin), Ifex (Ifosfamide), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Kyprolis (Carfilzomib), Leflunomide (SU101), Lartruvo (Olaratumab), Leukeran (Chlorambucil), Leukine (Sargramostim), Leustatin (Cladribine), Libtayo (Cemiplimab), Lupron (Leuprolide), Lupron Depot (Leuprolide), Lupron DepotPED (Leuprolide), Lysodren (Mitotane), Marqibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Ninlaro (Ixazomib), Nipent (Pentostatin), Nolvadex (Tamoxifen), Novantrone (Mitoxantrone), Oncaspar (Pegaspargase), Oncovin (Vincristine), Ontak (Denileukin Diftitox), Onxol (Paclitaxel Injection), Panretin (Alitretinoin), Paraplatin (Carboplatin), Perjeta (Pertuzumab Injection), Platinol (Cisplatin), Platinol (Cisplatin Injection), PlatinoIAQ (Cisplatin), PlatinoIAQ (Cisplatin Injection), Pomalyst (Pomalidomide), Portrazza (Necitumumab), Prednisone Intensol (Prednisone), Proleukin (Aldesleukin), Purinethol (Mercaptopurine), Reclast (Zoledronic acid), Revlimid (Lenalidomide), Removab (Catumaxomab), Rheumatrex (Methotrexate), Rituxan (Rituximab), RoferonA alfaa (Interferon alfa-2a), Rubex (Doxorubicin), Sandostatin (Octreotide), Sandostatin LAR Depot (Octreotide), Sarclisa (Isatuximab-irfc), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b Injection (Sylatron)), Synribo (Omacetaxine Injection), Tabloid (Thioguanine), Taflinar (Dabrafenib), Tarceva (Erlotinib), Targretin Capsules (Bexarotene), Tasigna (Decarbazine), Taxol (Paclitaxel Injection), Taxotere (Docetaxel), Tecentriq (Atezolizumab), Temodar (Temozolomide), Temodar (Temozolomide Injection), Tepadina (Thiotepa), Thalomid (Thalidomide), TheraCys BCG (BCG), Thioplex (Thiotepa), TICE BCG (BCG), Toposar (Etoposide Injection), Torisel (Temsirolimus), Treanda (Bendamustine hydrochloride), Tremelimumab, Trelstar (Triptorelin Injection), Trexall (Methotrexate), Trisenox (Arsenic trioxide), Tykerb (lapatinib), Unituxin (Dinutuximab), Valstar (Valrubicin Intravesical), Vantas (Histrelin Implant), Vectibix (Panitumumab), Velban (Vinblastine), Velcade (Bortezomib), Vepesid (Etoposide), Vepesid (Etoposide Injection), Vesanoid (Tretinoin), Vidaza (Azacitidine), Vincasar PFS (Vincristine), Vincrex (Vincristine), Votrient (Pazopanib), Vumon (Teniposide), Wellcovorin IV (Leucovorin Injection), Xalkori (Crizotinib), Xeloda (Capecitabine), Xtandi (Enzalutamide), Yervoy (Ipilimumab Injection), Zaltrap (Ziv-aflibercept Injection), Zanosar (Streptozocin), Zelboraf (Vemurafenib), Zevalin (Ibritumomab Tiuxetan), Zoladex (Goserelin), Zolinza (Vorinostat), Zometa (Zoledronic acid), Zortress (Everolimus), Zytiga (Abiraterone), Nimotuzumab and immune checkpoint inhibitors such as nivolumab, pembrolizumab/MK-3475, pidilizumab and AMP-224 targeting PD-1; and BMS-935559, MEDI4736, MPDL3280A and MSB0010718C targeting.

Further embodiments of the disclosure are methods of treating neurodegenerative disorders, metabolic disorders, and diseases associated with the aging process.

A further embodiment of the present disclosure are compounds of the invention (that is, compounds of Structures 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, and 52) or their pharmaceutically acceptable salts wherein one or more hydrogen is substituted with a deuterium atom. Particular compounds of the disclosure include the compounds of Structures 3, 51 and 52.

Additional embodiments of the disclosure are pharmaceutical compositions comprising a compound as disclosed herein (that is, compounds of Structures 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, and 52); particularly the compounds of Structures 3, 51 and 52, or a pharmaceutically acceptable salt thereof and a pharmaceutically acceptable excipient.

### Preparation of the Compounds:

The starting materials and reagents used in each step in the preparation are known and can be readily prepared or purchased from commercial sources.

The compound obtained in each step can also be used for the next reaction as a reaction mixture thereof, or after obtaining a crude product thereof. Alternatively, the compound obtained in each step can be isolated and/or purified from the reaction mixture by a separation means such as concentration, crystallization, recrystallization, distillation, solvent extraction, fractionation, chromatography and the like according to a conventional method.

In each reaction step, while the reaction time varies depending on the reagents and solvents to be used, unless otherwise specified, it is generally 1 min to 48 hr, preferably 10 min to 8 hr.

In the reaction of each step, while the reaction temperature varies depending on the reagents and solvents to be used, unless otherwise specified, it is generally −78° C. to 300° C., preferably −78° C. to 150° C.

In the reaction of each step, unless otherwise specified, a reagent is used in 0.5 equivalent to 20 equivalents, preferably 0.8 equivalent to 5 equivalents, relative to the substrate. When a reagent is used as a catalyst, the reagent is used in 0.001 equivalent to 1 equivalent, preferably 0.01 equivalent to 0.2 equivalent, relative to the substrate. When the reagent is also a reaction solvent, the reagent is used in a solvent amount.

In the reaction of each step, unless otherwise specified, it is performed without solvent or by dissolving or suspending in a suitable solvent. Specific examples of the solvent include the following. Alcohols: methanol, ethanol, tert-butyl alcohol, 2-methoxyethanol and the like; ethers: diethyl ether, diphenyl ether, tetrahydrofuran, 1,2-dimethoxyethane and the like; aromatic hydrocarbons: chlorobenzene, toluene, xylene and the like; saturated hydrocarbons: cyclohexane, hexane and the like; amides: N, N-dimethylformamide, N-methylpyrrolidone and the like; halogenated hydrocarbons: dichloromethane, carbon tetrachloride and the like; nitriles: acetonitrile and the like; sulfoxides: dimethyl sulfoxide and the like; aromatic organic bases: pyridine and the like; acid anhydrides: acetic anhydride and the like; organic acids: formic acid, acetic acid, trifluoroacetic acid and the like; inorganic acids: hydrochloric acid, sulfuric acid and the like; esters: ethyl acetate and the like; ketones: acetone, methyl ethyl ketone and the like; and water.

Two or more kinds of the above-mentioned solvents may be used by mixing at an appropriate ratio.

Unless otherwise specified, the reaction of each step is performed according to a known method, for example, the methods described in “Reactions and Syntheses: In the Organic Chemistry Laboratory 2nd Edition” (Lutz F. Tietze, Theophil Eicher, Ulf Diederichsen, Andreas Speicher, Nina Schützenmeister) Wiley, 2015; “Organic Syntheses Collective Volumes 1-12” (John Wiley & Sons Inc); “Comprehensive Organic Transformations, Third Edition” (Richard C. Larock) Wiley, 2018 and the like.

In each step, protection or deprotection of a functional group is performed by a known method, for example, the methods described in “Protective Groups in Organic Synthesis, 4th Ed.” (Theodora W. Greene, Peter G. M. Wuts) Wiley-Interscience, 2007; “Protecting Groups 3rd Ed.” (P. J. Kocienski) Thieme, 2004 and the like.

Deuterated LONP1 inhibitors of the present invention can be prepared using chemical reactions known to a person of ordinary skill in the art using deuterated starting materials or reagents. Deuterium-containing reagents are well known in the art and can be prepared using known procedures or purchased from commercial sources. The deuterated compounds obtained can be characterized by analytical techniques known to persons of ordinary skill in the art. For example, nuclear magnetic resonance (“NMR”) can be used to determine a compound's structure while mass spectroscopy (“MS”) can be used to determine the amount of deuterium atom in the compound by comparison to its non-deuterated form.

In certain embodiments, Compound 1 can be produced from compound (1-1) by the method shown in Scheme 1. Amino acid (1-1) is coupled with morpholine (1-2) to produce amide (1-3), which is then deprotected to generate carboxylic acid (1-4). Subsequent coupling with the amine of a protected boronic acid compound (1-5) produces amide (1-6). Removal of the BOC protecting group yields the corresponding amine (1-7) as the hydrochloride salt, which is then coupled with carboxylic acid (1-8) to form amide (1-9). Deprotection of (1-9) with methylboronic acid (1-10) produces Compound 1.

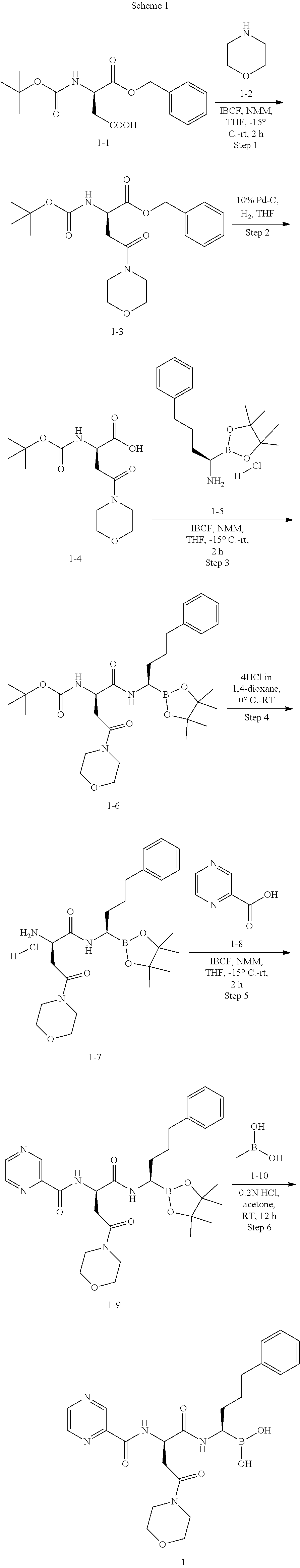

In other embodiments, Compound 2 can be produced from compound (2-1) by the method shown in Scheme 2. Amino acid (2-1) is coupled with pyrrolidone to form amide (2-2), which is then deprotected to generate the carboxylic acid compound (2-3). Coupling with the amine of a protected boronic acid compound (2-4) produces amide (2-5). Removal of the BOC protecting group yields the corresponding amine (2-6) as the hydrochloride salt, which is then coupled with carboxylic acid (2-7) to form amide (2-8). Deprotection of (2-8) with methylboronic acid (2-9) produces Compound 2.

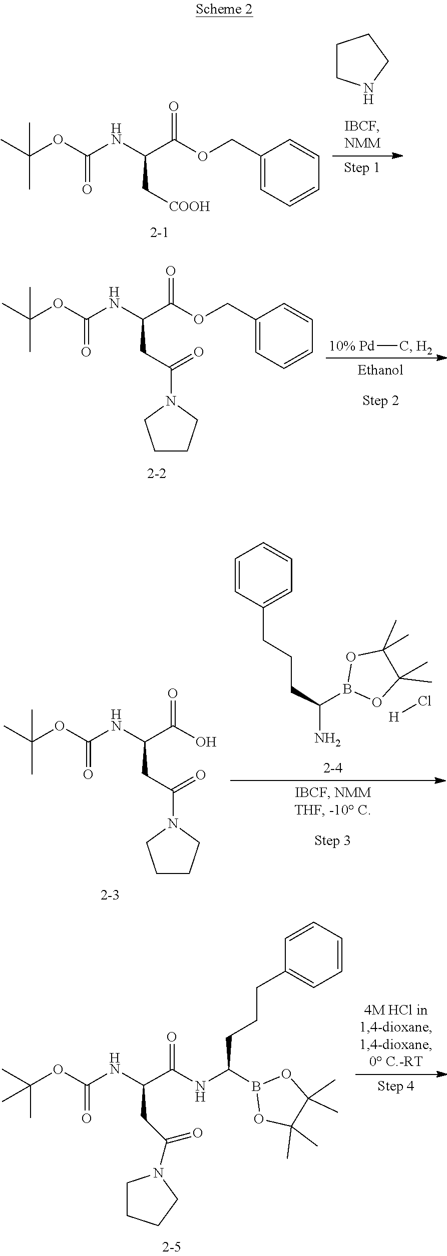

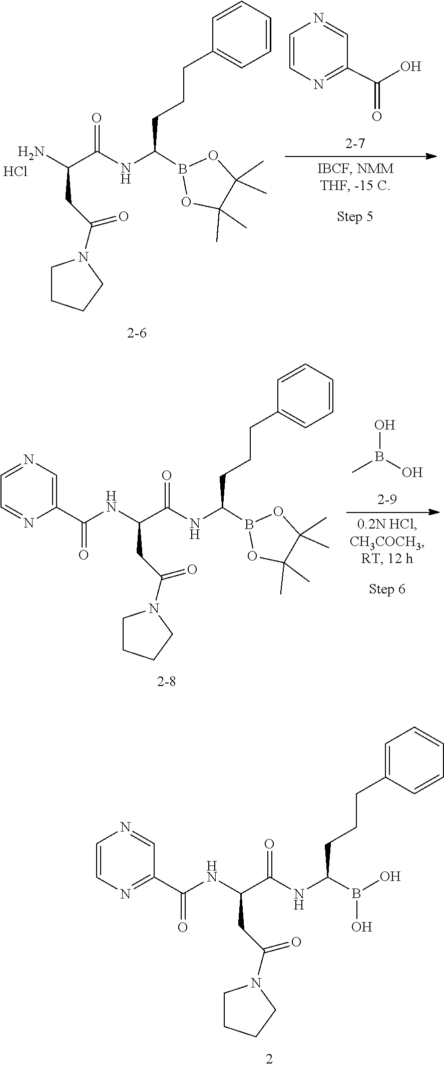

In further embodiments, Compound 3 can be produced from compounds (3-1) and (3-2) by the method shown in Scheme 3. Compounds (3-1) and (3-2) can be coupled to give compound (3-3), which undergoes saponification to result in compound (3-4). Compound (3-4) is protected to produce compound (3-5), which is coupled with the amine of a protected boronic acid compound (3-6) to produce a diastereomeric mixture of compound (3-7). Removal of the BOC protecting group yields the corresponding amine (3-8) as the hydrochloride salt, which is coupled with carboxylic acid (3-9) to form amide (3-10). Deprotection of (3-10) with methylboromic acid (3-11) produces Compound 3.

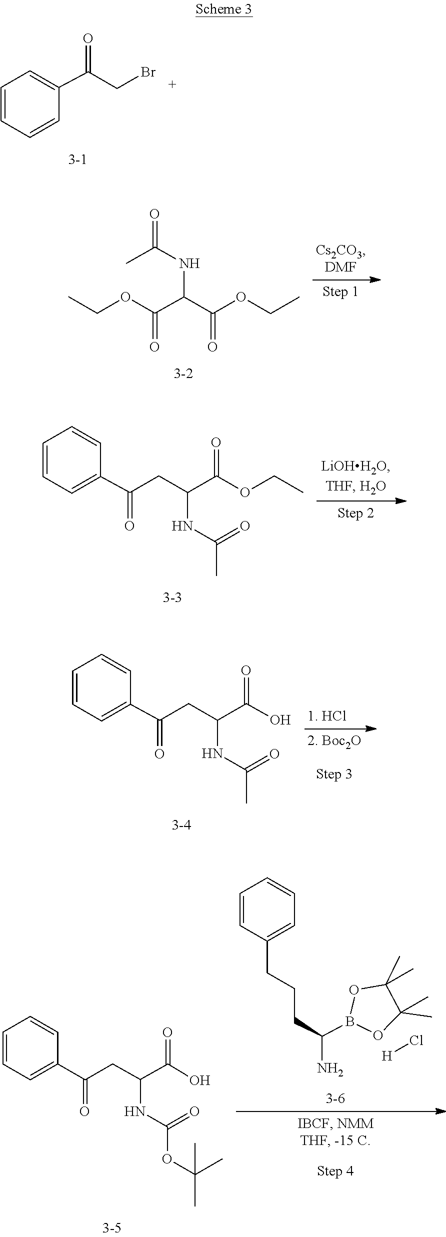

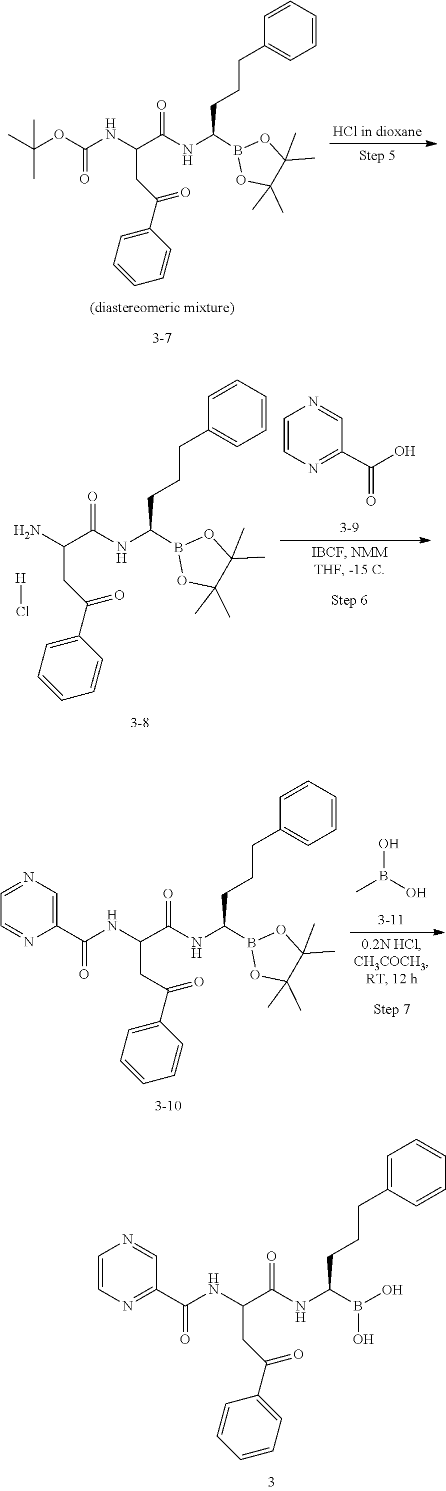

In another embodiment, Compound 4 can be produced from compound (4-1) by the method shown in Scheme 4. Carboxylic acid (4-1) is coupled with the amine of a protected boronic acid compound (4-2) to produce amide (4-3), which is then deprotected to generate amine (4-4) as the hydrochloride salt. Subsequent coupling with carboxylic acid (4-5) produces amide (4-6). Deprotection of (4-6) with methylboronic acid (4-7) produces Compound 4.

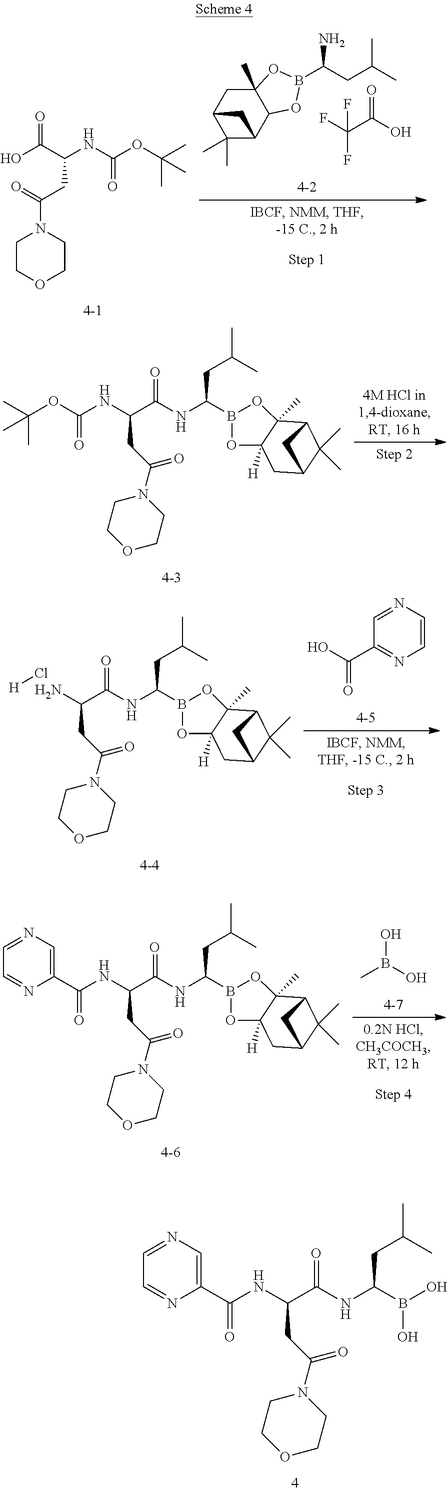

In another embodiment, Compound 5 can be produced from compound (5-1) by the method shown in Scheme 5. Carboxylic acid (5-1) is coupled with the amine of a protected boronic acid compound (5-2) to produce amide (5-3), which is then deprotected to generate amine (5-4) as the hydrochloride salt Subsequent coupling with carboxylic acid (5-5) produces amide (5-6). Deprotection of (5-6) with methylboronic acid (5-7) produces Compound 5.

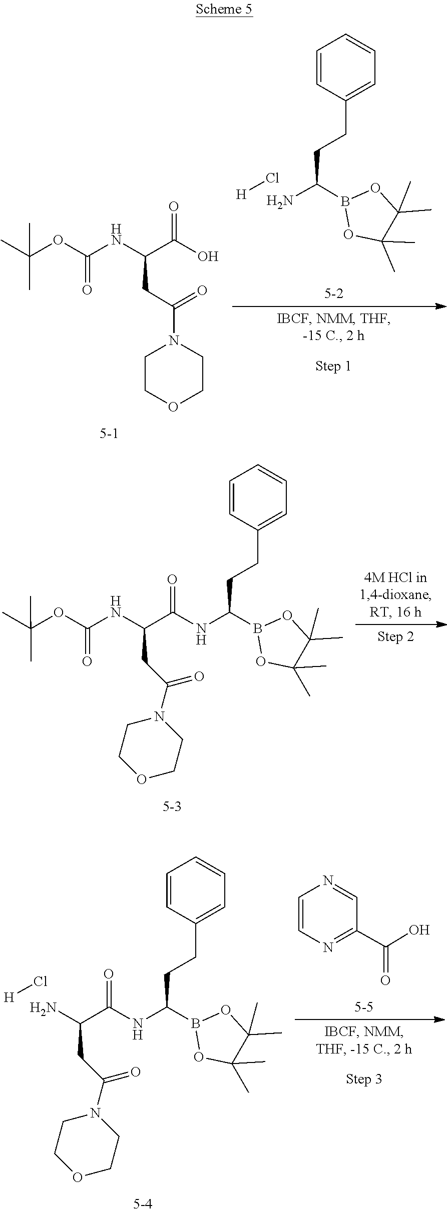

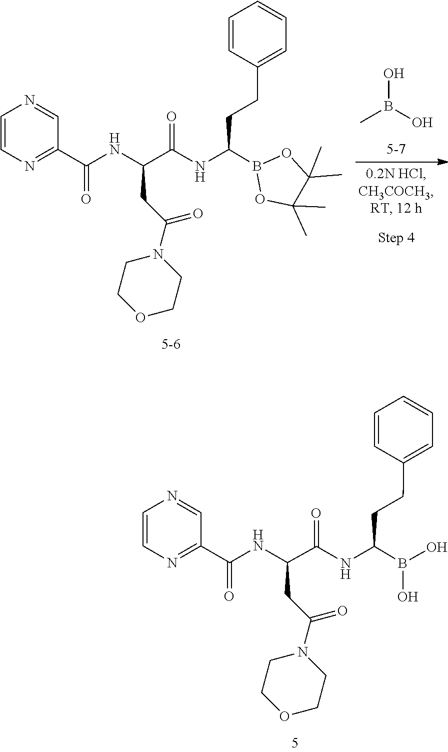

In another embodiment, Compound 6 can be produced from compound (6-1) by the method shown in Scheme 6. Carboxylic acid (6-1) is coupled with the amine of a protected boronic acid compound (6-2) to produce amide (6-3), which is then deprotected to generate amine (6-4) as the hydrochloride salt. Subsequent coupling with carboxylic acid (6-5) produces amide (6-6). Deprotection of (6-6) with methylboronic acid (6-7) produces Compound 6.

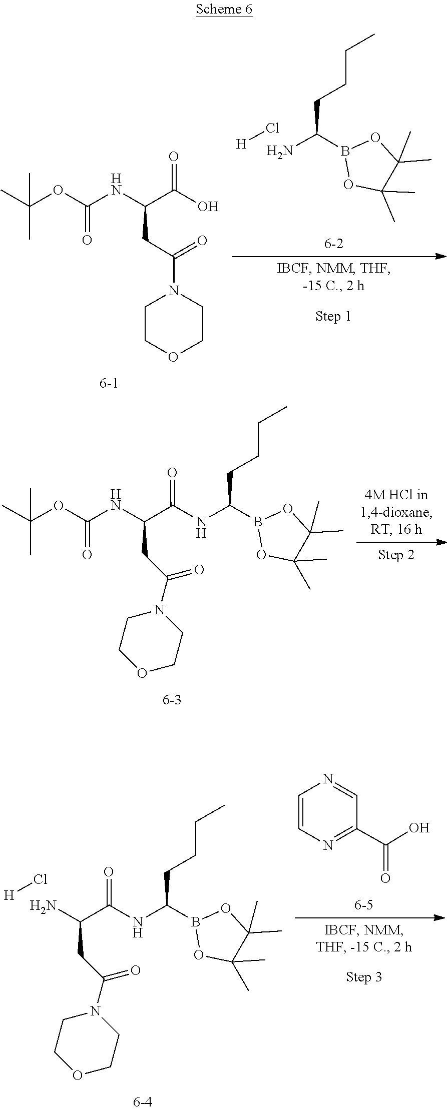

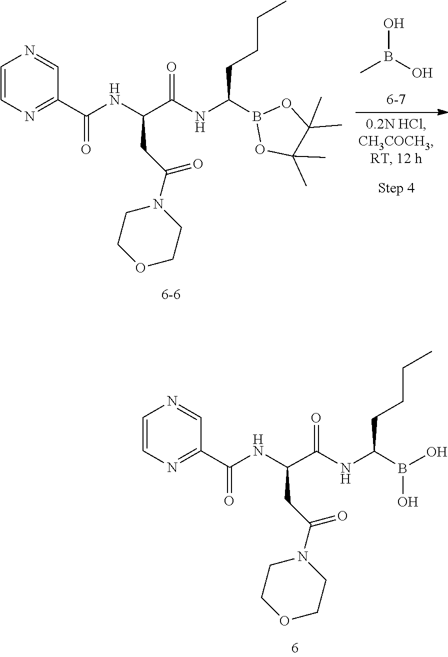

In another embodiment, Compound 7 can be produced from compound (7-1) by the method shown in Scheme 8. Carboxylic acid (7-1) is coupled with the amine of a protected boronic acid compound as the trifluoroacetic acid salt (7-2) to produce amide (7-3). Removal of the BOC protecting group yields the corresponding amine (7-4) as the hydrochloride salt. Subsequent coupling with carboxylic acid (7-5) produces amide (7-6). Deprotection of (7-6) with methylboronic acid (7-7) produces Compound 7.

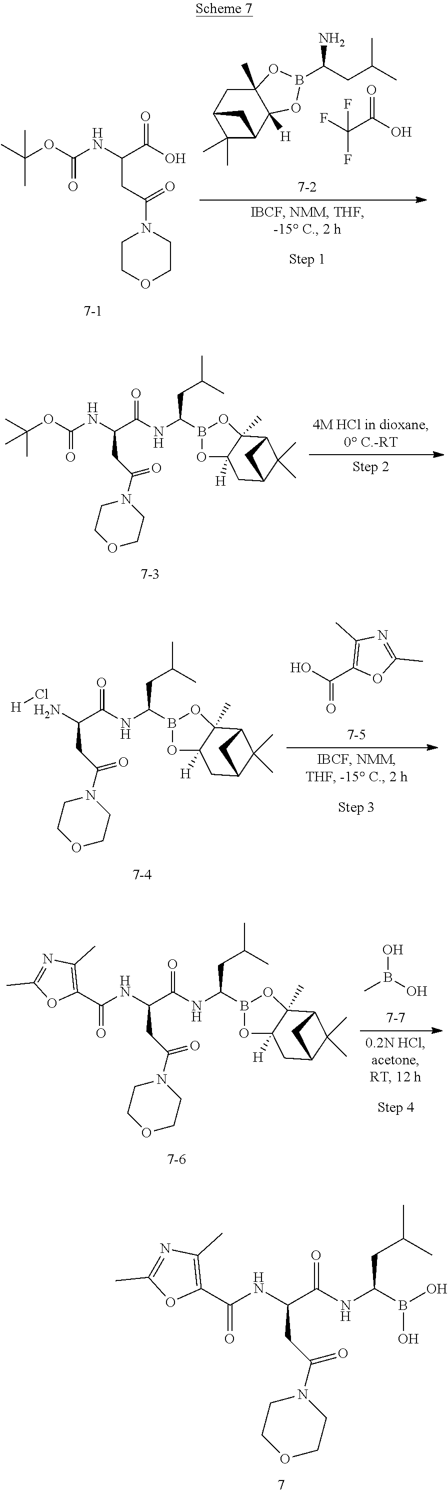

In another embodiment, Compound 8 can be produced from compound (8-1) by the method shown in Scheme 8. Carboxylic acid (8-1) is coupled with the dimethylamine (9-2) to produce amide (8-3), which is then deprotected to generate carboxylic acid (8-4). Subsequent coupling with amine of a protected boronic acid compound (8-5) produces amide (8-6). Removal of the BOC protecting group yields the corresponding amine (8-7) as the hydrochloride salt, which is then coupled with carboxylic acid (8-8) to form amide (8-9). Deprotection of (8-9) with methylboronic acid (8-10) produces Compound 8.

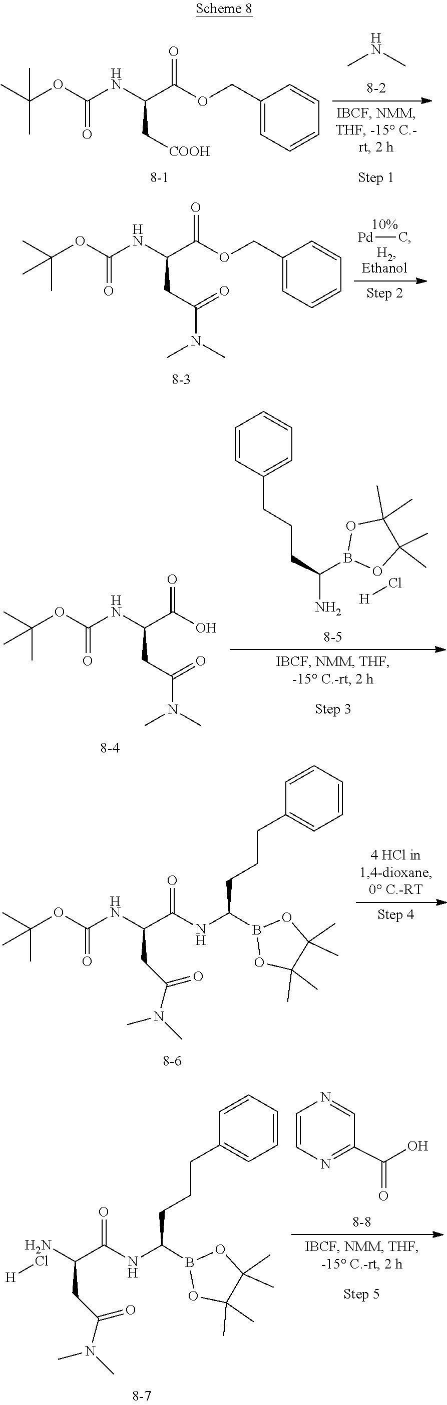

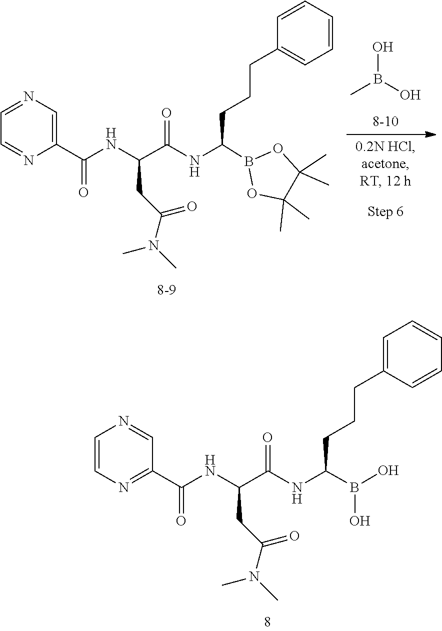

In another embodiment, Compound 9 can be produced from compound (9-1) by the method shown in Scheme 9. Carboxylic acid (9-1) is coupled with the ethylamine (10-2) to produce amide (9-3), which is deprotected to generate carboxylic acid (9-4). Subsequent coupling with the amine of a protected boronic acid compound (9-5) produces amide (9-6). Removal of the BOC protecting group yields the corresponding amine (9-7) as the hydrochloride salt, which is then coupled with carboxylic acid (9-8) to form amide (9-9). Deprotection of (9-9) with methylboronic acid (9-10) produces Compound 9.

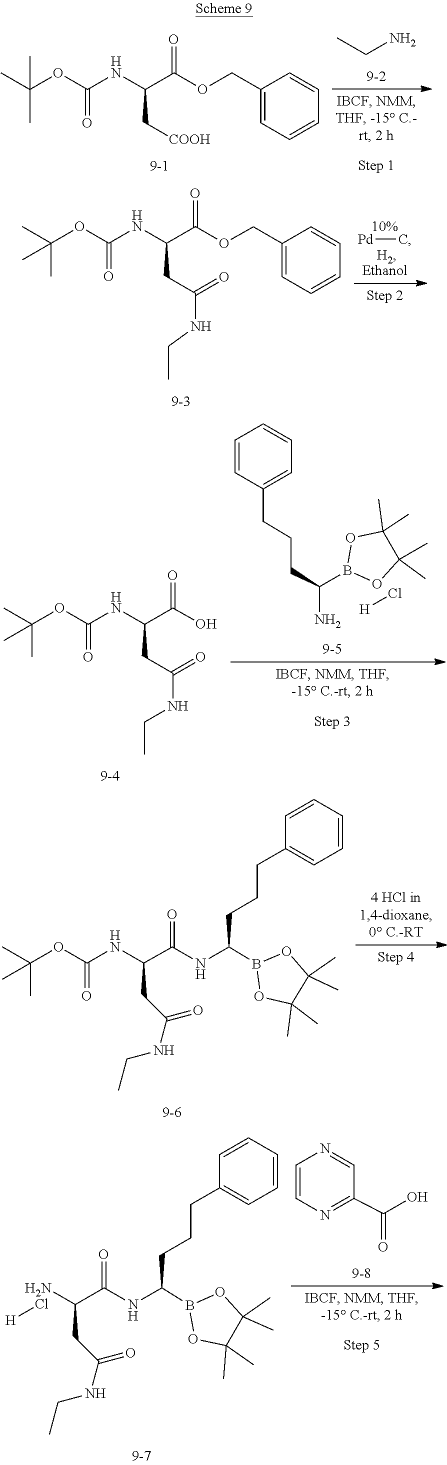

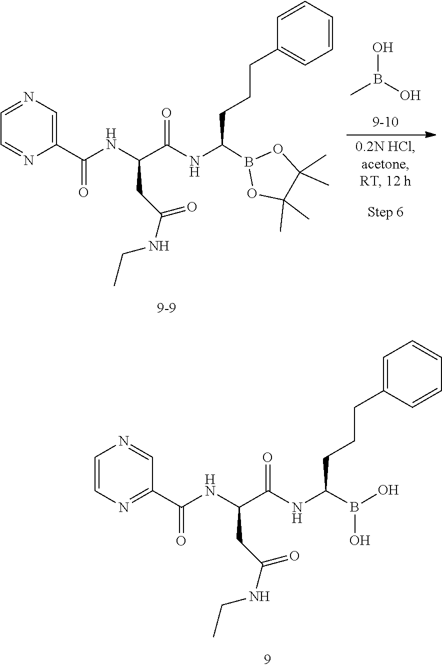

In another embodiment, Compound 11 can be produced from compound (11-1) by the method shown in Scheme 11. Carboxylic acid (11-1) is coupled with piperidine (11-2) to produce amide (11-3), which is then deprotected to generate carboxylic acid (11-4). Subsequent coupling with the amine of a protected boronic acid compound (11-5) produces amide (11-6). Removal of the BOC protecting group yields the corresponding amine (11-7) as the hydrochloride salt. Subsequent coupling with carboxylic acid (11-8) produces amide (11-9). Deprotection of (11-9) with methylboronic acid (11-10) produces Compound 11.

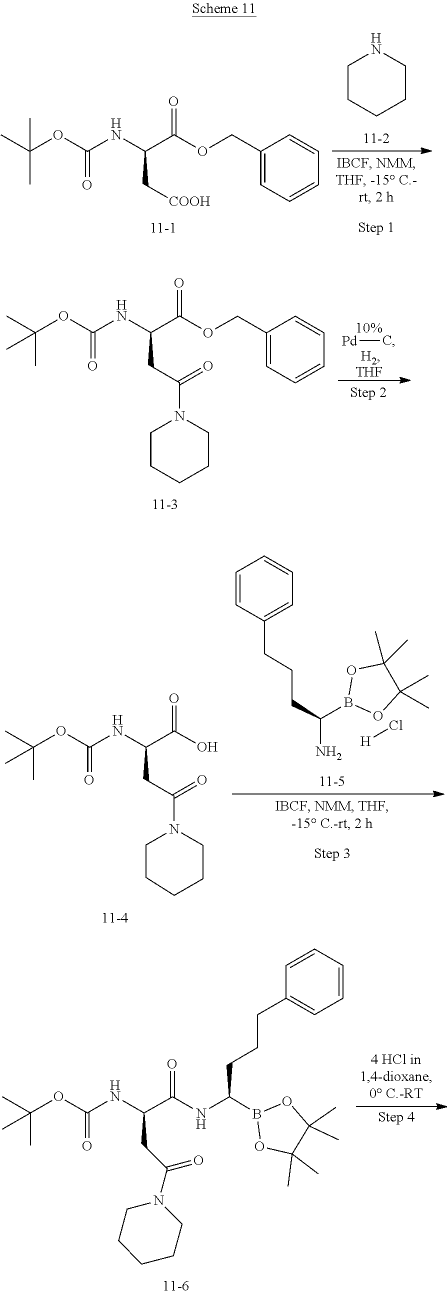

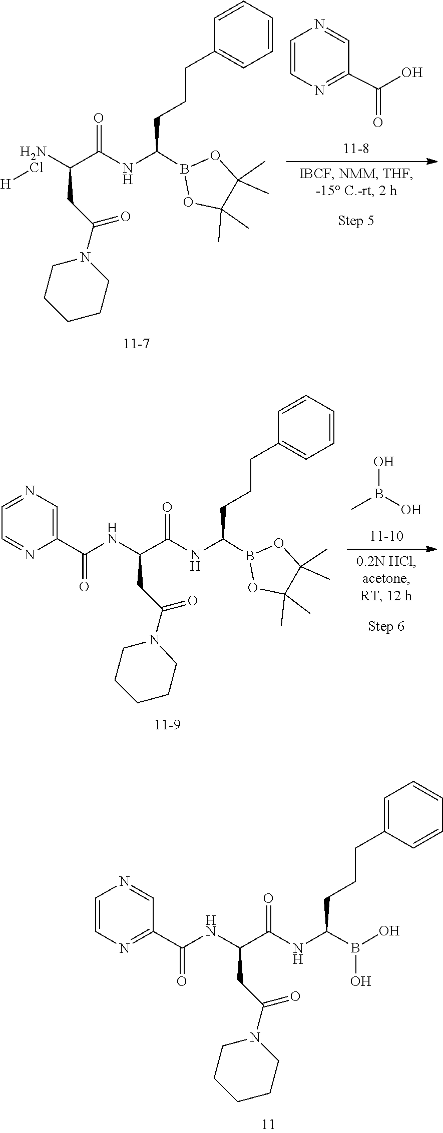

In another embodiment, Compound 19 can be produced from compound (19-1) by the method shown in Scheme 19. Amine (19-1) is coupled with acetic anhydride (19-2) to afford amide (19-3). Subsequent deprotection of (19-3) with methylboronic acid (19-4) produces Compound 9.

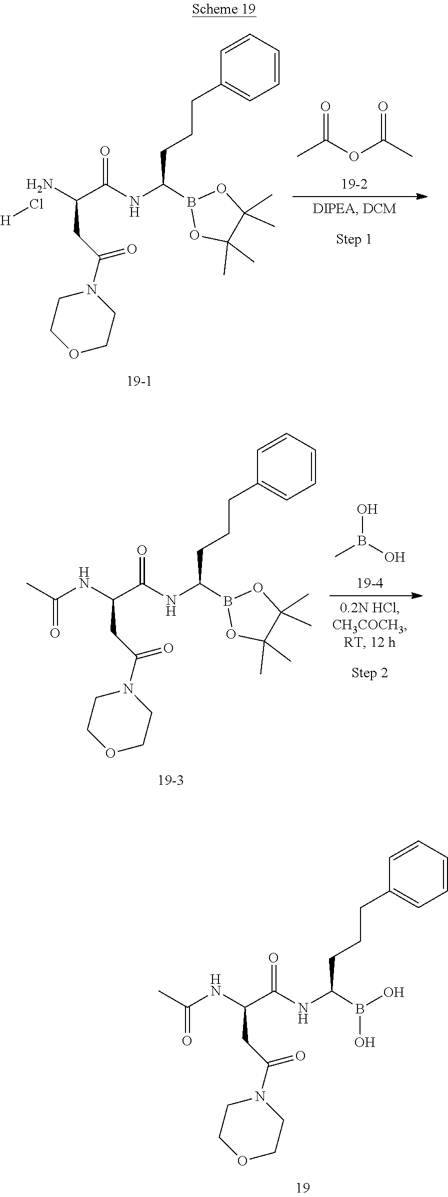

In another embodiment, Compound 20 can be produced from compound (20-1) by the method shown in Scheme 20. Amine (20-1) is coupled with compound (20-2) to afford amide (20-3). Subsequent deprotection of (20-3) with methylboronic acid (20-4) produces Compound 20.

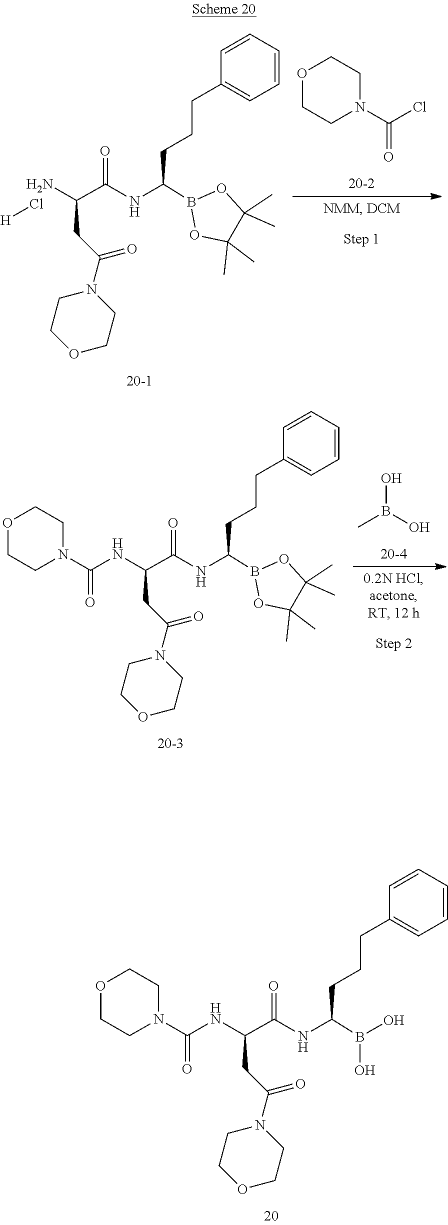

In another embodiment, Compound 21 can be produced from compound (21-1) by the method shown in Scheme 21. Deprotection of (21-1) with a mixture comprising sodium periodate and ammonium acetate produces Compound 21.

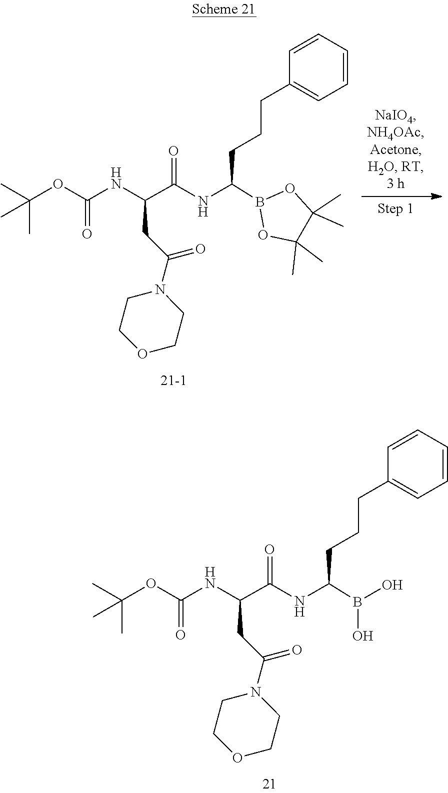 [SMILES: CC(C)(C)OC(=O)N[C@H](CC(=O)N1CCOCC1)C(=O)N[C@@H](CCCc1ccccc1)B1OC(C)(C)C(C)(C)O1 ; CC(C)(C)OC(=O)N[C@H](CC(=O)N1CCOCC1)C(=O)N[C@@H](CCCc1ccccc1)B(O)O]

In another embodiment, Compound 23 can be produced from compound (23-1) by the method shown in Scheme 23. Carboxylic acid (23-1) is coupled morpholine (23-2) to produce amide (23-3), which is then deprotected to generate carboxylic acid (23-4). Subsequent coupling with the amine of a protected boronic acid compound (23-5) produces amide (23-6). Removal of the BOC protecting group yields the corresponding amine (23-7) as the hydrochloride salt. Subsequent coupling with carboxylic acid (23-8) produces amide (23-9). Deprotection of (23-9) with methylboronic acid (23-10) produces Compound 23.

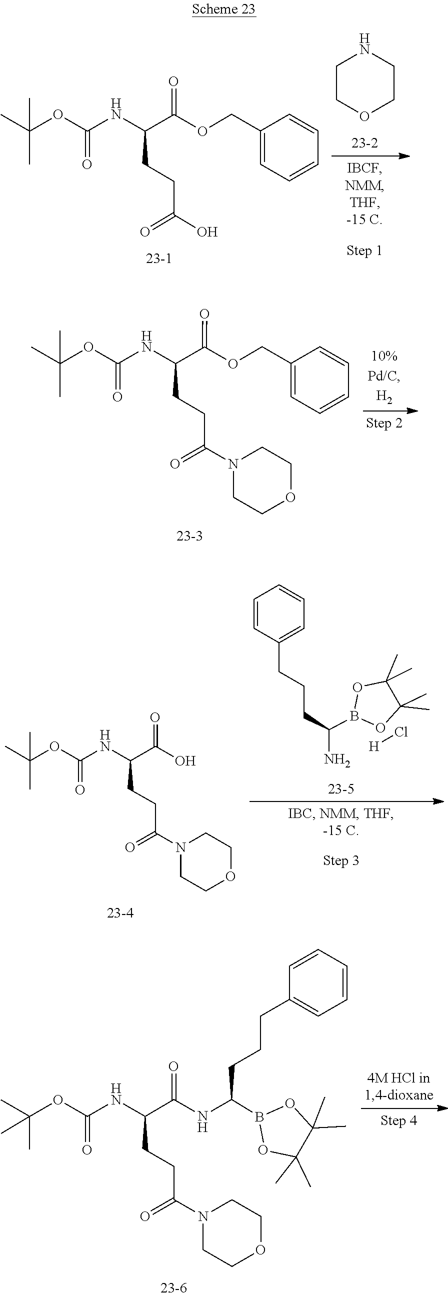

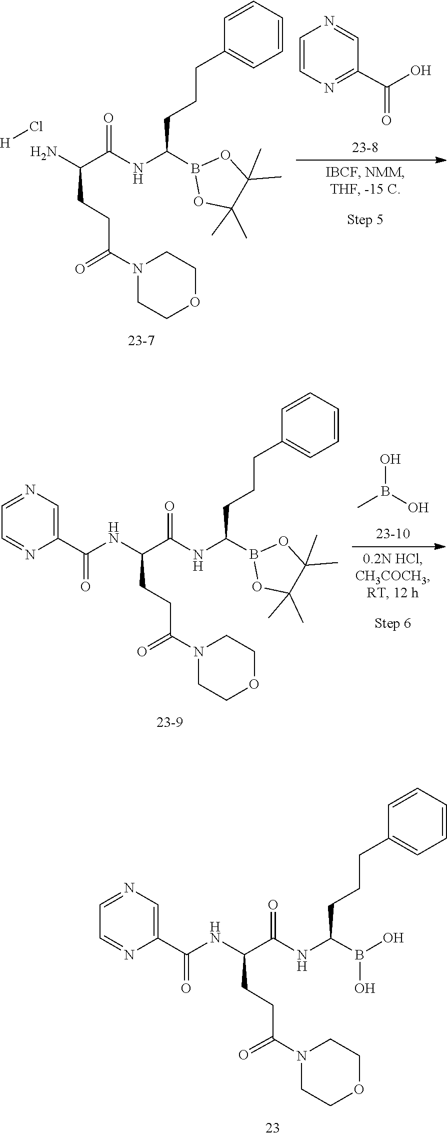

In another embodiment, Compound 31 can be produced from compound (31-1) by the method shown in Scheme 31. Carboxylic acid (31-1) is coupled with the amine of a protected boronic acid compound (31-2) to produce amide (31-3), which is then deprotected to generate amine (31-4) as the hydrochloride salt. Subsequent coupling with carboxylic acid (31-5) produces amide (31-6). Deprotection of (31-6) with methylboronic acid (31-7) produces Compound 31.

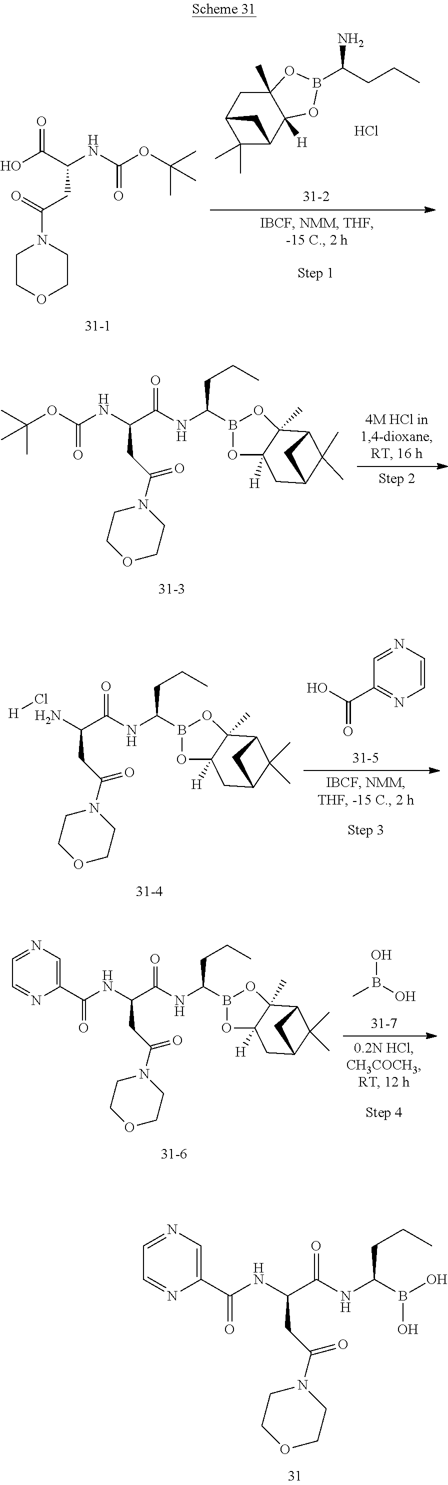

In another embodiment, Compound 32 can be produced from compound (32-1) by the method shown in Scheme 32. Carboxylic acid (32-1) is coupled with the amine of a protected boronic acid compound (32-2) to produce amide (32-3), which is then deprotected to generate amine (32-4) as the hydrochloride salt. Subsequent coupling with carboxylic acid (32-5) produces amide (32-6). Deprotection of (32-6) with methylboronic acid (32-7) produces Compound 32.

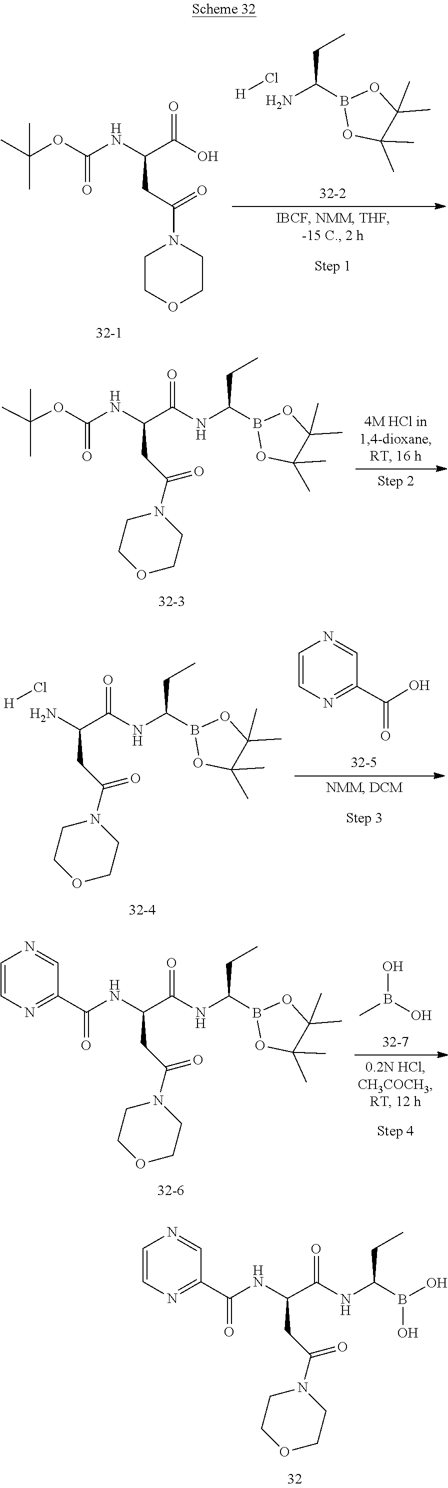

In another embodiment, Compound 33 can be produced from compound (33-1) by the method shown in Scheme 33. Amine (33-1) is coupled with compound (33-2) to afford amide (33-3). Subsequent deprotection of (33-3) with methylboronic acid (33-4) produces Compound 33.

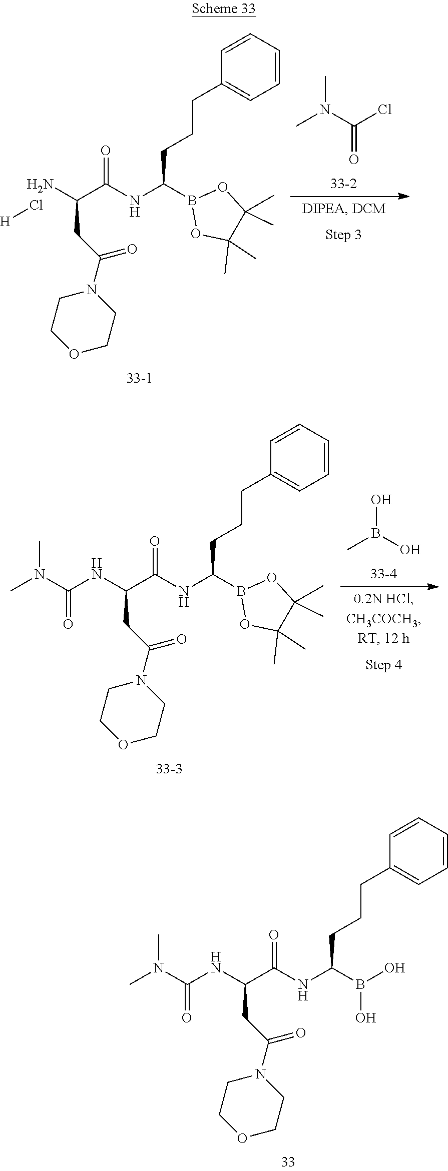

In other embodiments, Compound 37 can be produced from compound (37-1) by the method shown in Scheme 37. Coupling of the carboxylic acid compound (37-1) with the amine of a protected boronic acid compound (37-2) produces amide (37-3). Removal of the BOC protecting group yields the corresponding amine (37-4) as the hydrochloride salt, which is coupled with carboxylic acid (37-5) to form amide (37-6). Deprotection of (37-6) with methylboronic acid (37-7) produces Compound 37.

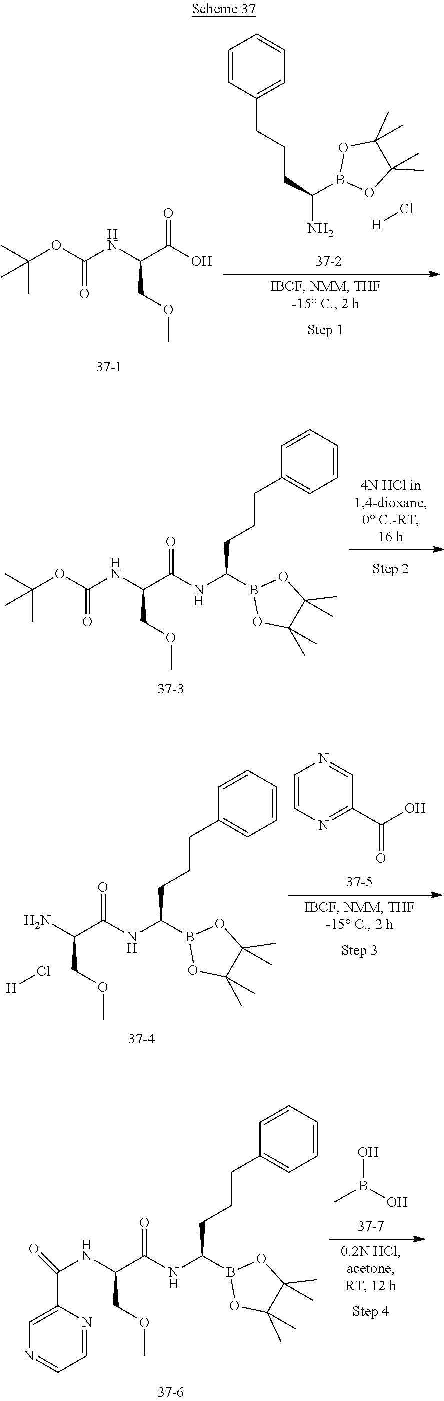

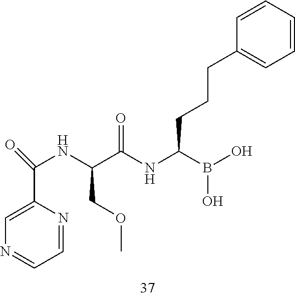 [SMILES: COC[C@@H](NC(=O)c1cnccn1)C(=O)N[C@@H](CCCc1ccccc1)B(O)O]

In another embodiment, Compound 38 can be produced from compound (38-1) by the method shown in Scheme 38. Amino acid (38-1) is coupled with the amine of a protected boronic acid compound (38-2) to produce amide 38-3, which is then deprotected to generate amine (38-4) as the hydrochloride salt. Subsequent coupling with carboxylic acid (38-5) produces amide (38-6). Deprotection of (38-6) with methylboronic acid (38-7) produces Compound 38.

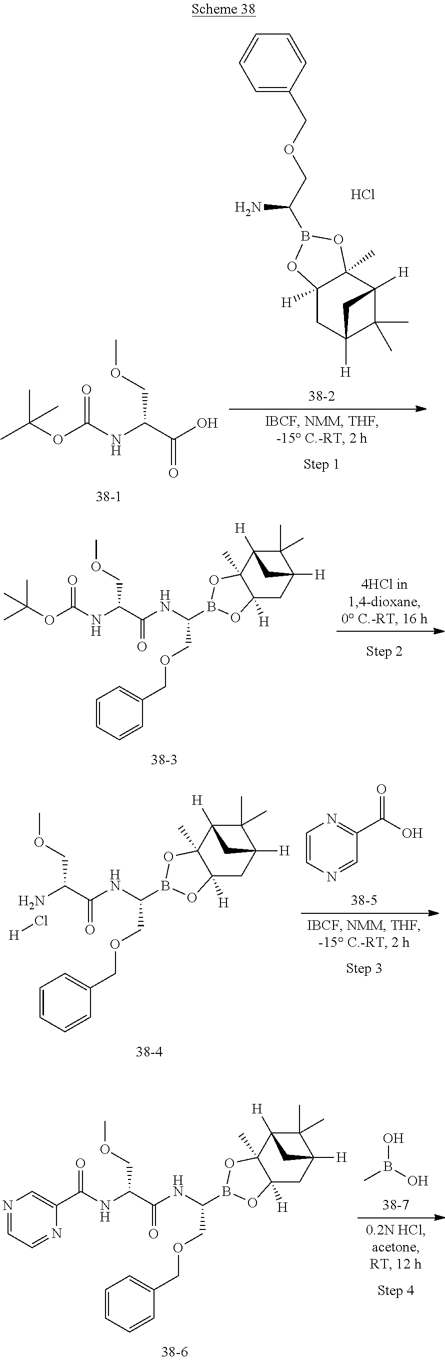

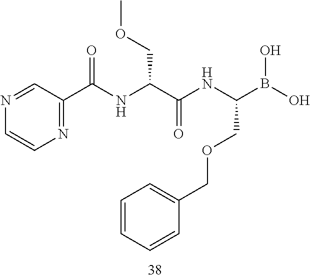 [SMILES: COC[C@@H](NC(=O)c1cnccn1)C(=O)N[C@@H](COCc1ccccc1)B(O)O]

In another embodiment, Compound 39 can be produced from compound (39-1) by the method shown in Scheme 41. Amino acid (39-1) is coupled with the amine of a protected boronic acid compound (39-2) to produce amide (39-3), which is then deprotected to generate amine (39-4) as the hydrochloride salt. Subsequent coupling with carboxylic acid (39-5) produces amide (39-6). Deprotection of (39-6) with methylboronic acid (39-7) produces Compound 39.

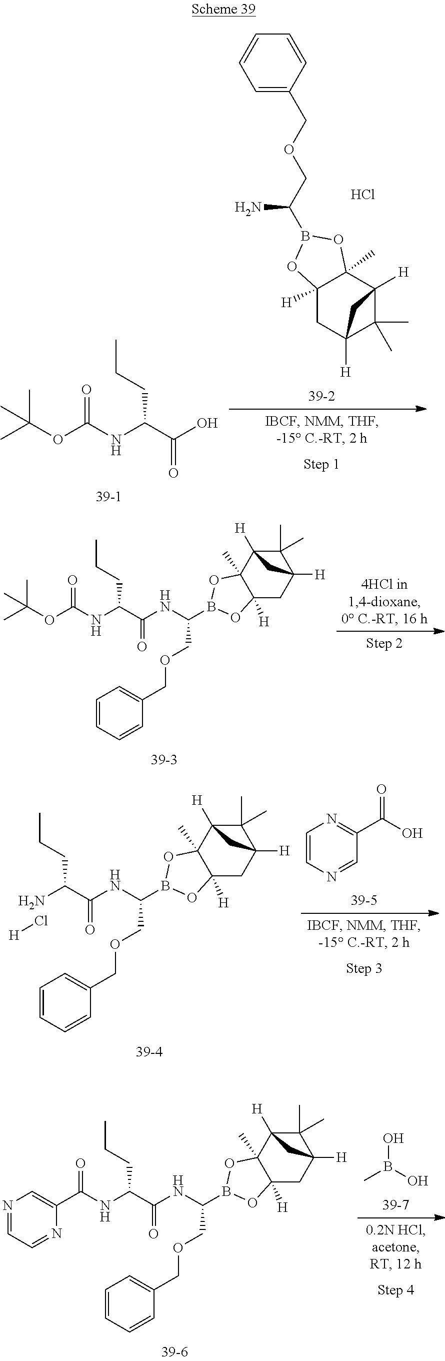

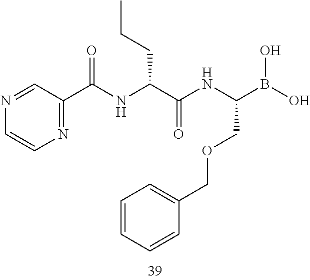 [SMILES: CCC[C@@H](NC(=O)c1cnccn1)C(=O)N[C@@H](COCc1ccccc1)B(O)O]

In another embodiment, Compound 40 can be produced from compound (40-1) by the method shown in Scheme 40. Amino acid (40-1) is coupled with the amine of a protected boronic acid compound (40-2) to produce amide (40-3), which is then deprotected to generate amine (40-4) as the hydrochloride salt. Subsequent coupling with carboxylic acid (40-5) produces amide (40-6). Deprotection of (40-6) with methylboronic acid (40-7) produces Compound 40.

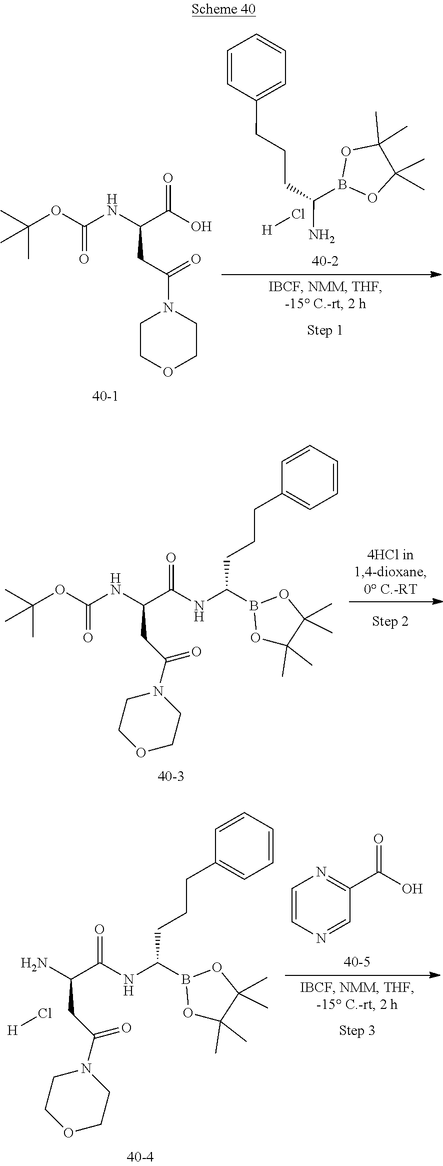

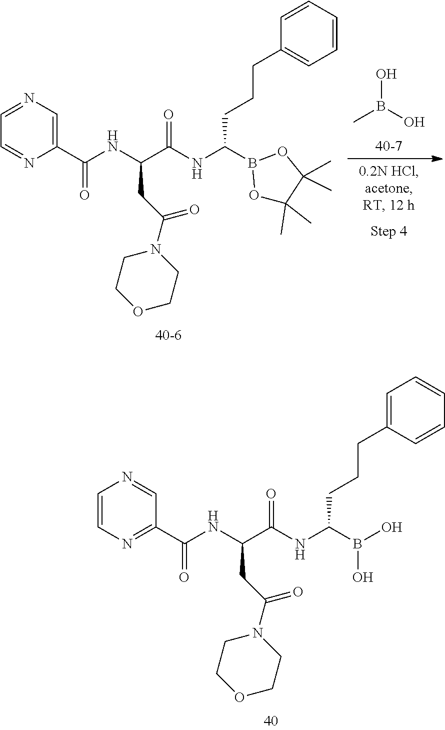

In another embodiment, Compound 41 can be produced from compound (41-1) by the method shown in Scheme 41. Amine (41-1) is coupled with compound (41-2) to afford amide (41-3). Subsequent deprotection of (41-3) with methylboronic acid (41-4) produces Compound 41.

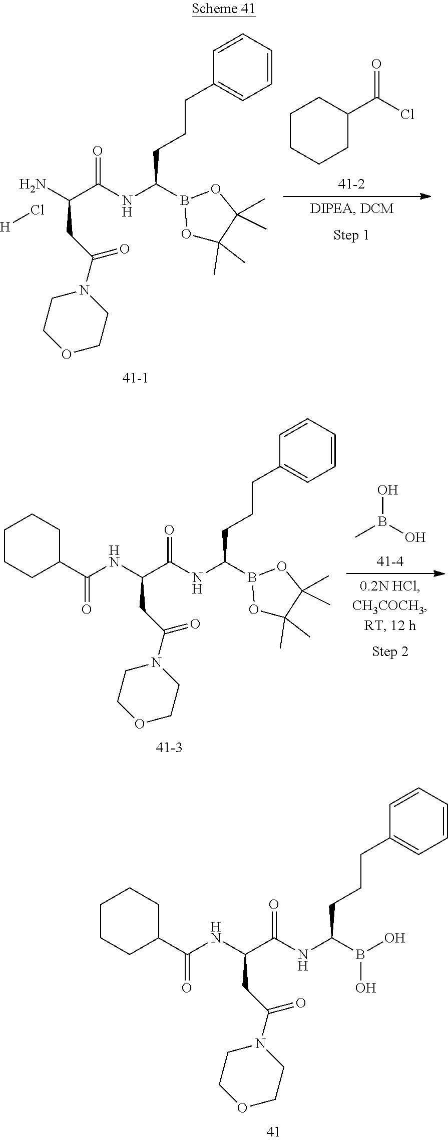

In another embodiment, Compound 42 can be produced from compound (42-1) by the method shown in Scheme 42. Amine (42-1) is coupled with compound (42-2) to afford amide (42-3).

Subsequent deprotection of (42-3) with methylboronic acid (42-4) produces Compound 42.

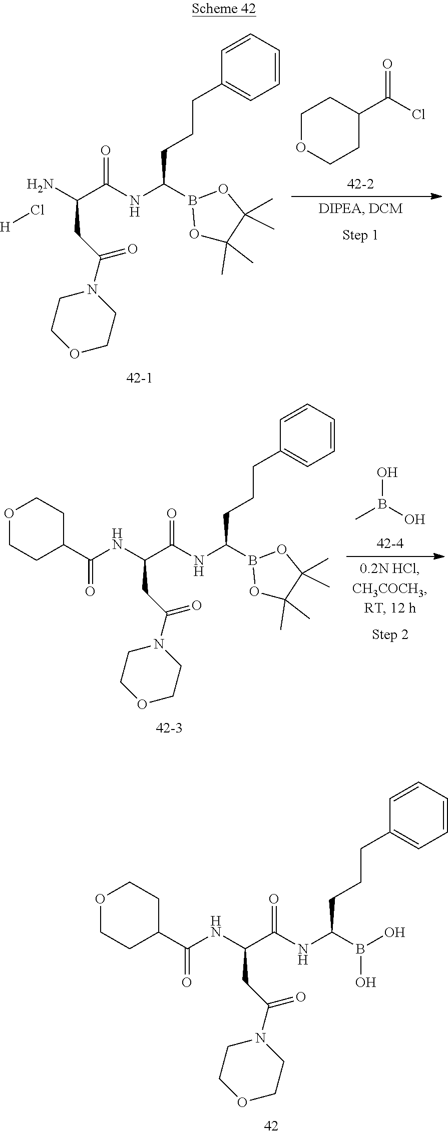

In another embodiment, Compound 43 can be produced from compound (43-1) by the method shown in Scheme 43. Amine (43-1) is coupled with compound (43-2) to afford sulfonamide (43-3). Subsequent deprotection of (43-3) with methylboronic acid (43-4) produces Formula 43.

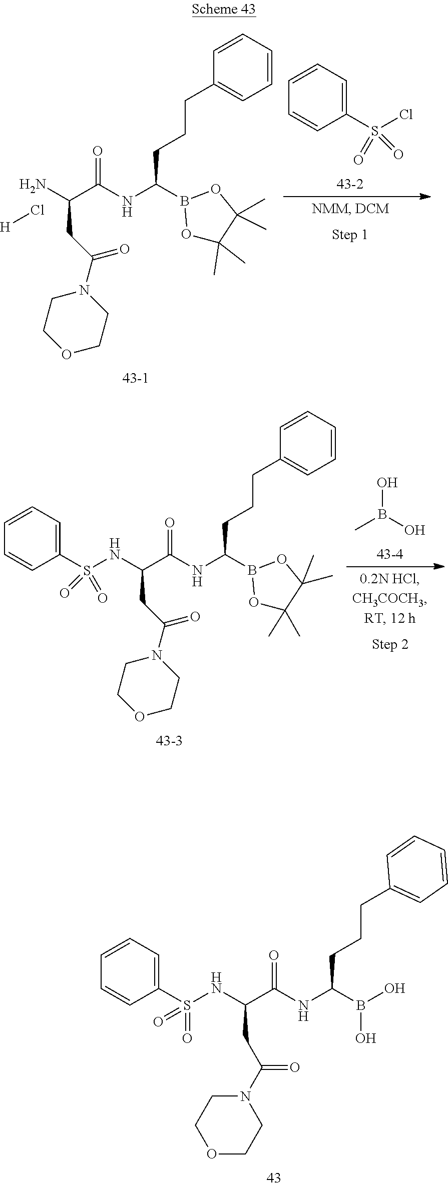

In another embodiment, Compound 44 can be produced from compound (44-1) by the method shown in Scheme 44. Amine (44-1) is coupled with compound (44-2) to afford amide (44-3). Subsequent deprotection of (44-3) with methylboronic acid (44-4) produces Compound 44.

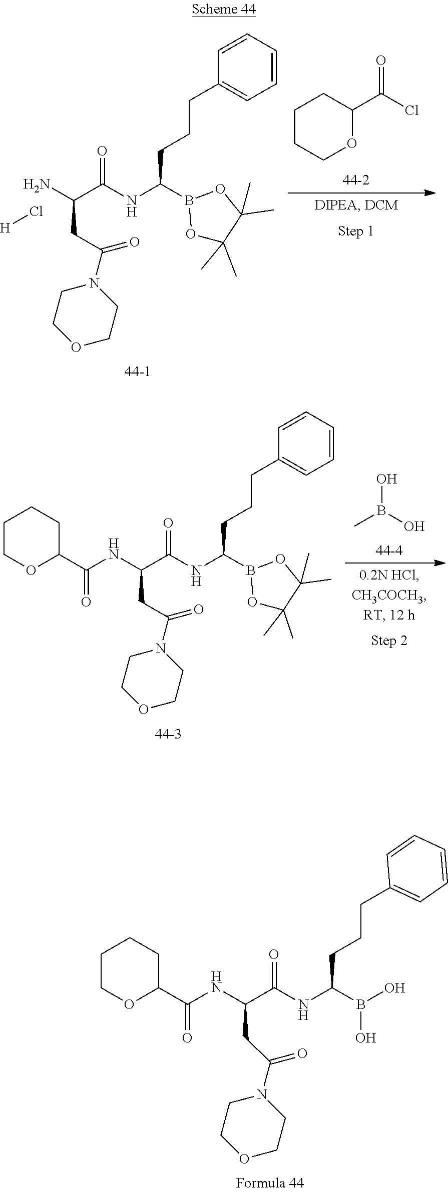

In another embodiment, Compound 45 can be produced from compound (45-1) by the method shown in Scheme 45. Amine (45-1) is coupled with morpholine to produce amide (45-3), which is deprotected to produce carboxylic acid (45-4), and then coupled with the amine of a protected boronic acid compound (45-5) to produce amide (45-6). Amide (45-6) is deprotected to generate amine (45-7) as the hydrochloride salt. Subsequent coupling with carboxylic acid (45-8) produces amide (45-9). Deprotection of (45-9) with methylboronic acid (45-10) produces Compound 45.

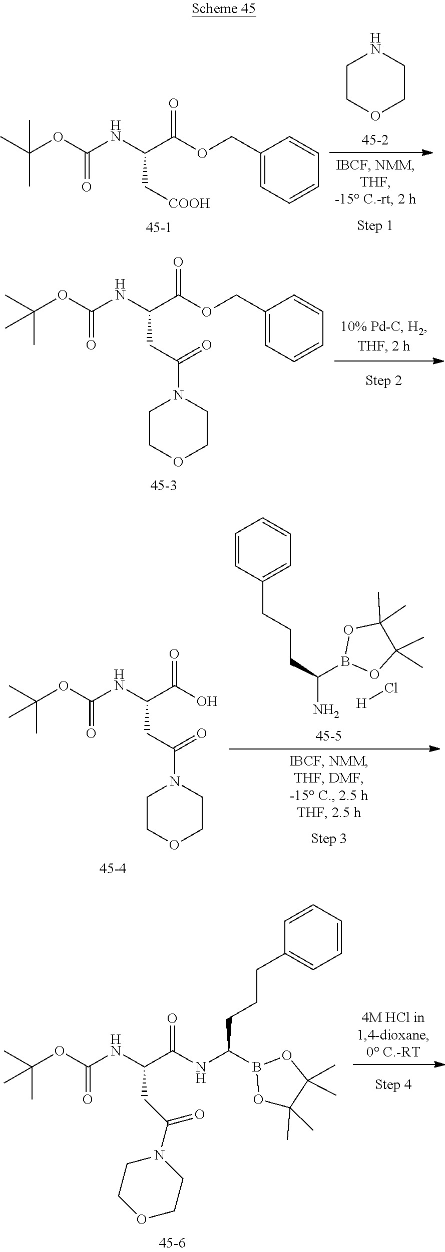

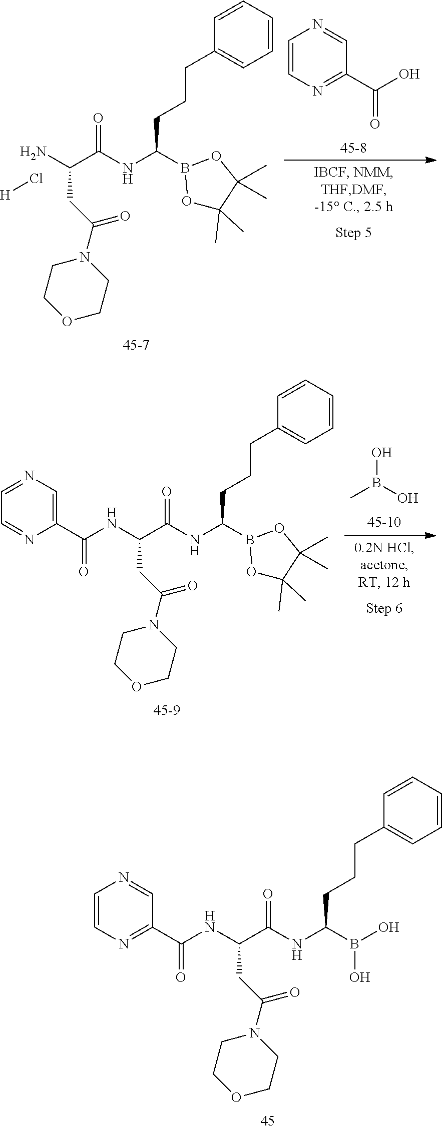

In another embodiment, Compound 46 can be produced from compound (46-1) by the method shown in Scheme 46. Amino acid (46-1) is coupled with the amine of a protected boronic acid compound (46-2) to produce amide (46-3), which is then deprotected to generate amine (46-4) as the hydrochloride salt. Subsequent coupling with carboxylic acid (46-5) produces amide (46-6). Deprotection of (46-6) with methylboronic acid (46-7) produces Compound 46.

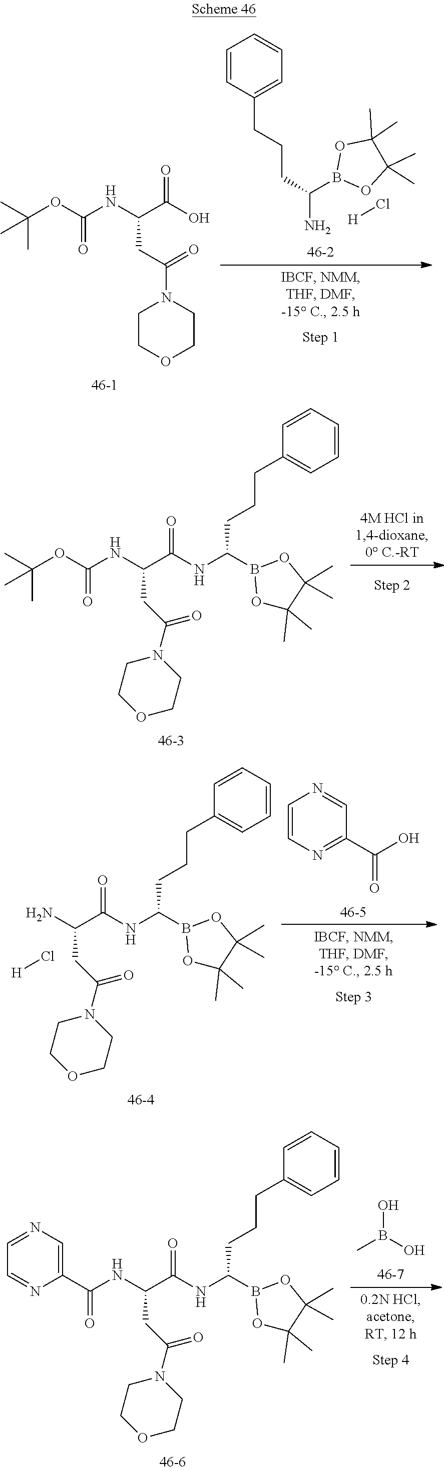

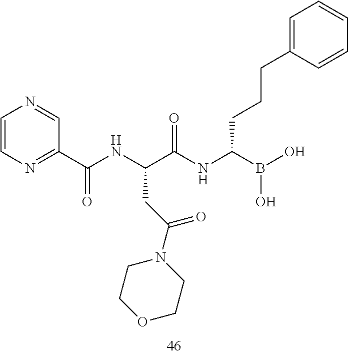 [SMILES: O=C(N[C@@H](CC(=O)N1CCOCC1)C(=O)N[C@H](CCCc1ccccc1)B(O)O)c1cnccn1]

### Dosage Forms, Medicaments and Pharmaceuticals:

The compounds, molecules or agents of the disclosure may be used to treat (e.g. cure, alleviate or prevent) one or more diseases, infections or disorders. Thus, in accordance with the disclosure, the compounds and molecules may be manufactured into medicaments or may be incorporated or formulated into pharmaceutical compositions.

The molecules, compounds and compositions of the disclosure may be administered by any convenient route, for example, methods of administration include intradermal, intramuscular, intraperitoneal, intravenous, subcutaneous, intranasal, epidural, oral, sublingual, intranasal, intravaginal, transdermal, rectally, by inhalation, or topically to the skin. Delivery systems are also known to include, for example, encapsulation in liposomes, microgels, microparticles, microcapsules, capsules, etc. Any other suitable delivery system known in the art is also envisioned in use. Administration can be systemic or local. The mode of administration may be left to the discretion of the practitioner.

The dosage administered will, of course, vary depending upon known factors, such as the pharmacodynamic properties of the particular active agent; the chosen mode and route of administration; the age, health and weight of the recipient; the nature of the disease or disorder to be treated; the extent of the symptoms; any simultaneous or concurrent treatments; the frequency of treatment; and the effect desired.

Depending on known factors, such as those noted above, the required dosage of the active agent may be administered in a single daily dose, or the total daily dosage may be administered in divided doses of e.g. two, three, or four times daily. Suitably, the therapeutic treatment regime according to the disclosure is devised for a single daily dose or for a divided daily dose of two doses.

The ‘effective amount’ or ‘therapeutically effective amount’ is meant to describe an amount of compound or a composition of the disclosure that is effective in curing, inhibiting, alleviating, reducing or preventing the adverse effects of the diseases or disorders to be treated, or the amount necessary to achieve a physiological or biochemically-detectable effect. Thus, at the effective amount, the compound or agent is able to produce the desired therapeutic, ameliorative, inhibitory or preventative effect in relation to disease or disorder. Beneficially, an effective amount of the compound or composition of the disclosure may have the effect of inhibiting CDK2. Diseases or disorders which may benefit from CDK2 inhibition include, for example, proliferative diseases or disorders and cancer.

The ‘effective amount’ or ‘therapeutically effective amount’ is meant to describe an amount of compound or a composition of the disclosure that is effective in curing, inhibiting, alleviating, reducing or preventing the adverse effects of the diseases or disorders to be treated, or the amount necessary to achieve a physiological or biochemically-detectable effect. Thus, at the effective amount, the compound or agent is able to produce the desired therapeutic, ameliorative, inhibitory or preventative effect in relation to disease or disorder. Beneficially, an effective amount of the compound or composition of the disclosure may have the effect of inhibiting LONP1. Diseases or disorders which may benefit from LONP1 inhibition include, for example, proliferative diseases or disorders and cancer.

When administered to a subject, a compound of the disclosure is suitably administered as a component of a composition that comprises a pharmaceutically acceptable carrier or vehicle. One or more additional pharmaceutical acceptable carrier (such as diluents, adjuvants, excipients or vehicles) may be combined with the compound of the disclosure in a pharmaceutical composition. Suitable pharmaceutical carriers are described in “Remington's Pharmaceutical Sciences” by E. W. Martin. Pharmaceutical formulations and compositions of the disclosure are formulated to conform to regulatory standards and according to the chosen route of administration.

Acceptable pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilising, thickening, lubricating and colouring agents may be used. When administered to a subject, the pharmaceutically acceptable vehicles are generally sterile. Water is a suitable vehicle when the compound is to be administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or buffering agents.

The medicaments and pharmaceutical compositions of the disclosure can take the form of solutions, suspensions, emulsion, tablets, pills, pellets, powders, gels, capsules (for example, capsules containing liquids or powders), modified-release formulations (such as slow or sustained-release formulations), suppositories, emulsions, aerosols, sprays, suspensions, or any other form suitable for use. Other examples of suitable pharmaceutical vehicles are described in Remington's Pharmaceutical Sciences, Alfonso R. Gennaro ed., Mack Publishing Co. Easton, Pa., 19th ed., 1995, see for example pages 1447-1676.

Suitably, the therapeutic compositions or medicaments of the disclosure are formulated in accordance with routine procedures as a pharmaceutical composition adapted for oral administration (more suitably for humans). Compositions for oral delivery may be in the form of tablets, lozenges, aqueous or oily suspensions, granules, powders, emulsions, capsules, syrups, or elixirs, for example. Thus, in one embodiment, the pharmaceutically acceptable vehicle is a capsule, tablet or pill.

Orally administered compositions may contain one or more agents, for example, sweetening agents such as fructose, aspartame or saccharin; flavouring agents such as peppermint, oil of wintergreen, or cherry; colouring agents; and preserving agents, to provide a pharmaceutically palatable preparation. When the composition is in the form of a tablet or pill, the compositions may be coated to delay disintegration and absorption in the gastrointestinal tract, so as to provide a sustained release of active agent over an extended period of time. Selectively permeable membranes surrounding an osmotically active driving compound are also suitable for orally administered compositions. In these dosage forms, fluid from the environment surrounding the capsule is imbibed by the driving compound, which swells to displace the agent or agent composition through an aperture. These dosage forms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade. For oral formulations, the location of release may be the stomach, the small intestine (the duodenum, the jejunum, or the ileum), or the large intestine. One skilled in the art is able to prepare formulations that will not dissolve in the stomach yet will release the material in the duodenum or elsewhere in the intestine. Suitably, the release will avoid the deleterious effects of the stomach environment, either by protection of the compound (or composition) or by release of the compound (or composition) beyond the stomach environment, such as in the intestine. To ensure full gastric resistance a coating impermeable to at least pH 5.0 would be essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac, which may be used as mixed films.

While it can be beneficial to provide therapeutic compositions and/or compounds of the disclosure in a form suitable for oral administration, for example, to improve patient compliance and for ease of administration, in some embodiments, compounds or compositions of the disclosure may cause undesirable side-effects, such as intestinal inflammation which may lead to premature termination of a therapeutic treatment regime. Thus, in some embodiments, the therapeutic treatment regime is adapted to accommodate ‘treatment holidays’, e.g. one or more days of non-administration. For example, treatment regimens and therapeutic methods of the disclosure may comprise a repetitive process comprising administration of the therapeutic composition or compound for a number of consecutive days, followed by a treatment holiday of one or more consecutive days. For example, a treatment regime of the disclosure may comprise a repetitive cycle of administration of the therapeutic composition or compound for between 1 and 49 consecutive days, between 2 and 42 days, between 3 and 35 days, between 4 and 28 days, between 5 and 21 days, between 6 and 14 days, or between 7 and 10 days; followed by a treatment holiday of between 1 and 14 consecutive days, between 1 and 12 days, between 1 and 10 days, or between 1 and 7 days (e.g. 1, 2, 3, 4, 5, 6 or 7 days).

To aid dissolution of the therapeutic agent into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. Potential nonionic detergents that could be included in the formulation as surfactants include: lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose and carboxymethyl cellulose. These surfactants, when used, could be present in the formulation of the compound or derivative either alone or as a mixture in different ratios.

Typically, compositions for intravenous administration comprise sterile isotonic aqueous buffer. Where necessary, the compositions may also include a solubilising agent.

Another suitable route of administration for the therapeutic compositions of the disclosure is via pulmonary or nasal delivery.

Additives may be included to enhance cellular uptake of the therapeutic agent of the disclosure, such as the fatty acids oleic acid, linoleic acid and linolenic acid.

The therapeutic agents of the disclosure may also be formulated into compositions for topical application to the skin of a subject.

Where the disclosure provides more than one active compound/agent for use in combination, generally, the agents may be formulated separately or in a single dosage form, depending on the prescribed most suitable administration regime for each of the agents concerned. When the therapeutic agents are formulated separately, the pharmaceutical compositions of the disclosure may be used in a treatment regime involving simultaneous, separate or sequential administration with the other one or more therapeutic agent. The other therapeutic agent(s) may comprise a compound of the disclosure or a therapeutic agent known in the art.

Specific and general embodiments of the disclosure will now be described by way of the following non-limiting examples.

In embodiments, a pharmaceutical composition of this invention further comprises a second therapeutic agent. The second therapeutic agent may be selected from any pharmaceutically active compound; preferably the second therapeutic agent is known to correct mitochondrial dysfunction. Alternatively, the compounds of the invention and second therapeutic agent may be administered together (within less than 24 hours of one another, consecutively or simultaneously) but in separate pharmaceutical compositions. If the second therapeutic agent acts synergistically with the compounds of this invention, the therapeutically effective amount of such compounds and/or the second therapeutic agent may be less than such amount required when either is administered alone.

### EXAMPLES

The examples and preparations provided below further illustrate and exemplify the compounds of the present invention and methods of preparing such compounds. It is to be understood that the scope of the present invention is not limited in any way by the scope of the following examples and preparations.

The structures of the compounds are confirmed by either elemental analysis or NMR, where peaks assigned to the characteristic protons in the title compound are presented where appropriate. ¹H NMR shift (OH) are given in parts per million (ppm) down field from an internal reference standard.

### Example 1: Synthesis of ((R)-1-((R)-4-morpholino-4-oxo-2-(pyrazine-2-carboxamido) butanamido)-4-phenylbutyl)boronic acid

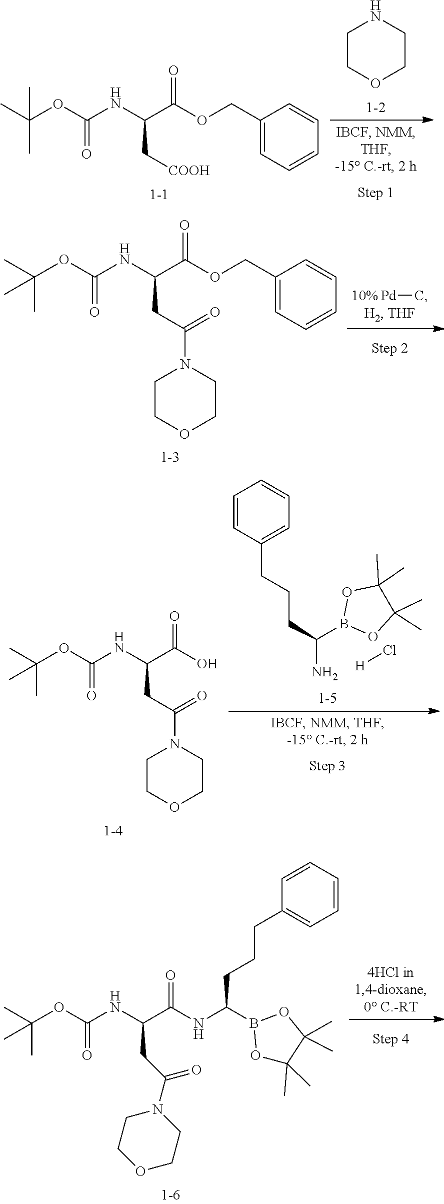

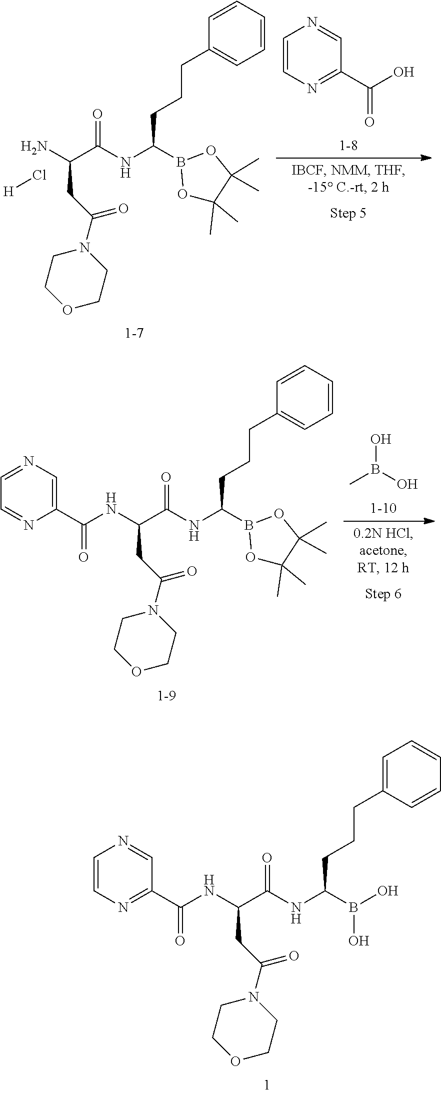

Synthesis of benzyl (R)-2-((tert-butoxycarbonyl)amino)-4-morpholino-4-oxobutanoate, [Step 1]: To a stirred solution of (R)-4-(benzyloxy)-3-((tert-butoxycarbonyl)amino)-4-oxobutanoic acid (1-1, 2.0 g, 6.3 mmol) in tetrahydrofuran (25 mL) was added isobutyl chloroformate (IBCF) (0.8 mL, 6.3 mmol) and N-methylmorpholine (NMM) (0.7 mL, 6.3 mmol) at −15° C. The reaction mixture was stirred at the same temperature for 30 minutes. Then morpholine (1-2, 0.5 mL, 5.7 mmol) followed by NMM (0.6 mL, 5.7 mmol) was added to the reaction mixture at −15° C., which was gradually warmed to 0° C. and stirred for 2 hours. LCMS of the reaction mass confirmed the formation of the desired product. The reaction mixture was neutralized with aqueous 0.1 N HCl solution and extracted with ethyl acetate several times. The organic layers were combined and washed with a 5% potassium carbonate solution, water, brine, dried over sodium sulfate, filtered, and concentrated under reduced pressure to obtain the product. The product was purified through combiflash column chromatography to afford benzyl (R)-2-((tert-butoxycarbonyl)amino)-4-morpholino-4-oxobutanoate (1-3, 2.0 g). LCMS (ESI) 1 [M+H]⁺=393.

Synthesis of (R)-2-((tert-butoxycarbonyl)amino)-4-morpholino-4-oxobutanoic acid, [Step 2]: To a stirred solution of benzyl (R)-2-((tert-butoxycarbonyl)amino)-4-morpholino-4-oxobutanoate (1-3, 2.0 g, 5.1 mmol) in tetrahydrofuran (50 mL) was added nitrogen gas for 10 minutes. Then 10% Pd—C(400 mg, 3.7 mmol) was added and the reaction mixture was hydrogenated under a hydrogen balloon for 3 hours. The reaction was monitored by TLC, and upon completion, the reaction mixture was filtered over celite using excess ethyl acetate. The solvent was removed by concentration under reduced pressure and provided (R)-2-((tert-butoxycarbonyl)amino)-4-morpholino-4-oxobutanoic acid (1-4, 1.5 g). [M−H]⁺=301.

Synthesis of tert-butyl ((R)-4-morpholino-1, 4-dioxo-1-(((R)-4-phenyl-1-(4, 4, 5, 5-tetramethyl-1, 3, 2-dioxaborolan-2-yl)butyl)amino)butan-2-yl)carbamate, [Step 3]: To a stirred solution of (R)-2-((tert-butoxycarbonyl)amino)-4-morpholino-4-oxobutanoic acid (1-4, 450 mg, 1.5 mmol) in tetrahydrofuran (8 mL) was added isobutyl chloroformate (IBCF) (0.2 mL, 1.5 mmol) and N-methylmorpholine (NMM) (0.2 mL, 1.5 mmol) at −15° C. The reaction mixture was stirred at the same temperature for 30 minutes. Then (R)-4-phenyl-1-(4, 4, 5, 5-tetramethyl-1, 3, 2-dioxaborolan-2-yl)butan-1-amine hydrochloride (1-5, 420 mg, 1.4 mmol) in dimethylformamide (1 mL) was added to the reaction mixture followed by NMM (0.15 mL, 1.4 mmol) at −15° C. The reaction mixture was gradually warmed to 0° C. and stirred for 2 hours. LCMS of the reaction mass confirmed the formation of the desired product, and the reaction mixture was neutralized with an aqueous solution of 0.1 N HCl and extracted with ethyl acetate. The organic layers were combined and washed with 5% potassium carbonate solution, water, brine, dried over sodium sulfate, filtered, and concentrated under reduced pressure to afford tert-butyl ((R)-4-morpholino-1, 4-dioxo-1-(((R)-4-phenyl-1-(4, 4, 5, 5-tetramethyl-1, 3, 2-dioxaborolan-2-yl)butyl)amino)butan-2-yl)carbamate (1-6, 635 mg). 1 [M−H]: 558.

Synthesis of (R)-2-amino-4-morpholino-4-oxo-N—((R)-4-phenyl-1-(4, 4, 5, 5-tetramethyl-1, 3, 2-dioxaborolan-2-yl)butyl)butanamide hydrochloride, [Step 4]: To a solution of tert-butyl ((R)-4-morpholino-1, 4-dioxo-1-(((R)-4-phenyl-1-(4, 4, 5, 5-tetramethyl-1, 3, 2-dioxaborolan-2-yl)butyl)amino)butan-2-yl)carbamate (1-6, 635 mg, 1.1 mmol) was added 4 M HCl in dioxane (3.0 mL, 11.0 mmol) at 0° C. The reaction mixture was gradually warmed to ambient temperature and stirred for 16 hours. TLC showed complete consumption of starting material and the reaction mixture was concentrated under reduced pressure to obtain (R)-2-amino-4-morpholino-4-oxo-N—((R)-4-phenyl-1-(4, 4, 5, 5-tetramethyl-1, 3, 2-dioxaborolan-2-yl)butyl)butanamide hydrochloride (1-7, 560 mg). The product was used without purification.

Synthesis of N—((R)-4-morpholino-1, 4-dioxo-1-(((R)-4-phenyl-1-(4, 4, 5, 5-tetramethyl-1, 3, 2-dioxaborolan-2-yl)butyl)amino)butan-2-yl) pyrazine-2-carboxamide, [Step 5]: To a stirred solution of pyrazine-2-carboxylic acid (1-8, 155 mg, 1.3 mmol) in tetrahydrofuran (8 mL) was added isobutyl chloroformate (IBCF) (0.2 mL, 1.3 mmol) and N-methylmorpholine (NMM) (0.15 mL, 1.3 mmol) at −15° C. The reaction mixture was stirred at the same temperature for 30 minutes. Then (R)-2-amino-4-morpholino-4-oxo-N—((R)-4-phenyl-1-(4, 4, 5, 5-tetramethyl-1, 3, 2-dioxaborolan-2-yl)butyl)butanamide hydrochloride (1-7, 560 mg, 1.1 mmol) in dimethylformamide (1 mL) was added to the reaction mixture, followed by NMM (0.1 mL, 1.1 mmol). The reaction mixture was gradually warmed to 0° C. and stirred for 2 hours. LCMS of the reaction mixture confirmed formation of the product, and the reaction mixture was neutralized with aqueous 0.1 N HCl solution and extracted with ethyl acetate. The organic layers were combined and washed with 5% potassium carbonate solution, water, brine, dried over sodium sulfate, filtered and evaporated under reduced pressure. The product was purified by reverse-phase prep-HPLC and lyophilized to afford N—((R)-4-morpholino-1, 4-dioxo-1-(((R)-4-phenyl-1-(4, 4, 5, 5-tetramethyl-1, 3, 2-dioxaborolan-2-yl)butyl)amino)butan-2-yl) pyrazine-2-carboxamide (1-9, 45 mg). 1 [M−H]⁺=564. ¹H NMR (400 MHZ, MeOD) δ 9.26 (br s, 1H), 8.82-8.81 (m, 1H), 8.70 (br s, 1H), 7.21-7.11 (m, 5H), 5.30 (br s, 1H), 3.68-3.52 (m, 8H), 3.31-3.27 (m, 1H), 3.05-2.90 (m, 1H), 2.62-2.59 (m, 3H), 1.68-1.35 (m, 4H), 1.18-1.17 (m, 4H).

Synthesis of ((R)-1-((R)-4-morpholino-4-oxo-2-(pyrazine-2-carboxamido)butanamido)-4-phenylbutyl)boronic acid, [Step 6]: To a stirred solution of N—((R)-4-morpholino-1, 4-dioxo-1-(((R)-4-phenyl-1-(4, 4, 5, 5-tetramethyl-1, 3, 2-dioxaborolan-2-yl)butyl)amino)butan-2-yl) pyrazine-2-carboxamide (1-9, 45 mg, 0.1 mmol) and methylboronic acid (1-10, 50 mg, 0.8 mmol) in acetone (2 mL) was added 0.2 N HCl (2 mL) and the reaction mixture was stirred at ambient temperature overnight. TLC and LCMS showed the complete disappearance of the starting material and the reaction mixture was concentrated under reduced pressure. The product was redissolved in a mixture of acetone and deionized water, and lyophilized to obtain ((R)-1-((R)-4-morpholino-4-oxo-2-(pyrazine-2-carboxamido)butanamido)-4-phenylbutyl)boronic acid (Compound 1, 38 mg). [M−H]⁺=482. ¹H NMR (400 MHZ, DMSO-d₆. D₂O exchange) δ 9.15 (s, 1H), 8.84 (d, 1H), 8.71 (br s, 1H), 7.20-7.09 (m, 5H), 4.80 (t, 1H), 3.52-3.36 (m, 8H), 3.13-2.85 (m, 3H), 2.75-2.65 (m, 1H), 1.46-1.44 (m, 5H).

### Example 2: ((R)-1-((R)-4-oxo-2-(pyrazine-2-carboxamido)-4-(pyrrolidin-1-yl)butanamido)-4-phenylbutyl)boronic acid

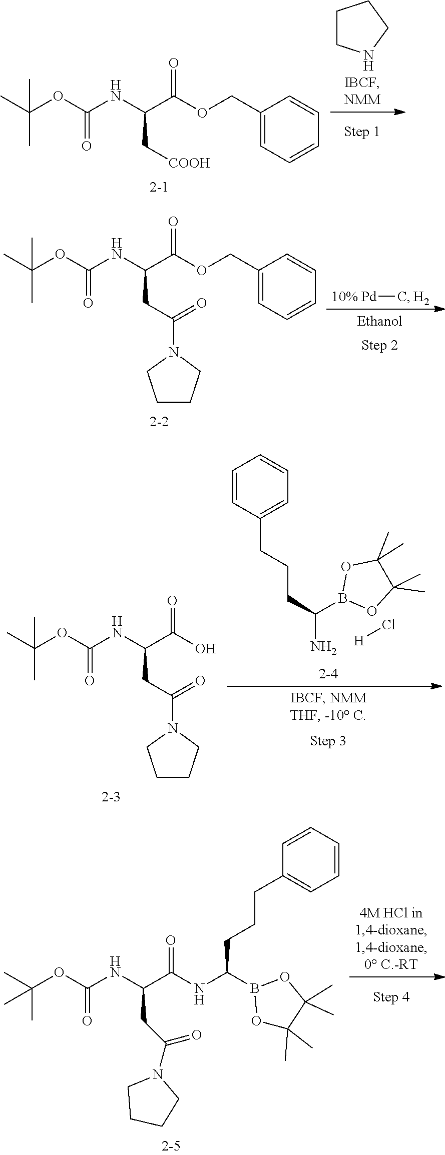

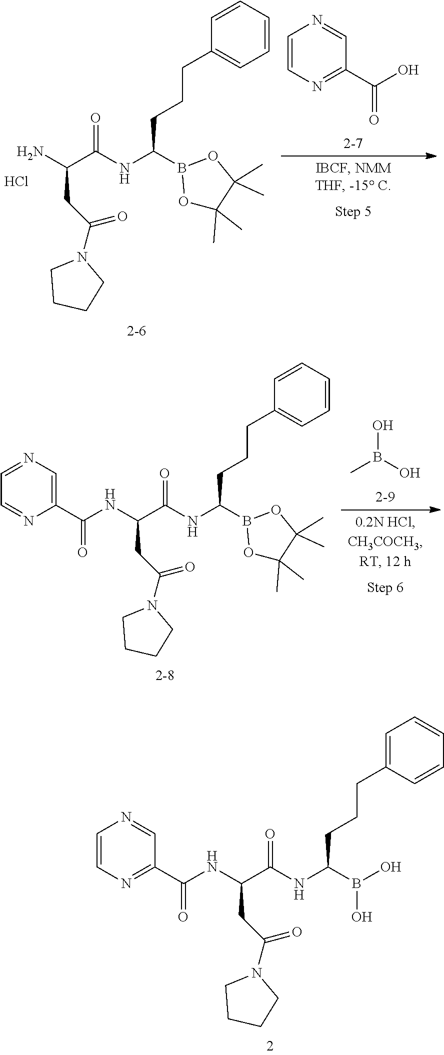

Synthesis of benzyl (R)-2-((tert-butoxycarbonyl)amino)-4-oxo-4-(pyrrolidin-1-yl)butanoate, [Step 1]: To a solution of (R)-4-(benzyloxy)-3-((tert-butoxycarbonyl)amino)-4-oxobutanoic acid (2-1, 3.5 g, 11.0 mmol) in tetrahydrofuran (20 mL) was added isobutyl chloroformate (IBCF) (1.5 mL, 11.0 mmol) and N-methylmorpholine (NMM) (1.6 mL, 12.0 mmol) dropwise at −10° C. The reaction mixture was stirred at the same temperature for 30 minutes. Then pyrrolidine (0.8 mL, 9.8 mmol) and NMM (1.6 mL, 11.8 mmol) was added to the reaction mixture at −10° C., which was gradually warmed to ambient temperature and stirred over 2 hours. After the reaction was completed, as measure by LCMS, the reaction mixture was diluted with ethyl acetate and the organic layer was washed with aqueous 0.1 N HCl (twice), 10% aqueous potassium carbonate (twice), water (twice), brine (twice), dried over sodium sulfate, and concentrated under reduced pressure at ambient temperature. The product was purified by column chromatography using 20% ethyl acetate in hexane as eluent to afford benzyl (R)-2-((tert-butoxycarbonyl)amino)-4-oxo-4-(pyrrolidin-1-yl)butanoate (2-2, 3300 mg). [M+H]⁺=377.

Synthesis of (R)-2-((tert-butoxycarbonyl)amino)-4-oxo-4-(pyrrolidin-1-yl)butanoic acid, [Step 2]: A 2-necked round-bottom flask was purged with nitrogen, and a solution of benzyl (R)-2-((tert-butoxycarbonyl)amino)-4-oxo-4-(pyrrolidin-1-yl)butanoate (2-2, 3.3 g, 8.8 mmol) in tetrahydrofuran (50 mL) was added to the flask, followed by 10% Pd—C(280 mg, 2.6 mmol). The atmosphere in the flask was exchanged with hydrogen gas and the reaction mixture was stirred overnight at ambient temperature under a hydrogen balloon. The reaction mixture was filtered through celite and washed with tetrahydrofuran. The combined filtrate was concentrated under reduced pressure to give the product that was purified by silica gel column chromatography using 20% ethyl acetate in hexanes as eluent to yield (R)-2-((tert-butoxycarbonyl)amino)-4-oxo-4-(pyrrolidin-1-yl)butanoic acid (2-3, 1.8 g). [M+H]⁺=287. ¹H NMR (400 MHZ, DMSO-d₆) δ 12.51 (s, 1H), 6.76 (d, 1H), 4.32 (q, 1H), 3.40-3.32 (m, 2H), 3.26 (t, 2H), 2.71-2.62 (m, 2H), 1.89-1.82 (m, 2H), 1.79-1.72 (m, 2H), 1.37 (s, 9H).

Synthesis of tert-butyl ((R)-1, 4-dioxo-1-(((R)-4-phenyl-1-(4, 4, 5, 5-tetramethyl-1, 3, 2-dioxaborolan-2-yl)butyl)amino)-4-(pyrrolidin-1-yl)butan-2-yl)carbamate, [Step 3]: To a solution of (R)-2-((tert-butoxycarbonyl)amino)-4-oxo-4-(pyrrolidin-1-yl)butanoic acid (2-3, 240 mg, 0.8 mmol) in tetrahydrofuran (7 mL) at −10° C., was added a mixture of isobutyl chloroformate (IBCF) (0.1 mL, 0.8 mmol) and N-methylmorpholine (NMM) (0.1 mL, 0.8 mmol) dropwise, and the reaction mixture was stirred for 30 minutes at −10° C. To this reaction mixture was added a mixture of (R)-4-phenyl-1-(4, 4, 5, 5-tetramethyl-1, 3, 2-dioxaborolan-2-yl)butan-1-amine hydrochloride (2-4, 240 mg, 0.8 mmol) and NMM (0.1 mL, 0.8 mmol) in a mixture of tetrahydrofuran: dimethylformamide (3 mL: 1 mL), the mixture was stirred for 2 hours, and gradually brought to ambient temperature. After the reaction was completed as monitored by LC-MS, the reaction mixture was diluted with ethyl acetate and the organic layer was washed with aqueous 0.1 N HCl (twice), 10% aqueous potassium carbonate (twice), water (twice), brine (twice), dried over sodium sulfate and concentrated under reduced pressure at ambient temperature to afford tert-butyl ((R)-1, 4-dioxo-1-(((R)-4-phenyl-1-(4, 4, 5, 5-tetramethyl-1, 3, 2-dioxaborolan-2-yl)butyl)amino)-4-(pyrrolidin-1-yl)butan-2-yl)carbamate (2-5, 400 mg). The product was used without purification. [M−H]⁻=542.

Synthesis of (R)-2-amino-4-oxo-N—((R)-4-phenyl-1-(4, 4, 5, 5-tetramethyl-1, 3, 2-dioxaborolan-2-yl)butyl)-4-(pyrrolidin-1-yl)butanamide hydrochloride, [Step 4]: To a solution of tert-butyl ((R)-1, 4-dioxo-1-(((R)-4-phenyl-1-(4, 4, 5, 5-tetramethyl-1, 3, 2-dioxaborolan-2-yl)butyl)amino)-4-(pyrrolidin-1-yl)butan-2-yl)carbamate (2-5, 400 mg, 0.7 mmol) in 1, 4-dioxane (4 mL) was added dropwise 4 M HCl in dioxane (1.8 mL, 7.4 mmol). The solution was stirred for 12 hours and concentrated under reduced pressure to yield the product, (R)-2-amino-4-oxo-N—((R)-4-phenyl-1-(4, 4, 5, 5-tetramethyl-1, 3, 2-dioxaborolan-2-yl)butyl)-4-(pyrrolidin-1-yl)butanamide hydrochloride (2-6, 350 mg), which was used without purification. [M−H]⁻=443.

Synthesis of N—((R)-1, 4-dioxo-1-(((R)-4-phenyl-1-(4, 4, 5, 5-tetramethyl-1, 3, 2-dioxaborolan-2-yl)butyl)amino)-4-(pyrrolidin-1-yl)butan-2-yl) pyrazine-2-carboxamide, [Step 5]: To a solution of pyrazine-2-carboxylic acid (2-7, 160 mg, 1.3 mmol) in tetrahydrofuran (7 mL) at −10° C. was added a mixture of isobutyl chloroformate (IBCF) (0.2 mL, 1.3 mmol) and N-methylmorpholine (NMM) (0.12 mL, 1.3 mmol) dropwise and the reaction mixture was stirred for 30 minutes at −10° C. To this reaction mixture was added a mixture of (R)-2-amino-4-oxo-N—((R)-4-phenyl-1-(4, 4, 5, 5-tetramethyl-1, 3, 2-dioxaborolan-2-yl)butyl)-4-(pyrrolidin-1-yl)butanamide hydrochloride (2-6, 550 mg, 1.2 mmol) and NMM (0.17 mL, 1.3 mmol) in a mixture of tetrahydrofuran: dimethylformamide (3 mL: 1 mL) and the reaction mixture was stirred for 2 hours and gradually brought to ambient temperature. The reaction was monitored by LC-MS, and when completed, the reaction mixture was diluted with ethyl acetate and the organic layer was washed with aqueous 0.1 N HCl (twice), 10% aqueous sodium carbonate (twice), water (twice), brine (twice), dried over sodium sulfate, and concentrated under reduced pressure to afford the product. The product was purified by reverse-phase prep-HPLC and lyophilized to yield N—((R)-1, 4-dioxo-1-(((R)-4-phenyl-1-(4, 4, 5, 5-tetramethyl-1, 3, 2-dioxaborolan-2-yl)butyl)amino)-4-(pyrrolidin-1-yl)butan-2-yl) pyrazine-2-carboxamide (2-8, 60 mg). [M−H]⁻=549.

Synthesis of ((R)-1-((R)-4-oxo-2-(pyrazine-2-carboxamido)-4-(pyrrolidin-1-yl)butanamido)-4-phenylbutyl)boronic acid, [Step 6]: To a solution of N—((R)-1, 4-dioxo-1-(((R)-4-phenyl-1-(4, 4, 5, 5-tetramethyl-1, 3, 2-dioxaborolan-2-yl)butyl)amino)-4-(pyrrolidin-1-yl)butan-2-yl) pyrazine-2-carboxamide (2-8, 60 mg, 0.11 mmol) in acetone (2 mL) was added methylboronic acid (2-9, 65 mg, 1.1 mmol) followed by aqueous 0.2 M HCl (2 mL) dropwise. The reaction mixture was allowed to stir at ambient temperature overnight, and was concentrated under reduced pressure at ambient temperature. The product was redissolved in a mixture of acetonitrile and deionized water, and lyophilized to obtain ((R)-1-((R)-4-oxo-2-(pyrazine-2-carboxamido)-4-(pyrrolidin-1-yl)butanamido)-4-phenylbutyl)boronic acid (Compound 2, 45 mg). [M−H]⁻=466. ¹H NMR (400 MHz, Methanol-d₄) δ 9.24 (d, 1H), 8.80 (d, 1H), 8.69 (d, 1H), 7.20 (t, 2H), 7.14 (d, 2H), 7.10 (d, 1H), 5.27 (s, 1H), 3.50 (q, 2H), 3.37 (q, 2H), 3.21 (d, 1H), 3.19 (d, 1H), 2.97 (d, 1H), 2.92 (d, 1H), 2.59 (t, 3H), 1.96 (q, 2H), 1.89-1.84 (m, 2H), 1.66-1.64 (m, 2H), 1.55-1.49 (m, 2H).

### Example 3: Synthesis of ((1R)-1-(4-oxo-4-phenyl-2-(pyrazine-2-carboxamido)butanamido)-4-phenylbutyl)boronic acid

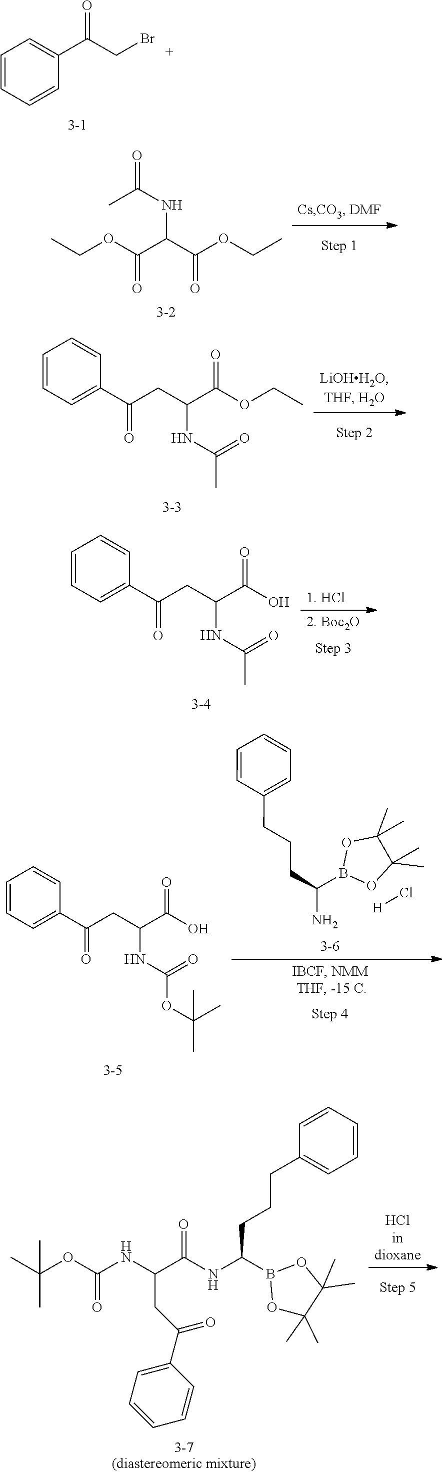

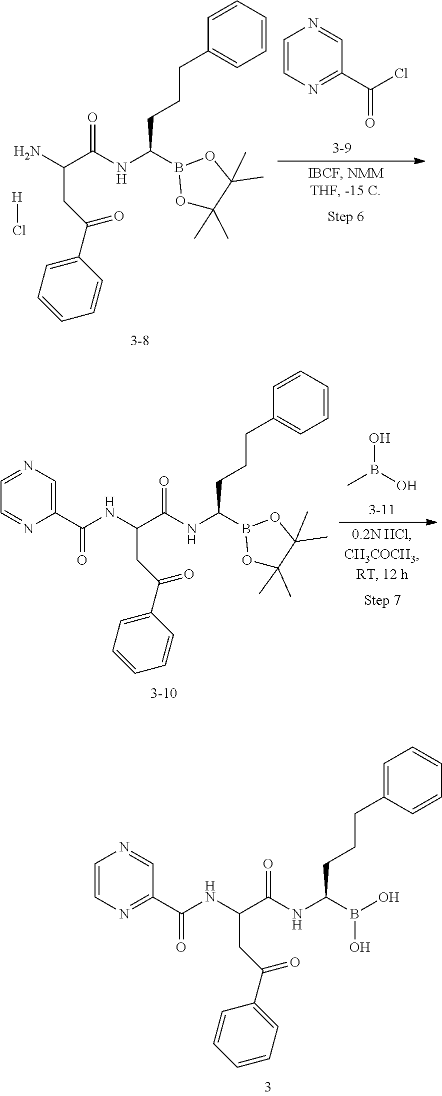

Synthesis of ethyl 2-acetamido-4-oxo-4-phenylbutanoate, [Step 1]: To a stirred solution of 2-bromo-1-phenylethan-1-one (3-1, 5.0 g, 25.1 mmol) and diethyl 2-acetamidomalonate (3-2, 5.5 g, 25.1 mmol) in dimethylformamide (50 mL) was added cesium carbonate (20.5 g, 62.8 mmol) and the reaction mixture was stirred at ambient temperature for 16 hours. TLC showed disappearance of the starting material and the formation of two new spots. The reaction mixture was diluted with ethyl acetate and washed with water and brine. The organic phase was dried over sodium sulfate, filtered, and concentrated under reduced pressure to give the product. The product was purified by column chromatography over silica gel using ethyl acetate in hexanes as eluent and yielded ethyl 2-acetamido-4-oxo-4-phenylbutanoate (3-3, 3.50 g). [M+H]⁺=264. ¹H NMR (400 MHZ, DMSO) δ 8.29 (d, 1H), 7.97-7.95 (m, 2H), 7.68-7.64 (m, 1H), 7.56-7.52 (m, 2H), 4.75 (q, 1H), 4.10-4.02 (m, 2H), 3.46 (d, 2H), 1.82 (s, 3H), 1.14 (t, 3H).

Synthesis of 2-acetamido-4-oxo-4-phenylbutanoic acid, [Step 2]: To a stirred solution of ethyl 2-acetamido-4-oxo-4-phenylbutanoate (3-3, 3.50 g, 13.3 mmol) in a mixture of tetrahydrofuran: water (50 mL: 1 mL) was added lithium hydroxide (LiOH·H₂O) (670 mg, 16.0 mmol) and the reaction mixture was stirred at ambient temperature for 16 hours. The reaction mixture was concentrated under reduced pressure and the residue was acidified with 1N HCl to pH=3. The mixture was extracted with ethyl acetate (thrice) and the combined organic phase was dried over sodium sulfate, filtered, and concentrated under reduced pressure to yield 2-acetamido-4-oxo-4-phenylbutanoic acid (3-4, 3.0 g). [M+H]⁺=236.

Synthesis of 2-((tert-butoxycarbonyl)amino)-4-oxo-4-phenylbutanoic acid, [Step 3]: 6M HCl (100 mL, 638 mmol) was added to 2-acetamido-4-oxo-4-phenylbutanoic acid (3-4, 3.0 g, 12.8 mmol) and the mixture was heated at 100° C. for 24 hours. The reaction mixture was cooled to ambient temperature and washed with diethyl ether (3×50 mL). The aqueous portion was separated and concentrated under reduced pressure to yield 2-amino-4-oxo-4-phenylbutanoic acid (1.50 g).

To a stirred suspension of 2-amino-4-oxo-4-phenylbutanoic acid (1.50 g, 7.8 mmol) in a mixture of tetrahydrofuran: water (25 mL: 60 mL) was added sodium bicarbonate (2.0 g, 23.3 mmol) in ice cold conditions, followed by di-tert-butyl decarbonate (Boc₂O) (3.6 mL, 15.5 mmol) dropwise. The reaction mixture was stirred at ambient temperature for 24 hours. The reaction mixture was washed with diethyl ether (3×50 mL), acidified with 3M HCl, and extracted with ethyl acetate (3×60 mL). The organic phases were combined, dried over sodium sulfate, filtered, and concentrated under reduces pressure to yield 2-((tert-butoxycarbonyl)amino)-4-oxo-4-phenylbutanoic acid (3-5, 2.00 g). [M+H]⁺=294.

Synthesis of tert-butyl (1, 4-dioxo-4-phenyl-1-(((R)-4-phenyl-1-(4, 4, 5, 5-tetramethyl-1, 3, 2-dioxaborolan-2-yl)butyl)amino)butan-2-yl)carbamate, [Step 4]: To a stirred solution of 2-(tert-butoxycarbonyl)amino)-4-oxo-4-phenylbutanoic acid (3-5, 300 mg, 1.0 mmol) in tetrahydrofuran (10 mL), was added isobutyl chloroformate (IBCF) (0.23 mL, 1.8 mmol) and N-methylmorpholine (NMM) (0.15 mL, 1.1 mmol) at −15° C. The reaction mixture was stirred at that temperature for 1 hour, and (R)-4-phenyl-1-(4, 4, 5, 5-tetramethyl-1, 3, 2-dioxaborolan-2-yl)butan-1-amine (3-6, 380 mg, 1.23 mmol) and NMM (0.15 mL, 1.13 mmol) were added, and the reaction mixture was stirred at ambient temperature for 2 hours. The reaction mixture was diluted with ethyl acetate and washed with 0.1M HCl (2×30 mL), 5% potassium carbonate (2×30 mL), water (2×30 mL), and brine. The organic phase was dried over sodium sulfate, filtered, and concentrated under reduced pressure to yield tert-butyl (1, 4-dioxo-4-phenyl-1-(((R)-4-phenyl-1-(4, 4, 5, 5-tetramethyl-1, 3, 2-dioxaborolan-2-yl)butyl)amino)butan-2-yl)carbamate (3-7, 400 mg). [M−H]⁺=550.

Synthesis of 2-amino-4-oxo-4-phenyl-N—((R)-4-phenyl-1-(4, 4, 5, 5-tetramethyl-1, 3, 2-dioxaborolan-2-yl)butyl)butanamide hydrochloride, [Step 5]: To a stirred solution of tert-butyl (1, 4-dioxo-4-phenyl-1-(((R)-4-phenyl-1-(4, 4, 5, 5-tetramethyl-1, 3, 2-dioxaborolan-2-yl)butyl)amino)butan-2-yl)carbamate (3-7, 400 mg, 0.7 mmol) in 1, 4-dioxane (4 mL), was added 4M dioxane-HCl (0.52 g, 14.5 mmol) in ice cold conditions, and the reaction mixture was stirred at ambient temperature for 2 hours. The reaction mixture was concentrated under reduced pressure to yield 2-amino-4-oxo-4-phenyl-N—((R)-4-phenyl-1-(4, 4, 5, 5-tetramethyl-1, 3, 2-dioxaborolan-2-yl)butyl)butanamide hydrochloride (3-8, 350 mg). The product was used without purification.

Synthesis of N-(1, 4-dioxo-4-phenyl-1-(((R)-4-phenyl-1-(4, 4, 5, 5-tetramethyl-1, 3, 2-dioxaborolan-2-yl)butyl)amino)butan-2-yl) pyrazine-2-carboxamide, [Step 6]: To a stirred solution of pyrazine-2-carboxylic acid (3-9, 100 mg, 0.8 mmol) in tetrahydrofuran (10 mL), was added isobutyl chloroformate (IBCF) (0.12 mL, 0.9 mmol) and N-methylmorpholine (NMM) (0.12 mL, 0.9 mmol) at −15° C., and the reaction mixture was stirred at that temperature for 1 hours. Then 2-amino-4-oxo-4-phenyl-N—((R)-4-phenyl-1-(4, 4, 5, 5-tetramethyl-1, 3, 2-dioxaborolan-2-yl)butyl)butanamide hydrochloride (3-8, 360 mg, 0.8 mmol) and NMM (0.12 mL, 0.9 mmol) were added and the reaction mixture was stirred at ambient temperature for 2 h. The reaction mixture was diluted with ethyl acetate and washed with 0.1M HCl (2×30 mL), 5% potassium carbonate (2×30 mL), water (2×30 mL), and brine. The organic phase was dried over sodium sulfate, filtered, and concentrated under reduced pressure to give the product, which was purified by reverse-phase prep-HPLC to yield N-(1, 4-dioxo-4-phenyl-1-(((R)-4-phenyl-1-(4, 4, 5, 5-tetramethyl-1, 3, 2-dioxaborolan-2-yl)butyl)amino)butan-2-yl) pyrazine-2-carboxamide (3-10, 150 mg). [M−H]⁺=555.

Synthesis of ((1R)-1-(4-oxo-4-phenyl-2-(pyrazine-2-carboxamido)butanamido)-4-phenylbutyl)boronic acid, [Step 7]: To a stirred solution N-(1, 4-dioxo-4-phenyl-1-(((R)-4-phenyl-1-(4, 4, 5, 5-tetramethyl-1, 3, 2-dioxaborolan-2-yl)butyl)amino)butan-2-yl) pyrazine-2-carboxamide (3-10, 60 mg, 0.1 mmol) in acetone (1 mL), was added 0.2N HCl (1.0 mL, 0.2 mmol) and methylboronic acid (3-11, 100 mg, 1.6 mmol) and the reaction mixture was stirred at ambient temperature overnight. The reaction mixture was concentrated under reduced pressure and the product was re-dissolved in a mixture of acetone and deionized water, and lyophilized to obtain the product. The product was purified by reverse-phase prep-HPLC and lyophilized to yield ((1R)-1-(4-oxo-4-phenyl-2-(pyrazine-2-carboxamido)butanamido)-4-phenylbutyl)boronic acid (mixture of diastereomers) (Compound 3, 30 mg). [M+H]⁺=473. ¹H NMR (400 MHZ, DMSO d₆+2 drop D₂O) at 80° C., δ 9.15 (br s, 1H), 8.82 (br s, 1H), 8.69 (br s, 1H), 7.91 (d, 2H), 7.63-7.59 (m, 1H), 7.49-7.47 (m, 2H), 7.21-7.07 (m, 5H), 5.02-4.99 (m, 1H), 3.60-3.49 (m, 2H), 3.32 (br s, 2H), 3.13 (br s, 1H), 1.53-1.48 (m, 4H).

### Example 4: Synthesis of ((R)-3-methyl-1-((R)-4-morpholino-4-oxo-2-(pyrazine-2-carboxamido)butanamido)butyl)boronic acid

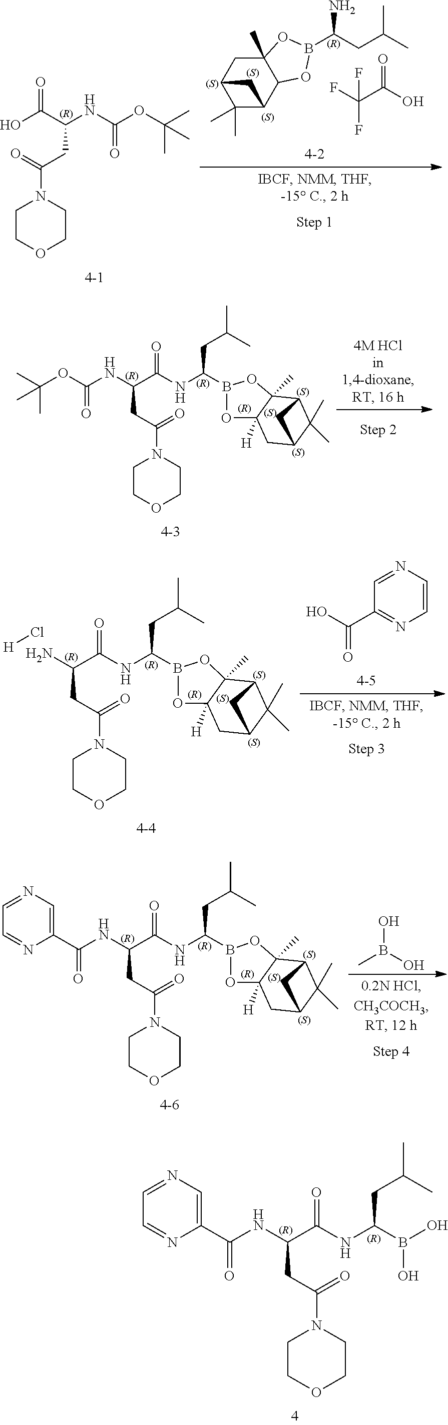

Synthesis of tert-butyl ((R)-1-(((R)-3-methyl-1-((3aS, 4S, 6S, 7aR)-3a, 5, 5-trimethylhexahydro-4, 6-methanobenzo[d][1, 3, 2]dioxaborol-2-yl)butyl)amino)-4-morpholino-1, 4-dioxobutan-2-yl)carbamate, [Step 1]: To a stirred solution of (R)-2-((tert-butoxycarbonyl)amino)-4-morpholino-4-oxobutanoic acid (4-1, 880 mg, 3 mmol) in THF (15 mL) was added NMM (0.4 mL, 3 mmol) followed by IBCF (0.4 mL, 3 mmol) at −15° C. and the reaction mixture was stirred at this temperature for 1 h. A mixture of (R)-3-methyl-1-((3aR, 4R, 6S, 7aS)-3a, 5, 5-trimethylhexahydro-4, 6-methanobenzo[d][1, 3, 2]dioxaborol-2-yl)butan-1-amine 2, 2, 2-trifluoroacetate (4-2, 1 g, 2.6 mmol) and NMM (0.4 mL, 3 mmol) were added to it and stirred at RT for 2 h. The reaction mixture was diluted with EtOAc and washed subsequently with 0.1 N HCl, 5% K₂CO₃, water and brine. The organic phase was dried over Na₂SO₄ and evaporated to get tert-butyl ((R)-1-(((R)-3-methyl-1-((3aR, 4R, 6R, 7aS)-3a, 5, 5-trimethylhexahydro-4, 6-methanobenzo[d][1, 3, 2]dioxaborol-2-yl)butyl)amino)-4-morpholino-1, 4-dioxobutan-2-yl)carbamate (4-3, 1.4 g). This product was used for forwarding step without further purification. [M+H]⁺=550.

Synthesis of (R)-2-amino-N—((R)-3-methyl-1-((3aS, 4S, 6S, 7aR)-3a, 5, 5-trimethylhexahydro-4, 6-methanobenzo[d][1, 3, 2]dioxaborol-2-yl)butyl)-4-morpholino-4-oxobutanamide hydrochloride, [Step 2]: To a stirred solution of tert-butyl ((R)-1-(((R)-3-methyl-1-((3aR, 4R, 6R, 7aS)-3a, 5, 5-trimethylhexahydro-4, 6-methanobenzo[d][1, 3, 2]dioxaborol-2-yl)butyl)amino)-4-morpholino-1, 4-dioxobutan-2-yl)carbamate (4-3, 1.4 g, 2.5 mmol) in 1, 4-dioxane (6 mL) was added 4M HCl in 1, 4-dioxane (6 mL, 25 mmol) in ice cold condition and stirred at RT for 2 h. Volatiles were evaporated under reduced pressure below 35° C. and triturated with n-pentane to yield (R)-2-amino-N—((R)-3-methyl-1-((3aS, 4S, 6S, 7aR)-3a, 5, 5-trimethylhexahydro-4, 6-methanobenzo[d][1, 3, 2]dioxaborol-2-yl)butyl)-4-morpholino-4-oxobutanamide hydrochloride (4-4, 1.2 g). This product was used for next step without further purification. [M+H]⁺=450.

Synthesis of N—((R)-1-(((R)-3-methyl-1-((3aS, 4S, 6S, 7aR)-3a, 5, 5-trimethylhexahydro-4, 6-methanobenzo[d][1, 3, 2]dioxaborol-2-yl)butyl)amino)-4-morpholino-1, 4-dioxobutan-2-yl) pyrazine-2-carboxamide, [Step 3]: To a stirred solution of pyrazine-2-carboxylic acid (4-5, 340 mg, 2.7 mmol) in THF (10 mL) was added NMM (0.4 mL, 2.7 mmol) followed by IBCF (0.4 mL, 2.7 mmol) at −15° C. and stirred at this temperature for 30 min. A mixture of (R)-2-amino-N—((R)-3-methyl-1-((3aS, 4S, 6S, 7aR)-3a, 5, 5-trimethylhexahydro-4, 6-methanobenzo[d][1, 3, 2]dioxaborol-2-yl)butyl)-4-morpholino-4-oxobutanamide hydrochloride (4-4, 1.2 g, 2.5 mmol) and NMM (0.4 mL, 2.7 mmol)) was added and the reaction mixture was stirred at RT for 2 h. The reaction mixture was diluted with EtOAc and washed subsequently with 0.1N HCl (2×15 mL), 5% K₂CO₃ (2×15 mL), water (2×15 mL) and brine. The organic phase was dried over Na₂SO₄ and evaporated. The product was purified via prep HPLC purification and lyophilized to afford N—((R)-1-(((R)-3-methyl-1-((3aS, 4S, 6S, 7aR)-3a, 5, 5-trimethylhexahydro-4, 6-methanobenzo[d][1, 3, 2]dioxaborol-2-yl)butyl)amino)-4-morpholino-1, 4-dioxobutan-2-yl) pyrazine-2-carboxamide (4-6, 300 mg). [M−H]⁻=555.

Synthesis of ((R)-3-methyl-1-((R)-4-morpholino-4-oxo-2-(pyrazine-2-carboxamido) butanamido)butyl)boronic acid, [Step 4]: To a solution of N—((R)-1-(((R)-3-methyl-1-((3aS, 4S, 6S, 7aR)-3a, 5, 5-trimethylhexahydro-4, 6-methanobenzo[d][1, 3, 2]dioxaborol-2-yl)butyl)amino)-4-morpholino-1, 4-dioxobutan-2-yl) pyrazine-2-carboxamide (4-6, 300 mg, 0.5 mmol) in acetone (4 mL) was added methylboronic acid (320 mg, 5.4 mmol) followed by dropwise addition of 0.2 HCl (4 mL). The reaction mixture was stirred at room temperature for overnight. Volatiles were evaporated under reduced pressure and purified via prep HPLC purification and lyophilized to afford ((R)-3-methyl-1-((R)-4-morpholino-4-oxo-2-(pyrazine-2-carboxamido)butanamido)butyl) boronic acid (Compound 4, 90 mg). [M−H]⁻=420; ¹H NMR (400 MHZ, MeOD) δ 9.25 (s, 1H), 8.80 (d, 1H), 8.69 (s, 1H), 5.30 (t, 1H), 3.68-3.61 (m, 4H), 3.55-3.51 (m, 4H), 3.28-3.27 (m, 1H), 3.02-2.97 (m, 1H), 2.70 (t, 1H), 1.67-1.66 (m, 1H), 1.35 (t, 2H), 0.80-0.87 (m, 6H).

### Example 5: Synthesis of ((R)-1-((R)-4-morpholino-4-oxo-2-(pyrazine-2-carboxamido) butanamido)-3-phenylpropyl)boronic acid

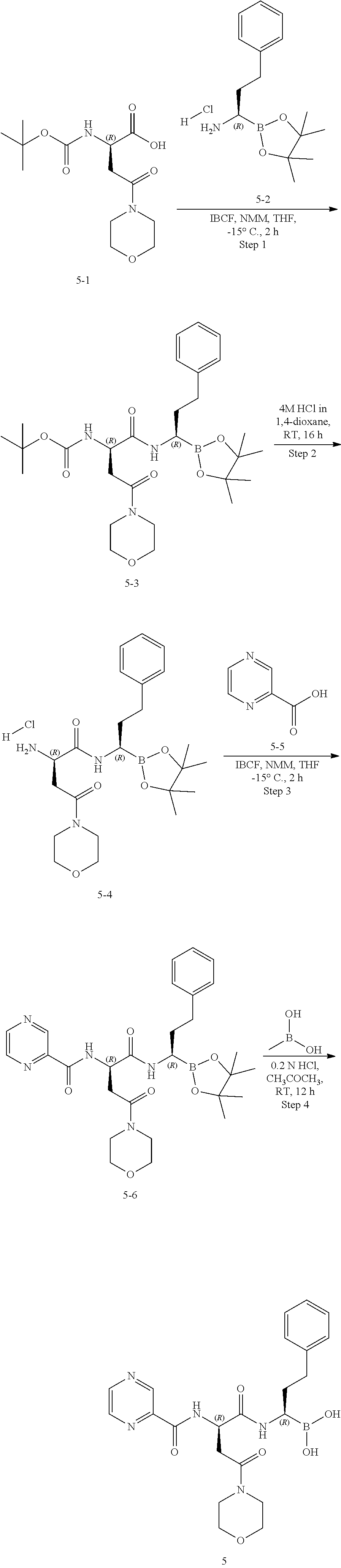

Synthesis of tert-butyl ((R)-4-morpholino-1, 4-dioxo-1-(((R)-3-phenyl-1-(4, 4, 5, 5-tetramethyl-1, 3, 2-dioxaborolan-2-yl)propyl)amino)butan-2-yl)carbamate, [Step 1]: To a stirred solution of (R)-2-((tert-butoxycarbonyl)amino)-4-morpholino-4-oxobutanoic acid (5-1, 890 mg, 3 mmol) in THF (15 mL) was added NMM (0.40 mL, 3 mmol) followed by IBCF (0.40 mL, 3 mmol) at −15° C. and stirred at this temperature for 1 h. A mixture of (R)-3-phenyl-1-(4, 4, 5, 5-tetramethyl-1, 3, 2-dioxaborolan-2-yl) propan-1-amine hydrochloride (5-2, 800 mg, 2.69 mmol) and NMM (0.40 mL, 2.96 mmol) was added and stirred at RT for 2 h. The reaction mixture was diluted with EtOAc and washed subsequently with 0.1N HCl, 5% K₂CO₃, water and brine. The organic phase was dried over Na₂SO₄ and evaporated to get tert-butyl ((R)-4-morpholino-1, 4-dioxo-1-(((R)-3-phenyl-1-(4, 4, 5, 5-tetramethyl-1, 3, 2-dioxaborolan-2-yl)propyl)amino)butan-2-yl)carbamate (5-3, 1.4 g). The product was used for next step without further purification. [M−H]⁻=544.

Synthesis of (R)-2-amino-4-morpholino-4-oxo-N—((R)-3-phenyl-1-(4, 4, 5, 5-tetramethyl-1, 3, 2-dioxaborolan-2-yl)propyl)butanamide hydrochloride, [Step 2]: To a stirred solution of tert-butyl ((R)-4-morpholino-1, 4-dioxo-1-(((R)-3-phenyl-1-(4, 4, 5, 5-tetramethyl-1, 3, 2-dioxaborolan-2-yl)propyl)amino)butan-2-yl)carbamate (5-3, 1.4 mg, 2.6 mmol) in 1, 4-dioxane (6 mL) was added 4N HCl in 1, 4-dioxane (6 mL, 26 mmol) in ice cold condition and stirred at RT for 2 h. Volatiles were removed under reduced pressure and triturated with n-pentane to get (R)-2-amino-4-morpholino-4-oxo-N—((R)-3-phenyl-1-(4, 4, 5, 5-tetramethyl-1, 3, 2-dioxaborolan-2-yl)propyl)butanamide hydrochloride (5-4, 1 g). This product was used for next step without further purification. [M−H]⁻=444.

Synthesis of N—((R)-4-morpholino-1, 4-dioxo-1-(((R)-3-phenyl-1-(4, 4, 5, 5-tetramethyl-1, 3, 2-dioxaborolan-2-yl)propyl)amino)butan-2-yl) pyrazine-2-carboxamide, [Step 3]: To a stirred solution of pyrazine-2-carboxylic acid (5-5, 280 mg, 2.3 mmol) in THF (10 mL) was added NMM (0.3 mL, 2.3 mmol) followed by IBCF (0.3 mL, 2.3 mmol) at −15° C. and stirred at this temperature for 30 min. A mixture of (R)-2-amino-4-morpholino-4-oxo-N—((R)-3-phenyl-1-(4, 4, 5, 5-tetramethyl-1, 3, 2-dioxaborolan-2-yl)propyl)butanamide hydrochloride (5-4, 1 g, 2 mmol) and NMM (0.3 mL, 2.3 mmol) were added and stirred at RT for 2 h. The reaction mixture was diluted with EtOAc and washed subsequently with 0.1N HCl (2×15 mL), 5% K₂CO₃ (2×15 mL), water (2×15 mL) and brine, dried over Na₂SO₄ and evaporated. The product was purified via prep HPLC purification and lyophilized to afford N—((R)-4-morpholino-1, 4-dioxo-1-(((R)-3-phenyl-1-(4, 4, 5, 5-tetramethyl-1, 3, 2-dioxaborolan-2-yl)propyl)amino)butan-2-yl) pyrazine-2-carboxamide (5-6, 150 mg). [M−H]⁻=550.4.

Synthesis of ((R)-1-((R)-4-morpholino-4-oxo-2-(pyrazine-2-carboxamido)butanamido)-3-phenylpropyl)boronic acid, [Step 4]: To a solution of N—((R)-4-morpholino-1, 4-dioxo-1-(((R)-3-phenyl-1-(4, 4, 5, 5-tetramethyl-1, 3, 2-dioxaborolan-2-yl)propyl)amino)butan-2-yl) pyrazine-2-carboxamide (150 mg, 0.3 mmol) in acetone (2 mL) was added methylboronic acid (160 mg, 2.7 mmol) followed by dropwise addition of 0.2N HCl (2 mL) and stirred at room temperature for overnight. Volatiles were removed under reduced pressure and lyophilized. The product was purified via prep HPLC purification and lyophilized to afford ((R)-1-((R)-4-morpholino-4-oxo-2-(pyrazine-2-carboxamido)butanamido)-3-phenylpropyl)boronic acid (Compound 5, 50 mg). [M−H]⁻=468; ¹H NMR (400 MHZ, MeOD) δ 9.27 (d, 1H), 8.81 (d, 1H), 8.70-8.69 (m, 1H), 7.22-7.16 (m, 4H), 7.09 (t, 1H), 5.31 (t, 1H), 3.65 (t, 2H), 3.63-3.6 (m, 2H), 3.57-3.51 (m, 5H), 3.04-2.98 (m, 1H), 2.63 (t, 3H), 1.84-1.71 (m, 2H).

### Example 6: Synthesis of ((R)-1-((R)-4-morpholino-4-oxo-2-(pyrazine-2-carboxamido) butanamido)pentyl)boronic acid

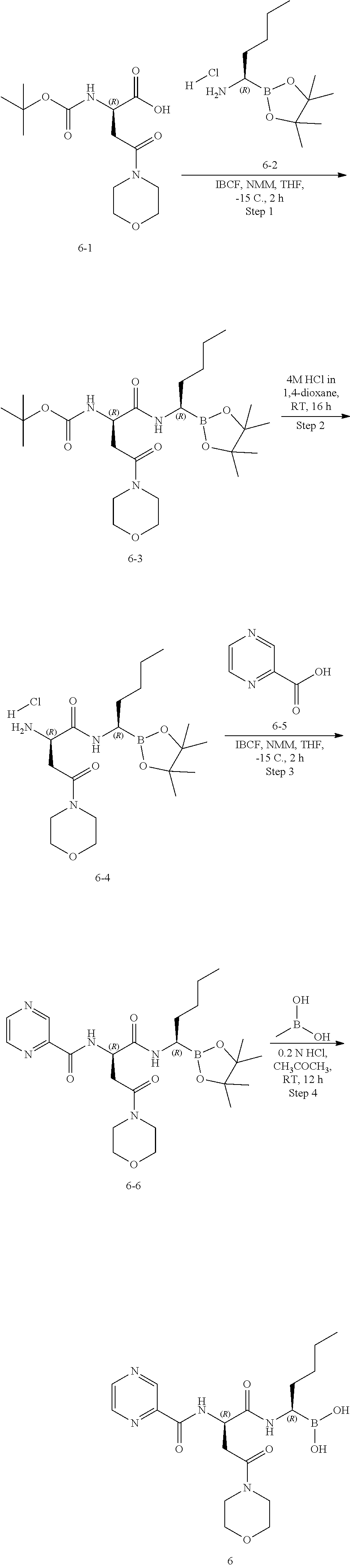

Synthesis of tert-butyl ((R)-4-morpholino-1, 4-dioxo-1-(((R)-1-(4, 4, 5, 5-tetramethyl-1, 3, 2-dioxaborolan-2-yl)pentyl)amino)butan-2-yl)carbamate, [Step 1]: To a stirred solution of (R)-2-((tert-butoxycarbonyl)amino)-4-morpholino-4-oxobutanoic acid (6-1, 1.1 g, 3.5 mmol) in THF (15 mL), NMM (0.4 mL, 3.5 mmol) and IBCF (0.5 mL, 3.5 mmol) were added at −15° C. and the reaction mixture was stirred at this temperature for 1 h. To the above solution, (R)-1-(4, 4, 5, 5-tetramethyl-1, 3, 2-dioxaborolan-2-yl) pentan-1-amine hydrochloride (800 mg, 3.2 mmol) and NMM (0.4 mL, 3.5 mmol) were added and the reaction mixture was stirred at RT for 2 h. After completion of the reaction (monitored by TLC and LC-MS), the reaction mixture was diluted with EtOAc and washed subsequently with 0.1 M HCl, 5% K₂CO₃, water and brine. The organic phase was dried over Na₂SO₄, filtered and evaporated to yield tert-butyl ((R)-4-morpholino-1, 4-dioxo-1—(((R)-1-(4, 4, 5, 5-tetramethyl-1, 3, 2-dioxaborolan-2-yl)pentyl)amino)butan-2-yl)carbamate (6-3, 1500 mg). The product was used for next step without further purification. [M+H]⁺=498.

Synthesis of (R)-2-amino-4-morpholino-4-oxo-N—((R)-1-(4, 4, 5, 5-tetramethyl-1, 3, 2-dioxaborolan-2-yl)pentyl)butanamide hydrochloride, [Step 2]: To a stirred solution of tert-butyl ((R)-4-morpholino-1, 4-dioxo-1-(((R)-1-(4, 4, 5, 5-tetramethyl-1, 3, 2-dioxaborolan-2-yl)pentyl)amino)butan-2-yl)carbamate (6-3, 1500 mg, 3 mmol) in 1, 4-dioxane (7 mL) was added 4 M HCl-dioxane (7 mL, 30 mmol) in ice cold condition and the reaction mixture was stirred at RT for 2 h. After completion of the reaction (monitored by LCMS) the solvent was evaporated from reaction mixture. The product was triturated with n-pentane to yield (R)-2-amino-4-morpholino-4-oxo-N—((R)-1-(4, 4, 5, 5-tetramethyl-1, 3, 2-dioxaborolan-2-yl)pentyl)butanamide hydrochloride (6-4, 1000 mg). This product was used for next step without further purification. [M−H]⁻=396.

Synthesis of N—((R)-4-morpholino-1, 4-dioxo-1-(((R)-1-(4, 4, 5, 5-tetramethyl-1, 3, 2-dioxaborolan-2-yl)pentyl)amino)butan-2-yl) pyrazine-2-carboxamide, [Step 3]: To a stirred solution of pyrazine-2-carboxylic acid (6-5, 190 mg, 1.5 mmol) in THF (8 mL), NMM (0.2 mL, 1.5 mmol) and IBCF (0.2 mL, 1.5 mmol) were added at −15° C. and the reaction mixture was stirred at this temperature for 30 min. Then ((R)-2-amino-4-morpholino-4-oxo-N—((R)-1-(4, 4, 5, 5-tetramethyl-1, 3, 2-dioxaborolan-2-yl)pentyl)butanamide hydrochloride (6-4, 600 mg, 1.4 mmol) and NMM (0.2 mL, 1.5 mmol) were added and the reaction mixture was stirred at RT for 2 h. After completion of the reaction (monitored by TLC and LC-MS), the reaction mixture was diluted with EtOAc and washed subsequently with 0.1 M HCl, 5% K₂CO₃, water and brine. The organic phase was dried over Na₂SO₄, filtered and evaporated to yield a brown gum. The product was purified by RP prep HPLC purification and the eluent was lyophilized to afford N—((R)-4-morpholino-1, 4-dioxo-1-(((R)-1-(4, 4, 5, 5-tetramethyl-1, 3, 2-dioxaborolan-2-yl)pentyl)amino)butan-2-yl) pyrazine-2-carboxamide (6-6, 60 mg). [M−H]⁻=502.

Synthesis of ((R)-1-((R)-4-morpholino-4-oxo-2-(pyrazine-2-carboxamido)butanamido) pentyl)boronic acid, [Step 4]: To a solution of N—((R)-4-morpholino-1, 4-dioxo-1-(((R)-1-(4, 4, 5, 5-tetramethyl-1, 3, 2-dioxaborolan-2-yl)pentyl)amino)butan-2-yl) pyrazine-2-carboxamide (6-6, 60 mg, 0.1 mmol) in acetone (2 mL) was added methylboronic acid (70 mg, 1 mmol), followed by dropwise addition of 0.2 HCl (2 mL). The reaction mixture was allowed to stirring at room temperature overnight. All the volatiles were evaporated at room temperature, and reaction mixture was re-dissolved in acetonitrile and deionized water and freeze-dried. The obtained product was purified through RP prep HPLC purification and lyophilized to afford ((R)-1-((R)-4-morpholino-4-oxo-2-(pyrazine-2-carboxamido)butanamido)pentyl)boronic acid (Compound 6, 40 mg). [M−H]⁻=420; ¹H NMR (400 MHZ, MeOD) δ 9.26 (s, 1H), 8.80 (d, 1H), 8.69 (s, 1H), 5.30 (d, 1H), 3.68-3.65 (m, 2H), 3.64-3.62 (m, 2H), 3.54-3.47 (m, 4H), 3.27 (s, 1H), 2.99 (dd, 1H), 2.57 (t, 1H), 1.51-1.48 (m, 1H), 1.46-1.44 (m, 1H), 1.31-1.28 (m, 4H), 0.88 (s, 3H).

### Example 7: Synthesis of ((R)-1-((R)-2-(2, 4-dimethyloxazole-5-carboxamido)-4-morpholino-4-oxobutanamido)-3-methylbutyl)boronic acid

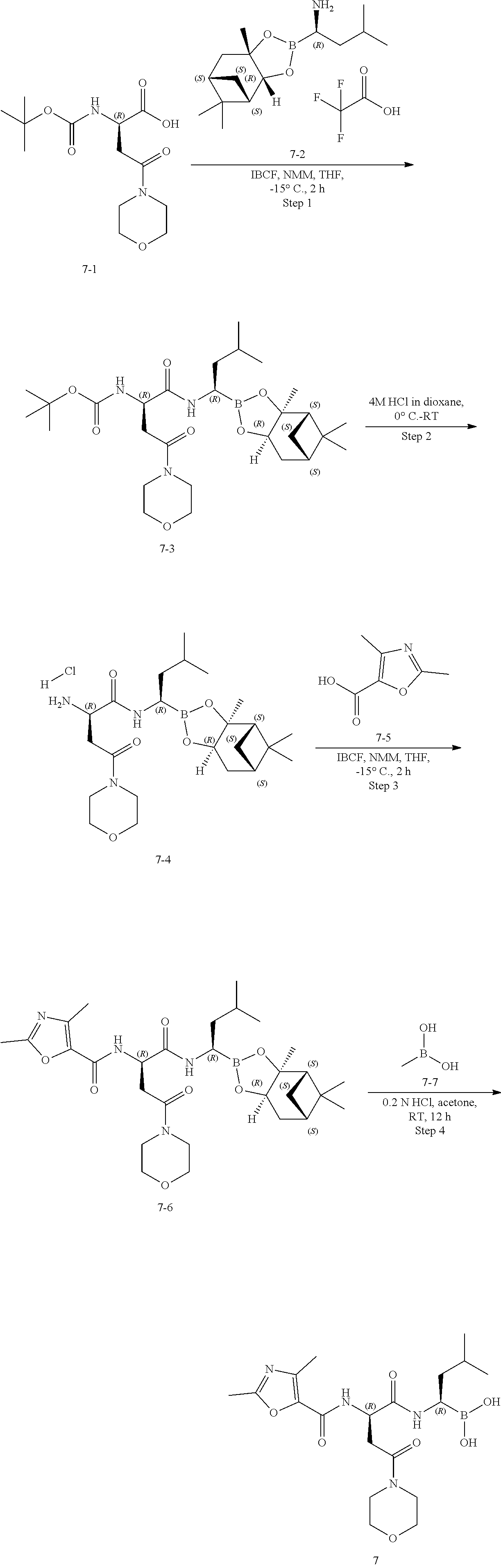

Synthesis of tert-butyl ((R)-1-(((R)-3-methyl-1-((3aS, 4S, 6S, 7aR)-3a, 5, 5-trimethylhexahydro-4, 6-methanobenzo[d][1, 3, 2]dioxaborol-2-yl)butyl)amino)-4-morpholino-1, 4-dioxobutan-2-yl)carbamate, [Step 1]: To a stirred solution of (R)-2-((tert-butoxycarbonyl)amino)-4-morpholino-4-oxobutanoic acid (7-1, 530 mg, 1.8 mmol) in THF (8 mL) was added IBCF (0.3 mL, 1.8 mmol) and NMM (0.3 mL, 2 mmol) at −15° C. The reaction mixture was stirred at same temperature for 30 min. Then (R)-3-methyl-1-((3aR, 4S, 6S, 7aS)-5, 5, 7a-trimethylhexahydro-4, 6-methanobenzo[d][1, 3, 2]dioxaborol-2-yl)butan-1-amine TFA salt (7-2, 600 mg, 1.6 mmol) in DMF (1 mL) followed by NMM (0.3 mL, 1.8 mmol) was added to the reaction mixture at −15° C. It was gradually warmed to 0° C. and stirred for 2 h. The reaction mixture was neutralized with saturated aqueous 0.1 N HCl solution and extracted with ethyl acetate. The combined organic layer was washed with 5% K₂CO₃ solution, water and brine. It was dried over Na₂SO₄, filtered and evaporated under reduced pressure to afford tert-butyl ((R)-1-(((R)-3-methyl-1-((3aS, 4S, 6S, 7aR)-3a, 5, 5-trimethylhexahydro-4, 6-methanobenzo[d][1, 3, 2]dioxaborol-2-yl)butyl)amino)-4-morpholino-1, 4-dioxobutan-2-yl)carbamate (7-3, 800 mg). [M−H]⁺=548.

Synthesis of (R)-2-amino-N—((R)-3-methyl-1-((3aS, 4S, 6S, 7aR)-3a, 5, 5-trimethylhexahydro-4, 6-methanobenzo[d][1, 3, 2]dioxaborol-2-yl)butyl)-4-morpholino-4-oxobutanamide, [Step 2]: To a solution of tert-butyl ((R)-1-(((R)-3-methyl-1-((3aS, 4S, 6S, 7aR)-3a, 5, 5-trimethylhexahydro-4, 6-methanobenzo[d][1, 3, 2]dioxaborol-2-yl)butyl)amino)-4-morpholino-1, 4-dioxobutan-2-yl)carbamate (7-3, 800 mg, 1.5 mmol) in 1, 4-dioxane (6 mL) was added 4 M HCl in 1, 4-dioxane (5 mL, 22 mmol) at 0° C. It was gradually warmed to 25° C. and stirred for 16 h. The volatiles were removed under reduced pressure to get (R)-2-amino-N—((R)-3-methyl-1-((3aS, 4S, 6S, 7aR)-3a, 5, 5-trimethylhexahydro-4, 6-methanobenzo[d][1, 3, 2]dioxaborol-2-yl)butyl)-4-morpholino-4-oxobutanamide (7-4, 500 mg). [M−H]⁺=449.

Synthesis of 2, 4-dimethyl-N—((R)-1-(((R)-3-methyl-1-((3aS, 4S, 6S, 7aR)-3a, 5, 5-trimethylhexahydro-4, 6-methanobenzo[d][1, 3, 2]dioxaborol-2-yl)butyl)amino)-4-morpholino-1, 4-dioxobutan-2-yl)oxazole-5-carboxamide, [Step 3]: To a stirred solution of 2, 4-dimethyloxazole-5-carboxylic acid (7-5, 175 mg, 1.2 mmol) in THF (8 mL) was added IBCF (0.3 mL, 1.8 mmol) and NMM (0.3 mL, 1.8 mmol) at −15° C. The reaction mixture was stirred at same temperature for 30 min. Then (R)-2-amino-N—((R)-3-methyl-1-((3aS, 4S, 6S, 7aR)-3a, 5, 5-trimethylhexahydro-4, 6-methanobenzo[d][1, 3, 2]dioxaborol-2-yl)butyl)-4-morpholino-4-oxobutanamide (7-4, 500 mg, 1.1 mmol) in DMF (1 mL) followed by NMM (0.3 mL, 1.8 mmol) were added to the reaction mixture at −15° C. It was gradually warmed to 0° C. and stirred for 2 h. It was neutralized with saturated aqueous 0.1 N HCl solution and extracted with ethyl acetate. The combined organic layer was washed with 5% K₂CO₃ solution, water and brine. It was dried over Na₂SO₄, filtered and evaporated under reduced pressure to afford 2, 4-dimethyl-N—((R)-1-(((R)-3-methyl-1-((3aS, 4S, 6S, 7aR)-3a, 5, 5-trimethylhexahydro-4, 6-methanobenzo[d][1, 3, 2]dioxaborol-2-yl)butyl)amino)-4-morpholino-1, 4-dioxobutan-2-yl)oxazole-5-carboxamide (7-6, 200 mg). [M−H]⁺=571.

Synthesis of ((R)-1-((R)-2-(2, 4-dimethyloxazole-5-carboxamido)-4-morpholino-4-oxobutanamido)-3-methylbutyl)boronic acid, [Step 4]: To a stirred solution of 2, 4-dimethyl-N—((R)-1-(((R)-3-methyl-1-((3aS, 4S, 6S, 7aR)-3a, 5, 5-trimethylhexahydro-4, 6-methanobenzo[d][1, 3, 2]dioxaborol-2-yl)butyl)amino)-4-morpholino-1, 4-dioxobutan-2-yl)oxazole-5-carboxamide (7-6, 100 mg, 0.2 mmol) and methylboronic acid (7-7, 155 mg, 2.6 mmol) in acetone (2 mL) was added 0.2 N HCl (2.0 mL) and the reaction mixture was stirred at RT overnight. The volatiles were evaporated and the residue was redissolved in acetone and deionized water and freeze-dried to obtain product. The material was purified by RP prep HPLC purification and lyophilized to afford ((R)-1-((R)-2-(2, 4-dimethyloxazole-5-carboxamido)-4-morpholino-4-oxobutanamido)-3-methylbutyl)boronic acid (Compound 7, 40 mg). [M−H]⁺=437; ¹H NMR (400 MHZ, MeOD) δ 5.20 (t, 1H), 3.68-3.66 (m, 2H), 3.62 (d, 2H), 3.55-3.51 (m, 4H), 3.17-3.11 (m, 1H), 3.01-2.96 (m, 1H), 2.71 (t, 1H), 2.48 (s, 3H), 2.39 (s, 3H), 1.98 (br s, 1H), 1.70-1.63 (m, 1H), 1.35 (t, 2H), 0.90 (d, 6H).

### Example 8: Synthesis of ((R)-1-((R)-4-(dimethylamino)-4-oxo-2-(pyrazine-2-carboxamido) butanamido)-4-phenylbutyl)boronic acid

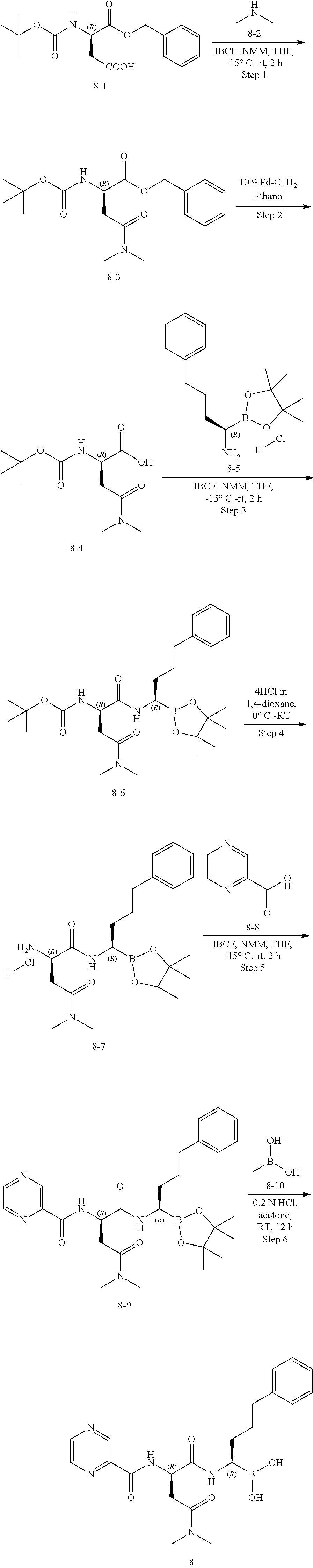

Synthesis of benzyl N²-(tert-butoxycarbonyl)-N⁴,N⁴-dimethyl-D-asparaginate, [Step 1]: To a stirred solution of (R)-4-(benzyloxy)-3-((tert-butoxycarbonyl)amino)-4-oxobutanoic acid (8-1, 1 g, 3.2 mmol) in THF (20 mL) was added IBCF (0.4 mL, 3.2 mmol) and NMM (0.4 mL, 3.2 mmol) at −15° C. Reaction mixture was stirred at same temperature for 30 min. Then dimethylamine (8-2, 2 M in THF) (1.4 ml, 2.9 mmol) followed by NMM (0.3 mL, 2.9 mmol) was added to reaction mixture at −15° C. It was gradually warmed to 0° C. and stirred for 2 h. LCMS of crude reaction mass confirmed the formation of desired product. It was neutralized with saturated aqueous 0.1 N HCl solution and extracted with ethyl acetate. The combined organic layer was washed with 5% K₂CO₃ solution, water and brine. It was dried over Na₂SO₄, filtered and evaporated under reduced pressure to get product. The product was purified by combi-flash column chromatography using 0-50% EtOAc in hexanes as eluent to afford benzyl N²-(tert-butoxycarbonyl)-N⁴,N⁴-dimethyl-D-asparaginate (8-3, 600 mg). [M+H]⁺=351; ¹H NMR (400 MHZ, DMSO-d₆) (400 MHZ, DMSO-d₆) δ 7.52-7.17 (m, 5H), 5.10 (s, 2H), 4.56-4.36 (m, 1H), 2.91 (s, 3H), 2.80 (s, 3H), 2.79-2.64 (m, 2H), 1.36 (s, 9H), 1.32-1.26 (m, 1H).

Synthesis of N²-(tert-butoxycarbonyl)-N⁴,N⁴-dimethyl-D-asparagine, [Step 2]: To a stirred solution of benzyl N²-(tert-butoxycarbonyl)-N⁴,N⁴-dimethyl-D-asparaginate (8-3, 1.0 g, 2.8 mmol) dissolved in THF (25 mL) was bubbled with nitrogen gas for 10 min. Then 10% Pd—C(400 mg) was added and the reaction mixture was hydrogenated under balloon pressure for 3 h. The progress of the reaction was monitored using TLC. Upon completion, the reaction mixture was filtered over celite bed and washed the bed with excess ethyl acetate to get N²-(tert-butoxycarbonyl)-N⁴,N⁴-dimethyl-D-asparagine (8-4, 700 mg). It was used in next step without further purification. [M+H]: 261; ¹H NMR (400 MHZ, DMSO-d₆) δ 12.48 (s, 1H), 6.70 (d, 1H), 4.31 (s, 1H), 3.97-3.69 (m, 2H), 2.93 (s, 3H), 2.80 (s, 3H), 1.38 (s, 9H).

Synthesis of tert-butyl ((R)-4-(dimethylamino)-1, 4-dioxo-1-(((R)-4-phenyl-1-(4, 4, 5, 5-tetramethyl-1, 3, 2-dioxaborolan-2-yl)butyl)amino)butan-2-yl)carbamate, [Step 3]: To a stirred solution of N²-(tert-butoxycarbonyl)-N⁴,N⁴-dimethyl-D-asparaginate (8-4, 200 mg, 0.8 mmol) in THF (8 mL) was added IBCF (0.1 mL, 0.8 mmol) and NMM (85 μmL, 0.8 mmol) at −15° C. The reaction mixture was stirred at same temperature for 30 min. Then (R)-4- phenyl-1-(4, 4, 5, 5-tetramethyl-1, 3, 2-dioxaborolan-2-yl)butan-1-amine hydrochloride (8-5, 220 mg, 0.7 mmol) in DMF (1 mL) followed by NMM (0.08 mL, 0.7 mmol) was added to reaction mixture at −15° C. It was gradually warmed to 0° C. and stirred for 2 h. LCMS of crude reaction mass confirmed the formation of desired product. It was neutralized with saturated aqueous 0.1 N HCl solution and extracted with ethyl acetate. The combined organic layer was washed with 5% K₂CO₃ solution, water, brine, dried over Na₂SO₄, filtered and evaporated under reduced pressure to afford tert-butyl ((R)-4-(dimethylamino)-1, 4-dioxo-1-(((R)-4-phenyl-1-(4, 4, 5, 5-tetramethyl-1, 3, 2-dioxaborolan-2-yl)butyl)amino)butan-2-yl)carbamate (8-6, 250 mg). The product was used directly without further purification. [M+H]: 518.

Synthesis of (R)-2-amino-N⁴,N⁴-dimethyl-N¹—((R)-4-phenyl-1-(4, 4, 5, 5-tetramethyl-1, 3, 2-dioxaborolan-2-yl)butyl) succinamide hydrochloride, [Step 4]: To a solution of tert-butyl ((R)-4-(dimethylamino)-1, 4-dioxo-1-(((R)-4-phenyl-1-(4, 4, 5, 5-tetramethyl-1, 3, 2-dioxaborolan-2-yl)butyl)amino)butan-2-yl)carbamate (8-6, 650 mg, 1.2 mmol) in 1, 4-dioxane (6 mL) was added 4 M HCl in 1, 4-dioxane (6.0 mL, 25 mmol) at 0° C. It was gradually warmed to 25° C. and stirred for 16 h. TLC showed complete consumption of starting material to form new polar spot. Volatiles were removed under reduced pressure to get (R)-2-amino-N⁴,N⁴-dimethyl-N¹—((R)-4-phenyl-1-(4, 4, 5, 5-tetramethyl-1, 3, 2-dioxaborolan-2-yl)butyl) succinamide hydrochloride (8-7, 550 mg, 1.2 mmol). The product was directly used for next step without purification.

Synthesis of (R)—N⁴,N⁴-dimethyl-N¹—((R)-4-phenyl-1-(4, 4, 5, 5-tetramethyl-1, 3, 2-dioxaborolan-2-yl)butyl)-2-(pyrazine-2-carboxamido) succinimide, [Step 5]: To a stirred solution of pyrazine-2-carboxylic acid (8-8, 165 mg, 1.3 mmol) in THF (8 mL) was added IBCF (0.2 mL, 1.3 mmol) and NMM (0.2 mL, 1.33 mmol) at −15° C. The reaction mixture was stirred at same temperature for 30 min. Then (R)-2-amino-N⁴,N⁴-dimethyl-N¹—((R)-4-phenyl-1-(4, 4, 5, 5-tetramethyl-1, 3, 2-dioxaborolan-2-yl)butyl) succinamide hydrochloride (8-7, 550 mg, 1.2 mmol) in DMF (1 mL) followed by NMM (0.13 mL, 1.2 mmol) was added to reaction mixture under same condition. It was gradually warmed to 0° C. and stirred for 2 h. LCMS of crude reaction mass confirmed the formation of desired product. It was neutralized with saturated aqueous (0.1 N) HCl solution and extracted with ethyl acetate. The combined organic layer was washed with 5% K₂CO₃ solution, water, brine, dried over Na₂SO₄, filtered and evaporated under reduced pressure. The material was purified by RP prep HPLC purification and lyophilized to afford (R)—N⁴,N⁴-dimethyl-N¹—((R)-4-phenyl-1-(4, 4, 5, 5-tetramethyl-1, 3, 2-dioxaborolan-2-yl)butyl)-2-(pyrazine-2-carboxamido) succinamide (8-9, 30 mg). LCMS (ESI) Calcd. for C₂₇H₃₈BN₅O₅: 523, [M−H]⁺=523.

Synthesis of ((R)-1-((R)-4-(dimethylamino)-4-oxo-2-(pyrazine-2-carboxamido)butanamido)-4-phenylbutyl)boronic acid, [Step 6]: To a stirred solution of (R)—N⁴,N⁴-dimethyl-N¹—((R)-4-phenyl-1-(4, 4, 5, 5-tetramethyl-1, 3, 2-dioxaborolan-2-yl)butyl)-2-(pyrazine-2-carboxamido) succinamide (8-9, 30 mg, 0.06 mmol) and methylboronic acid (34 mg, 0.6 mmol) in acetone (2 mL) was added 0.2 N HCl (2 mL) and the reaction mixture was stirred at RT overnight. TLC and LCMS showed full conversion of starting material with formation of new polar spot. The volatiles were evaporated and the residue was purified by RP prep HPLC purification to obtain ((R)-1-((R)-4-(dimethylamino)-4-oxo-2-(pyrazine-2-carboxamido) butanamido)-4-phenylbutyl)boronic acid (Compound 8, 7.0 mg). LCMS (ESI) Calcd. for C₂₁H₂₈BN₅O₅: 441, found [M−H]⁺=440; ¹H NMR (400 MHZ, Methanol-d₄) δ 9.24 (s, 1H), 8.80 (d, 1H), 8.71-8.65 (m, 1H), 7.24-7.05 (m, 5H), 5.26 (s, 1H), 3.05 (s, 3H), 2.90 (s, 3H), 2.58 (d, 3H), 1.77-1.41 (m, 6H).

### Example 9: Synthesis of ((R)-1-((R)-4-(ethylamino)-4-oxo-2-(pyrazine-2-carboxamido) butanamido)-4-phenylbutyl)boronic acid

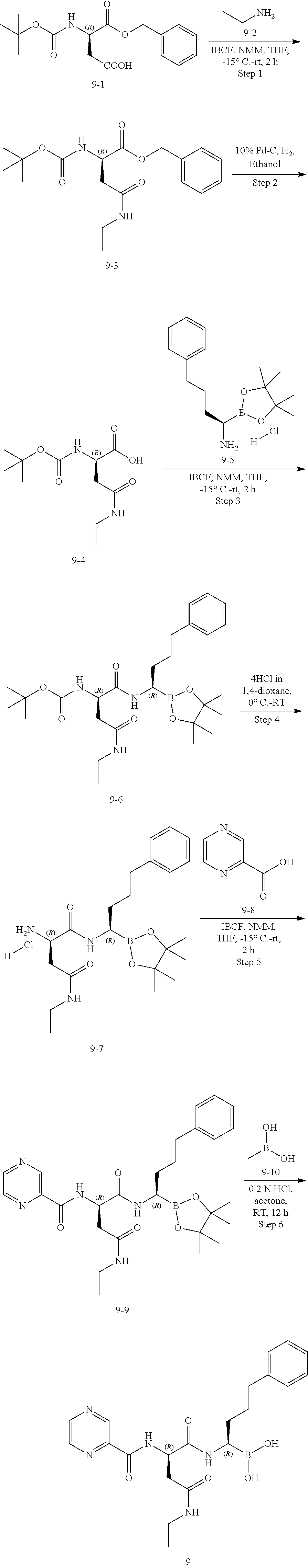

Synthesis of benzyl N²-(tert-butoxycarbonyl)-N⁴-ethyl-D-asparaginate, [Step 1]: To a solution of (R)-4-(benzyloxy)-3-((tert-butoxycarbonyl)amino)-4-oxobutanoic acid (9-1, 1.0 g, 3.3 mmol) in THF (10 mL), IBCF (0.5 mL, 3.3 mmol) and NMM (0.4 mL, 3.3 mmol) were added and placed under N₂ and cooled to −10° C. The reaction mixture gradually became opaque with a fine white precipitate. Then ethyl amine in 2 M THF (1.5 ml, 3.0 mmol) and NMM (0.3 mL, 3.0 mmol) were added at 0° C. The reaction mixture was stirred at room temperature for 2 h. After 2 h, starting material was consumed and desired mass was formed according to LCMS. The reaction mixture was diluted with ethyl acetate and was washed with 0.1N HCl solution (2 times), aq. 5% K₂CO₃ solution (2 times), water and brine. The organic phase was dried over Na₂SO₄, filtered, and concentrated under reduced pressure to get the compound. The compound was purified by combi-flash column chromatography using 0-50% EtOAc in hexanes as eluent to get benzyl N²-(tert-butoxycarbonyl)-N⁴-ethyl-D-asparaginate (9-3, 750 mg). [M+H]⁺=351; ¹H NMR (400 MHZ, DMSO-d₆) δ 7.86 (s, 1H), 7.34 (s, 5H), 7.14 (d, 1H), 5.09 (s, 2H), 4.40 (d, 1H), 3.15-2.92 (m, 2H), 2.65-2.51 (m, 1H), 2.48-2.34 (m, 1H), 1.45-1.22 (m, 9H), 0.97 (t, 3H).

Synthesis of N²-(tert-butoxycarbonyl)-N⁴-ethyl-D-asparagine, [Step 2]: To a stirred solution of benzyl N²-(tert-butoxycarbonyl)-N⁴-ethyl-D-asparaginate (9-3, 1.1 g, 3.1 mmol) dissolved in THF (5 mL) was bubbled with nitrogen gas for 10 min. Then 10% Pd—C(400 mg) was added and the reaction mixture was hydrogenated under balloon pressure for 3 h. The reaction was monitored by TLC. Upon completion, the reaction was filtered over celite with excess ethyl acetate to get N²-(tert-butoxycarbonyl)-N⁴-ethyl-D-asparagine (9-4, 800 mg, 97%) compound. The compound was used in next step without further purification. 9 [M+H]: 261; ¹H NMR (400 MHZ, DMSO-d₆) δ 12.52 (s, 1H), 7.89-7.76 (m, 1H), 6.89 (d, 1H), 4.26 (q, 1H), 3.10-2.98 (m, 2H), 2.50-2.35 (m, 2H), 1.37 (s, 9H), 0.99 (t, 3H).

Synthesis of tert-butyl ((R)-4-(ethylamino)-1, 4-dioxo-1-(((R)-4-phenyl-1-(4, 4, 5, 5-tetramethyl-1, 3, 2-dioxaborolan-2-yl)butyl)amino)butan-2-yl)carbamate, [Step 3]: To a stirred solution of N²-(tert-butoxycarbonyl)-N⁴-ethyl-D-asparagine (9-4, 200 mg, 0.8 mmol) in THF (8 mL) was added IBCF (0.1 mL, 0.8 mmol) and NMM (0.08 mL, 0.8 mmol) at −10° C. The reaction mixture was stirred at same temperature for 30 min. Then (R)-4-phenyl-1-(4, 4, 5, 5-tetramethyl-1, 3, 2-dioxaborolan-2-yl)butan-1-amine hydrochloride (9-5, 220 mg, 0.7 mmol) in DMF (1 mL) followed by NMM (0.08 mL, 0.7 mmol) was added to the reaction mixture at −15° C. It was gradually warmed to 0° C. and stirred for 2 h. LCMS of crude reaction mass confirmed the formation of desired product. It was neutralized with saturated aqueous 0.1 N HCl solution and extracted with ethyl acetate. The combined organic layer was washed with 5% K₂CO₃ solution, water, and brine, dried over Na₂SO₄, filtered and evaporated under reduced pressure to tert-butyl ((R)-4-(ethylamino)-1, 4-dioxo-1-(((R)-4-phenyl-1-(4, 4, 5, 5-tetramethyl-1, 3, 2-dioxaborolan-2-yl)butyl)amino)butan-2-yl)carbamate (9-6, 250 mg). The product was used directly without further purification. [M+H]: 518.

Synthesis of (R)-2-amino-N⁴-ethyl-N¹—((R)-4-phenyl-1-(4, 4, 5, 5-tetramethyl-1, 3, 2-dioxaborolan-2-yl)butyl) succinamide hydrochloride, [Step 4]: To a stirred solution of tert-butyl ((R)-4-(ethylamino)-1, 4-dioxo-1-(((R)-4-phenyl-1-(4, 4, 5, 5-tetramethyl-1, 3, 2-dioxaborolan-2-yl)butyl)amino)butan-2-yl)carbamate (9-6, 650 mg, 1.3 mmol) in 1, 4-dioxane (6 mL) was added 4 M HCl in 1, 4-dioxane (6.0 mL, 25.1 mmol) at 0° C. It was gradually warmed to 25° C. and stirred for 16 h. TLC showed complete consumption of starting material to form new polar spot. The volatiles were removed under reduced pressure to get (R)-2-amino-N⁴-ethyl-N¹—((R)-4-phenyl-1-(4, 4, 5, 5-tetramethyl-1, 3, 2-dioxaborolan-2-yl)butyl) succinamide hydrochloride (9-7, 550 mg). The product was directly used for next step without purification.

Synthesis of (R)—N⁴-ethyl-N¹—((R)-4-phenyl-1-(4, 4, 5, 5-tetramethyl-1, 3, 2-dioxaborolan-2-yl)butyl)-2-(pyrazine-2-carboxamido) succinimide, [Step 5]: To a stirred solution of pyrazine-2-carboxylic acid (9-8, 165 mg, 1.3 mmol) in THF (2 mL) was added IBCF (0.2 mL, 1.3 mmol) and NMM (0.15 mL, 1.3 mmol) at −15° C. The reaction mixture was stirred at same temperature for 30 min. Then (R)-2-amino-N⁴-ethyl-N¹—((R)-4-phenyl-1-(4, 4, 5, 5-tetramethyl-1, 3, 2-dioxaborolan-2-yl)butyl) succinamide hydrochloride (9-7, 550 mg, 1.2 mmol) in DMF (1 mL) followed by NMM (0.13 mL, 1.2 mmol) was added to the reaction mixture at same condition. It was gradually warmed to 0° C. and stirred for 2 h. LCMS of crude reaction mass confirmed the formation of desired product. It was neutralized with saturated aqueous 0.1 N HCl solution and extracted with ethyl acetate. The combined organic layer was washed with 5% K₂CO₃ solution, water, and brine, dried over Na₂SO₄, filtered and evaporated under reduced pressure. The material was purified by RP prep HPLC purification and lyophilized to afford (R)—N⁴-ethyl-N¹—((R)-4-phenyl-1-(4, 4, 5, 5-tetramethyl-1, 3, 2-dioxaborolan-2-yl)butyl)-2-(pyrazine-2-carboxamido) succinamide (9-9, 30 mg). [M−H]⁺=523.

Synthesis of ((R)-1-((R)-4-(ethylamino)-4-oxo-2-(pyrazine-2-carboxamido)butanamido)-4-phenylbutyl)boronic acid, [Step 6]: To a stirred solution of (R)—N⁴-ethyl-N¹—((R)-4-phenyl-1-(4, 4, 5, 5-tetramethyl-1, 3, 2-dioxaborolan-2-yl)butyl)-2-(pyrazine-2-carboxamido) succinamide (9-9, 30 mg, 0.06 mmol) and methylboronic acid (9-10, 35 mg, 0.6 mmol) in acetone (2 mL) was added 0.2 N HCl (2 mL) and the reaction mixture was stirred at RT overnight. TLC and LCMS showed full conversion of starting material with formation of new polar spot. The volatiles were evaporated and the residue was purified by RP prep HPLC purification and lyophilized to yield ((R)-1-((R)-4-(ethylamino)-4-oxo-2-(pyrazine-2-carboxamido)butanamido)-4-phenylbutyl)boronic acid (Compound 9, 13 mg). [M−H]⁺=440; ¹H NMR δ ¹H NMR (400 MHZ, Methanol-d₄) δ 9.23 (s, 1H), 8.80 (d, 1H), 8.72-8.66 (m, 1H), 7.24-7.05 (m, 5H), 5.23-5.12 (m, 1H), 3.23-3.06 (m, 2H), 2.93 (dd, 1H), 2.83 (dd, 1H), 2.63-2.54 (m, 3H), 1.81-1.36 (m, 4H), 1.07 (t, 3H).

### Example 10: Synthesis of ((R)-1-((R)-4-oxo-4-(piperidin-1-yl)-2-(pyrazine-2-carboxamido)butanamido)-4-phenylbutyl)boronic acid

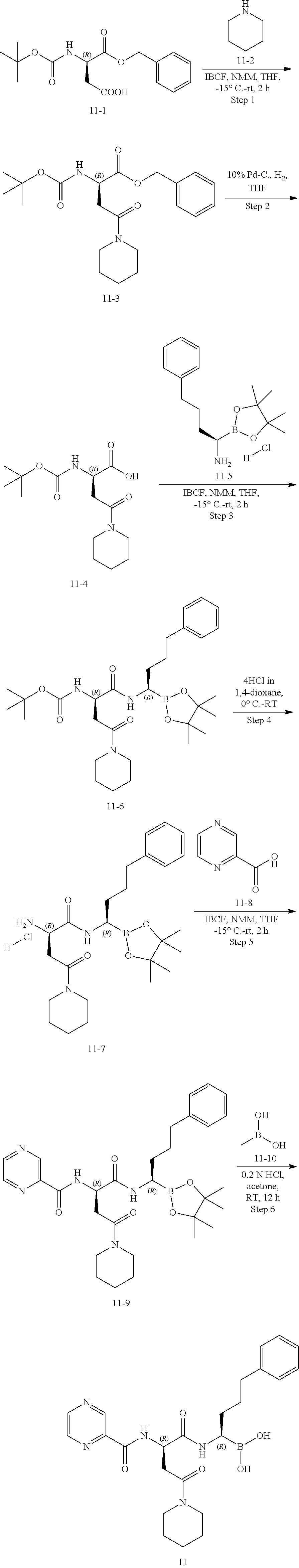

Synthesis of benzyl (R)-2-((tert-butoxycarbonyl)amino)-4-oxo-4-(piperidin-1-yl)butanoate, [Step 1]: To a stirred solution of (R)-4-(benzyloxy)-3-((tert-butoxycarbonyl)amino)-4-oxobutanoic acid (11-1, 1.0 g, 3.2 mmol) in THF (25 mL) was added IBCF (0.4 mL, 3.2 mmol) and NMM (0.4 mL, 3.3 mmol) at −15° C. The reaction mixture was stirred at same temperature for 30 min. Then piperidine (11-2, 0.3 mL, 2.9 mmol) followed by NMM (0.3 mL, 2.9 mmol) was added to the reaction mixture at −15° C. It was gradually warmed to 0° C. and stirred for 2 h. LCMS of the crude reaction mass confirmed the formation of desired product. It was neutralized with saturated aqueous 0.1 N HCl solution and extracted with ethyl acetate. The combined organic layer was washed with 5% K₂CO₃ solution, water and brine. It was dried over Na₂SO₄, filtered and evaporated under reduced pressure to get a residue. The product was purified through combiflash column chromatography to afford benzyl (R)-2-((tert-butoxycarbonyl)amino)-4-oxo-4-(piperidin-1-yl)butanoate (11-3, 1.0 g). [M+H]⁺=391.

Synthesis of (R)-2-((tert-butoxycarbonyl)amino)-4-oxo-4-(piperidin-1-yl)butanoic acid, [Step 2]: To a stirred solution of benzyl (R)-2-((tert-butoxycarbonyl)amino)-4-oxo-4-(piperidin-1-yl)butanoate (11-3, 750 mg, 1.9 mmol) dissolved in THF (20 mL) was bubbled with nitrogen gas for 10 min. Then 10% Pd—C(300 mg, 2.6 mmol) was added and the reaction mixture was hydrogenated under balloon pressure for 16 h. The reaction was monitored by TLC. Upon completion, the reaction was filtered over celite with excess ethyl acetate. Removal of solvent under reduced pressure provided (R)-2-((tert-butoxycarbonyl)amino)-4-oxo-4-(piperidin-1-yl)butanoic acid (11-4, 550 mg). [M+H]: 301.

Synthesis of tert-butyl ((R)-1, 4-dioxo-1-(((R)-4-phenyl-1-(4, 4, 5, 5-tetramethyl-1, 3, 2-dioxaborolan-2-yl)butyl)amino)-4-(piperidin-1-yl)butan-2-yl)carbamate, [Step 3]: To a stirred solution of (R)-2-((tert-butoxycarbonyl)amino)-4-oxo-4-(piperidin-1-yl)butanoic acid (11-4, 740 mg, 2.5 mmol) in THF (10 mL) was added IBCF (0.3 mL, 2.5 mmol) and NMM (0.3 mL, 2.5 mmol) at −15° C. The reaction mixture was stirred at same temperature for 30 min. Then (R)-4-phenyl-1-(4, 4, 5, 5-tetramethyl-1, 3, 2-dioxaborolan-2-yl)butan-1-amine hydrochloride (11-5, 700 mg, 2.2 mmol) in DMF (1 mL) followed by NMM (0.2 mL, 2.2 mmol) was added to the reaction mixture at −15° C. It was gradually warmed to 0° C. and stirred for 2 h. LCMS of crude reaction mass confirmed the formation of desired product. It was neutralized with saturated aqueous 0.1 N HCl solution and extracted with ethyl acetate. The combined organic layer was washed with 5% K₂CO₃ solution, water and brine, dried over Na₂SO₄, filtered and evaporated under reduced pressure to afford tert-butyl ((R)-1, 4-dioxo-1-(((R)-4-phenyl-1-(4, 4, 5, 5-tetramethyl-1, 3, 2-dioxaborolan-2-yl)butyl)amino)-4-(piperidin-1-yl)butan-2-yl)carbamate (11-6, 700 mg). [M+H]⁺=558.

Synthesis of (R)-2-amino-4-oxo-N—((R)-4-phenyl-1-(4, 4, 5, 5-tetramethyl-1, 3, 2-dioxaborolan-2-yl)butyl)-4-(piperidin-1-yl)butanamide hydrochloride, [Step 4]: To a solution of tert-butyl ((R)-1, 4-dioxo-1-(((R)-4-phenyl-1-(4, 4, 5, 5-tetramethyl-1, 3, 2-dioxaborolan-2-yl)butyl)amino)-4-(piperidin-1-yl)butan-2-yl)carbamate (11-6, 700 mg, 1.2 mmol) in 1, 4-dioxane (6 mL) was added 4 M HCl in dioxane (6.0 mL, 24.0 mmol) at 0° C. It was gradually warmed to 25° C. and stirred for 16 h. TLC showed complete consumption of starting material to form new polar spot. The volatiles were removed under reduced pressure to get (R)-2-amino-4-oxo-N—((R)-4-phenyl-1-(4, 4, 5, 5-tetramethyl-1, 3, 2-dioxaborolan-2-yl)butyl)-4-(piperidin-1-yl)butanamide hydrochloride (11-7, 560 mg). The product was directly used for next step without purification.

Synthesis of N—((R)-1, 4-dioxo-1-(((R)-4-phenyl-1-(4, 4, 5, 5-tetramethyl-1, 3, 2-dioxaborolan-2-yl)butyl)amino)-4-(piperidin-1-yl)butan-2-yl) pyrazine-2-carboxamide, [Step 5]: To a stirred solution of pyrazine-2-carboxylic acid (11-8, 135 mg, 1.1 mmol) in THF (8 mL) was added IBCF (0.13 mL, 1.1 mmol) and NMM (0.11 mL, 1.1 mmol) at −15° C. The reaction mixture was stirred at same temperature for 30 min. Then (R)-2-amino-4-oxo-N—((R)-4-phenyl-1-(4, 4, 5, 5-tetramethyl-1, 3, 2-dioxaborolan-2-yl)butyl)-4-(piperidin-1-yl)butanamide hydrochloride (11-7, 480 mg, 1.0 mmol) in DMF (1 mL) followed by NMM (0.1 mL, 1.0 mmol) was added to the reaction mixture under same condition. It was gradually warmed to 0° C. and stirred for 2 h. LCMS of the crude reaction mass confirmed the formation of desired product. It was neutralized with saturated aqueous 0.1 N HCl solution and extracted with ethyl acetate. The combined organic layer was washed with 5% K₂CO₃ solution, water and brine. It was dried over Na₂SO₄, filtered and evaporated under reduced pressure. The material was purified by RP prep HPLC purification and lyophilized to afford N—((R)-1, 4-dioxo-1-(((R)-4-phenyl-1-(4, 4, 5, 5-tetramethyl-1, 3, 2-dioxaborolan-2-yl)butyl)amino)-4-(piperidin-1-yl)butan-2-yl) pyrazine-2-carboxamide (11-9, 45 mg). 1 [M−H]⁺=563.

Synthesis of ((R)-1-((R)-4-oxo-4-(piperidin-1-yl)-2-(pyrazine-2-carboxamido)butanamido)-4-phenylbutyl)boronic acid, [Step 6]: To a stirred solution of N—((R)-1, 4-dioxo-1-(((R)-4-phenyl-1-(4, 4, 5, 5-tetramethyl-1, 3, 2-dioxaborolan-2-yl)butyl)amino)-4-(piperidin-1-yl)butan-2-yl) pyrazine-2-carboxamide (11-9, 45 mg, 0.08 mmol) and methylboronic acid (11-10, 45 mg, 0.8 mmol) in acetone (2 mL) was added 0.2 N HCl (2 mL) and the reaction mixture was stirred at RT overnight. TLC and LCMS showed full conversion of starting material with formation of new polar spot. The volatiles were evaporated and the residue was redissolved in acetone and deionized water and freeze-dried to obtain ((R)-1-((R)-4-oxo-4-(piperidin-1-yl)-2-(pyrazine-2-carboxamido)butanamido)-4-phenylbutyl)boronic acid (Compound 11, 22 mg). LCMS (ESI) Calcd. for C₂₄H₃₂BN₅O₅: 481, found [M−H]⁺=480; ¹H NMR (400 MHZ, MeOD) δ 9.24 (s, 1H), 8.79 (d, 1H), 8.68 (s, 1H), 7.21-7.08 (m, 5H), 5.27 (t, 1H), 3.51-3.45 (m, 4H), 2.99-2.98 (m, 2H), 2.60-2.56 (m, 3H), 1.65-1.48 (m, 10H).

### Example 11: Synthesis of ((R)-1-((R)-4-methoxy-2-(pyrazine-2-carboxamido)butanamido)-4-phenylbutyl)boronic acid

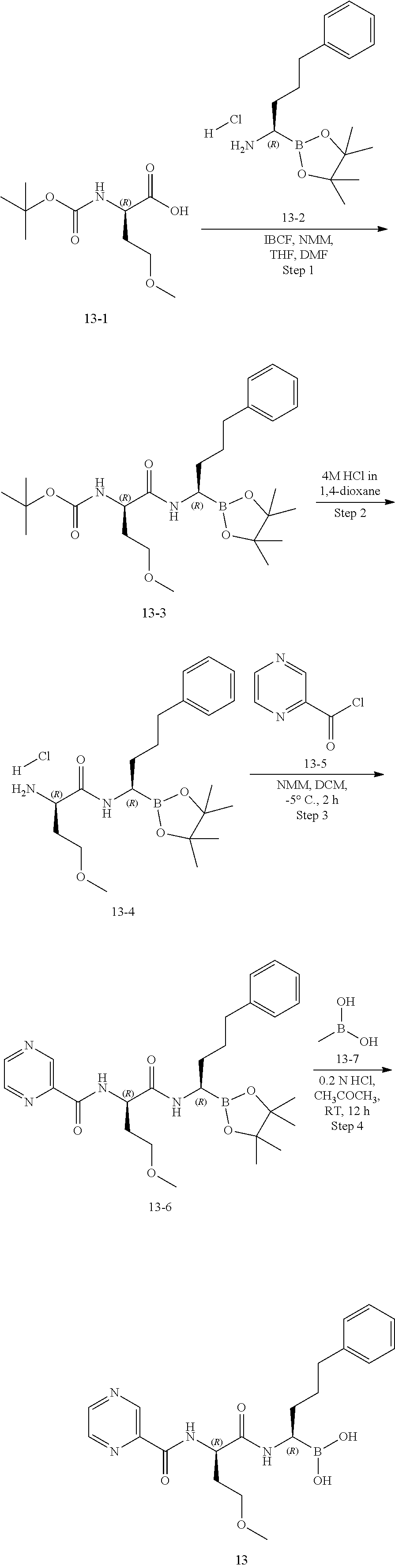

Synthesis of tert-butyl ((R)-4-methoxy-1-oxo-1-(((R)-4-phenyl-1-(4, 4, 5, 5-tetramethyl-1, 3, 2-dioxaborolan-2-yl)butyl)amino)butan-2-yl)carbamate, [Step 1]: To a stirred solution of N-(tert-butoxycarbonyl)-O-methyl-D-homoserine (13-1, 330 mg, 1.4 mmol) in tetrahydrofuran (8 mL) was added IBCF (0.2 mL, 1.4 mmol) and NMM (0.2 mL, 1.4 mmol) at −15° C. and stirred for 30 min. A solution of (R)-4-phenyl-1-(4, 4, 5, 5-tetramethyl-1, 3, 2-dioxaborolan-2-yl)butan-1-amine, hydrochloride (13-2, 400 mg, 1.3 mmol) in dimethylformamide (1 mL) was added to it followed by NMM (0.17 mL, 1.3 mmol) at −15° C. The reaction mixture was gradually warmed to 0° C. and stirred for 2 h. The reaction mass was diluted with ethyl acetate and washed with 0.1N HCl (twice), 5% aqueous K₂CO₃ solution (twice), water (twice) and brine, dried over anhydrous Na₂SO₄ and evaporated under reduced pressure to afford tert-butyl ((R)-4-methoxy-1-oxo-1-(((R)-4-phenyl-1-(4, 4, 5, 5-tetramethyl-1, 3, 2-dioxaborolan-2-yl)butyl)amino)butan-2-yl)carbamate (13-3, 500 mg). [M+H]⁺=491.

Synthesis of (R)-2-amino-4-methoxy-N—((R)-4-phenyl-1-(4, 4, 5, 5-tetramethyl-1, 3, 2-dioxaborolan-2-yl)butyl)butanamide hydrochloride, [Step 2]: To a solution of tert-butyl ((R)-4-methoxy-1-oxo-1-(((R)-4-phenyl-1-(4, 4, 5, 5-tetramethyl-1, 3, 2-dioxaborolan-2-yl)butyl)amino)butan-2-yl)carbamate (13-3, 500 mg, 1.0 mmol) in 1, 4-dioxane (6 mL) was added 4M HCl in 1, 4-dioxane (5.0 mL, 20.0 mmol) at 0° C. and stirred at 25° C. for 16 h. Volatiles were removed under reduced pressure to give (R)-2-amino-4-methoxy-N—((R)-4-phenyl-1-(4, 4, 5, 5-tetramethyl-1, 3, 2-dioxaborolan-2-yl)butyl)butanamide hydrochloride (13-4, 400 mg). [M+H]⁺=389.

Synthesis of N—((R)-4-methoxy-1-oxo-1-(((R)-4-phenyl-1-(4, 4, 5, 5-tetramethyl-1, 3, 2-dioxaborolan-2-yl)butyl)amino)butan-2-yl) pyrazine-2-carboxamide, [Step 3]: To a stirred solution of (R)-2-amino-4-methoxy-N—((R)-4-phenyl-1-(4, 4, 5, 5-tetramethyl-1, 3, 2-dioxaborolan-2-yl)butyl)butanamide hydrochloride (13-4, 400 mg, 1.0 mmol) in dichloromethane (5 mL) was added NMM (0.3 mL, 2.0 mmol) at −5° C. and stirred for 15 min. Pyrazine-2-carbonyl chloride (5, 160 mg, 1.1 mmol) was added to it and stirred at 0° C. for 2 h. The reaction mass was diluted with dichloromethane and washed with water and brine, dried over anhydrous Na₂SO₄ and evaporated under reduced pressure. The compound was purified by prep HPLC purification and lyophilized to afford N—((R)-4-methoxy-1-oxo-1-(((R)-4-phenyl-1-(4, 4, 5, 5-tetramethyl-1, 3, 2-dioxaborolan-2-yl)butyl)amino)butan-2-yl) pyrazine-2-carboxamide (13-6, 60 mg). [M−H]⁻=495.

Synthesis of ((R)-1-((R)-4-methoxy-2-(pyrazine-2-carboxamido)butanamido)-4-phenyl butyl)boronic acid, [Step 4]: To a stirred solution of N—((R)-4-methoxy-1-oxo-1-(((R)-4-phenyl-1-(4, 4, 5, 5-tetramethyl-1, 1, 3, 2-dioxaborolan-2-yl)butyl)amino)butan-2-yl) pyrazine-2-carboxamide (13-6, 50 mg, 0.10 mmol) and methylboronic acid (13-7, 60 mg, 1 mmol) in acetone (2 mL) was added 0.2N HCl (2 mL) and stirred at ambient temperature for overnight. Volatiles were removed under reduced pressure and purified by prep HPLC purification to afford ((R)-1-((R)-4-methoxy-2-(pyrazine-2-carboxamido)butanamido)-4-phenylbutyl)boronic acid (Compound 13, 24 mg). [M−H]⁻=413; ¹H NMR (400 MHZ, Methanol-d₄): δ 9.21 (s, 1H), 8.80 (d, 1H), 8.73-8.68 (m, 1H), 7.25-7.06 (m, 5H), 4.94 (t, 1H), 3.57-3.48 (m, 2H), 3.46-3.30 (m, 2H), 2.60 (q, 3H), 2.35-2.09 (m, 2H), 1.80-1.38 (m, 4H).

### Example 12: Synthesis of (R)-1-((R)-2-acetamido-4-morpholino-4-oxobutanamido)-4-phenylbutyl)boronic acid

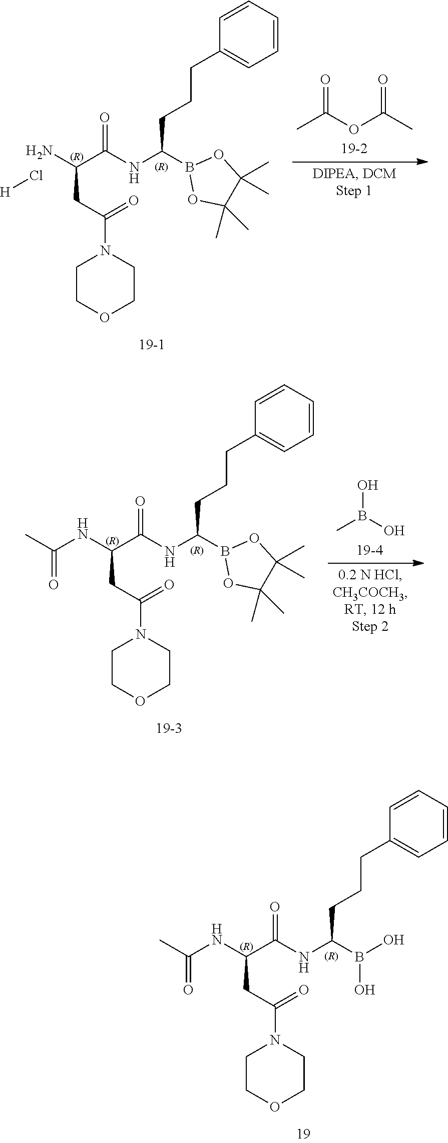

Synthesis of (R)-2-acetamido-4-morpholino-4-oxo-N—((R)-4-phenyl-1-(4, 4, 5, 5-tetramethyl-1, 3, 2-dioxaborolan-2-yl)butyl)butanamide, [Step 1]: To a stirred solution of (R)-2-amino-4-morpholino-4-oxo-N—((R)-4-phenyl-1-(4, 4, 5, 5-tetramethyl-1, 3, 2-dioxaborolan-2-yl)butyl)butanamide hydrochloride (19-1, 300 mg, 0.6 mmol) and acetic anhydride (19-2, 0.06 mL, 0.7 mmol) in DCM (4 mL), DIPEA (0.5 mL, 3 mmol) was added in ice cold condition and the reaction mixture was stirred at RT for 2 h. TLC and LCMS showed full conversion of starting material with formation of new spot. The reaction was diluted with DCM and washed with water and brine solution. The organic phase was dried over Na₂SO₄, filtered and evaporated to yield product. The material was purified by RP prep HPLC purification and lyophilized to afford (R)-2-acetamido-4-morpholino-4-oxo-N—((R)-4-phenyl-1-(4, 4, 5, 5-tetramethyl-1, 3, 2-dioxaborolan-2-yl)butyl)butanamide (19-3, 50 mg). [M−H]⁺=500.

Synthesis of (R)-1-((R)-2-acetamido-4-morpholino-4-oxobutanamido)-4-phenylbutyl) boronic acid, [Step 2]: To a stirred solution of (R)-2-acetamido-4-morpholino-4-oxo-N—((R)-4-phenyl-1-(4, 4, 5, 5-tetramethyl-1, 3, 2-dioxaborolan-2-yl)butyl)butanamide (19-3, 50 mg, 0.1 mmol) and methylboronic acid (19-4, 60 mg, 1.0 mmol) in acetone (3 mL) was added 0.2 N HCl (3.0 mL) and the reaction mixture was stirred at RT overnight. TLC and LCMS showed full conversion of starting material with formation of new polar spot. The volatiles were evaporated and the product was purified via RP prep HPLC purification and lyophilized to afford (R)-1-((R)-2-acetamido-4-morpholino-4-oxobutanamido)-4-phenylbutyl)boronic acid (Compound 19, 25 mg). [M−H]⁺=418; ¹H NMR (400 MHZ, MeOD) δ 7.24-7.10 (m, 5H), 5.00 (t, 1H), 3.66-3.59 (m, 4H), 3.52-3.48 (m, 4H), 3.0-2.86 (m, 2H), 2.64-2.57 (m, 3H), 1.97 (s, 3H), 1.71-1.47 (m, 4H).

### Example 13: Synthesis of ((R)-1-((R)-2-(morpholine-4-carboxamido)-4-morpholino-4-oxobutanamido)-4-phenylbutyl)boronic acid

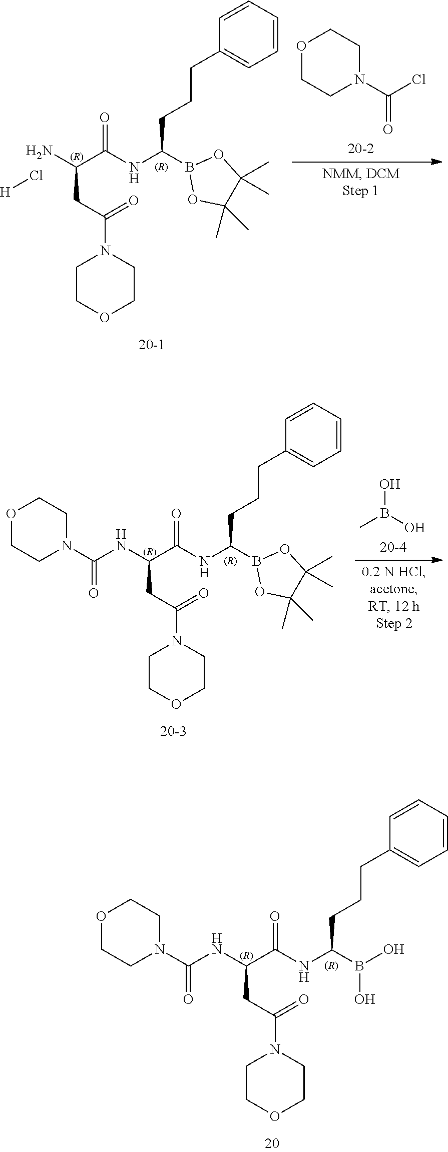

Synthesis of N—((R)-4-morpholino-1, 4-dioxo-1-(((R)-4-phenyl-1-(4, 4, 5, 5-tetramethyl-1, 3, 2-dioxaborolan-2-yl)butyl)amino)butan-2-yl) morpholine-4-carboxamide, [Step 1]: To a stirred solution of (R)-2-amino-4-morpholino-4-oxo-N—((R)-4-phenyl-1-(4, 4, 5, 5-tetramethyl-1, 3, 2-dioxaborolan-2-yl)butyl)butanamide hydrochloride (20-1, 250 mg, 0.5 mmol) and morpholine-4-carbonyl chloride (0.07 mL, 0.6 mmol) in DCM (4 mL), NMM (0.1 mL, 1 mmol) was added in ice cold condition and the reaction mixture was stirred at RT for 2 h. TLC and LCMS showed full conversion of starting material with formation of new polar spot. The reaction was diluted with DCM and washed with water and brine solution. The organic phase was dried over Na₂SO₄, filtered and evaporated to yield product. The material was purified by RP prep HPLC purification and lyophilized to afford N—((R)-4-morpholino-1, 4-dioxo-1-(((R)-4-phenyl-1-(4, 4, 5, 5-tetramethyl-1, 3, 2-dioxaborolan-2-yl)butyl)amino)butan-2-yl) morpholine-4-carboxamide (20-3, 52 mg). LCMS (ESI) [M−H]⁺=572; ¹H NMR (400 MHZ, MeOD) δ 7.24-7.09 (m, 5H), 4.96-4.92 (m, 1H), 3.65-3.59 (m, 8H), 3.51-3.49 (m, 4H), 3.40-3.32 (m, 4H), 3.01-2.87 (m, 2H), 2.62-2.57 (m, 3H), 1.70-1.48 (m, 4H), 1.19-1.14 (m, 5H).

Synthesis of ((R)-1-((R)-2-(morpholine-4-carboxamido)-4-morpholino-4-oxobutanamido)-4-phenylbutyl)boronic acid, [Step 2]: To a stirred solution of N—((R)-4-morpholino-1, 4-dioxo-1-(((R)-4-phenyl-1-(4, 4, 5, 5-tetramethyl-1, 3, 2-dioxaborolan-2-yl)butyl)amino)butan-2-yl) morpholine-4-carboxamide (20-3, 50 mg, 0.09 mmol) and methylboronic acid (20-4, 52 mg, 0.9 mmol) in acetone (3 mL) was added 0.2 N HCl (3.0 mL) and the reaction mixture was stirred at RT overnight. TLC and LCMS showed full conversion of starting material with formation of new polar spot. The volatiles were evaporated and the residue was redissolved in acetonitrile and deionized water and freeze-dried to obtain product. The material was purified by RP prep HPLC purification and lyophilized to afford ((R)-1-((R)-2-(morpholine-4-carboxamido)-4-morpholino-4-oxobutanamido)-4-phenylbutyl)boronic acid (Compound 20, 16 mg). [M−H]⁺=489; ¹H NMR (400 MHz, MeOD) δ 7.22-7.11 (m, 5H), 4.93 (t, 1H), 3.65-3.59 (m, 8H), 3.51-3.48 (m, 4H), 3.37-3.33 (m, 4H), 2.99-2.91 (m, 2H), 2.62-2.59 (m, 3H), 1.75-1.40 (m, 4H).

### Example 14: Synthesis of ((R)-1-((R)-2-((tert-butoxycarbonyl)amino)-4-morpholino-4-oxobutanamido)-4-phenylbutyl)boronic acid

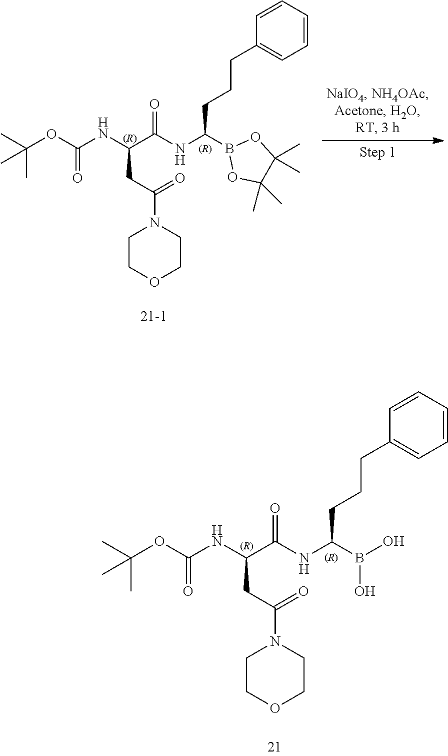

Synthesis of ((R)-1-((R)-2-((tert-butoxycarbonyl)amino)-4-morpholino-4-oxobutanamido)-4-phenylbutyl)boronic acid, [Step 1]: To a stirred solution tert-butyl ((R)-4-morpholino-1, 4-dioxo-1-(((R)-4-phenyl-1-(4, 4, 5, 5-tetramethyl-1, 3, 2-dioxaborolan-2-yl)butyl)amino)butan-2-yl)carbamate (21-1, 300 mg, 0.5 mmol) in acetone (6 mL) and water (6 mL) was added NH₄OAc (40 mg, 0.5 mmol) and stirred for 5 min. NalO₄ (115 mg, 0.5 mmol) was added to it portion wise and stirred for 3 h. Volatiles were removed under reduced pressure and partitioned between ethyl acetate and water. The organic layer was collected. The aqueous layer was further extracted with EtOAc (twice). The combined organic layer was dried over anhydrous Na₂SO₄ and evaporated under reduced pressure. The product was purified through RP prep HPLC purification to afford ((R)-1-((R)-2-((tert-butoxycarbonyl)amino)-4-morpholino-4-oxobutanamido)-4-phenylbutyl) boronic acid (Compound 21, 50 mg). [M−H]⁺=477; ¹H NMR (400 MHZ, MeOD) δ 7.26-7.13 (m, 5H), 4.74 (t, 1H), 3.72-3.60 (m, 4H), 3.52-2.49 (m, 4H), 2.92 (t, 2H), 2.65-2.55 (m, 3H), 1.71-1.50 (m, 4H), 1.45 (s, 9H).

### Example 15: Synthesis of ((R)-1-((R)-5-morpholino-5-oxo-2-(pyrazine-2-carboxamido) pentanamido)-4-phenylbutyl)boronic acid

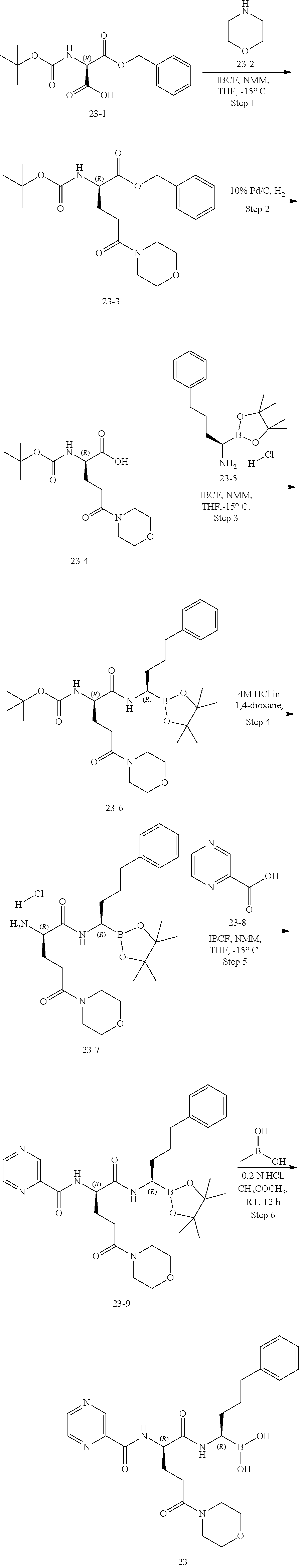

Synthesis of benzyl (R)-2-((tert-butoxycarbonyl)amino)-5-morpholino-5-oxopentanoate, [Step 1]: To a stirred solution of (R)-5-(benzyloxy)-4-((tert-butoxycarbonyl)amino)-5-oxopentanoic acid (23-1, 1.3 g, 3.8 mmol) in THF (50 mL) was added IBCF (0.5 mL, 3.8 mmol) and NMM (0.4 mL, 3.8 mmol) at −15° C. Reaction mixture was stirred at same temperature for 30 min. Then morpholine (23-2, 0.3 mL, 3.5 mmol) followed by NMM (0.4 mL, 3.5 mmol) was added to reaction mixture at −15° C. It was gradually warmed to 0° C. and stirred for 2 h. LCMS of crude reaction mass confirmed the formation of desired product. It was neutralized with saturated aqueous 0.1 N HCl solution and extracted with ethyl acetate. Combined organic layer was washed with 5% K₂CO₃ solution, water, brine, dried over Na₂SO₄, filtered and evaporated under reduced pressure to get product. Product was purified through combiflash column chromatography to afford benzyl (R)-2-((tert-butoxycarbonyl)amino)-5-morpholino-5-oxopentanoate (23-3, 1.10 g). ¹H NMR (400 MHZ, DMSO-d₆) δ 7.36-7.31 (m, 6H), 5.17-5.05 (m, 2H), 4.06-4.01 (m, 1H), 3.51-3.50 (m, 4H), 3.41-3.39 (m, 2H), 2.39-2.28 (m, 2H), 1.95-1.89 (m, 1H), 1.84-1.77 (m, 1H), 1.37 (s, 9H).

Synthesis of (R)-2-((tert-butoxycarbonyl)amino)-5-morpholino-5-oxopentanoic acid, [Step 2]: Benzyl (R)-2-((tert-butoxycarbonyl)amino)-5-morpholino-5-oxopentanoate (23-3, 1.10 g, 2.7 mmol)) was dissolved in THF (20 mL), 10% Pd—C(425 mg, 4 mmol) was added and hydrogenated under balloon pressure for 3 h. After completion, the reaction was filtered over Celite and washed with excess ethyl acetate. Removal of solvents in vacuo provided the product as (R)-2-((tert-butoxycarbonyl)amino)-5-morpholino-5-oxopentanoic acid (23-4, 800 mg). ¹H NMR (400 MHZ, DMSO-_{D6}) δ 12.5 (br s, 1H), 7.08 (d, 1H), 3.93-3.88 (m, 1H), 3.55-3.51 (m, 4H), 3.41 (d, 4H), 2.40-2.28 (m, 2H), 1.99-1.88 (m, 1H), 1.80-1.73 (m, 1H), 1.38 (s, 9H).

Synthesis of tert-butyl ((R)-5-morpholino-1, 5-dioxo-1-(((R)-4-phenyl-1-(4, 4, 5, 5-tetramethyl-1, 3, 2-dioxaborolan-2-yl)butyl)amino) pentan-2-yl)carbamate, [Step 3]: To a stirred solution of (R)-2-((tert-butoxycarbonyl)amino)-5-morpholino-5-oxopentanoic acid (23-4, 730 mg, 2.3 mmol) in THF (8 mL) was added IBCF (0.30 mL, 2.3 mmol) and NMM (0.3 mL, 2.3 mmol) at −15° C. Reaction mixture was stirred at same temperature for 30 min. (R)-4-phenyl-1-(4, 4, 5, 5-tetramethyl-1, 3, 2-dioxaborolan-2-yl)butan-1-amine: hydrochloride (23-5, 650 mg, 2.1 mmol) in DMF (1 mL) followed by NMM (0.2 mL, 2.1 mmol) was added to reaction mixture at −15° C. It was gradually warmed to 0° C. and stirred for 2 h. It was neutralized with saturated aqueous 0.1 N HCl solution and extracted with ethyl acetate. Combined organic layer was washed with 5% K₂CO₃ solution, water, brine, dried over Na₂SO₄, filtered and evaporated under reduced pressure to afford tert-butyl ((R)-5-morpholino-1, 5-dioxo-1-(((R)-4-phenyl-1-(4, 4, 5, 5-tetramethyl-1, 3, 2-dioxaborolan-2-yl)butyl)amino) pentan-2-yl)carbamate (23-6, 1.10 g). Product was used directly without further purification. [M−H]⁺=572.

Synthesis of (R)-2-amino-5-morpholino-5-oxo-N—((R)-4-phenyl-1-(4, 4, 5, 5-tetramethyl-1, 3, 2-dioxaborolan-2-yl)butyl) pentanamide, [Step 4]: To a solution of tert-butyl ((R)-5-morpholino-1, 5-dioxo-1-(((R)-4-phenyl-1-(4, 4, 5, 5-tetramethyl-1, 3, 2-dioxaborolan-2-yl)butyl)amino) pentan-2-yl)carbamate (23-6, 1.00 g, 1.7 mmol) in 1, 4-dioxane (5 mL) was added 4 M HCl in 1, 4-dioxane (6.5 mL, 26 mmol) at 0° C. It was gradually warmed to 25° C. and stirred for 16 h. Volatiles were removed under reduced pressure to get (R)-2-amino-5-morpholino-5-oxo-N—((R)-4-phenyl-1-(4, 4, 5, 5-tetramethyl-1, 3, 2-dioxaborolan-2-yl)butyl) pentanamide (23-7, 800 mg) which was directly used in next step. [M−H]⁺=472.

Synthesis of N—((R)-5-morpholino-1, 5-dioxo-1-(((R)-4-phenyl-1-(4, 4, 5, 5-tetramethyl-1, 3, 2-dioxaborolan-2-yl)butyl)amino) pentan-2-yl) pyrazine-2-carboxamide, [Step 5]: To a solution of pyrazine-2-carboxylic acid (23-8, 231 mg, 1.9 mmol) in THF (15 mL) under argon atmosphere was added IBCF (0.3 mL, 1.9 mmol) followed by NMM (0.3 mL, 1.9 mmol) at −15° C. and stirred for 45 min. (R)-2-amino-5-morpholino-5-oxo-N—((R)-4-phenyl-1-(4, 4, 5, 5-tetramethyl-1, 3, 2-dioxaborolan-2-yl)butyl) pentanamide (23-7, 800 mg, 1.7 mmol) followed by NMM (0.2 mL, 1.9 mmol) was added to it and stirred for 2 h. The reaction was quenched with water and diluted with EtOAc. Organic layer was collected and washed successively with 0.1 M aqueous HCl, 5% aqueous K₂CO₃ solution, water and brine, dried over anhydrous Na₂SO₄ and concentrated under reduced pressure to get product. The material was purified by prep HPLC purification and lyophilized to afford N—((R)-5-morpholino-1, 5-dioxo-1-(((R)-4-phenyl-1-(4, 4, 5, 5-tetramethyl-1, 3, 2-dioxaborolan-2-yl)butyl)amino) pentan-2-yl) pyrazine-2-carboxamide (23-9, 900 mg). [M−H]⁺=578.

Synthesis of ((R)-1-((R)-5-morpholino-5-oxo-2-(pyrazine-2-carboxamido) pentanamido)-4-phenylbutyl)boronic acid, [Step 6]: To a stirred solution of N—((R)-5-morpholino-1, 5-dioxo-1-(((R)-4-phenyl-1-(4, 4, 5, 5-tetramethyl-1, 3, 2-dioxaborolan-2-yl)butyl)amino) pentan-2-yl) pyrazine-2-carboxamide (23-9, 75 mg, 0.2 mmol) and methylboronic acid (116 mg, 1.9 mmol) in acetone (4 mL) was added 0.2 N HCl (4.0 mL) and the reaction mixture was stirred at RT overnight. TLC and LCMS showed full conversion of starting material with formation of new polar spot. The volatiles were evaporated and the residue was redissolved in acetone and deionized water and freeze-dried to obtain product. The material was purified by prep HPLC purification and lyophilized to afford ((R)-1-((R)-5-morpholino-5-oxo-2-(pyrazine-2-carboxamido) pentanamido)-4-phenylbutyl)boronic acid (Compound 23, 40 mg). [M−H]⁺=496; 1H NMR (400 MHZ, MeOD) δ 9.21 (s, 1H), 8.80 (d, 1H), 8.70 (t, 1H), 7.22-7.19 (m, 2H), 7.16-7.10 (m, 3H), 3.60-3.54 (m, 6H), 3.47-3.45 (m, 2H), 2.65-2.53 (m, 5H), 2.34-2.28 (m, 1H), 2.24-2.17 (m, 1H), 1.69-1.62 (m, 2H) 1.59-1.49 (m, 3H).

### Example 16: Synthesis of ((R)-1-((R)-4-morpholino-4-oxo-2-(pyrazine-2-carboxamido)butanamido)butyl)boronic acid

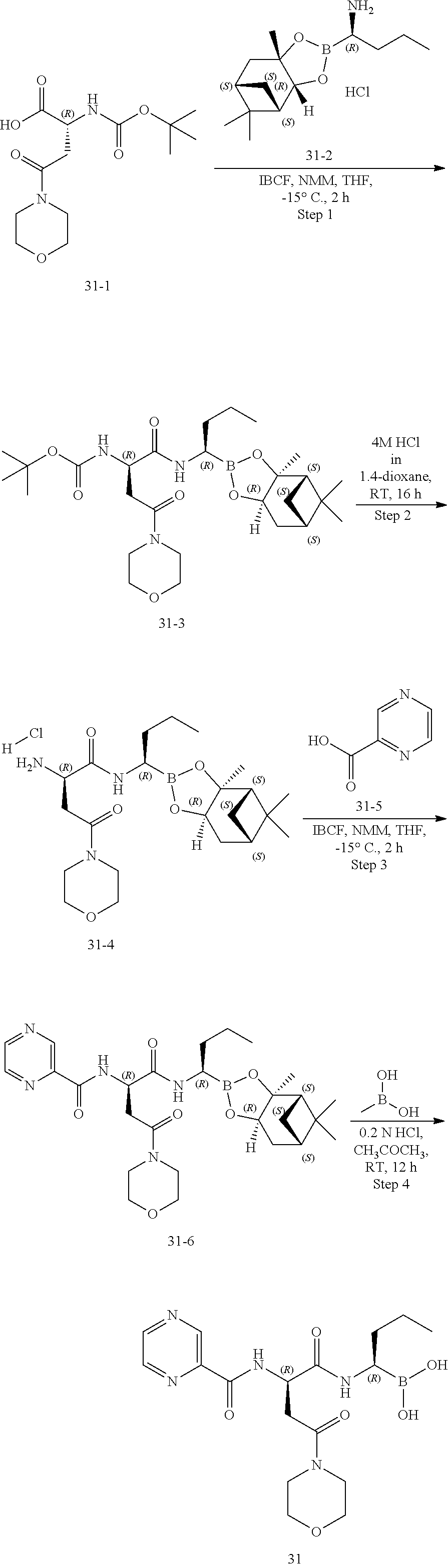

Synthesis of tert-butyl ((R)-4-morpholino-1, 4-dioxo-1-(((R)-1-((3aS, 4S, 6S, 7aR)-3a, 5, 5-trimethylhexa hydro-4, 6-methanobenzo[d][1, 3, 2]dioxaborol-2-yl)butyl)amino)butan-2-yl)carbamate, [Step 1]: To a stirred solution of (R)-2-((tert-butoxycarbonyl)amino)-4-morpholino-4-oxobutanoic acid (31-1, 690 mg, 2.3 mmol) in THF (10 mL), NMM (0.3 mL, 2.3 mmol) and IBCF (0.3 mL, 2.3 mmol) were added at −15° C. and the reaction mixture was stirred at this temperature for 1 h. To the above solution, (R)-BoroNva-(+)-Pinanediol hydrochloride (31-2, 600 mg, 2.1 mmol) and NMM (0.3 mL, 2.3 mmol) were added and the reaction mixture was stirred at RT for 2 h. After completion of the reaction (monitored by TLC and LC-MS), the reaction mixture was diluted with EtOAc and washed subsequently with 0.1 M HCl, 5% K₂CO₃, water and brine. The organic phase was dried over Na₂SO₄, filtered and evaporated to yield tert-butyl ((R)-4-morpholino-1, 4-dioxo-1-(((R)-1-((3aS, 4S, 6S, 7aR)-3a, 5, 5-trimethylhexahydro-4, 6-methanobenzo[d][1, 3, 2]dioxaborol-2-yl)butyl)amino)butan-2-yl)carbamate (31-3, 1100 mg). The product was used for next step without further purification. [M−H]⁻=535.

Synthesis of (R)-2-amino-4-morpholino-4-oxo-N—((R)-1-((3aS, 4S, 6S, 7aR)-3a, 5, 5-trimethylhexahydro-4, 6-methanobenzo[d][1, 3, 2]dioxaborol-2-yl)butyl)butanamide hydrochloride, [Step 2]: To a stirred solution of tert-butyl ((R)-4-morpholino-1, 4-dioxo-1-(((R)-1-((3aS, 4S, 6S, 7aR)-3a, 5, 5-trimethylhexa hydro-4, 6-methanobenzo[d][1, 3, 2]dioxaborol-2-yl)butyl)amino)butan-2-yl)carbamate (31-3, 1100 mg, 2 mmol) in 1, 4-dioxane (5 mL) was added 4 M HCl-dioxane (5 mL, 20 mmol) in ice cold condition and the reaction mixture was stirred at RT for 2 h. After completion of the reaction (monitored by LCMS) the solvent was evaporated from reaction mixture. The product was triturated with n-pentane to yield (R)-2-amino-4-morpholino-4-oxo-N—((R)-1-((3aS, 4S, 6S, 7aR)-3a, 5, 5-trimethylhexahydro-4, 6-methanobenzo[d][1, 3, 2]dioxaborol-2-yl)butyl)butanamide hydrochloride (31-4, 960 mg, 99%) as a brown gum. This product was used for next step without further purification. [M−H]⁻=434.

Synthesis of N—((R)-4-morpholino-1, 4-dioxo-1-(((R)-1-((3aS, 4S, 6S, 7aR)-3a, 5, 5-trimethylhexahydro-4, 6-methanobenzo[d][1, 3, 2]dioxaborol-2-yl)butyl)amino)butan-2-yl) pyrazine-2-carboxamide, [Step 3]: To a stirred solution of pyrazine-2-carboxylic acid (32-5, 280 mg, 2.2 mmol) in THF (10 mL), NMM (0.25 mL, 2.2 mmol) and IBCF (0.30 mL, 2.2 mmol) were added at −15° C. and the reaction mixture was stirred at this temperature for 30 min. Then (R)-2-amino-4-morpholino-4-oxo-N—((R)-1-((3aS, 4S, 6S, 7aR)-3a, 5, 5-trimethylhexahydro-4, 6-methanobenzo[d][1, 3, 2]dioxaborol-2-yl)butyl)butanamide hydrochloride (31-4, 960 mg, 2 mmol) and NMM (0.25 mL, 2.2 mmol) were added and the reaction mixture was stirred at RT for 2 h. After completion of the reaction (monitored by TLC and LC-MS), the reaction mixture was diluted with EtOAc and washed with 0.1 M HCl, 5% K₂CO₃, water and brine consecutively. The organic phase was dried over Na₂SO₄, filtered and evaporated to yield a brown gum. The product was purified by RP prep HPLC purification and the eluent was lyophilized to afford N—((R)-4-morpholino-1, 4-dioxo-1-(((R)-1-((3aS, 4S, 6S, 7aR)-3a, 5, 5-trimethylhexahydro-4, 6-methanobenzo[d][1, 3, 2]dioxaborol-2-yl)butyl)amino)butan-2-yl) pyrazine-2-carboxamide (31-6, 200 mg). [M−H]⁻=540.

Synthesis of ((R)-1-((R)-4-morpholino-4-oxo-2-(pyrazine-2-carboxamido)butanamido) butyl)boronic acid, [Step 4]: To a solution of N—((R)-4-morpholino-1, 4-dioxo-1-(((R)-1-((3aS, 4S, 6S, 7aR)-3a, 5, 5-trimethylhexahydro-4, 6-methanobenzo[d][1, 3, 2]dioxaborol-2-yl)butyl)amino)butan-2-yl) pyrazine-2-carboxamide (31-6, 200 mg, 0.37 mmol) in acetone (3 mL) was added methylboronic acid (220 mg, 3.7 mmol), followed by dropwise addition of 0.2 N HCl (3 mL). The reaction mixture was allowed to stirring at room temperature overnight. All the volatiles were evaporated at room temperature. The product was redissolved in acetonitrile and deionized water and freeze-dried to obtain product. The product was purified through RP prep HPLC purification and lyophilized to afford ((R)-1-((R)-4-morpholino-4-oxo-2-(pyrazine-2-carboxamido)butanamido)butyl)boronic acid (Compound 31, 60 mg). [M−H]⁻=406; ¹H NMR (400 MHz, MeOD) δ 9.26 (d, 1H), 8.80 (d, 1H), 8.70-8.69 (m, 1H), 5.29 (t, 1H), 3.68-3.61 (m, 4H), 3.58-3.49 (m, 4H), 3.32-3.27 (m, 1H), 2.99 (dd, 1H), 2.59 (t, 1H), 1.51-1.28 (m, 4H), 0.90 (t, 3H).

### Example 17: Synthesis of ((R)-1-((R)-4-morpholino-4-oxo-2-(pyrazine-2-carboxamido) butanamido)propyl)boronic acid

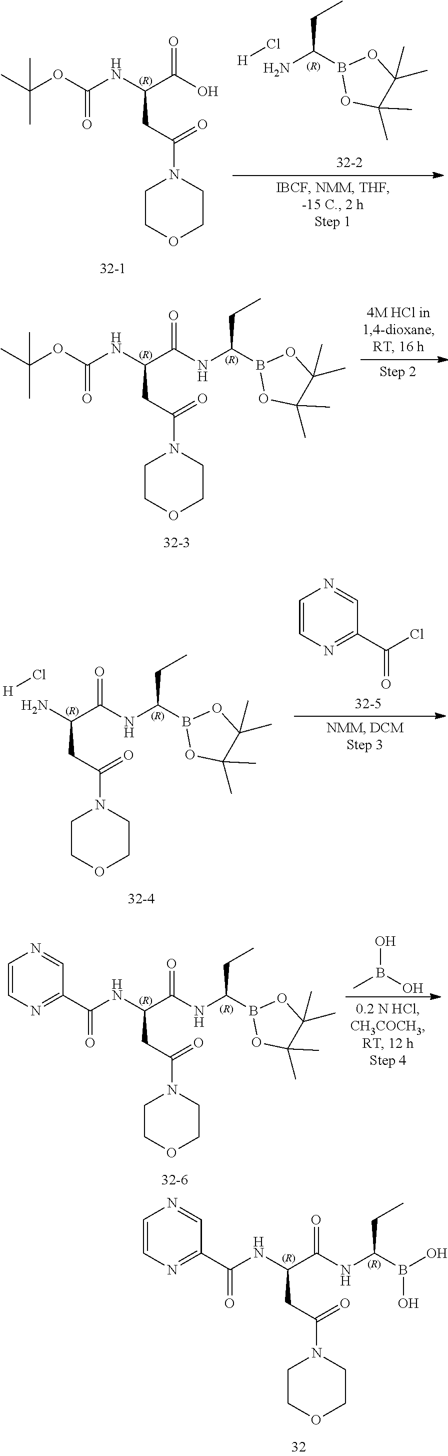

Synthesis of tert-butyl ((R)-4-morpholino-1, 4-dioxo-1-(((R)-1-(4, 4, 5, 5-tetramethyl-1, 3, 2-dioxaborolan-2-yl)propyl)amino)butan-2-yl)carbamate, [Step 1]: To a stirred solution of (R)-2-((tert-butoxycarbonyl)amino)-4-morpholino-4-oxobutanoic acid (32-1, 900 mg, 3 mmol) in THF (10 mL), NMM (0.4 mL, 3 mmol) and IBCF (0.4 mL, 3 mmol) were added at −15° C. and the reaction mixture was stirred at this temperature for 1 h. To the above solution, (R)-1-(4, 4, 5, 5-tetramethyl-1, 3, 2-dioxaborolan-2-yl) propan-1-amine hydrochloride (32-2, 600 mg, 2.7 mmol) and NMM (0.4 mL, 3 mmol) were added and the reaction mixture was stirred at RT for 2 h. After completion of the reaction (monitored by TLC and LC-MS), the reaction mixture was diluted with EtOAc and washed subsequently with 0.1 M HCl, 5% K₂CO₃, water and brine. The organic phase was dried over Na₂SO₄, filtered and evaporated to yield tert-butyl ((R)-4-morpholino-1, 4-dioxo-1-(((R)-1-(4, 4, 5, 5-tetramethyl-1, 3, 2-dioxaborolan-2-yl)propyl)amino)butan-2-yl)carbamate (32-3, 1250 mg). The product was used for next step without further purification. [M−H]⁻=468.

Synthesis of (R)-2-amino-4-morpholino-4-oxo-N—((R)-1-(4, 4, 5, 5-tetramethyl-1, 3, 2-dioxaborolan-2-yl)propyl)butanamide hydrochloride, [Step 2]: To a stirred solution of tert-butyl ((R)-4-morpholino-1, 4-dioxo-1-(((R)-1-(4, 4, 5, 5-tetramethyl-1, 3, 2-dioxaborolan-2-yl)propyl)amino)butan-2-yl)carbamate (32-3, 1250 mg, 2.7 mmol) in 1, 4-dioxane (7 mL), add 4 M HCl-dioxane (7 mL, 27 mmol) was added in ice cold condition and the reaction mixture was stirred at RT for 2 h. After completion of the reaction (monitored by LCMS) the solvent was evaporated from reaction mixture. The product was triturated with n-pentane to yield (R)-2-amino-4-morpholino-4-oxo-N—((R)-1-(4, 4, 5, 5-tetramethyl-1, 3, 2-dioxaborolan-2-yl)propyl)butanamide hydrochloride (32-4, 1000 mg). This product was used for next step without further purification. [M+H]⁺=3703.

Synthesis of N—((R)-4-morpholino-1, 4-dioxo-1-(((R)-1-(4, 4, 5, 5-tetramethyl-1, 3, 2-dioxaborolan-2-yl)propyl)amino)butan-2-yl) pyrazine-2-carboxamide, [Step 3]: To a stirred solution of (R)-2-amino-4-morpholino-4-oxo-N—((R)-1-(4, 4, 5, 5-tetramethyl-1, 3, 2-dioxaborolan-2-yl)propyl)butanamide hydrochloride (32-4, 250 mg, 0.6 mmol) in DCM (7 mL), NMM (0.2 mL, 1.2) was added at −15° C. and the reaction mixture was stirred at this temperature for 20 min. To the above solution, pyrazine-2-carbonyl chloride (32-5, 90 mg, 0.6 mmol) added and the reaction mixture was stirred at RT for 2 h. After completion of the reaction (monitored by TLC and LC-MS), the reaction mixture was diluted with DCM and washed subsequently with 0.1 M HCl, 5% K₂CO₃, water and brine. The organic phase was dried over Na₂SO₄, filtered and evaporated to obtain product. The product was purified by PREP-RP HPLC to afford N—((R)-4-morpholino-1, 4-dioxo-1-(((R)-1-(4, 4, 5, 5-tetramethyl-1, 3, 2-dioxaborolan-2-yl)propyl)amino)butan-2-yl) pyrazine-2-carboxamide (32-6, 30 mg). [M−H]⁻=474.

Synthesis of ((R)-1-((R)-4-morpholino-4-oxo-2-(pyrazine-2-carboxamido)butanamido) propyl)boronic acid, [Step 4]: To a solution of N—((R)-4-morpholino-1, 4-dioxo-1-(((R)-1-(4, 4, 5, 5-tetramethyl-1, 3, 2-dioxaborolan-2-yl)propyl)amino)butan-2-yl) pyrazine-2-carboxamide (32-6, 30 mg, 0.06 mmol) in acetone (1.5 mL) was added methylboronic acid (38 mg, 0.6 mmol), followed by dropwise addition of 0.2M HCl (1.5 mL). The reaction mixture was allowed to stirring at room temperature overnight. All the volatiles were evaporated at room temperature, and reaction mixture was re-dissolved in acetonitrile and deionized water and freeze-dried. The obtained product was purified through RP prep HPLC purification and lyophilized to afford ((R)-1-((R)-4-morpholino-4-oxo-2-(pyrazine-2-carboxamido)butanamido)propyl)boronic acid (Compound 32, 20 mg). [M−H]⁻=392; ¹H NMR (400 MHZ, MeOD) δ 9.26 (d, 1H), 8.80 (d, 1H), 8.69 (t, 1H), 5.30 (t, 1H), 3.63-3.61 (m, 2H), 3.58 (d, 2H), 3.55-3.51 (m, 4H), 3.02-2.97 (dd, 1H), 2.50 (t, 1H), 1.59-1.52 (m, 1H), 1.50-1.48 (m, 1H), 1.28 (s, 1H), 0.92 (t, 3H).

### Example 18: Synthesis of (R)-1-((R)-2-(3, 3-dimethylureido)-4-morpholino-4-oxobutanamido)-4-phenylbutyl)boronic acid

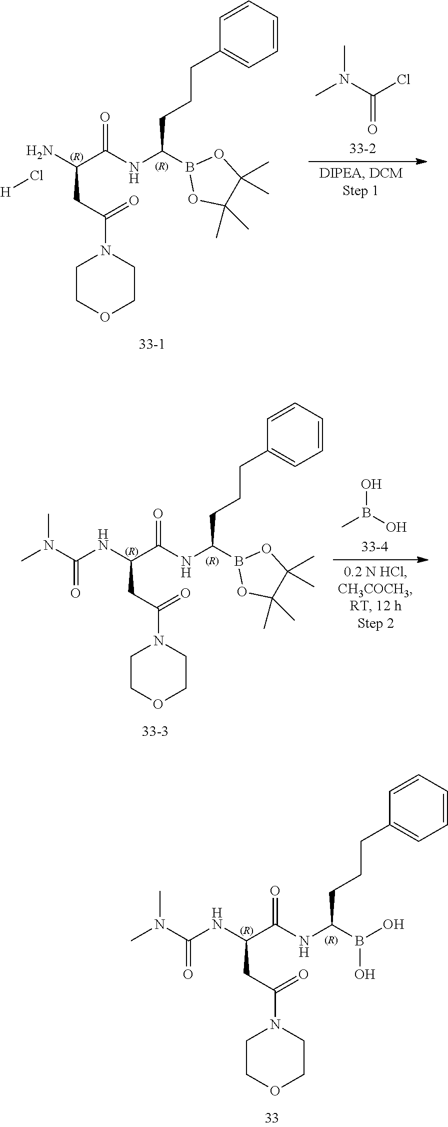

Synthesis of (R)-2-(3, 3-dimethylureido)-4-morpholino-4-oxo-N—((R)-4-phenyl-1-(4, 4, 5, 5-tetramethyl-1, 3, 2-dioxaborolan-2-yl)butyl)butanamide, [Step 1]: To a stirred solution of (R)-2-amino-4-morpholino-4-oxo-N—((R)-4-phenyl-1-(4, 4, 5, 5-tetramethyl-1, 3, 2-dioxaborolan-2-yl)butyl)butanamide hydrochloride (33-1, 300 mg, 0.6 mmol) and N,N-dimethylcarbamoyl chloride (33-2, 0.06 mL, 0.7 mmol) in DCM (3 mL), DIPEA (0.5 mL, 3.0 mmol) was added in ice cold condition and the reaction mixture was stirred at RT for 2 h. TLC and LCMS showed full conversion of starting material with formation of new polar spot. The reaction mixture was diluted with DCM and washed with water and brine solution. The organic phase was dried over Na₂SO₄, filtered and evaporated to yield product. The material was purified by RP prep HPLC purification and lyophilized to afford (R)-2-(3, 3-dimethylureido)-4-morpholino-4-oxo-N—((R)-4-phenyl-1-(4, 4, 5, 5-tetramethyl-1, 3, 2-dioxaborolan-2-yl)butyl)butanamide (33-3, 46 mg). [M−H]⁺=530.

Synthesis of (R)-1-((R)-2-(3, 3-dimethylureido)-4-morpholino-4-oxobutanamido)-4-phenylbutyl)boronic acid, [Step 2]: To a stirred solution of (R)-2-(3, 3-dimethylureido)-4-morpholino-4-oxo-N—((R)-4-phenyl-1-(4, 4, 5, 5-tetramethyl-1, 3, 2-dioxaborolan-2-yl)butyl)butanamide (33-3, 46 mg, 0.1 mmol) and methylboronic acid (52 mg, 1.0 mmol) in acetone (3 mL) was added 0.2 N HCl (3.0 mL) and the reaction mixture was stirred at RT overnight. TLC and LCMS showed full conversion of starting material with formation of new polar spot. The volatiles were evaporated and the residue was purified via RP prep HPLC purification and lyophilized to afford (R)-1-((R)-2-(3, 3-dimethylureido)-4-morpholino-4-oxobutanamido)-4-phenylbutyl)boronic acid (Compound 33, 22 mg). [M−H]⁺=447; ¹H NMR (400 MHZ, MeOD) δ 7.24-7.11 (m, 5H), 4.92 (t, 1H), 3.65-3.59 (m, 4H), 3.51-3.48 (m, 4H), 3.03-3.01 (m, 1H), 2.91-2.86 (m, 7H), 2.62-2.56 (m, 3H), 1.70-1.48 (m, 4H).

### Example 19: Synthesis of ((R)-1-((R)-4-(methylsulfonyl)-2-(pyrazine-2-carboxamido) butanamido)-4-phenylbutyl)boronic acid

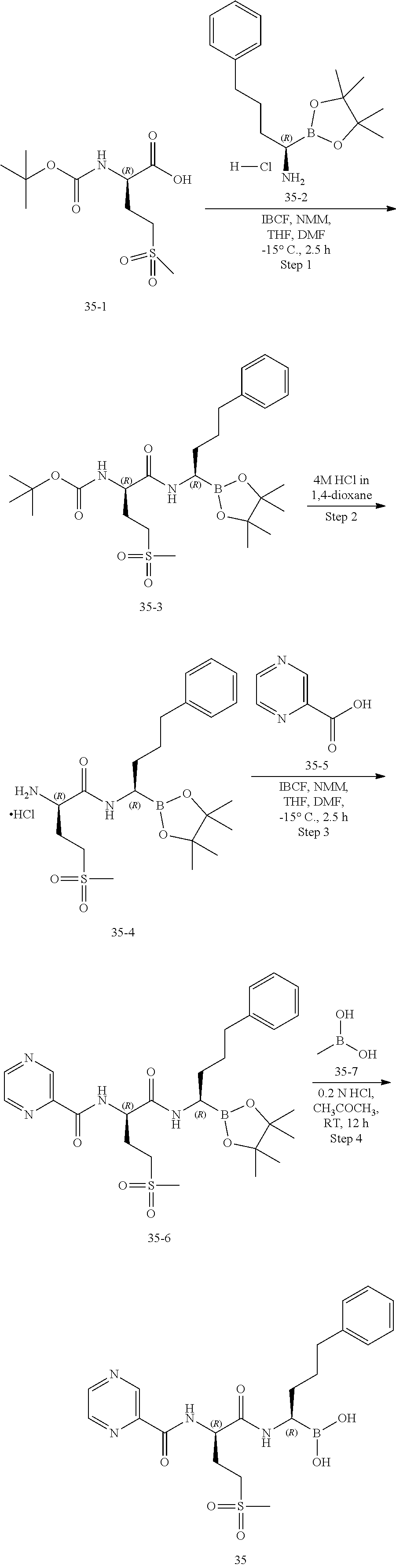

Synthesis of tert-butyl ((R)-4-(methylsulfonyl)-1-oxo-1-(((R)-4-phenyl-1-(4, 4, 5, 5-tetramethyl-1, 3, 2-dioxaborolan-2-yl)butyl)amino)butan-2-yl)carbamate, [Step 1]: To a stirred solution of (R)-2-((tert-butoxycarbonyl)amino)-4-(methylsulfonyl)butanoic acid (35-1, 495 mg, 1.7 mmol) in THF (5 mL) was added IBCF (0.24 mL, 1.7 mmol) followed by NMM (0.19 mL, 1.7 mmol) at −15° C. and stirred for 30 min. A solution of (1R)-4-phenyl-1-(4, 4, 5, 5-tetramethyl-1, 3, 2-dioxaborolan-2-yl)butan-1-amine hydrochloride (35-2, 500 mg, 1.6 mmol) in dimethylformamide (1 mL) was added dropwise followed by NMM (0.18 mL, 1.6 mmol) to the reaction mixture at −15° C. and then allowed to warm to 0° C. and stirred for 2 h. The reaction mixture was diluted with ethyl acetate and washed with 0.1N HCl (twice), 5% aqueous K₂CO₃ solution (twice), water (twice) and brine, dried over anhydrous Na₂SO₄ and concentrated under reduced pressure to afford tert-butyl ((R)-4-(methylsulfonyl)-1-oxo-1-(((R)-4-phenyl-1-(4, 4, 5, 5-tetramethyl-1, 3, 2-dioxaborolan-2-yl)butyl)amino)butan-2-yl)carbamate (35-3, 650 mg). [M−H]⁻=537.

Synthesis of (R)-2-amino-4-(methylsulfonyl)-N—((R)-4-phenyl-1-(4, 4, 5, 5-tetramethyl-1, 3, 2-dioxaborolan-2-yl)butyl)butanamide, [Step 2]: To a stirred solution of tert-butyl ((R)-4-(methylsulfonyl)-1-oxo-1-(((R)-4-phenyl-1-(4, 4, 5, 5-tetramethyl-1, 3, 2-dioxaborolan-2-yl)butyl)amino)butan-2-yl)carbamate (35-3, 800 mg, 1.5 mmol) in 1, 4-dioxane (8 mL) was added 4M HCl in 1, 4-dioxane (3.7 mL) dropwise at ice cold condition. The reaction mixture was stirred at ambient temperature for 12 h. Volatiles were evaporated under reduced pressure to afford (R)-2-amino-4-(methylsulfonyl)-N—((R)-4-phenyl-1-(4, 4, 5, 5-tetramethyl-1, 3, 2-dioxaborolan-2-yl)butyl)butanamide (35-4, 700 mg). [M−H]⁻=437.

Synthesis of N—((R)-4-(methylsulfonyl)-1-oxo-1-(((R)-4-phenyl-1-(4, 4, 5, 5-tetramethyl-1, 3, 2-dioxaborolan-2-yl)butyl)amino)butan-2-yl) pyrazine-2-carboxamide, [Step 3]: To a stirred solution of pyrazine-2-carboxylic acid (35-5, 220 mg, 1.7 mmol) in THF (8 mL) was added IBCF (0.23 mL, 1.7 mmol) followed by NMM (0.19 mL, 1.7 mmol) at −15° C. and stirred for 30 min. A solution of (2R)-2-amino-4-methylsulfonyl-N-[(1R)-4-phenyl-1-(4, 4, 5, 5-tetramethyl-1, 3, 2-dioxaborolan-2-yl)butyl]butanamide hydrochloride (35-4, 760 mg, 1.60 mmol) in dimethylformamide (1 mL) was added dropwise followed by NMM (0.18 mL, 1.6 mmol) to the reaction mixture at −15° C. and then allowed to warm to 0° C. and stirred for 2 h. The reaction mixture was diluted with ethyl acetate and washed with 0.1N HCl (twice), 5% aqueous K₂CO₃ solution (twice), water (twice) and brine, dried over anhydrous Na₂SO₄ and concentrated under reduced pressure. The product was purified by prep HPLC purification to afford N—((R)-4-(methylsulfonyl)-1-oxo-1-(((R)-4-phenyl-1-(4, 4, 5, 5-tetramethyl-1, 3, 2-dioxaborolan-2-yl)butyl)amino)butan-2-yl) pyrazine-2-carboxamide (35-6, 60 mg). [M−H]⁻=543.

Synthesis of ((R)-1-((R)-4-(methylsulfonyl)-2-(pyrazine-2-carboxamido)butanamido)-4-phenylbutyl)boronic acid, [Step 4]: To a solution of N—((R)-4-(methylsulfonyl)-1-oxo-1-(((R)-4-phenyl-1-(4, 4, 5, 5-tetramethyl-1, 3, 2-dioxaborolan-2-yl)butyl)amino)butan-2-yl) pyrazine-2-carboxamide (35-6, 55 mg, 0.1 mmol) and methylboronic acid (35-7, 60 mg, 1.0 mmol) in acetone (2 mL) was added 0.2 N HCl (2 mL) and stirred at ambient temperature for 16 h. All volatiles were evaporated under reduced pressure and purified by prep HPLC purification and lyophilized to give ((R)-1-((R)-4-(methylsulfonyl)-2-(pyrazine-2-carboxamido)butanamido)-4-phenylbutyl)boronic acid (Compound 35, 32 mg). LCMS (ESI) Calcd. for C₂₀H₂₇BN₄O₆S: 462, found [M−H]⁻=461. ¹H NMR (400 MHZ, CD3OD) δ 9.22 (s, 1H), 8.79 (d, 1H), 8.70 (s, 1H), 7.22-7.07 (m, 5H), 4.99 (t, 1H), 3.29-3.22 (m, 2H), 2.97 (s, 3H), 2.69 (t, 1H), 2.64-2.34 (m, 4H), 1.66-1.57 (m, 4H).

### Example 20: Synthesis of ((R)-1-((R)-3-methoxy-2-(pyrazine-2-carboxamido)propanamido)-4-phenylbutyl)boronic acid

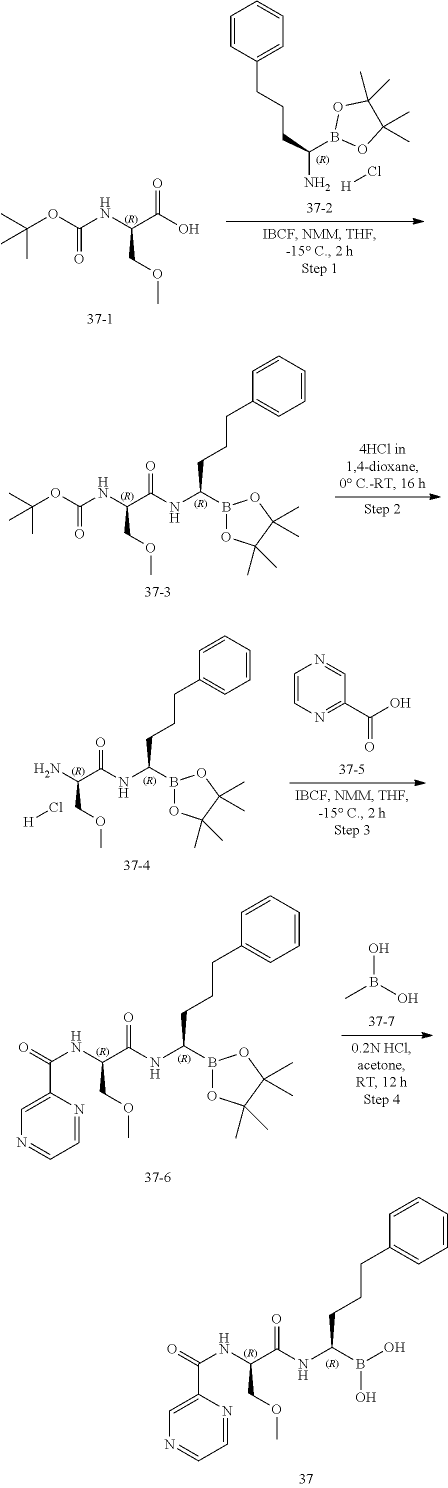

Synthesis of tert-butyl ((R)-3-methoxy-1-oxo-1-(((R)-4-phenyl-1-(4, 4, 5, 5-tetramethyl-1, 3, 2-dioxaborolan-2-yl)butyl)amino) propan-2-yl)carbamate, [Step 1]: To a stirred solution of N-(tert-butoxycarbonyl)-O-methyl-D-serine (37-1, 695 mg, 3.2 mmol) in tetrahydrofuran (8 mL) was added isobutyl chloroformate (IBCF) (0.4 mL, 3.2 mmol) and N-methylmorpholine (NMM) (0.35 mL, 3.2 mmol) at −15° C. The reaction mixture was stirred at the same temperature for 30 minutes. To the reaction mixture was added (R)-4-phenyl-1-(4, 4, 5, 5-tetramethyl-1, 3, 2-dioxaborolan-2-yl)butan-1-amine (37-2, 900 mg, 2.9 mmol) in dimethylformamide (1 mL) followed by NMM (0.3 mL, 2.9 mmol) at −15° C. The reaction mixture was gradually warmed to 0° C. and stirred for 2 hours. The reaction mixture was neutralized with aqueous 0.1 N HCl and extracted with ethyl acetate. The combined organic layers were washed with 5% potassium carbonate, water, brine, dried over anhydrous sodium sulfate, filtered, and concentrated under reduced pressure to afford tert-butyl ((R)-3-methoxy-1-oxo-1-(((R)-4-phenyl-1-(4, 4, 5, 5-tetramethyl-1, 3, 2-dioxaborolan-2-yl)butyl)amino) propan-2-yl)carbamate (37-3, 1.3 g). [M−H]=475.

Synthesis of (R)-2-amino-3-methoxy-N—((R)-4-phenyl-1-(4, 4, 5, 5-tetramethyl-1, 3, 2-dioxaborolan-2-yl)butyl) propanamide hydrochloride, [Step 2]: To a solution of tert-butyl ((R)-3-methoxy-1-oxo-1-(((R)-4-phenyl-1-(4, 4, 5, 5-tetramethyl-1, 3, 2-dioxaborolan-2-yl)butyl) amino) propan-2-yl)carbamate (37-3, 1.3 g, 2.7 mmol) in 1, 4-dioxane (10 mL) was added 4 M HCl in dioxane (8.0 mL, 27.3 mmol) at 0° C. The reaction mixture was gradually warmed to 25° C. and stirred for 16 hours. The reaction mixture was concentrated under reduced pressure to obtain (R)-2-amino-3-methoxy-N—((R)-4-phenyl-1-(4, 4, 5, 5-tetramethyl-1, 3, 2-dioxaborolan-2-yl)butyl) propanamide hydrochloride (37-4, 980 mg). The product was used directly in the next step without purification.

Synthesis of N—((R)-3-methoxy-1-oxo-1-(((R)-4-phenyl-1-(4, 4, 5, 5-tetramethyl-1, 3, 2-dioxaborolan-2-yl)butyl)amino) propan-2-yl) pyrazine-2-carboxamide, [Step 3]: To a stirred solution of pyrazine-2-carboxylic acid (37-5, 325 mg, 2.6 mmol) in tetrahydrofuran (8 mL) was added isobutyl chloroformate (IBCF) (0.34 mL, 2.6 mmol) and N-methylmorpholine (NMM) (0.3 mL, 2.6 mmol) at −15° C. The reaction mixture was stirred at the same temperature for 30 minutes. To the reaction mixture was added (R)-2-amino-3-methoxy-N—((R)-4-phenyl-1-(4, 4, 5, 5-tetramethyl-1, 3, 2-dioxaborolan-2-yl)butyl) propanamide hydrochloride (37-4, 980 mg, 2.4 mmol) in dimethylformamide (1 mL) followed by NMM (0.3 mL, 2.4 mmol) at −15° C. The reaction mixture was gradually warmed to 0° C. and stirred for 2 hours. The reaction mixture was neutralized with aqueous 0.1 N HCl and extracted with ethyl acetate. The combined organic layers were washed with 5% potassium carbonate solution, water, brine, dried over anhydrous sodium sulfate, filtered, and concentrated under reduced pressure. The material was purified by RP prep HPLC purification and lyophilized to afford N—((R)-3-methoxy-1-oxo-1-(((R)-4-phenyl-1-(4, 4, 5, 5-tetramethyl-1, 3, 2-dioxaborolan-2-yl)butyl)amino) propan-2-yl) pyrazine-2-carboxamide (37-6, 110 mg). [M−H]⁺=481; ¹H NMR (400 MHZ, MeOD) δ 9.23 (br s, 1H), 8.80 (d, 1H), 8.70 (br s, 1H), 7.22-7.09 (m, 5H), 4.98 (t, 1H), 3.89-3.86 (m, 1H), 3.78-3.75 (m, 1H), 3.37 (s, 3H), 2.65-2.57 (m, 3H), 1.67-1.35 (m, 4H), 1.19-1.16 (m, 3H).

Synthesis of ((R)-1-((R)-3-methoxy-2-(pyrazine-2-carboxamido)propanamido)-4-phenyl butyl)boronic acid, [Step 4]: To a stirred solution of N—((R)-3-methoxy-1-oxo-1-(((R)-4-phenyl-1-(4, 4, 5, 5-tetramethyl-1, 3, 2-dioxaborolan-2-yl)butyl)amino) propan-2-yl) pyrazine-2-carboxamide (37-6, 110 mg, 0.23 mmol) and methylboronic acid (37-7, 135 mg, 2.3 mmol) in acetone (4 mL) was added 0.2 N HCl (4 mL) and the reaction mixture was stirred at ambient temperature overnight. TLC and LCMS showed the complete disappearance of the starting material, and the reaction mixture was concentrated under reduced pressure. The product was purified by reverse-phase prep-HPLC and lyophilized to afford ((R)-1-((R)-3-methoxy-2-(pyrazine-2-carboxamido)propanamido)-4-phenylbutyl)boronic acid (Compound 37, 49 mg). LCMS (ESI) Calcd. for C₁₉H₂₅BN₄O₅: 400, found [M−H]⁺=399; ¹H NMR (400 MHZ, MeOD) δ 9.23 (br s, 1H), 8.81 (d, 1H), 8.70-8.69 (m, 1H), 7.22-7.08 (m, 5H), 4.98 (t, 1H), 3.89-3.85 (m, 1H), 3.78-3.75 (m, 1H), 3.37 (s, 3H), 2.65-2.56 (m, 3H), 1.68-1.46 (m, 4H).

### Example 21: Synthesis of ((R)-2-(benzyloxy)-1-((R)-3-methoxy-2-(pyrazine-2-carboxamido) propanamido)ethyl)boronic acid

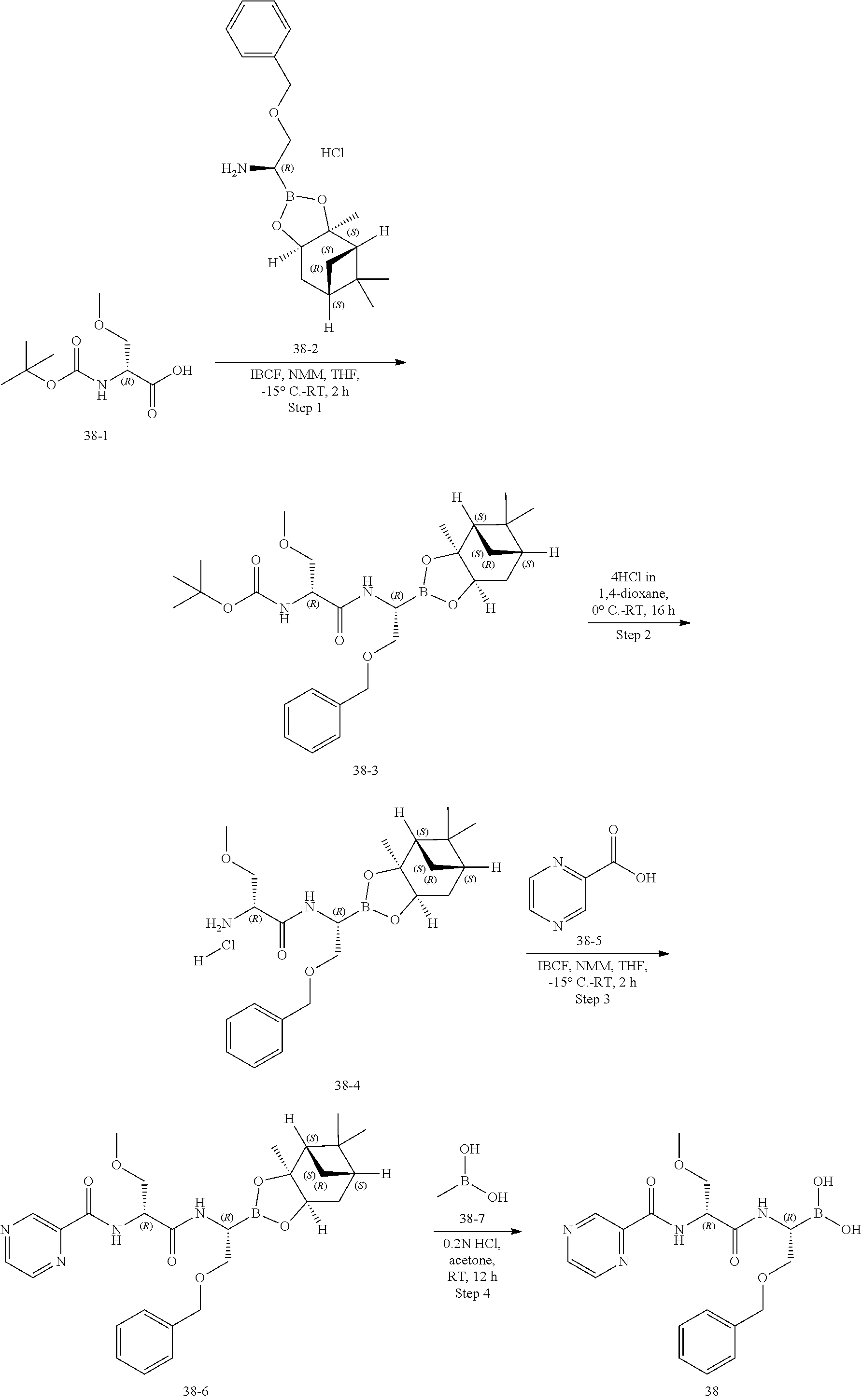

Synthesis of tert-butyl ((R)-1-(((R)-2-(benzyloxy)-1-((3aS, 4S, 6S, 7aR)-3a, 5, 5-trimethylhexahydro-4, 6-methanobenzo[d][1, 3, 2]dioxaborol-2-yl)ethyl)amino)-3-methoxy-1-oxopropan-2-yl)carbamate, [Step 1]: To a stirred solution of in N-(tert-butoxycarbonyl)-O-methyl-D-serine (38-1, 65 mg, 0.3 mmol) in THF (3 mL), was added IBCF (0.04 mL, 0.3 mmol) followed by NMM (0.04 mL, 0.3 mmol) at −15° C. and the reaction mixture was stirred at that temperature for 1 h. ((R)-2-(benzyloxy)-1-((3aS, 4S, 6S, 7aR)-3a, 5, 5-trimethylhexahydro-4, 6-methanobenzo[d][1, 3, 2]dioxaborol-2-yl) ethan-1-amine hydrochloride (38-2, 100 mg, 0.3 mmol) and NMM (0.04 mL, 0.3 mmol) were added and the reaction mixture was stirred at RT for 2 h.

TLC and LCMS showed full consumption of starting material with formation of new spot. The reaction mixture was diluted with EtOAc and washed subsequently with 0.1M HCl (2×30 mL), 5% K₂CO₃ (2×30 mL), water (2×30 mL) and brine. The organic phase was dried over Na₂SO₄, filtered and evaporated to yield tert-butyl ((R)-1-(((R)-2-(benzyloxy)-1-((3aS, 4S, 6S, 7aR)-3a, 5, 5-trimethylhexahydro-4, 6-methanobenzo[d][1, 3, 2]dioxaborol-2-yl)ethyl)amino)-3-methoxy-1-oxopropan-2-yl)carbamate (38-3, 120 mg). The product was used for next step without further purification. [M+H]⁺=531.

Synthesis of (R)-2-amino-N—((R)-2-(benzyloxy)-1-((3aS, 4S, 6S, 7aR)-3a, 5, 5-trimethylhexahydro-4, 6-methanobenzo[d][1, 3, 2]dioxaborol-2-yl)ethyl)-3-methoxy propanamide hydrochloride, [Step 2]: To a stirred solution of tert-butyl ((R)-1-(((R)-2-(benzyloxy)-1-((3aS, 4S, 6S, 7aR)-3a, 5, 5-trimethylhexahydro-4, 6-methanobenzo[d][1, 3, 2]dioxaborol-2-yl)ethyl)amino)-3-methoxy-1-oxopropan-2-yl)carbamate (38-3, 350 mg, 0.7 mmol) in 1, 4-dioxane (4 mL), 4M HCl in dioxane (3.3 mL, 13.2 mmol) was added in ice cold condition and the reaction mixture was stirred at RT for 2 h. TLC showed full conversion of starting material with formation of new polar spot. LCMS showed the formation of desired product. The solvent was evaporated to yield (R)-2-amino-N—((R)-2-(benzyloxy)-1-((3aS, 4S, 6S, 7aR)-3a, 5, 5-trimethylhexahydro-4, 6-methanobenzo[d][1, 3, 2]dioxaborol-2-yl)ethyl)-3-methoxypropanamide hydrochloride (38-4, 300 mg). [M+H]⁺=431.

Synthesis of N—((R)-1-(((R)-2-(benzyloxy)-1-((3aS, 4S, 6S, 7aR)-3a, 5, 5-trimethylhexahydro-4, 6-methanobenzo[d][1, 3, 2]dioxaborol-2-yl)ethyl)amino)-3-methoxy-1-oxopropan-2-yl) pyrazine-2-carboxamide, [Step 3]: To a stirred solution of in pyrazine-2-carboxylic acid (38-5, 100 mg, 0.8 mmol) in THF (5 mL) was added IBCF (0.11 mL, 0.8 mmol) followed by NMM (0.11 mL, 0.8 mmol) at −15° C. and the reaction mixture was stirred at that temperature for 1 h. (R)-2-amino-N—((R)-2-(benzyloxy)-1-((3aS, 4S, 6S, 7aR)-3a, 5, 5-trimethylhexahydro-4, 6-methanobenzo[d][1, 3, 2]dioxaborol-2-yl)ethyl)-3-methoxypropanamide hydrochloride (38-4, 350 mg, 0.8 mmol) and NMM (0.11 mL, 0.8 mmol) were added and the reaction mixture was stirred at RT for 2 h. TLC and LCMS showed full consumption of starting material with formation of new spot. The reaction mixture was diluted with EtOAc and washed subsequently with 0.1 M HCl (2×30 mL), 5% K₂CO₃ (2×30 mL), water (2×30 mL) and brine. The organic phase was dried over Na₂SO₄, filtered and evaporated to yield a brown gum. The product was purified by prep HPLC purification and lyophilized to afford N—((R)-1-(((R)-2-(benzyloxy)-1-((3aS, 4S, 6S, 7aR)-3a, 5, 5-trimethylhexahydro-4, 6-methanobenzo[d][1, 3, 2]dioxaborol-2-yl)ethyl)amino)-3-methoxy-1-oxopropan-2-yl) pyrazine-2-carboxamide (38-6, 80 mg). [M+H]⁺=537.

Synthesis of ((R)-2-(benzyloxy)-1-((R)-3-methoxy-2-(pyrazine-2-carboxamido) propanamido)ethyl)boronic acid, [Step 4]: To a stirred solution of N—((R)-1-(((R)-2-(benzyloxy)-1-((3aS, 4S, 6S, 7aR)-3a, 5, 5-trimethylhexahydro-4, 6-methanobenzo[d][1, 3, 2]dioxaborol-2-yl)ethyl)amino)-3-methoxy-1-oxopropan-2-yl) pyrazine-2-carboxamide (38-6, 80 mg, 0.15 mmol) and methylboronic acid (135 mg, 2.2 mmol) in acetone (2 mL) was added 0.2N HCl (1.0 mL, 0.1 mmol) and the reaction mixture was stirred at RT overnight. TLC and LCMS showed full conversion of starting material with formation of new polar spot. The volatiles were evaporated and the residue was purified via prep HPLC purification and lyophilized to afford ((R)-2-(benzyloxy)-1-((R)-3-methoxy-2-(pyrazine-2-carboxamido)propanamido)ethyl)boronic acid (Compound 38, 40 mg). [M−H]⁺=401; ¹H NMR (400 MHZ, MeOD) δ 9.24 (s, 1H), 8.80 (d, 1H), 8.70 (d, 1H), 7.34-7.23 (m, 5H), 5.06 (t, 1H), 4.49 (d, 2H), 3.90-3.86 (m, 1H), 3.81-3.77 (m, 1H), 3.59-3.56 (m, 1H), 3.46-3.43 (m, 1H), 3.39 (br s, 3H), 2.95 (d, 1H).

### Example 22: Synthesis ((R)-2-(benzyloxy)-1-((R)-2-(pyrazine-2-carboxamido) pentanamido)ethyl)boronic

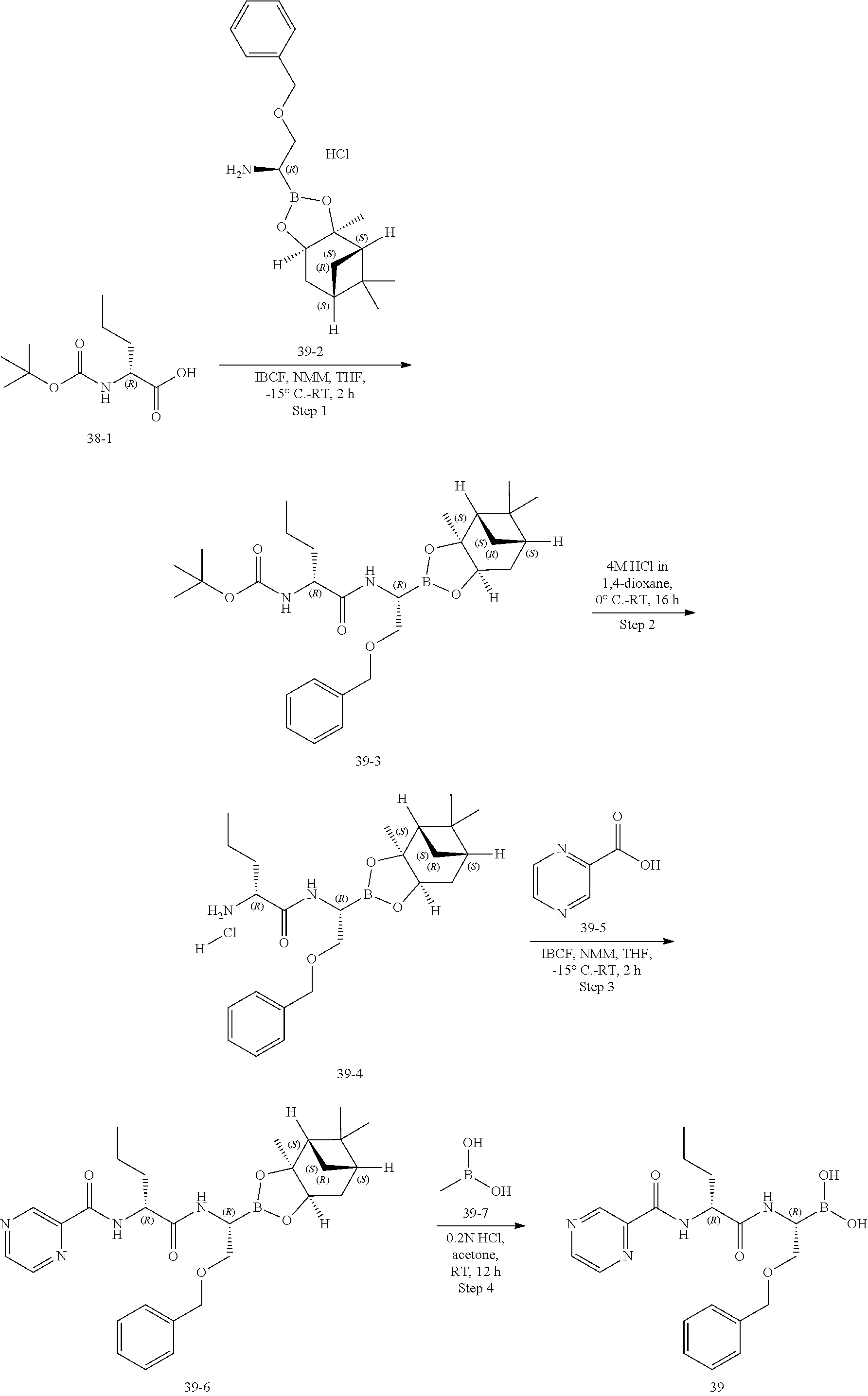

Synthesis of tert-butyl ((R)-1-(((R)-2-(benzyloxy)-1-((3aS, 4S, 6S, 7aR)-3a, 5, 5-trimethylhexahydro-4, 6-methanobenzo[d][1, 3, 2]dioxaborol-2-yl)ethyl)amino)-1-oxopentan-2-yl)carbamate, [Step 1]: To a stirred solution of in (R)-2-((tert-butoxycarbonyl)amino) pentanoic acid (39-1, 65 mg, 0.3 mmol) in THF (3 mL) was added IBCF (0.04 mL, 0.3 mmol) followed by NMM (0.04 mL, 0.3 mmol) at −15° C. and the reaction mixture was stirred at that temperature for 1 h. ((R)-2-(benzyloxy)-1-((3aS, 4S, 6S, 7aR)-3a, 5, 5-trimethylhexahydro-4, 6-methanobenzo[d][1, 3, 2]dioxaborol-2-yl) ethan-1-amine hydrochloride (39-2, 100 mg, 0.3 mmol) and NMM (0.04 mL, 0.3 mmol) were added and the reaction mixture was stirred at RT for 2 h. TLC and LCMS showed full consumption of starting material with formation of new spot. The reaction mixture was diluted with EtOAc and washed subsequently with 0.1M HCl (2×30 mL), 5% K₂CO₃ (2×30 mL), water (2×30 mL) and brine. The organic phase was dried over Na₂SO₄, filtered and evaporated to yield tert-butyl ((R)-1-(((R)-2-(benzyloxy)-1-((3aS, 4S, 6S, 7aR)-3a, 5, 5-trimethylhexahydro-4, 6-methanobenzo[d][1, 3, 2]dioxaborol-2-yl)ethyl)amino)-1-oxopentan-2-yl)carbamate (39-3, 120 mg, 0.2 mmol). The product was used for next step without further purification. [M+H]⁺=529.

Synthesis of (R)-2-amino-N—((R)-2-(benzyloxy)-1-((3aS, 4S, 6S, 7aR)-3a, 5, 5-trimethylhexahydro-4, 6-methanobenzo[d][1, 3, 2]dioxaborol-2-yl)ethyl) pentanamide hydrochloride, [Step 2]: To a stirred solution of tert-butyl ((R)-1-(((R)-2-(benzyloxy)-1-((3aS, 4S, 6S, 7aR)-3a, 5, 5-trimethylhexahydro-4, 6-methanobenzo[d][1, 3, 2]dioxaborol-2-yl)ethyl)amino)-1-oxopentan-2-yl)carbamate (39-3, 350 mg, 0.7 mmol) in 1, 4-dioxane (4 mL), 4M HCl in dioxane (3.3 mL, 13.2 mmol) was added in ice cold condition and the reaction mixture was stirred at RT for 2 h. TLC showed full conversion of starting material with formation of new polar spot. LCMS showed the formation of desired product. The solvent was evaporated to yield (R)-2-amino-N—((R)-2-(benzyloxy)-1-((3aS, 4S, 6S, 7aR)-3a, 5, 5-trimethylhexahydro-4, 6-methanobenzo[d][1, 3, 2]dioxaborol-2-yl)ethyl) pentanamide hydrochloride (39-4, 300 mg). [M+H]⁺=430.

Synthesis of N—((R)-1-(((R)-2-(benzyloxy)-1-((3aS, 4S, 6S, 7aR)-3a, 5, 5-trimethylhexahydro-4, 6-methanobenzo[d][1, 3, 2]dioxaborol-2-yl)ethyl)amino)-1-oxopentan-2-yl) pyrazine-2-carboxamide, [Step 3]: To a stirred solution of in pyrazine-2-carboxylic acid (39-5, 100 mg, 0.8 mmol) in THF (5 mL) was added IBCF (0.11 mL, 0.8 mmol) and NMM (0.11 mL, 0.8 mmol) at −15° C. and the reaction mixture was stirred at that temperature for 1 h. (R)-2-amino- N—((R)-2-(benzyloxy)-1-((3aS, 4S, 6S, 7aR)-3a, 5, 5-trimethylhexahydro-4, 6-methanobenzo[d][1, 3, 2]dioxaborol-2-yl)ethyl) pentanamide hydrochloride (39-4, 350 mg, 0.7 mmol) and NMM (0.11 mL, 0.8 mmol) were added and the reaction mixture was stirred at RT for 2 h. TLC and LCMS showed full consumption of starting material with formation of new spot. The reaction mixture was diluted with EtOAc and washed subsequently with 0.1M HCl (2×30 mL), 5% K₂CO₃ (2×30 mL), water (2×30 mL) and brine. The organic phase was dried over Na₂SO₄, filtered and evaporated and purified via prep HPLC purification to yield N—((R)-1-(((R)-2-(benzyloxy)-1-((3aS, 4S, 6S, 7aR)-3a, 5, 5-trimethylhexahydro-4, 6-methanobenzo[d][1, 3, 2]dioxaborol-2-yl)ethyl)amino)-1-oxopentan-2-yl) pyrazine-2-carboxamide (39-6, 90 mg). [M+H]⁺=534.

Synthesis of ((R)-2-(benzyloxy)-1-((R)-2-(pyrazine-2-carboxamido) pentanamido)ethyl) boronic acid [Step 4]: To a stirred solution of N—((R)-1-(((R)-2-(benzyloxy)-1-((3aS, 4S, 6S, 7aR)-3a, 5, 5-trimethylhexahydro-4, 6-methanobenzo[d][1, 3, 2]dioxaborol-2-yl)ethyl)amino)-1-oxopentan-2-yl) pyrazine-2-carboxamide (39-6, 90 mg, 0.2 mmol) and methylboronic acid (39-7, 150 mg, 2.5 mmol) in acetone (2 mL), 0.2 N HCl (1.0 mL, 0.1 mmol) was added and the reaction mixture was stirred at RT overnight. TLC and LCMS showed full conversion of starting material with formation of new polar spot. The volatiles were evaporated and the residue was purified via prep HPLC purification and lyophilized to afford ((R)-2-(benzyloxy)-1-((R)-2-(pyrazine-2-carboxamido) pentanamido)ethyl)boronic acid (Compound 39, 30 mg). [M−H]⁺=399; ¹H NMR (400 MHz, MeOD) δ 9.23 (s, 1H), 8.80 (d, 1H), 8.70 (s, 1H), 7.35-7.23 (m, 5H), 4.88 (t, 1H), 4.49 (br s, 2H), 3.60-3.56 (m, 1H), 3.42 (t, 1H), 2.93-2.89 (m, 1H), 1.94-1.90 (m, 2H), 1.50-1.43 (br s, 2H), 0.98 (t, 3H).

### Example 23: Synthesis of ((S)-1-((R)-4-morpholino-4-oxo-2-(pyrazine-2-carboxamido) butanamido)-4-phenylbutyl)boronic acid

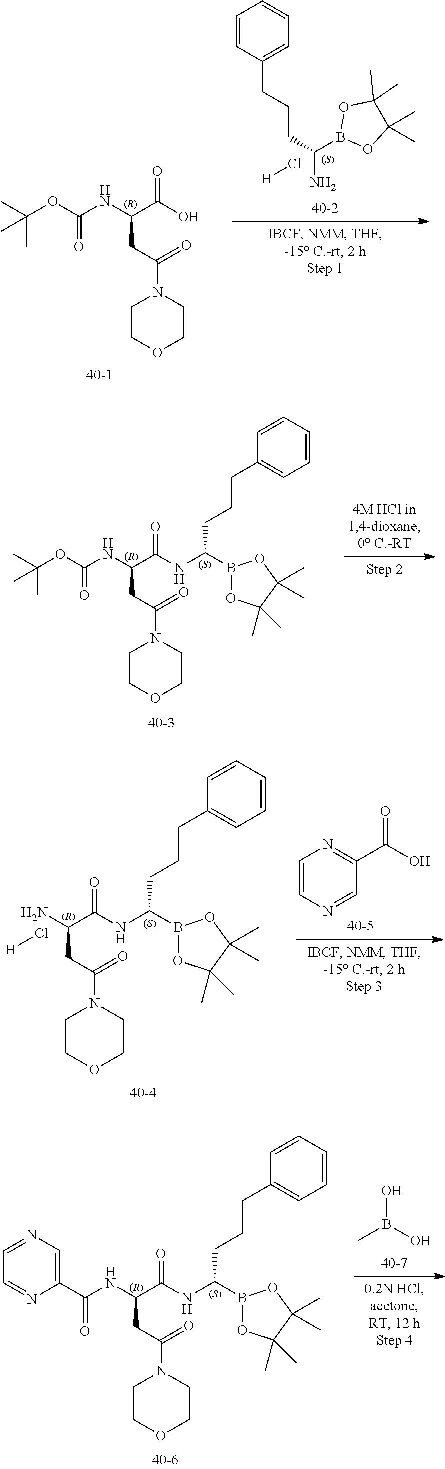

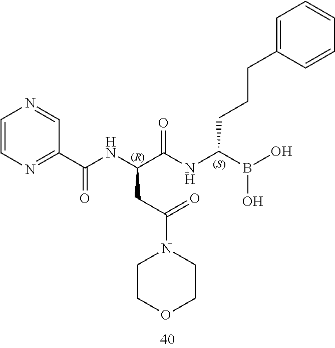

Synthesis of tert-butyl ((R)-4-morpholino-1, 4-dioxo-1-(((S)-4-phenyl-1-(4, 4, 5, 5-tetramethyl-1, 3, 2-dioxaborolan-2-yl)butyl)amino)butan-2-yl)carbamate, [Step 1]: To a stirred solution of (R)-2-((tert-butoxycarbonyl)amino)-4-morpholino-4-oxobutanoic acid (40-1, 750 mg, 2.4 mmol) in THF (8 mL) was added IBCF (0.3 mL, 2.4 mmol) and NMM (0.3 mL, 2.4 mmol) at −15° C. Reaction mixture was stirred at same temperature for 30 min. Then(S)-4-phenyl-1-(4, 4, 5, 5-tetramethyl-1, 3, 2-dioxaborolan-2-yl)butan-1-amine hydrochloride (40-2, 700 mg, 2.2 mmol) in DMF (1 mL) followed by NMM (0.2 mL, 2.2 mmol) was added to the reaction mixture at −15° C. It was gradually warmed to 0° C. and stirred for 2 h. LCMS of crude reaction mass confirmed the formation of desired product. It was neutralized with saturated aqueous 0.1 N HCl solution and extracted with ethyl acetate. Combined organic layer was washed with 5% K₂CO₃ solution, water, brine, dried over Na₂SO₄, filtered and evaporated under reduced pressure to afford tert-butyl ((R)-4-morpholino-1, 4-dioxo-1-(((S)-4-phenyl-1-(4, 4, 5, 5-tetramethyl-1, 3, 2-dioxaborolan-2-yl)butyl)amino)butan-2-yl)carbamate (40-3, 700 mg). [M+H]⁺=560.

Synthesis of (R)-2-amino-4-morpholino-4-oxo-N—((S)-4-phenyl-1-(4, 4, 5, 5-tetramethyl-1, 3, 2-dioxaborolan-2-yl)butyl)butanamide hydrochloride, [Step 2]: To a solution of tert-butyl ((R)-4-morpholino-1, 4-dioxo-1-(((S)-4-phenyl-1-(4, 4, 5, 5-tetramethyl-1, 3, 2-dioxaborolan-2-yl)butyl)amino)butan-2-yl)carbamate (40-3, 700 mg, 1.2 mmol) in 1, 4-dioxane (6 mL) was added 4 M HCl in dioxane (6.0 mL, 25.0 mmol) at 0° C. It was gradually warmed to 25° C. and stirred for 16 h. TLC showed complete consumption of starting material to form new polar spot. Volatiles were removed under reduced pressure to get (R)-2-amino-4-morpholino-4-oxo-N—((S)-4-phenyl-1-(4, 4, 5, 5-tetramethyl-1, 3, 2-dioxaborolan-2-yl)butyl)butanamide hydrochloride (40-4, 600 mg). The product was directly used for next step without purification.

Synthesis of N—((R)-4-morpholino-1, 4-dioxo-1-(((S)-4-phenyl-1-(4, 4, 5, 5-tetramethyl-1, 3, 2-dioxaborolan-2-yl)butyl)amino)butan-2-yl) pyrazine-2-carboxamide, [Step 3]: To a stirred solution of pyrazine-2-carboxylic acid (40-5, 165 mg, 1.3 mmol) in THF (8 mL) was added IBCF (0.2 mL, 1.3 mmol) and NMM (0.15 mL, 1.3 mmol) at −15° C. The reaction mixture was stirred at same temperature for 30 min. Then (R)-2-amino-4-morpholino-4-oxo-N—((S)-4-phenyl-1-(4, 4, 5, 5-tetramethyl-1, 3, 2-dioxaborolan-2-yl)butyl)butanamide hydrochloride (40-4, 600 mg, 1.2 mmol) in DMF (1 mL) followed by NMM (0.1 mL, 1.2 mmol) was added to the reaction mixture under same condition. It was gradually warmed to 0° C. and stirred for 2 h. LCMS of the crude reaction mass confirmed the formation of desired product. It was neutralized with saturated aqueous 0.1 N HCl solution and extracted with ethyl acetate. The combined organic layer was washed with 5% K₂CO₃ solution, water, brine, dried over Na₂SO₄, filtered and evaporated under reduced pressure. The material was purified by RP prep HPLC purification and lyophilized to afford N—((R)-4-morpholino-1, 4-dioxo-1-(((S)-4-phenyl-1-(4, 4, 5, 5-tetramethyl-1, 3, 2-dioxaborolan-2-yl)butyl) amino)butan-2-yl) pyrazine-2-carboxamide (40-6, 80 mg). [M−H]⁺=564.

Synthesis of ((S)-1-((R)-4-morpholino-4-oxo-2-(pyrazine-2-carboxamido)butanamido)-4-phenylbutyl)boronic acid, [Step 4]: To a stirred solution of N—((R)-4-morpholino-1, 4-dioxo-1-(((S)-4-phenyl-1-(4, 4, 5, 5-tetramethyl-1, 3, 2-dioxaborolan-2-yl)butyl)amino)butan-2-yl) pyrazine-2-carboxamide (40-6, 70 mg, 0.1 mmol) and methylboronic acid (40-7, 74 mg, 1.0 mmol) in acetone (4 mL) was added 0.2 N HCl (4 mL) and the reaction mixture was stirred at RT overnight. TLC and LCMS showed full conversion of starting material with formation of new polar spot. The volatiles were evaporated and the product was purified by RP prep HPLC purification and lyophilized to afford ((S)-1-((R)-4-morpholino-4-oxo-2-(pyrazine-2-carboxamido)butanamido)-4-phenylbutyl)boronic acid (Compound 40, 16 mg). [M−H]⁺=482; ¹H NMR (400 MHZ, MeOD) δ 9.25 (s, 1H), 8.79 (d, 1H), 8.68 (s, 1H), 7.21-7.07 (m, 5H), 5.28 (t, 1H), 3.66 (t, 2H), 3.61-3.47 (m, 6H), 2.98-2.93 (m, 1H), 2.65 (t, 1H), 2.58-2.57 (m, 2H), 1.65-1.39 (m, 5H).

### Example 24: Synthesis of ((R)-1-((R)-2-(cyclohexanecarboxamido)-4-morpholino-4-oxobutanamido)-4-phenylbutyl)boronic acid

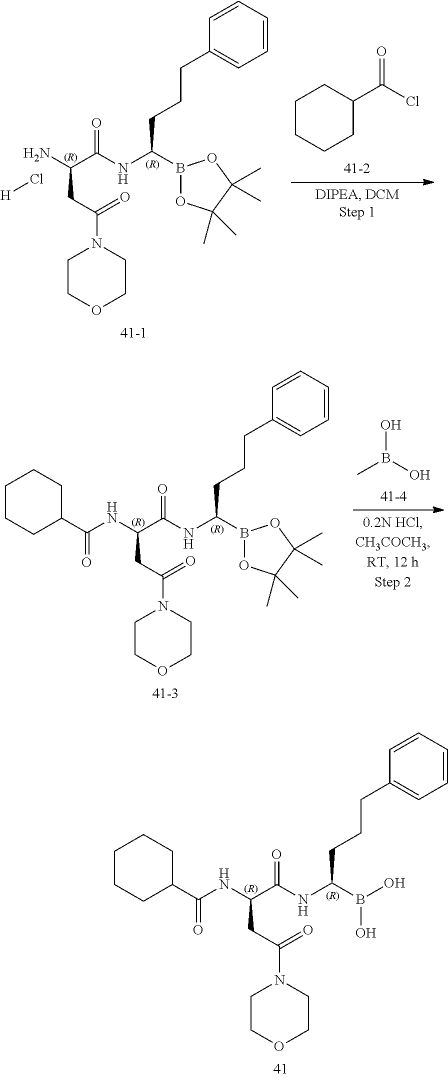

Synthesis of N—((R)-4-morpholino-1, 4-dioxo-1-(((R)-4-phenyl-1-(4, 4, 5, 5-tetramethyl-1, 3, 2-dioxaborolan-2-yl)butyl)amino)butan-2-yl) cyclohexanecarboxamide, [Step 1]: To a stirred solution of (R)-2-amino-4-morpholino-4-oxo-N—((R)-4-phenyl-1-(4, 4, 5, 5-tetramethyl-1, 3, 2-dioxaborolan-2-yl)butyl)butanamide hydrochloride (41-1, 300 mg, 0.6 mmol) and cyclohexane carbonyl chloride (41-2, 0.1 mL, 0.7 mmol) in DCM (4 mL), DIPEA (0.5 mL, 3. mmol) was added in ice cold condition and the reaction mixture was stirred at RT for 2 h. TLC and LCMS showed full conversion of starting material with formation of new polar spot. The reaction was diluted with DCM and washed with water and brine solution. The organic phase was dried over Na₂SO₄, filtered and evaporated to yield product. The product was purified by RP prep HPLC purification and lyophilized to afford N—((R)-4-morpholino-1, 4-dioxo-1-(((R)-4-phenyl-1-(4, 4, 5, 5-tetramethyl-1, 3, 2-dioxaborolan-2-yl)butyl)amino)butan-2-yl) cyclohexanecarboxamide (41-3, 45 mg). [M−H]⁺=569.

Synthesis of ((R)-1-((R)-2-(cyclohexanecarboxamido)-4-morpholino-4-oxobutanamido)-4-phenylbutyl)boronic acid, [Step 2]: To a stirred solution of N—((R)-4-morpholino-1, 4-dioxo-1-(((R)-4-phenyl-1-(4, 4, 5, 5-tetramethyl-1, 3, 2-dioxaborolan-2-yl)butyl)amino)butan-2-yl) cyclohexanecarboxamide (41-3, 45 mg, 0.1 mmol) and methylboronic acid (41-4, 45 mg, 0.8 mmol) in acetone (3 mL) was added 0.2 N HCl (3.0 mL) and the reaction mixture was stirred at RT overnight. TLC and LCMS showed full conversion of starting material with formation of new polar spot. The volatiles were evaporated and the residue was redissolved in acetone and deionized water and freeze-dried to obtain product. The material was purified by RP prep HPLC purification and lyophilized to afford ((R)-1-((R)-2-(cyclohexanecarboxamido)-4-morpholino-4-oxobutanamido)-4-phenylbutyl)boronic acid (Compound 41, 17 mg). [M−H]⁺=486; ¹H NMR (400 MHz, MeOD) δ 7.24-7.10 (m, 5H), 4.97 (br s, 1H), 3.65-3.59 (m, 4H), 3.50-3.48 (m, 4H), 2.92-2.90 (m, 2H), 2.61-2.59 (m, 3H), 2.19 (t, 1H), 1.78-1.67 (m, 7H), 1.46-1.28 (m, 7H).

### Example 25: Synthesis of ((R)-1-((R)-4-morpholino-4-oxo-2-(tetrahydro-2H-pyran-4-carboxamido)butanamido)-4-phenylbutyl)boronic acid

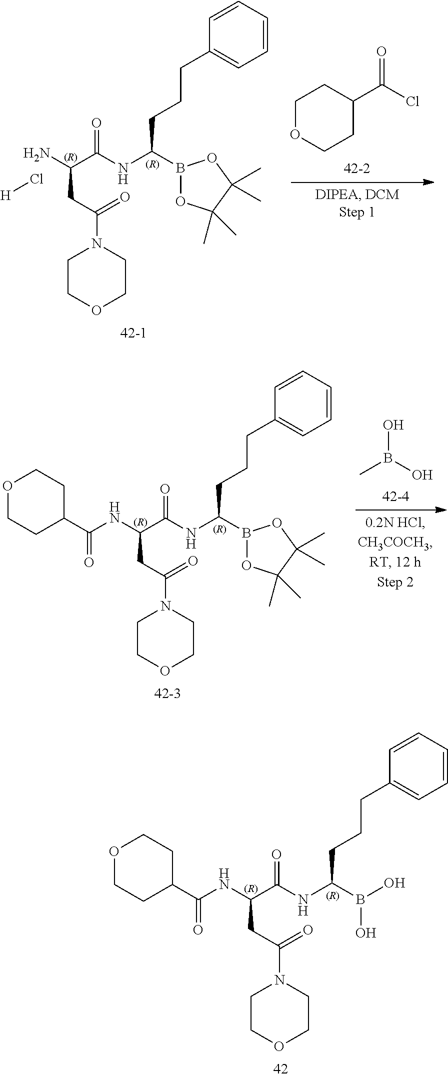

Synthesis of N—((R)-4-morpholino-1, 4-dioxo-1-(((R)-4-phenyl-1-(4, 4, 5, 5-tetramethyl-1, 3, 2-dioxaborolan-2-yl)butyl)amino)butan-2-yl) tetrahydro-2H-pyran-4-carboxamide, [Step 1]: To a stirred solution of (R)-2-amino-4-morpholino-4-oxo-N—((R)-4-phenyl-1-(4, 4, 5, 5-tetramethyl-1, 3, 2-dioxaborolan-2-yl)butyl)butanamide hydrochloride (42-1, 300 mg, 0.6 mmol) and tetrahydro-2H-pyran-4-carbonyl chloride (42-2, 0.08 mL, 0.7 mmol) in DCM (4 mL), DIPEA (0.5 mL, 3 mmol) was added in ice cold condition and the reaction mixture was stirred at RT for 2 h. TLC and LCMS showed full conversion of starting material with formation of new polar spot. The reaction was diluted with DCM and washed with water and brine solution. The organic phase was dried over Na₂SO₄, filtered and evaporated to yield product. The material was purified by RP prep HPLC purification and lyophilized to afford N—((R)-4-morpholino-1, 4-dioxo-1-(((R)-4-phenyl-1-(4, 4, 5, 5-tetramethyl-1, 3, 2-dioxaborolan-2-yl)butyl)amino)butan-2-yl) tetrahydro-2H-pyran-4-carboxamide (42-3, 75 mg). [M−H]⁺=570.

Synthesis of ((R)-1-((R)-4-morpholino-4-oxo-2-(tetrahydro-2H-pyran-4-carboxamido) butanamido)-4-phenylbutyl)boronic acid, [Step 2]: To a stirred solution of N—((R)-4-morpholino-1, 4-dioxo-1-(((R)-4-phenyl-1-(4, 4, 5, 5-tetramethyl-1, 3, 2-dioxaborolan-2-yl)butyl)amino)butan-2-yl) tetrahydro-2H-pyran-4-carboxamide (42-3, 75 mg, 0.13 mmol) and methylboronic acid (42-4, 80 mg, 1.3 mmol) in acetone (4 mL) was added 0.2 N HCl (4 mL) and the reaction mixture was stirred at RT overnight. TLC and LCMS showed full conversion of starting material with formation of new polar spot. The volatiles were evaporated and the residue was redissolved in acetone and deionized water and freeze-dried to obtain product. The material was purified by RP prep HPLC purification and lyophilized to afford ((R)-1-((R)-4-morpholino-4-oxo-2-(tetrahydro-2H-pyran-4-carboxamido)butanamido)-4-phenylbutyl) boronic acid (Compound 42, 49 mg). [M−H]⁺=488; ¹H NMR (400 MHZ, MeOD) δ 7.24-7.12 (m, 5H), 4.97 (t, 1H), 3.95-3.92 (m, 2H), 3.67-3.59 (m, 4H), 3.51-3.37 (m, 6H), 2.92 (d, 2H), 2.60-2.45 (m, 4H), 1.72-1.47 (m, 8H).

### Example 26: Synthesis of ((R)-1-((R)-4-morpholino-4-oxo-2-(phenylsulfonamido) butanamido)-4-phenylbutyl)boronic acid

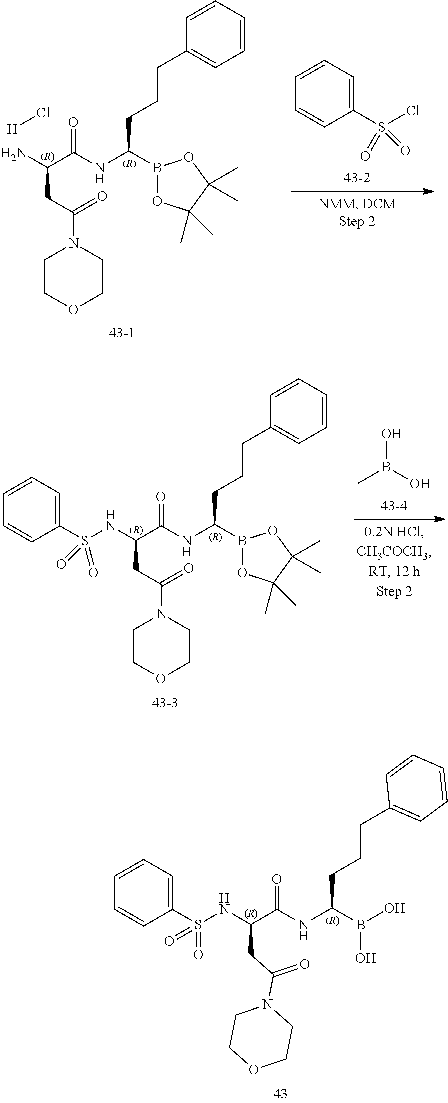

Synthesis of (R)-4-morpholino-4-oxo-N—((R)-4-phenyl-1-(4, 4, 5, 5-tetramethyl-1, 3, 2-dioxaborolan-2-yl)butyl)-2-(phenylsulfonamido)butanamide, [Step 1]: To a stirred solution of (R)-2-amino-4-morpholino-4-oxo-N—((R)-4-phenyl-1-(4, 4, 5, 5-tetramethyl-1, 3, 2-dioxaborolan-2-yl)butyl)butanamide hydrochloride (43-1, 300 mg, 0.6 mmol) and benzenesulfonyl chloride (43-2, 0.08 mL, 0.67 mmol) in DCM (4 mL) was added NMM (0.33 mL, 3 mmol) in ice cold condition and stirred at RT for 2 h. TLC and LCMS showed complete conversion of starting material to desired product. The reaction was diluted with DCM and washed with water and brine, dried over Na₂SO₄ and evaporated. The product was purified via prep HPLC purification and lyophilized to afford mixture of (R)-4-morpholino-4-oxo-N—((R)-4-phenyl-1-(4, 4, 5, 5-tetramethyl-1, 3, 2-dioxaborolan-2-yl)butyl)-2-(phenylsulfonamido)butanamide and ((R)-1-((R)-4-morpholino-4-oxo-2-(phenylsulfonamido)butanamido)-4-phenylbutyl)boronic acid (43-3, 47 mg). [M−H]⁺=598.

Synthesis of ((R)-1-((R)-4-morpholino-4-oxo-2-(phenylsulfonamido)butanamido)-4-phenyl butyl)boronic acid, [Step 2]: To a stirred solution of (R)-4-morpholino-4-oxo-N—((R)-4-phenyl-1-(4, 4, 5, 5-tetramethyl-1, 3, 2-dioxaborolan-2-yl)butyl)-2-(phenylsulfonamido)butanamide [mixture with ((R)-1-((R)-4-morpholino-4-oxo-2-(phenylsulfonamido)butanamido)-4-phenylbutyl)boronic acid] (43-3, 47 mg, 0.08 mmol) and methylboronic acid (43-4, 47 mg, 0.8 mmol) in acetone (3 mL) was added 0.2 N HCl (3.0 mL) and stirred at RT for overnight. TLC and LCMS showed full conversion of starting material to desired product. Volatiles were evaporated under reduced pressure and lyophilized. The product was purified via prep HPLC purification and lyophilized to afford ((R)-1-((R)-4-morpholino-4-oxo-2-(phenylsulfonamido)butanamido)-4-phenylbutyl)boronic acid (Compound 43, 31 mg). [M−H]⁺=516; ¹H NMR (400 MHZ, MeOD) δ 7.87-7.85 (m, 2H), 7.60-7.51 (m, 3H), 7.26-7.13 (m, 5H), 4.45 (t, 1H), 3.58-3.53 (m, 4H), 3.43-3.33 (m, 4H), 2.76-2.69 (m, 2H), 2.61-2.56 (m, 3H), 1.61-1.59 (m, 2H), 1.50-1.20 (m, 2H).

### Example 27: Synthesis of ((1R)-1-((2R)-4-morpholino-4-oxo-2-(tetrahydro-2H-pyran-2-carboxamido)butanamido)-4-phenylbutyl)boronic acid

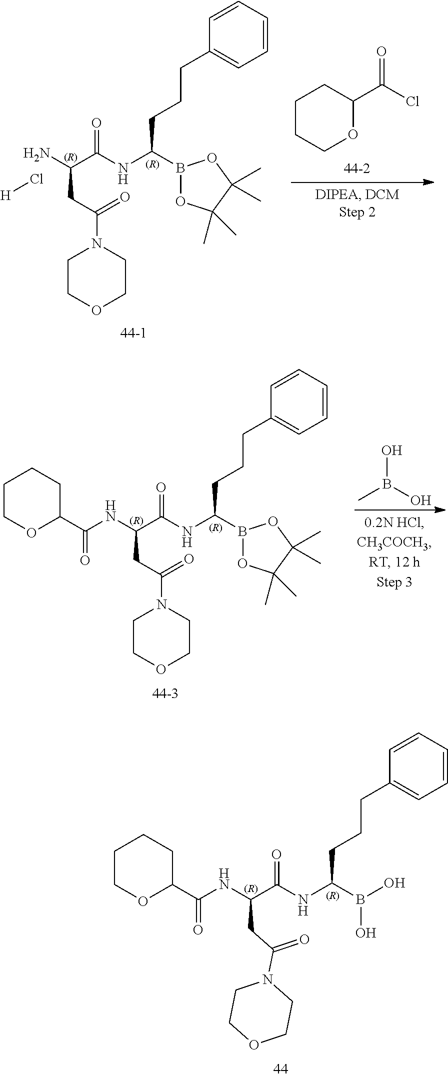

Synthesis of N—((R)-4-morpholino-1, 4-dioxo-1-(((R)-4-phenyl-1-(4, 4, 5, 5-tetramethyl-1, 3, 2-dioxaborolan-2-yl)butyl)amino)butan-2-yl) tetrahydro-2H-pyran-2-carboxamide, [Step 1]: To a stirred solution of (R)-2-amino-4-morpholino-4-oxo-N—((R)-4-phenyl-1-(4, 4, 5, 5-tetramethyl-1, 3, 2-dioxaborolan-2-yl)butyl)butanamide hydrochloride (44-1, 250 mg, 0.5 mmol) and tetrahydropyran-2-carbonyl chloride (44-2, 82 mg, 0.5 mmol) in DCM (4 mL), DIPEA (0.4 mL, 2.5 mmol) was added in ice cold condition and the reaction mixture was stirred at RT for 2 h. The reaction was diluted with DCM and washed with water and brine solution. The organic phase was dried over Na₂SO₄, filtered and evaporated to yield product. The material was purified by prep HPLC purification and lyophilized to afford N—((R)-4-morpholino-1, 4-dioxo-1-(((R)-4-phenyl-1-(4, 4, 5, 5-tetramethyl-1, 3, 2-dioxaborolan-2-yl)butyl)amino)butan-2-yl) tetrahydro-2H-pyran-2-carboxamide (44-3, 60 mg). [M−H]⁺=571.

Synthesis of ((1R)-1-((2R)-4-morpholino-4-oxo-2-(tetrahydro-2H-pyran-2-carboxamido) butanamido)-4-phenylbutyl)boronic acid, [Step 2]: To a stirred solution of N—((R)-4-morpholino-1, 4-dioxo-1-(((R)-4-phenyl-1-(4, 4, 5, 5-tetramethyl-1, 3, 2-dioxaborolan-2-yl)butyl)amino)butan-2-yl) tetrahydro-2H-pyran-2-carboxamide (44-3, 60 mg, 0.1 mmol) and methylboronic acid (63 mg, 1 mmol) in acetone (4 mL) was added 0.2 N HCl (4.0 mL) and the reaction mixture was stirred at RT overnight. The volatiles were evaporated and the residue was redissolved in acetone and deionized water and freeze-dried to obtain product. The material was purified by prep HPLC purification and lyophilized to afford ((1R)-1-((2R)-4-morpholino-4-oxo-2-(tetrahydro-2H-pyran-2-carboxamido)butanamido)-4-phenylbutyl)boronic acid (Compound 44, 25 mg). [M−H]⁺=488; ¹H NMR (400 MHZ, MeOD) δ 7.23-7.12 (m, 5H), 5.05 (br s, 1H), 4.05 (d, 1H), 3.78-3.88 (m, 1H), 3.66-3.59 (m, 4H), 3.51-3.48 (m, 6H), 3.28-3.05 (m, 2H), 2.94-2.84 (m, 1H), 2.63-2.58 (m, 3H), 1.97-1.88 (m.3H) 1.67-1.50 (m, 10H).

### Example 28: Synthesis of ((R)-1-((S)-4-morpholino-4-oxo-2-(pyrazine-2-carboxamido) butanamido)-4-phenylbutyl)boronic acid

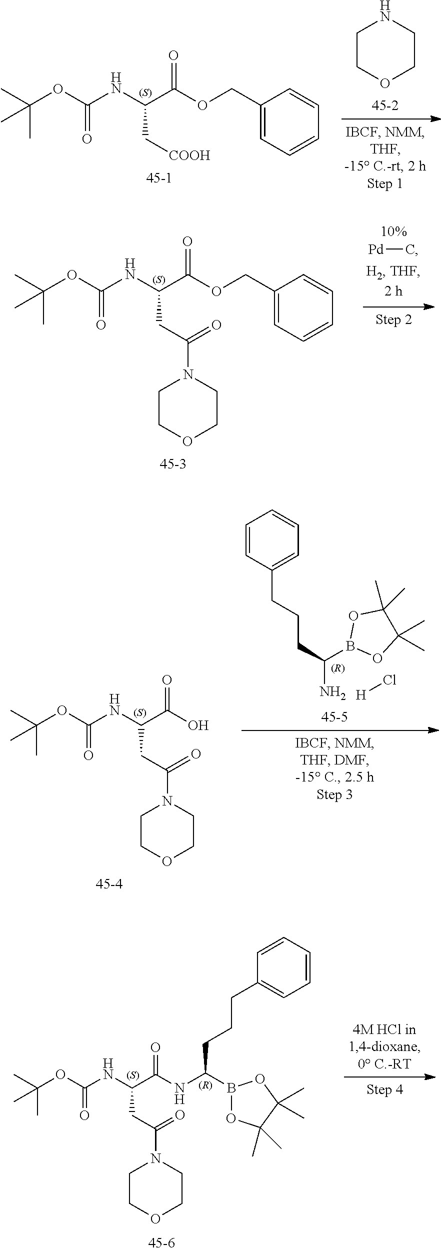

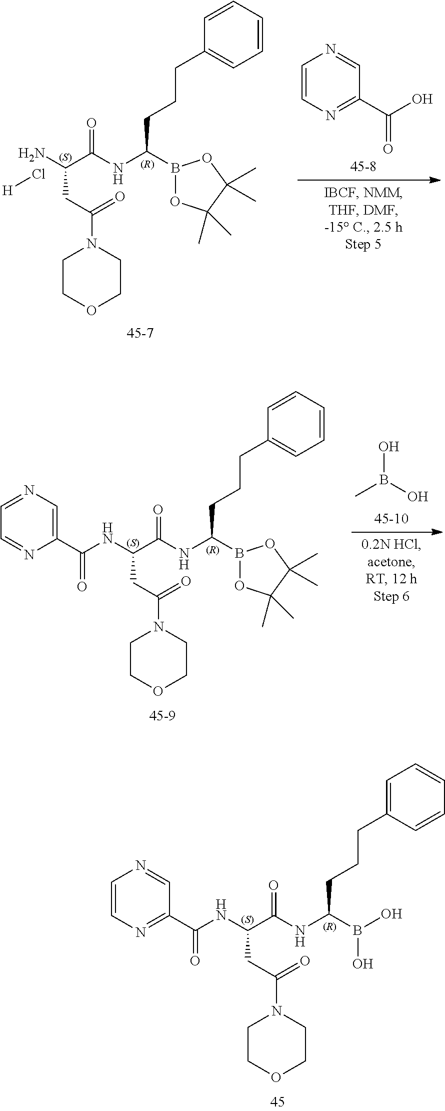

Synthesis of benzyl(S)-2-((tert-butoxycarbonyl)amino)-4-morpholino-4-oxobutanoate, [Step 1]: To a stirred solution of(S)-4-(benzyloxy)-3-((tert-butoxycarbonyl)amino)-4-oxobutanoic acid (45-1, 3.0 g, 9.4 mmol) in THF (25 mL) was added IBCF (1.2 mL, 9.4 mmol) and NMM (1.0 mL, 9.4 mmol) at −15° C. The reaction mixture was stirred at same temperature for 30 min. Then morpholine (45-2, 0.75 mL, 8.6 mmol) followed by NMM (0.9 mL, 8.6 mmol) was added to the reaction mixture at −15° C. It was gradually warmed to 0° C. and stirred for 2 h. LCMS of crude reaction mass confirmed the formation of desired product. It was neutralized with saturated aqueous 0.1 N HCl solution and extracted with ethyl acetate. Combined organic layer was washed with 5% K₂CO₃ solution, water, brine, dried over Na₂SO₄, filtered and evaporated under reduced pressure to get product. The product was purified through combiflash column chromatography to afford benzyl(S)-2-((tert-butoxycarbonyl)amino)-4-morpholino-4-oxobutanoate (45-3, 3.2 g). [M+H]⁺=393.

Synthesis of(S)-2-((tert-butoxycarbonyl)amino)-4-morpholino-4-oxobutanoic acid, [Step 2]: To a stirred solution of benzyl(S)-2-((tert-butoxycarbonyl)amino)-4-morpholino-4-oxobutanoate (45-3, 3.4 g, 8.6 mmol) dissolved in THF (30 mL) was bubbled with nitrogen gas for 10 min. Then 10% Pd—C(1.3 g, 11.9 mmol) was added and the reaction mixture was hydrogenated under balloon pressure for 16 h. The reaction was monitored by TLC. Upon completion, the reaction was filtered over celite with excess ethyl acetate. Removal of solvent under reduced pressure provided(S)-2-((tert-butoxycarbonyl)amino)-4-morpholino-4-oxobutanoic acid (45-4, 2.4 mg). [M+H]=301.

Synthesis of tert-butyl ((S)-4-morpholino-1, 4-dioxo-1-(((R)-4-phenyl-1-(4, 4, 5, 5-tetramethyl-1, 3, 2-dioxaborolan-2-yl)butyl)amino)butan-2-yl)carbamate, [Step 3]: To a stirred solution of(S)-2-((tert-butoxycarbonyl)amino)-4-morpholino-4-oxobutanoic acid (45-4, 695 mg, 2.3 mmol) in THF (10 mL) was added IBCF (0.3 mL, 2.3 mmol) and NMM (0.3 mL, 2.3 mmol) at −15° C. Reaction mixture was stirred at same temperature for 30 min. Then (R)-4-phenyl-1-(4, 4, 5, 5-tetramethyl-1, 3, 2-dioxaborolan-2-yl)butan-1-amine hydrochloride (45-5, 650 mg, 2.1 mmol) in DMF (1 mL) followed by NMM (0.2 mL, 2.1 mmol) was added to the reaction mixture at −15° C. It was gradually warmed to 0° C. and stirred for 2 h. LCMS of crude reaction mass confirmed the formation of desired product. It was neutralized with saturated aqueous 0.1 N HCl solution and extracted with ethyl acetate. Combined organic layer was washed with 5% K₂CO₃ solution, water, brine, dried over Na₂SO₄, filtered and evaporated under reduced pressure to afford tert-butyl ((S)-4-morpholino-1, 4-dioxo-1-(((R)-4-phenyl-1-(4, 4, 5, 5-tetramethyl-1, 3, 2-dioxaborolan-2-yl)butyl)amino)butan-2-yl)carbamate (45-6, 700 mg) which is directly used in next step. [M−H]=558.

Synthesis of(S)-2-amino-4-morpholino-4-oxo-N—((R)-4-phenyl-1-(4, 4, 5, 5-tetramethyl-1, 3, 2-dioxaborolan-2-yl)butyl)butanamide hydrochloride, [Step 4]: To a solution of tert-butyl ((S)-4-morpholino-1, 4-dioxo-1-(((R)-4-phenyl-1-(4, 4, 5, 5-tetramethyl-1, 3, 2-dioxaborolan-2-yl)butyl)amino)butan-2-yl)carbamate (45-6, 700 mg, 1.2 mmol) in 6 mL of 1, 4-dioxane was added 4 M HCl in dioxane (6.0 mL, 24.0 mmol) at 0° C. It was gradually warmed to 25° C. and stirred for 16 h. TLC showed complete consumption of starting material to form new polar spot. Volatiles were removed under reduced pressure to get(S)-2-amino-4-morpholino-4-oxo-N—((R)-4-phenyl-1-(4, 4, 5, 5-tetramethyl-1, 3, 2-dioxaborolan-2-yl)butyl)butanamide hydrochloride (45-7, 560 mg). Product was directly used for next step without purification. [M−H]=458.

Synthesis of N—((S)-4-morpholino-1, 4-dioxo-1-(((R)-4-phenyl-1-(4, 4, 5, 5-tetramethyl-1, 3, 2-dioxaborolan-2-yl)butyl)amino)butan-2-yl) pyrazine-2-carboxamide, [Step 5]: To a stirred solution of pyrazine-2-carboxylic acid (45-8, 165 mg, 1.3 mmol) in THF (8 mL) was added IBCF (0.17 mL, 1.3 mmol) and NMM (0.17 mL, 1.3 mmol) at −15° C. The reaction mixture was stirred at same temperature for 30 min. Then(S)-2-amino-4-morpholino-4-oxo-N—((R)-4-phenyl-1-(4, 4, 5, 5-tetramethyl-1, 3, 2-dioxaborolan-2-yl)butyl)butanamide hydrochloride (45-7, 600 mg, 1.2 mmol) in DMF (1 mL) followed by NMM (0.14 mL, 1.2 mmol) was added to the reaction mixture under same condition. It was gradually warmed to 0° C. and stirred for 2 h. LCMS of the crude reaction mass confirmed the formation of desired product. It was neutralized with saturated aqueous 0.1 N HCl solution and extracted with ethyl acetate. The combined organic layer was washed with 5% K₂CO₃ solution, water, brine, dried over Na₂SO₄, filtered and evaporated under reduced pressure. The material was purified by RP prep HPLC purification and lyophilized to afford N—((S)-4-morpholino-1, 4-dioxo-1-(((R)-4-phenyl-1-(4, 4, 5, 5-tetramethyl-1, 3, 2-dioxaborolan-2-yl)butyl)amino)butan-2-yl) pyrazine-2-carboxamide (45-9, 105 mg). [M−H]⁺=564.

Synthesis of ((R)-1-((S)-4-morpholino-4-oxo-2-(pyrazine-2-carboxamido)butanamido)-4-phenylbutyl)boronic acid, [Step 6]: To a stirred solution of N—((S)-4-morpholino-1, 4-dioxo-1-(((R)-4-phenyl-1-(4, 4, 5, 5-tetramethyl-1, 3, 2-dioxaborolan-2-yl)butyl)amino)butan-2-yl) pyrazine-2-carboxamide (45-9, 100 mg, 0.17 mmol) and methylboronic acid (105 mg, 1.7 mmol) in acetone (4 mL) was added 0.2 N HCl (4 mL) and the reaction mixture was stirred at RT overnight. TLC and LCMS showed full conversion of starting material with formation of new polar spot. The volatiles were evaporated and the residue was redissolved in acetone and deionized water and freeze-dried to obtain the product. The material was purified by RP prep HPLC purification and lyophilized to afford ((R)-1-((S)-4-morpholino-4-oxo-2-(pyrazine-2-carboxamido)butanamido)-4-phenylbutyl)boronic acid (Compound 45, 62 mg). [M−H]⁺=483; ¹H NMR (400 MHZ, MeOD) δ 9.25 (s, 1H), 8.79 (d, 1H), 8.68 (s, 1H), 7.21-7.07 (m, 5H), 5.28 (t, 1H), 3.66 (t, 2H), 3.61-3.47 (m, 6H), 2.98-2.93 (m, 1H), 2.64 (t, 1H), 2.58-2.55 (m, 2H), 1.65-1.28 (m, 5H).

### Example 29: Synthesis of ((S)-1-((S)-4-morpholino-4-oxo-2-(pyrazine-2-carboxamido) butanamido)-4-phenylbutyl)boronic acid

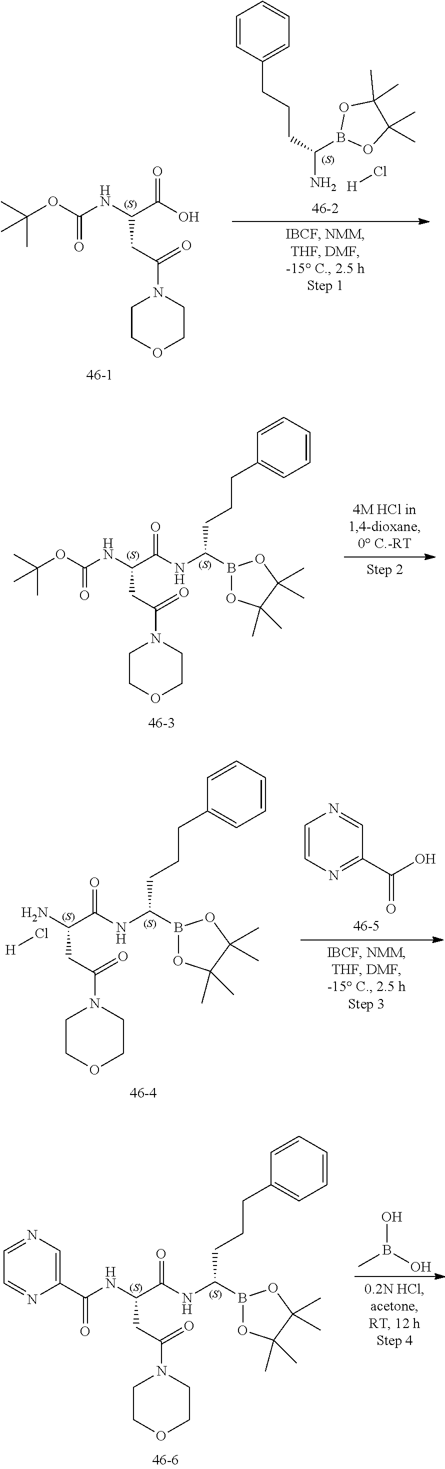

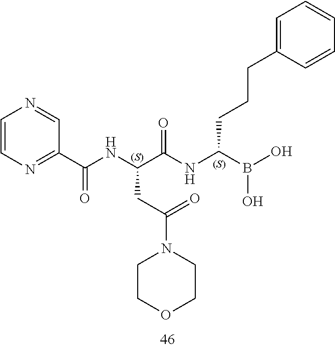

Synthesis of tert-butyl ((S)-4-morpholino-1, 4-dioxo-1-(((S)-4-phenyl-1-(4, 4, 5, 5-tetramethyl-1, 3, 2-dioxaborolan-2-yl)butyl)amino)butan-2-yl)carbamate, [Step 1]: To a stirred solution of(S)-2-((tert-butoxycarbonyl)amino)-4-morpholino-4-oxobutanoic acid (46-1, 610 mg, 2.0 mmol) in THF (10 mL) was added IBCF (0.25 mL, 2.0 mmol) and NMM (0.26 mL, 2.0 mmol) at −15° C. Reaction mixture was stirred at same temperature for 30 min. Then(S)-4-phenyl-1-(4, 4, 5, 5-tetramethyl-1, 3, 2-dioxaborolan-2-yl)butan-1-amine hydrochloride (46-2, 570 mg, 1.8 mmol) in DMF (1 mL) followed by NMM (0.2 mL, 1.8 mmol) was added to the reaction mixture at −15° C. It was gradually warmed to 0° C. and stirred for 2 h. LCMS of crude reaction mass confirmed the formation of desired product. It was neutralized with saturated aqueous 0.1 N HCl solution and extracted with ethyl acetate. Combined organic layer was washed with 5% K₂CO₃ solution, water, brine, dried over Na₂SO₄, filtered and evaporated under reduced pressure to afford tert-butyl ((S)-4-morpholino-1, 4-dioxo-1-(((S)-4-phenyl-1-(4, 4, 5, 5-tetramethyl-1, 3, 2-dioxaborolan-2-yl)butyl)amino)butan-2-yl)carbamate (46-3, 700 mg), which is directly used in next step. [M−H]=559.

Synthesis of(S)-2-amino-4-morpholino-4-oxo-N—((S)-4-phenyl-1-(4, 4, 5, 5-tetramethyl-1, 3, 2-dioxaborolan-2-yl)butyl)butanamide hydrochloride, [Step 2]: To a solution of tert-butyl ((S)-4-morpholino-1, 4-dioxo-1-(((S)-4-phenyl-1-(4, 4, 5, 5-tetramethyl-1, 3, 2-dioxaborolan-2-yl)butyl)amino)butan-2-yl)carbamate (46-3, 700 mg, 1.2 mmol) in 6 mL of 1, 4-dioxane was added 4 M HCl in dioxane (6.0 mL, 24.0 mmol) at 0° C. It was gradually warmed to 25° C. and stirred for 16 h. TLC showed complete consumption of starting material to form new polar spot. Volatiles were removed under reduced pressure to get(S)-2-amino-4-morpholino-4-oxo-N—((S)-4-phenyl-1-(4, 4, 5, 5-tetramethyl-1, 3, 2-dioxaborolan-2-yl)butyl)butanamide hydrochloride (46-4, 560 mg). Product was directly used for next step without purification. [M−H]=458.

Synthesis of N—((S)-4-morpholino-1, 4-dioxo-1-(((S)-4-phenyl-1-(4, 4, 5, 5-tetramethyl-1, 3, 2-dioxaborolan-2-yl)butyl)amino)butan-2-yl) pyrazine-2-carboxamide, [Step 3]: To a stirred solution of pyrazine-2-carboxylic acid (46-5, 165 mg, 1.3 mmol) in THF (8 mL) was added IBCF (0.17 mL, 1.3 mmol) and NMM (0.17 mL, 1.3 mmol) at −15° C. The reaction mixture was stirred at same temperature for 30 min. Then(S)-2-amino-4-morpholino-4-oxo-N—((S)-4-phenyl-1-(4, 4, 5, 5-tetramethyl-1, 3, 2-dioxaborolan-2-yl)butyl)butanamide hydrochloride (46-4, 600 mg, 1.2 mmol) in DMF (1 mL) followed by NMM (0.14 mL, 1.2 mmol) was added to the reaction mixture under same condition. It was gradually warmed to 0° C. and stirred for 2 h. LCMS of the crude reaction mass confirmed the formation of desired product. It was neutralized with saturated aqueous 0.1 N HCl solution and extracted with ethyl acetate. The combined organic layer was washed with 5% K₂CO₃ solution, water, brine, dried over Na₂SO₄, filtered and evaporated under reduced pressure. The material was purified by RP prep HPLC purification and lyophilized to afford N—((S)-4-morpholino-1, 4-dioxo-1-(((S)-4-phenyl-1-(4, 4, 5, 5-tetramethyl-1, 3, 2-dioxaborolan-2-yl)butyl)amino)butan-2-yl) pyrazine-2-carboxamide (46-6, 120 mg). [M−H]⁺=564.

Synthesis of ((S)-1-((S)-4-morpholino-4-oxo-2-(pyrazine-2-carboxamido)butanamido)-4-phenylbutyl)boronic acid, [Step 4]: To a stirred solution of N—((S)-4-morpholino-1, 4-dioxo-1-(((S)-4-phenyl-1-(4, 4, 5, 5-tetramethyl-1, 3, 2-dioxaborolan-2-yl)butyl)amino)butan-2-yl) pyrazine-2-carboxamide (46-6, 120 mg, 0.21 mmol) and methylboronic acid (127 mg, 2.1 mmol) in acetone (4 mL) was added 0.2 N HCl (4 mL) and the reaction mixture was stirred at RT overnight. TLC and LCMS showed full conversion of starting material with formation of new polar spot. The volatiles were evaporated and the residue was redissolved in acetone and deionized water and freeze-dried to obtain to get product. The material was purified by RP prep HPLC purification and lyophilized to afford ((S)-1-((S)-4-morpholino-4-oxo-2-(pyrazine-2-carboxamido)butanamido)-4-phenylbutyl)boronic acid (Compound 46, 45 mg). [M−H]⁺=482; ¹H NMR (400 MHZ, MeOD) 9.25 (s, 1H), 8.79 (d, 1H), 8.68 (s, 1H), 7.19-7.09 (m, 5H), 5.28 (t, 1H), 3.66-3.61 (m, 4H), 3.59-3.51 (m, 4H), 3.01-3.00 (m, 1H), 2.60-2.56 (m, 4H), 1.65-1.63 (m, 4H).

### Example 30: Synthesis of ((R)-1-((R)-4-morpholino-4-oxo-2-(pyrazine-2-carboxamido) butanamido)-2-phenylethyl)boronic acid

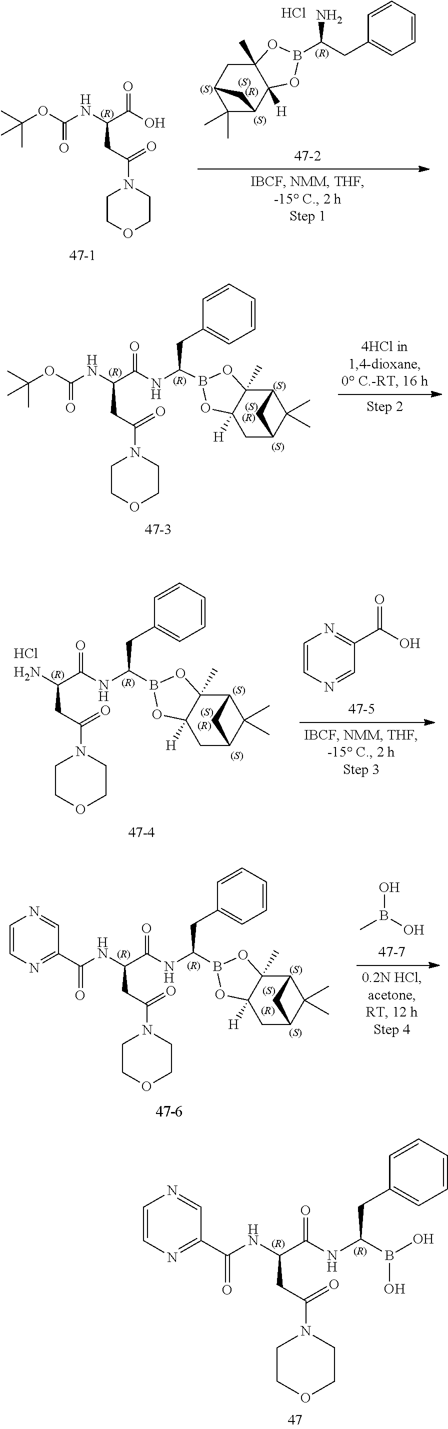

Synthesis of tert-butyl ((R)-4-morpholino-1, 4-dioxo-1-(((R)-2-phenyl-1-((3aS, 4S, 6S, 7aR)-3a, 5, 5-trimethylhexahydro-4, 6-methanobenzo[d][1, 3, 2]dioxaborol-2-yl)ethyl)amino) butan-2-yl)carbamate, [Step 1]: To a stirred solution of (R)-2-((tert-butoxycarbonyl)amino)-4-morpholino-4-oxobutanoic acid (47-1, 590 mg, 2 mmol) in tetrahydrofuran (10 mL) was added NMM (0.3 mL, 2 mmol) followed by IBCF (0.3 mL, 2 mmol) at −15° C. and stirred for 1 h. A solution of (1R)-2-phenyl-1-((4S, 6S, 7aS)-5, 5, 7a-trimethylhexahydro-4, 6-methanobenzo[d][1, 3, 2]dioxaborol-2-yl) ethan-1-amine hydrochloride (47-2, 600 mg, 1.8 mmol) in dimethylformamide (1.5 mL) and NMM (0.3 mL, 2 mmol) were added and stirred at ambient temperature for 2 h. The reaction mixture was diluted with ethyl acetate and washed with 0.1M HCl, 5% K₂CO₃, water and brine, dried over anhydrous Na₂SO₄ and evaporated to give tert-butyl ((R)-4-morpholino-1, 4-dioxo-1-(((R)-2-phenyl-1-((3aS, 4S, 6S, 7aR)-3a, 5, 5-trimethylhexahydro-4, 6-methanobenzo[d][1, 3, 2]dioxaborol-2-yl)ethyl)amino)butan-2-yl)carbamate (47-3, 800 mg). [M−H]⁻=583.

Synthesis of (R)-2-amino-4-morpholino-4-oxo-N—((R)-2-phenyl-1-((3aS, 4S, 6S, 7aR)-3a, 5, 5-trimethylhexahydro-4, 6-methanobenzo[d][1, 3, 2]dioxaborol-2-yl)ethyl)butanamide hydrochloride, [Step 2]: To a stirred solution of tert-butyl ((R)-4-morpholino-1, 4-dioxo-1-(((R)-2-phenyl-1-((3aS, 4S, 6S, 7aR)-3a, 5, 5-trimethylhexahydro-4, 6-methanobenzo[d][1, 3, 2]dioxaborol-2-yl)ethyl)amino)butan-2-yl)carbamate (47-3, 800 mg, 1.4 mmol) in 1, 4-dioxane (4 mL) was added 4M HCl in 1, 4-dioxane (4 mL, 14 mmol) in ice cold condition and stirred at ambient temperature for 2 h. Volatiles were removed under reduced pressure and triturated with n-pentane to give (R)-2-amino-4-morpholino-4-oxo-N—((R)-2-phenyl-1-((3aS, 4S, 6S, 7aR)-3a, 5, 5-trimethylhexahydro-4, 6-methanobenzo[d][1, 3, 2]dioxaborol-2-yl)ethyl)butanamide hydrochloride (47-4, 700 mg). [M−H]⁻=482.

Synthesis of N—((R)-4-morpholino-1, 4-dioxo-1-(((R)-2-phenyl-1-((3aS, 4S, 6S, 7aR)-3a, 5, 5-trimethyl hexahydro-4, 6-methanobenzo[d][1, 3, 2]dioxaborol-2-yl)ethyl)amino)butan-2-yl) pyrazine-2-carboxamide, [Step 3]: To a stirred solution of pyrazine-2-carboxylic acid (47-5, 220 mg, 1.7 mmol) in tetrahydrofuran (10 mL) was added NMM (0.2 mL, 1.5 mmol) followed by IBCF (0.2 mL, 1.4 mmol) at −15° C. and stirred for 30 min. A solution of (R)-2-amino-4-morpholino-4-oxo-N—((R)-2-phenyl-1-((3aS, 4S, 6S, 7aR)-3a, 5, 5-trimethylhexahydro-4, 6-methanobenzo[d][1, 3, 2]dioxaborol-2-yl)ethyl)butanamide hydrochloride (47-4, 700 mg, 1.4 mmol) in dimethylformamide (1 mL) and NMM (0.2 mL, 1.5 mmol) were added and stirred at RT for 2 h. The reaction mixture was diluted with ethyl acetate and washed with 0.1M HCl, 5% K₂CO₃, water and brine, dried over anhydrous Na₂SO₄ and concentrated under reduced pressure. The compound was purified by prep HPLC purification and lyophilized to afford N—((R)-4-morpholino-1, 4-dioxo-1-(((R)-2-phenyl-1-((3aS, 4S, 6S, 7aR)-3a, 5, 5-trimethyl hexahydro-4, 6-methanobenzo[d][1, 3, 2]dioxaborol-2-yl)ethyl)amino)butan-2-yl) pyrazine-2-carboxamide (47-6, 100 mg). [M−H]⁻=588.

Synthesis of ((R)-1-((R)-4-morpholino-4-oxo-2-(pyrazine-2-carboxamido)butanamido)-2-phenylethyl)boronic acid, [Step 4]: To a solution of N—((R)-4-morpholino-1, 4-dioxo-1-(((R)-2-phenyl-1-((3aS, 4S, 6S, 7aR)-3a, 5, 5-trimethyl hexahydro-4, 6-methanobenzo[d][1, 3, 2]dioxaborol-2-yl)ethyl)amino)butan-2-yl) pyrazine-2-carboxamide (47-6, 100 mg, 0.2 mmol) in acetone (4 mL) was added methylboronic acid (47-7, 200 mg, 3.4 mmol), followed by dropwise addition of 0.2N HCl (4 mL) and stirred at ambient temperature for overnight. Volatiles were removed under reduced pressure and purified by prep HPLC purification and lyophilized to afford ((R)-1-((R)-4-morpholino-4-oxo-2-(pyrazine-2-carboxamido)butanamido)-2-phenylethyl)boronic acid (Compound 47, 75 mg). [M−H]⁻=454; ¹H NMR (400 MHZ, CD3OD): δ 9.23 (d, 1H), 9.80 (d, 1H), 8.71 (q, 1H), 7.24-7.17 (m, 4H), 7.10 (d, 1H), 5.25 (t, 1H), 3.68-3.63 (m, 4H), 3.58-3.56 (m, 2H), 3.53 (t, 2H), 3.25 (d, 1H), 3.00 (dd, 1H), 2.87-2.81 (m, 2H), 2.66-2.63 (1H).

### Example 31: Synthesis of ((R)-1-((R)-2-benzamido-3-methoxypropanamido)-4-phenyl butyl)boronic acid

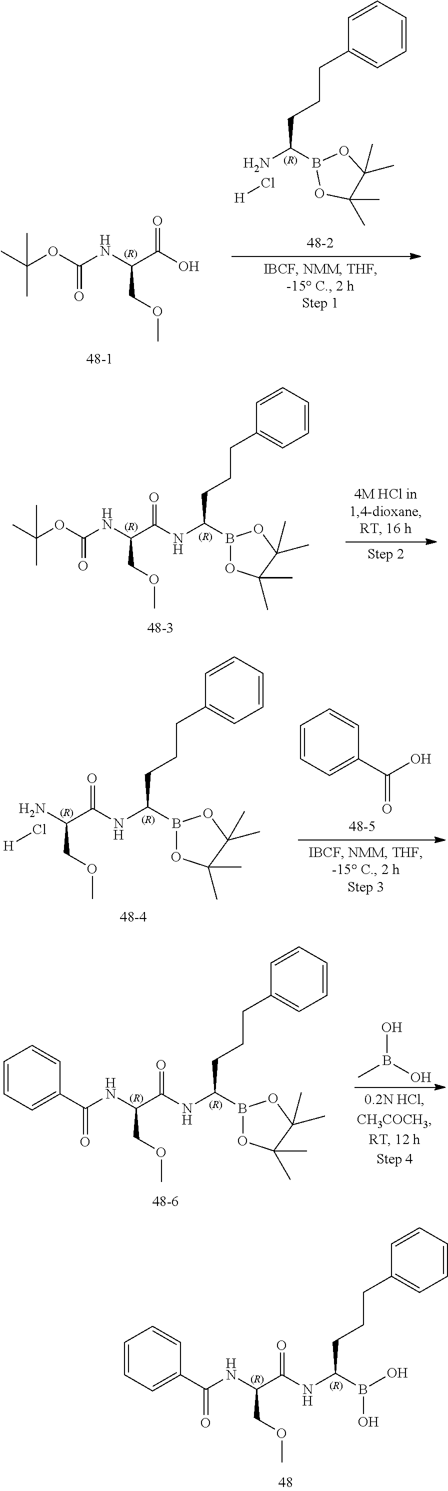

Synthesis of tert-butyl ((R)-3-methoxy-1-oxo-1-(((R)-4-phenyl-1-(4, 4, 5, 5-tetramethyl-1, 3, 2-dioxaborolan-2-yl)butyl)amino) propan-2-yl)carbamate, [Step 1]: To a stirred solution of N-(tert-butoxycarbonyl)-O-methyl-D-serine (48-1, 770 mg, 3.5 mmol) in THF (15 mL) were added NMM (0.4 mL, 3.5 mmol), IBCF (0.4 mL, 3.2 mmol) at −15° C. and the reaction mixture was stirred at this temperature for 1 h. To this solution were added (R)-4-phenyl-1-(4, 4, 5, 5-tetramethyl-1, 3, 2-dioxaborolan-2-yl)butan-1-amine hydrochloride (48-2, 1 g, 3.2 mmol) and NMM (0.4 mL, 3.5 mmol) and the reaction mixture was stirred at ambient temperature for 2 h. Reaction mixture was diluted with ethyl acetate and washed subsequently with 0.1 M HCl, 5% K₂CO₃, water and brine. The organic extracts was dried over Na₂SO₄, filtered and evaporated to afford tert-butyl ((R)-3-methoxy-1-oxo-1-(((R)-4-phenyl-1-(4, 4, 5, 5-tetramethyl-1, 3, 2-dioxaborolan-2-yl)butyl) amino) propan-2-yl)carbamate (48-3, 1.5 g). The product was used for next step without further purification. [M−H]⁻=475.

Synthesis of (R)-2-amino-3-methoxy-N—((R)-4-phenyl-1-(4, 4, 5, 5-tetramethyl-1, 3, 2-dioxaborolan-2-yl)butyl) propanamide hydrochloride, [Step 2]: To a stirred solution of tert-butyl ((R)-3-methoxy-1-oxo-1-(((R)-4-phenyl-1-(4, 4, 5, 5-tetramethyl-1, 3, 2-dioxaborolan-2-yl)butyl)amino) propan-2-yl)carbamate (48-3, 300 mg, 0.6 mmol) in 1, 4-dioxane (2 mL), add 4 M HCl-dioxane (2 mL, 8 mmol) was added in ice cold condition and the reaction mixture was stirred at ambient temperature for 2 h. Product was triturated with n-pentane to give (R)-2-amino-3-methoxy-N—((R)-4-phenyl-1-(4, 4, 5, 5-tetramethyl-1, 3, 2-dioxaborolan-2-yl)butyl) propanamide hydrochloride (48-4, 250 mg). The product was used for next step without further purification. [M−H]⁻=375.

Synthesis of N—((R)-3-methoxy-1-oxo-1-(((R)-4-phenyl-1-(4, 4, 5, 5-tetramethyl-1, 3, 2-dioxaborolan-2-yl)butyl)amino) propan-2-yl)benzamide, [Step 3]: To a stirred solution of benzoic acid (48-5, 75 mg, 0.6 mmol) in THF (7 mL), NMM (0.07 mL, 0.7 mmol), IBCF (0.08 mL, 0.6 mmol) were added at −15° C. and the reaction mixture was stirred at this temperature for 30 min. To this solution were added (R)-2-amino-3-methoxy-N—((R)-4-phenyl-1-(4, 4, 5, 5-tetramethyl-1, 3, 2-dioxaborolan-2-yl)butyl) propanamide hydrochloride (48-4, 250 mg, 0.6 mmol) and NMM (0.07 mL, 0.7 mmol) and the reaction mixture was stirred at ambient temperature for 2 h. The reaction mixture was diluted with ethyl acetate and washed subsequently with 0.1 M HCl, 5% K₂CO₃, water and brine. The organic phase was dried over Na₂SO₄, filtered and evaporated under reduced pressure. The product was purified by RP prep HPLC purification and the eluent was lyophilized to afford N—((R)-3-methoxy-1-oxo-1-(((R)-4-phenyl-1-(4, 4, 5, 5-tetramethyl-1, 3, 2-dioxaborolan-2-yl)butyl)amino) propan-2-yl)benzamide (48-6, 50 mg). [M−H]⁻=479.

Synthesis of ((R)-1-((R)-2-benzamido-3-methoxypropanamido)-4-phenylbutyl)boronic acid, [Step 4]: To a stirred solution of N—((R)-3-methoxy-1-oxo-1-(((R)-4-phenyl-1-(4, 4, 5, 5-tetramethyl-1, 3, 2-dioxaborolan-2-yl)butyl)amino) propan-2-yl)benzamide (48-6, 50 mg, 0.1 mmol) in acetone (3 mL) was added methylboronic acid (60 mg, 1 mmol) followed by dropwise addition of 0.2 N HCl (3 mL). The reaction mixture was allowed to stir at ambient temperature for 16 h. All the volatiles were evaporated at room temperature and residue was dissolved in acetonitrile and deionized water and freeze-dried to obtain product. The product was purified through RP prep HPLC purification and lyophilized to afford ((R)-1-((R)-2-benzamido-3-methoxypropanamido)-4-phenylbutyl)boronic acid (Compound 48, 35 mg). [M−H]⁻=397; ¹H NMR (400 MHZ, MeOD) δ 7.85 (d, 2H), 7.55 (t, 1H), 7.46 (t, 2H), 7.22-7.09 (m, 5H), 4.98 (t, 1H), 3.80-3.74 (m, 2H), 3.37 (s, 3H), 2.63-2.58 (m, 3H), 1.70-1.63 (m, 2H), 1.57-1.51 (2H).

### Example 32: Synthesis of ((R)-1-((R)-3-methoxy-2-(6-methoxypicolinamido)propanamido)-4-phenylbutyl)boronic acid

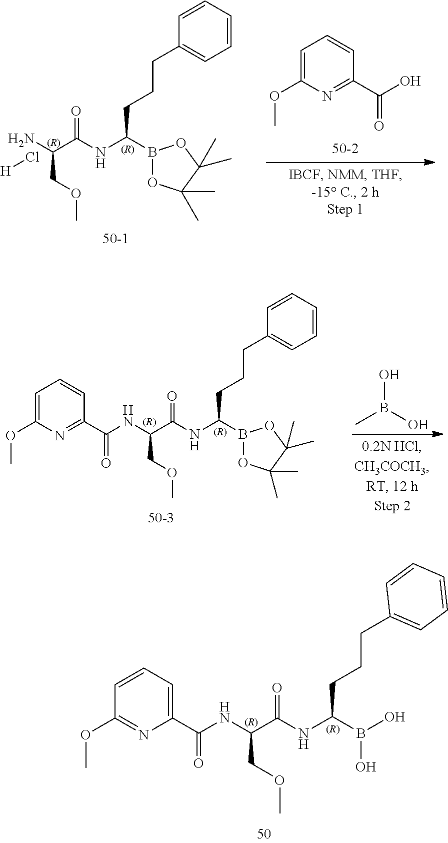

Synthesis of 6-methoxy-N—((R)-3-methoxy-1-oxo-1-(((R)-4-phenyl-1-(4, 4, 5, 5-tetramethyl-1, 3, 2-dioxaborolan-2-yl)butyl)amino) propan-2-yl) picolinamide, [Step 1]: To a stirred solution of 6-methoxypicolinic acid (50-2, 140 mg, 0.9 mmol) in THF (10 mL), NMM (0.1 mL, 0.8 mmol), IBCF (0.1 mL, 0.7 mmol) were added at −15° C. and the reaction mixture was stirred at this temperature for 30 min. Then (R)-2-amino-3-methoxy-N—((R)-4-phenyl-1-(4, 4, 5, 5-tetramethyl-1, 3, 2-dioxaborolan-2-yl)butyl) propanamide hydrochloride (50-1, 300 mg, 0.7 mmol) and NMM (0.1 mL, 0.8 mmol) were added and the reaction mixture was stirred at ambient temperature for 2 h. Reaction mixture was diluted with ethyl acetate and washed subsequently with 0.1 M HCl, 5% K₂CO₃, water and brine. The organic extracts was dried over Na₂SO₄, filtered and evaporated under reduced pressure. The product was purified by reverse phase prep HPLC to afford 6-methoxy-N—((R)-3-methoxy-1-oxo-1-(((R)-4-phenyl-1-(4, 4, 5, 5-tetramethyl-1, 3, 2-dioxaborolan-2-yl)butyl)amino) propan-2-yl) picolinamide (50-3, 50 mg). [M−H]⁻=510.

Synthesis of ((R)-1-((R)-3-methoxy-2-(6-methoxypicolinamido)propanamido)-4-phenyl butyl)boronic acid, [Step 2]: To a solution of 6-methoxy-N—((R)-3-methoxy-1-oxo-1-(((R)-4-phenyl-1-(4, 4, 5, 5-tetramethyl-1, 3, 2-dioxaborolan-2-yl)butyl)amino) propan-2-yl) picolinamide (50-3, 50 mg, 0.1 mmol) in acetone (3 mL) was added methylboronic acid (60 mg, 1 mmol) followed by dropwise addition of 0.2 N HCl (2 mL). The reaction mixture was allowed to stirring at ambient temperature for 16 h. All the volatiles were evaporated under reduced pressure and residue was dissolved in acetonitrile and deionized water and freeze-dried to obtain product. The product was purified through RP prep HPLC purification to afford ((R)-1-((R)-3-methoxy-2-(6-methoxypicolinamido)propanamido)-4-phenylbutyl)boronic acid (Compound 50, 35 mg). [M−H]⁻=428; ¹H NMR (400 MHZ, MeOD) δ 7.83 (t, 1H), 7.68 (d, 1H), 7.22-7.14 (m, 4H), 7.11 (d, 1H), 7.01 (d, 1H), 4.92 (s, 1H), 3.99 (s, 3H), 3.90-3.86 (m, 1H), 3.77-3.73 (m, 1H), 3.38 (s, 3H), 2.66 (d, 1H), 2.62-2.60 (m, 2H), 1.69-1.58 (m, 2H), 1.57-1.49 (m, 2H).

### Example 33: Synthesis of ((R)-1-((R)-2-(N-methylpyrazine-2-carboxamido) pentanamido)-4-phenylbutyl)boronic acid

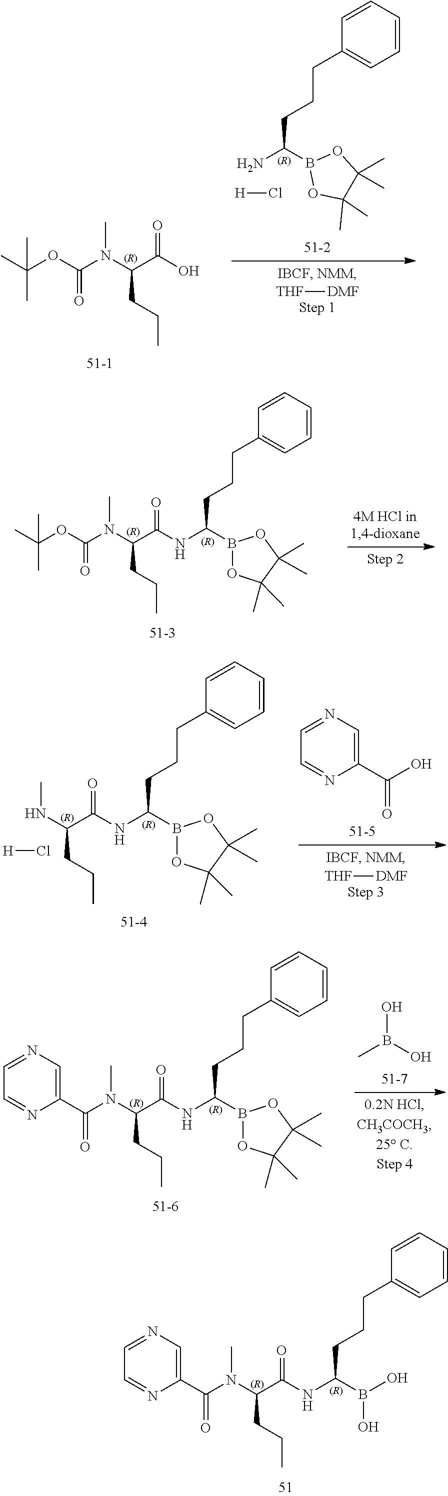

Synthesis of tert-butyl methyl ((R)-1-oxo-1-(((R)-4-phenyl-1-(4,4,5,5-tetramethyl-1,3,2-dioxaborolan-2-yl)butyl)amino) pentan-2-yl)carbamate [Step 1]: To a solution of (R)-2-((tert-butoxycarbonyl) (methyl)amino) pentanoic acid (51-1, 204 mg, 0.9 mmol) in THF (3 mL) was added IBCF (0.1 mL, 0.9 mmol) followed by NMM (0.1 mL, 0.9 mmol) at −15° C. After 45 min, a solution of (R)-4-phenyl-1-(4,4,5,5-tetramethyl-1,3,2-dioxaborolan-2-yl)butan-1-amine hydrochloride (51-2, 250 mg, 0.8 mmol) in DMF (0.5 mL) was added dropwise followed by NMM (0.1 mL, 0.8 mmol). After stirring for 1 h at the same temperature, the reaction was diluted with EtOAc, and washed successively with 0.1 N aq. HCl, 5% aq. K₂CO₃, water and brine. The organic phase was dried over anhy. Na₂SO₄, filtered and concentrated under reduced pressure to obtain crude tert-butyl methyl ((R)-1-oxo-1-(((R)-4-phenyl-1-(4,4,5,5-tetramethyl-1,3,2-dioxaborolan-2-yl)butyl)amino) pentan-2-yl)carbamate (51-3, 300 mg, 77%), which was used for the next step without further purification. [M−H]⁻=487.1.

Synthesis of (R)-2-(methylamino)-N—((R)-4-phenyl-1-(4,4,5,5-tetramethyl-1,3,2-dioxaborolan-2-yl)butyl) pentanamide hydrochloride [Step 2]: To a solution of crude tert-butyl methyl ((R)-1-oxo-1-(((R)-4-phenyl-1-(4,4,5,5-tetramethyl-1,3,2-dioxaborolan-2-yl)butyl)amino) pentan-2-yl)carbamate (51-3, 300 mg, 0.6 mmol) in 1,4-dioxane (3 mL) was added HCl (4M in 1,4-dioxane) (1.5 mL, 6.1 mmol) at 0° C., and the mixture was stirred at 25° C. for 16 h. The reaction mixture was concentrated under reduced pressure to afford crude (R)-2-(methylamino)-N—((R)-4-phenyl-1-(4,4,5,5-tetramethyl-1,3,2-dioxaborolan-2-yl)butyl) pentanamide hydrochloride (51-4, 250 mg, crude), which was used in the next step without further purification. [M−H]⁻=387.4.

Synthesis of N-methyl-N—((R)-1-oxo-1-(((R)-4-phenyl-1-(4,4,5,5-tetramethyl-1,3,2-dioxaborolan-2-yl)butyl)amino) pentan-2-yl) pyrazine-2-carboxamide [Step 3]: To a solution of pyrazine-2-carboxylic acid (51-5, 100 mg, 0.8 mmol) in THF (5 mL) was added IBCF (0.1 mL, 0.8 mmol) followed by NMM (0.1 mL, 0.8 mmol) at −15° C. After 45 min, a solution of crude (R)-2-(methylamino)-N—((R)-4-phenyl-1-(4,4,5,5-tetramethyl-1,3,2-dioxaborolan-2-yl)butyl) pentanamide hydrochloride (51-4, 250 mg, 0.7 mmol) in DMF (0.5 mL) was added dropwise followed by NMM (0.1 mL, 0.7 mmol). After stirring for 1 h at the same temperature, the reaction was diluted with EtOAc and washed successively with 0.1 N aq. HCl, 5% aq. K₂CO₃, water and brine. The organic phase was dried over anhy. Na₂SO₄, filtered and concentrated under reduced pressure to get crude N-methyl-N—((R)-1-oxo-1-(((R)-4-phenyl-1-(4,4,5,5-tetramethyl-1,3,2-dioxaborolan-2-yl)butyl)amino) pentan-2-yl) pyrazine-2-carboxamide (51-6, 300 mg, 83%) which was used in the next step without further purification. [M−H]⁻=493.2.

Synthesis of ((R)-1-((R)-2-(N-methylpyrazine-2-carboxamido) pentanamido)-4-phenylbutyl) boronic acid [Step 4]: To an ice-cold solution of crude N-methyl-N—((R)-1-oxo-1-(((R)-4-phenyl-1-(4,4,5,5-tetramethyl-1,3,2-dioxaborolan-2-yl)butyl)amino) pentan-2-yl) pyrazine-2-carboxamide (51-6, 300 mg, 0.6 mmol) and methylboronic acid (51-7, 545 mg, 9.1 mmol) in acetone (12 mL) was added freshly prepared 0.2N aq. HCl (12 mL, 0.6 mmol), and the mixture was stirred at 25° C. After 16 h, LCMS indicated complete consumption of starting material with the formation of desired product. The reaction mixture was concentrated under reduced pressure, and then lyophilized. The material was purified by prep HPLC (RP), and lyophilized to afford ((R)-1-((R)-2-(N-methylpyrazine-2-carboxamido) pentanamido)-4-phenylbutyl)boronic acid (Compound 51, 70 mg), as a mixture of rotamers. [M−H]⁻=411.3. ¹H NMR (400 MHZ, MeOD): δ 8.92-8.85 (m, 1H), 8.70-8.49 (m, 2H), 7.23-7.11 (m, 5H), 5.34-4.87 (m, 1H), 3.05 (s, 3H), 2.67-2.62 (m, 3H), 2.00-1.88 (m, 2H), 1.70-1.65 (m, 2H), 1.56-1.41 (m, 3H), 1.25-1.15 (m, 1H), 1.03-0.84 (m, 3H).

### Example 34: Synthesis of ((R)-1-((R)—N-methyl-2-(N-methylpyrazine-2-carboxamido) pentanamido)-4-phenylbutyl)boronic acid

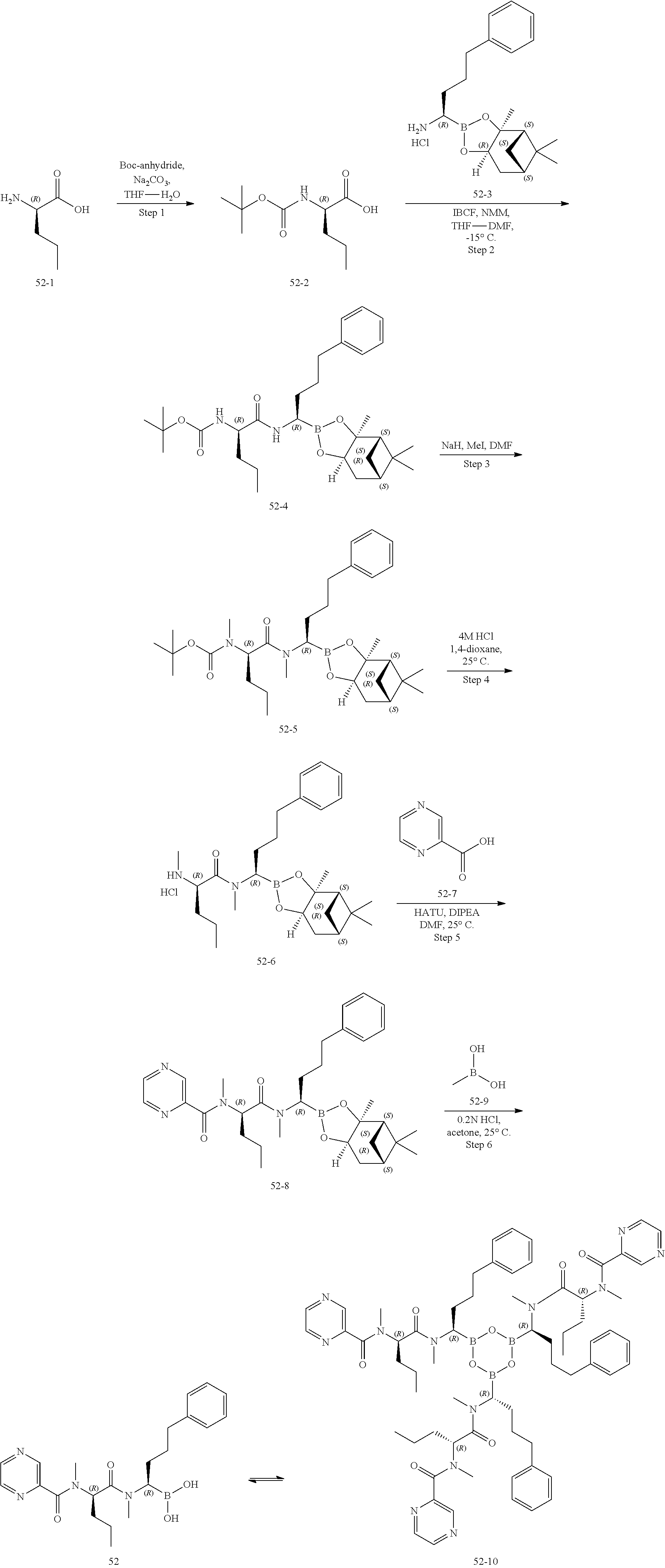

Synthesis of (R)-2-((tert-butoxycarbonyl)amino) pentanoic acid, [Step 1]: To a solution of (R)-2-aminopentanoic acid (52-1, 5.0 g, 42.7 mmol) and sodium carbonate (4.5 g, 42.7 mmol) in THF (60 mL)-water (60 mL) was added Boc anhydride (11.0 mL, 46.9 mmol) at 0° C. and the reaction mixture was stirred at ambient temperature for 16 h. The reaction was quenched with aqueous citric acid, and extracted with 10% MeOH in DCM. The organic phase was washed with brine, dried over anhydrous Na₂SO₄, filtered and concentrated under reduced pressure to afford (R)-2-((tert-butoxycarbonyl)amino) pentanoic acid (52-2, 7.0 g), which was used in the next step without further purification. ¹H NMR (400 MHZ, DMSO-d₆): δ_{H} 0.77 (s, 1H), 7.03 (d, 1H), 3.86-3.84 (m, 1H), 1.61-1.50 (m, 2H), 1.37-1.20 (m, 11H), 0.85 (t, 3H).

Synthesis of tert-butyl ((R)-1-oxo-1-(((R)-4-phenyl-1-((3aS,4S,6S,7aR)-3a,5,5-trimethyl hexahydro-4,6-methanobenzo[d][1,3,2]dioxaborol-2-yl)butyl)amino) pentan-2-yl)carbamate, [Step 2]: To a solution of (R)-2-((tert-butoxycarbonyl)amino) pentanoic acid (52-2, 131 mg, 0.6 mmol) and (R)-4-phenyl-1-((3aS,4S,6S,7aR)-3a,5,5-trimethylhexahydro-4,6-methanobenzo[d][1,3,2]dioxaborol-2-yl)butan-1-amine hydrochloride (52-3, 200 mg, 0.6 mmol) in DMF (5 mL) was added HATU (314 mg, 0.8 mmol) followed by DIPEA (0.2 mL, 1.4 mmol) and the reaction mixture was stirred at ambient temperature. After 2 h, the reaction mixture was quenched with cold brine and extracted with ethyl acetate. The organic phase was dried over anhydrous Na₂SO₄, filtered and concentrated under reduced pressure to afford tert-butyl ((R)-1-oxo-1-(((R)-4-phenyl-1-((3aS,4S,6S,7aR)-3a,5,5-trimethylhexahydro-4,6-methanobenzo[d][1,3,2]dioxaborol-2-yl)butyl)amino) pentan-2-yl)carbamate (52-4, 250 mg), which was used in the next step without further purification. [M−H]⁻=525.5.

Synthesis of tert-butyl methyl ((R)-1-(methyl ((R)-4-phenyl-1-((3aS,4S,6S,7aR)-3a,5,5-trimethylhexahydro-4,6-methanobenzo[d][1,3,2]dioxaborol-2-yl)butyl)amino)-1-oxopentan-2-yl)carbamate, [Step 3]: To an ice-cold solution of tert-butyl ((R)-1-oxo-1-(((R)-4-phenyl-1-((3aS, 4S,6S,7aR)-3a,5,5-trimethylhexahydro-4,6-methanobenzo[d][1,3,2]dioxaborol-2-yl)butyl)amino) pentan-2-yl)carbamate (52-4, 250 mg, 0.5 mmol) in DMF (10 mL) was added NaH (95 mg, 60% in mineral oil, 2.4 mmol) and stirred for 15 min. To this solution was added iodomethane (0.2 mL, 2.4 mmol) and the reaction mixture was stirred at ambient temperature. After 16 h, the reaction mixture was quenched with cold water and extracted with ethyl acetate (twice). Combined organic extract was washed with brine, dried over anhydrous Na₂SO₄, filtered and concentrated under reduced pressure to afford tert-butyl methyl ((R)-1-(methyl ((R)-4-phenyl-1-((3aS,4S,6S,7aR)-3a,5,5-trimethylhexahydro-4,6-methanobenzo[d][1,3,2]dioxaborol-2-yl)butyl)amino)-1-oxopentan-2-yl)carbamate (52-5, 220 mg), which was used in the next step without further purification. [M+H]⁺=555.1.

Synthesis of (R)—N-methyl-2-(methylamino)-N—((R)-4-phenyl-1-((3aS,4S,6S,7aR)-3a,5,5-trimethylhexahydro-4,6-methanobenzo[d][1,3,2]dioxaborol-2-yl)butyl) pentanamide hydrochloride, [Step 4]: To an ice-cold solution of tert-butyl methyl ((R)-1-(methyl ((R)-4-phenyl-1-((3aS,4S,6S,7aR)-3a,5,5-trimethylhexahydro-4,6-methanobenzo[d][1,3,2]dioxaborol-2-yl)butyl)amino)-1-oxopentan-2-yl)carbamate (52-5, 220 mg, 0.4 mmol) in 1,4-dioxane (5 mL) was added 4M HCl in dioxane (5.0 mL, 13.7 mmol), and the reaction mixture was stirred at ambient temperature. After 16 h, the reaction mixture was concentrated under reduced pressure to afford (R)—N-methyl-2-(methylamino)-N—((R)-4-phenyl-1-((3aS,4S,6S,7aR)-3a,5,5-trimethylhexahydro-4,6-methanobenzo[d][1,3,2]dioxaborol-2-yl)butyl) pentanamide hydrochloride (52-6, 180 mg), which was used in the next step without further purification. [M−H]⁻=453.5.

Synthesis of N-methyl-N—((R)-1-(methyl ((R)-4-phenyl-1-((3aS,4S,6S,7aR)-3a,5,5-trimethylhexahydro-4,6-methanobenzo[d][1,3,2]dioxaborol-2-yl)butyl)amino)-1-oxopentan-2-yl) pyrazine-2-carboxamide, [Step 5]: To a solution of (R)—N-methyl-2-(methylamino)-N—((R)-4-phenyl-1-((3aS,4S,6S,7aR)-3a,5,5-trimethylhexahydro-4,6-methanobenzo[d][1,3,2]dioxaborol-2-yl)butyl) pentanamide hydrochloride (52-6, 180 mg, 0.4 mmol) and pyrazine-2-carboxylic acid (52-7, 55 mg, 0.4 mmol) was added HATU (209 mg, 0.6 mmol) followed by DIPEA (0.2 mL, 0.9 mmol) and the reaction mixture was stirred at ambient temperature for 1 h. The reaction mixture was quenched with cold water and extracted with ethyl acetate (twice). Combined organic extract was washed with brine, dried over anhydrous Na₂SO₄, filtered and concentrated under reduced pressure. The compound was purified by reverse phase preparative HPLC to afford N-methyl-N—((R)-1-(methyl ((R)-4-phenyl-1-((3aS,4S,6S,7aR)-3a,5,5-trimethylhexahydro-4,6-methanobenzo[d][1,3,2]dioxaborol-2-yl)butyl)amino)-1-oxopentan-2-yl) pyrazine-2-carboxamide (52-8, 60 mg). [M−H]⁻=559.5.

Synthesis of ((R)-1-((R)—N-methyl-2-(N-methylpyrazine-2-carboxamido) pentanamido)-4-phenylbutyl)boronic acid, [Step 6]: To an ice-cold solution of N-methyl-N—((R)-1-(methyl ((R)-4-phenyl-1-((3aS,4S,6S,7aR)-3a,5,5-trimethylhexahydro-4,6-methanobenzo[d][1,3,2]dioxaborol-2-yl)butyl)amino)-1-oxopentan-2-yl) pyrazine-2-carboxamide (52-8, 40 mg, 0.1 mmol) and methylboronic acid (52-9, 64 mg, 1.1 mmol) in acetone (2 mL) was added 0.2N HCl (2.0 mL, 0.4 mmol), and the reaction mixture was allowed to warm to ambient temperature. After 16 h, the reaction mixture was concentrated under reduced pressure and lyophilized. The compound was purified by reverse phase preparative HPLC and lyophilized to afford ((R)-1-((R)—N-methyl-2-(N-methylpyrazine-2-carboxamido) pentanamido)-4-phenylbutyl)boronic acid (Compound 52, 16 mg). [M−H]⁻=425.5, and 1223.7 for the corresponding trimer (52-10). ¹H NMR (400 MHZ, DMSO-d₆): δ_{H} 8.79-8.76 (m, 2H), 8.66 (s, 1H), 7.26-7.12 (m, 5H), 5.44-5.42 (m, 1H), 3.08 (s, 3H), 2.73 (s, 3H), 2.58-2.54 (m, 2H), 2.50-2.42 (m, 1H), 1.74-1.71 (m, 2H), 1.60-1.50 (m, 4H), 1.31-1.28 (m, 2H), 0.92 (t, 3H). Note: Extra peak present.

HPLC and LCMS data demonstrate that Compound 52 remains in equilibrium with its trimer 52-10 in solid phase, and dissociates to the monomer in solution.

### Example 35

### General Procedures for the Preparation of Compounds

The following general procedures are provided to prepare compounds that are prepared using similar reaction conditions.

General Procedure A: Amide Formation from an Amine and a Carboxylic Acid. To a stirred solution of a carboxylic acid containing compound in tetrahydrofuran (THF) was added isobutyl chloroformate (IBCF, 1 equivalent) and 4-methylmorpholine (NMM, 1 equivalent) at −15° C. The reaction mixture was stirred at the same temperature for about 30 minutes. The corresponding amine (0.9-1.1 equivalents) in dimethylformamide (DMF) was added followed by NMM (0.9-1.1 equivalents) at −15° C. The reaction mixture was gradually warmed to 0° C. and stirred for about 2 hours. The resulting product was neutralized with aqueous 0.1 N HCl solution and extracted several times with ethyl acetate. The organic layers were combined and washed with a solution of 5% potassium carbonate, water, brine, and dried over anhydrous sodium sulfate. The mixture was filtered, concentrated under reduced pressure, and purified through combiflash column chromatography to afford the corresponding amide product.

The following compounds described in the above reactions schemes were prepared using General Procedure A for amide formation using an amine and a carboxylic acid: 2-3, 2-9, 3-2, 3-8, 4-10, 5-6, 6-6, 7-6, 8-6, 9-3, 9-9, 10-3, 10-9, 12-3, 12-9, 24-3, 24-9, 32-6, 39-6, 40-6, 41-6, 42-6, 47-3, 47-9, 48-6.

General Procedure B: Amide Formation from an Amine and an Acid Chloride, Anhydride, or Sulfonyl Chloride. To a stirred solution of the amine compound 0.6 mmol) and acetic anhydride (1.1 equivalents) in dichloromethane was added an ice cooled solution of diisopropylethylamine (DIPEA) (5 equivalents) and the reaction mixture was stirred at room temperature for about 2 hours. Thin layer chromatography showed complete disappearance of the starting material. The reaction mixture was diluted with dichloromethane (DCM) and washed with water and brine solution. The organic phase was dried over anhydrous sodium sulfate, filtered, and concentrated under reduced pressure. The product was purified by reverse-phase prep-HPLC and lyophilized to afford the desired amide product.

In General Procedure B, acetic anhydride can be replaced with an acyl chloride (e.g., morpholine-4-carbonyl chloride) or a sulfonyl chloride (e.g., benzenesulfonyl chloride), and the DIPEA can be replaced with another base (e.g., N-methylmorpholine, NMM).

The following compounds described in the above reactions schemes were prepared using General Procedure B for amide formation using an amine and an acid chloride, anhydride, or sulfonyl chloride: 20-3, 21-3, 33-6, 34-3, 43-3, 44-3, 45-3, 46-3.

General Procedure C: Hydrogenolysis of Benzyl Esters. To a stirred solution of the benzyl ester containing compound in tetrahydrofuran (THF) was added nitrogen gas for 10 minutes. Then 10% Pd—C(0.7 equivalents) was added and the reaction mixture was hydrogenated under a hydrogen balloon from 3 to 12 hours and monitored by thin layer chromatography. The reaction mixture was filtered over celite using excess ethyl acetate. The solvents were removed by concentration under reduced pressure to afford the corresponding carboxylic acid product.

The following compounds described in the above reaction schemes were prepared using General Procedure C for hydrogenolysis of benzyl esters: 2-4, 3-3, 5-1, 7-1, 8-1, 9-4, 10-4, 12-4, 24-4, 32-1, 33-1, 47-4, 48-1.

General Procedure D: Amide Formation with a Protected Boronic Acid. To a stirred solution of a carboxylic acid containing compound in tetrahydrofuran was added isobutyl chloroformate (IBCF) (1 equivalent) and N-methylmorpholine (NMM) (1 equivalent) at −15° C. The reaction mixture was stirred at the same temperature for about 30 minutes. Then the protected boronic acid compound bearing an amine group (1 equivalent) in dimethylformamide was added to the reaction mixture followed by NMM (1 equivalent) at −15° C. The reaction mixture was gradually warmed to 0° C. and stirred for about 2 hours. LCMS of the reaction mass confirmed the formation of the desired product, and the reaction mixture was neutralized with an aqueous solution of 0.1 N HCl and extracted with ethyl acetate. The organic layers were combined and washed with 5% potassium carbonate solution, water, brine, dried over sodium sulfate, filtered, and concentrated under reduced pressure to afford the coupled product.

The following compounds described in the above reaction schemes were prepared using General Procedure D for amide formation with a protected boronic acid: 2-6, 3-5, 4-7, 5-3, 6-3, 7-3, 8-3, 9-6, 10-6, 12-6, 22-1, 24-6, 32-3, 33-3, 39-3, 40-3, 41-3, 42-3, 47-6, 48-3.

General Procedure E. Removal of a BOC Protection Group. To a solution of a BOC-protected compound was added 4 M HCl in dioxane (10 equivalents) at 0° C. The reaction mixture was gradually warmed to ambient temperature and stirred for about 16 hours. Thin layer chromatography showed complete consumption of the starting material and the reaction mixture was concentrated under reduced pressure to obtain the desired product as the hydrochloride salt. The product was used without purification.

The following compounds described in the above reaction schemes were prepared using General Procedure E for removal of a BOC protection group: 2-7, 3-6, 4-8, 5-4, 6-4, 7-4, 8-4, 9-7, 10-7, 12-7, 20-1, 21-1, 24-7, 32-4, 33-4, 39-4, 40-4, 41-4, 42-4, 47-7, 48-4.

General Procedure F: Hydrolysis of a Boronate Ester: To a stirred solution of the boronate ester and methylboronic acid (8 equivalents) in acetone was added an equivalent volume of 0.2 N HCl and the reaction mixture was stirred at ambient temperature overnight. Thin layer chromatography showed the complete disappearance of the starting material and the reaction mixture was concentrated under reduced pressure. The product was redissolved in a mixture of acetone and deionized water, and lyophilized to obtain the boronic acid product.

The following compounds described in the above reaction schemes were prepared using General Procedure F for hydrolysis of a boronate ester: Compound 1, Compound 2, Compound 3, Compound 4, Compound 5, Compound 6, Compound 7, Compound 8, Compound 9, Compound 11, Compound 19, Compound 20, Compound 23, Compound 31, Compound 32, Compound 33, Compound 37, Compound 38, Compound 39, Compound 40, Compound 41, Compound 42, Compound 43, Compound 44, Compound 45, Compound 46.

General Procedure G: Oxidative Removal of a Boronate Ester: To a stirred solution of a boronate ester in a mixture of (1:1) acetone and water was added ammonium acetate (1 equivalent) and the reaction mixture was stirred for 5 minutes. Sodium periodate (NalO₄) (1 equivalent) was added portion-wise and reaction mixture was stirred for 3 hours. The reaction mixture was concentrated under reduced pressure and partitioned between ethyl acetate and water. The organic layer was collected and the aqueous layer was further extracted with ethyl acetate (twice). The combined organic layers were dried over anhydrous sodium sulfate and concentrated under reduced pressure. The product was purified through reverse-phase prep-HPLC to afford the desired boronic acid product.

The following compounds described in the above reaction schemes were prepared using General Procedure G for oxidative removal of a boronate ester: Compound 21.

### Exemplary Reaction Conditions for the Preparation of Compounds

The following reaction conditions are provided for the preparation of Compound 1, Compound 2, Compound 3, and Compound 28.

Characterization of Compounds Prepared from General Procedure A

Compound (4-6): N—((R)-1-(((R)-3-methyl-1-((3aS,4S,6S,7aR)-3a,5,5-trimethylhexahydro-4,6-methanobenzo[d][1,3,2]dioxaborol-2-yl)butyl)amino)-4-morpholino-1,4-dioxobutan-2-yl) pyrazine-2-carboxamide: LCMS (ESI) Calcd. for C₂₈H₄₂BN₅O₆: 555.5, found: [M−H]⁻=554.5.

Compound (5-6): N—((R)-4-morpholino-1,4-dioxo-1-(((R)-3-phenyl-1-(4,4,5,5-tetramethyl-1,3,2-dioxaborolan-2-yl)propyl)amino)butan-2-yl) pyrazine-2-carboxamide: LCMS (ESI) Calcd. for C₂₈H₃₈BN₅O₆: 551.4, found: [M−H]⁻=550.4.

Compound (6-6): N—((R)-4-morpholino-1,4-dioxo-1-(((R)-1-((3aS,4S,6S,7aR)-3a,5,5-trimethylhexahydro-4,6-methanobenzo[d][1,3,2]dioxaborol-2-yl)butyl)amino)butan-2-yl) pyrazine-2-carboxamide: LCMS (ESI) Calcd. for C₂₂H₃₈BN₃O₅: 541.4, found: [M−H]⁻=540.4.

Compound (7-6): 2,4-dimethyl-N—((R)-1-(((R)-3-methyl-1-((3aS,4S,6S,7aR)-3a,5,5-trimethylhexahydro-4,6-methanobenzo[d][1,3,2]dioxaborol-2-yl)butyl)amino)-4-morpholino-1,4-dioxobutan-2-yl)oxazole-5-carboxamide: LCMS (ESI) Calcd. for C₂₉H₄₅BN₄O₇: 572.4, found: [M−H]⁺=571.4.

Compound (8-3): Benzyl N²-(tert-butoxycarbonyl)-N⁴,N⁴-dimethyl-D-asparaginate: LCMS (ESI) Calcd. for C₁₈H₂₆N₂O₅:349.8, found: [M+H]⁺+350.8. ¹H NMR (400 MHZ, DMSO-d₆) (400 MHZ, DMSO-d₆) δ 7.52-7.17 (m, 5H), 5.10 (s, 2H), 4.56-4.36 (m, 1H), 2.91 (s, 3H), 2.80 (s, 3H), 2.79-2.64 (m, 2H), 1.36 (s, 9H), 1.32-1.26 (m, 1H).

Compound (8-9): (R)—N⁴,N⁴-dimethyl-N¹—((R)-4-phenyl-1-(4,4,5,5-tetramethyl-1,3,2-dioxaborolan-2-yl)butyl)-2-(pyrazine-2-carboxamido) succinimide: LCMS (ESI) Calcd. for C₂₇H₃₈BN₅O₅: 523.5, found: [M−H]⁺=522.5.

Compound (9-3): Benzyl N²-(tert-butoxycarbonyl)-N⁴-ethyl-D-asparaginate: LCMS (ESI) Calcd. For C₁₈H₂₆N₂O₅:349.8, found: [M+H]⁺=350.8. ¹H NMR (400 MHZ, DMSO-d₆) δ 7.86 (s, 1H), 7.34 (s, 5H), 7.14 (d, 1H), 5.09 (s, 2H), 4.40 (d, 1H), 3.15-2.92 (m, 2H), 2.65-2.51 (m, 1H), 2.48-2.34 (m, 1H), 1.45-1.22 (m, 9H), 0.97 (t, 3H).

Compound (9-9): (R)—N⁴-ethyl-N¹—((R)-4-phenyl-1-(4,4,5,5-tetramethyl-1,3,2-dioxaborolan-2-yl)butyl)-2-(pyrazine-2-carboxamido) succinimide: LCMS (ESI) Calcd. for C₂₇H₃₈BN₅O₅: 523.6, found: [M−H]⁺=522.6.

Compound (11-3): Benzyl (R)-2-((tert-butoxycarbonyl)amino)-4-oxo-4-(piperidin-1-yl)butanoate: LCMS (ESI) Calcd. for C₂₁H₃₀N₂O₅:389.8, found: [M+H]⁺=390.8.

Compound (11-9): N—((R)-1,4-dioxo-1-(((R)-4-phenyl-1-(4,4,5,5-tetramethyl-1,3,2-dioxaborolan-2-yl)butyl)amino)-4-(piperidin-1-yl)butan-2-yl) pyrazine-2-carboxamide: LCMS (ESI) Calcd. for C₃₀H₄₂BN₅O₅: 563.6, found: [M−H]⁺=562.6.

Compound (23-3): (R)-2-((tert-butoxycarbonyl)amino)-5-morpholino-5-oxopentanoate: ¹H NMR (400 MHZ, DMSO-D₆) δ 7.36-7.31 (m, 6H), 5.17-5.05 (m, 2H), 4.06-4.01 (m, 1H), 3.51-3.50 (m, 4H), 3.41-3.39 (m, 2H), 2.39-2.28 (m, 2H), 1.95-1.89 (m, 1H), 1.84-1.77 (m, 1H), 1.37 (s, 9H).

Compound (23-9): N—((R)-5-morpholino-1,5-dioxo-1-(((R)-4-phenyl-1-(4,4,5,5-tetramethyl-1,3,2-dioxaborolan-2-yl)butyl)amino) pentan-2-yl) pyrazine-2-carboxamide: LCMS (ESI) Calcd. for C₃₀H₄₂BN₅O₆: 579, found: [M−H]⁺=578.

Compound (31-6): N—((R)-4-morpholino-1,4-dioxo-1-(((R)-1-(4,4,5,5-tetramethyl-1,3,2-dioxaborolan-2-yl)pentyl)amino)butan-2-yl) pyrazine-2-carboxamide: LCMS (ESI) Calcd. for C₂₄H₃₈BN₅O₆: 503.4, found: [M−H]⁻=502.4.

Compound (38-6): N—((R)-1-(((R)-2-(benzyloxy)-1-((3aS,4S,6S,7aR)-3a,5,5-trimethylhexahydro-4,6-methanobenzo[d][1,3,2]dioxaborol-2-yl)ethyl)amino)-3-methoxy-1-oxopropan-2-yl) pyrazine-2-carboxamide: LCMS (ESI) Calcd. for C₂₈H₃₇BN₄O₆: 536.0, found: [M+H]⁺=537.0.

Compound (39-6): N—((R)-1-(((R)-2-(benzyloxy)-1-((3aS,4S,6S,7aR)-3a,5,5-trimethylhexahydro-4,6-methanobenzo[d][1,3,2]dioxaborol-2-yl)ethyl)amino)-1-oxopentan-2-yl) pyrazine-2-carboxamide: LCMS (ESI) Calcd. for C₂₉H₃₉BN₄O₅: 532.5, found: [M+H]⁺=533.5.

Compound (40-6): N—((R)-4-morpholino-1,4-dioxo-1-(((S)-4-phenyl-1-(4,4,5,5-tetramethyl-1,3,2-dioxaborolan-2-yl)butyl)amino)butan-2-yl) pyrazine-2-carboxamide: LCMS (ESI) Calcd. for C₂₉H₄₀BN₅O₆: 565.4, found: [M−H]⁺=564.4.

Compound (45-3): Benzyl(S)-2-((tert-butoxycarbonyl)amino)-4-morpholino-4-oxobutanoate: LCMS (ESI) Calcd. for C₂₀H₂₈N₂O₆:392.0, found: [M+H]⁺=393.0.

Compound (45-9): N—((S)-4-morpholino-1,4-dioxo-1-(((R)-4-phenyl-1-(4,4,5,5-tetramethyl-1,3,2-dioxaborolan-2-yl)butyl)amino)butan-2-yl) pyrazine-2-carboxamide: LCMS (ESI) Calcd. for C₂₉H₄₀BN₅O₆: 565.4, found: [M−H]⁺=564.4.

Compound (46-6): N—((S)-4-morpholino-1,4-dioxo-1-(((S)-4-phenyl-1-(4,4,5,5-tetramethyl-1,3,2-dioxaborolan-2-yl)butyl)amino)butan-2-yl) pyrazine-2-carboxamide: LCMS (ESI) Calcd. for C₂₉H₄₀BN₅O₆: 565.3, found: [M−H]⁺=564.3.

Characterization of Compounds Prepared from General Procedure B

Compound (19-3): (R)-2-acetamido-4-morpholino-4-oxo-N—((R)-4-phenyl-1-(4,4,5,5-tetramethyl-1,3,2-dioxaborolan-2-yl)butyl)butanamide: LCMS (ESI) Calcd. for C₂₆H₄₀BN₃O₆: 501.1, found: [M−H]⁺=500.1.

Compound (20-3): N—((R)-4-morpholino-1,4-dioxo-1-(((R)-4-phenyl-1-(4,4,5,5-tetramethyl-1,3,2-dioxaborolan-2-yl)butyl)amino)butan-2-yl) morpholine-4-carboxamide: LCMS (ESI) Calcd. for C₂₉H₄₅BN₄O₇: 572.5, found: [M−H]⁺=571.5. ¹H NMR (400 MHZ, MeOD) δ 7.24-7.09 (m, 5H), 4.96-4.92 (m, 1H), 3.65-3.59 (m, 8H), 3.51-3.49 (m, 4H), 3.40-3.32 (m, 4H), 3.01-2.87 (m, 2H), 2.62-2.57 (m, 3H), 1.70-1.48 (m, 4H), 1.19-1.14 (m, 5H).

Compound (32-6): N—((R)-4-morpholino-1,4-dioxo-1-(((R)-1-(4,4,5,5-tetramethyl-1,3,2-dioxaborolan-2-yl)propyl)amino)butan-2-yl) pyrazine-2-carboxamide: LCMS (ESI) Calcd. for C₂₂H₃₄BN₅O₆: 475.3, found: [M−H]⁻=474.3.

Compound (33-3): (R)-2-(3,3-dimethylureido)-4-morpholino-4-oxo-N—((R)-4-phenyl-1-(4,4,5,5-tetramethyl-1,3,2-dioxaborolan-2-yl)butyl)butanamide: LCMS (ESI) Calcd. for C₂₇H₄₃BN₄O₆: 530.5, found: [M−H]⁺=529.5.

Compound (41-3): N—((R)-4-morpholino-1,4-dioxo-1-(((R)-4-phenyl-1-(4,4,5,5-tetramethyl-1,3,2-dioxaborolan-2-yl)butyl)amino)butan-2-yl) cyclohexanecarboxamide: LCMS (ESI) Calcd. for C₃₁H₄₈BN₃O₆: 569.7, found: [M−H]⁺=568.7.

Compound (42-3): N—((R)-4-morpholino-1,4-dioxo-1-(((R)-4-phenyl-1-(4,4,5,5-tetramethyl-1,3,2-dioxaborolan-2-yl)butyl)amino)butan-2-yl) tetrahydro-2H-pyran-4-carboxamide: LCMS (ESI) Calcd. for C₃₀H₄₆BN₃O₇: 571.4, found: [M−H]⁺=570.4.

Compound (43-3): (R)-4-morpholino-4-oxo-N—((R)-4-phenyl-1-(4,4,5,5-tetramethyl-1,3,2-dioxaborolan-2-yl)butyl)-2-(phenylsulfonamido)butanamide: LCMS (ESI) Calcd. for C₃₀H₄₂BN₃O₇S: 599.3, found: [M−H]⁺=598.

Compound (44-3): N—((R)-4-morpholino-1,4-dioxo-1-(((R)-4-phenyl-1-(4,4,5,5-tetramethyl-1,3,2-dioxaborolan-2-yl)butyl)amino)butan-2-yl) tetrahydro-2H-pyran-2-carboxamide: LCMS (ESI) Calcd. for C₃₀H₄₆BN₃O₇: 571.5, found: [M−H]⁺=570.5.

Characterization of Compounds Prepared from General Procedure C

Compound (8-4): N²-(tert-butoxycarbonyl)-N⁴,N⁴-dimethyl-D-asparagine: LCMS (ESI) Calcd. for C₁₁H₂₀N₂O₅:259.8, found: [M+H]⁺=260.8. ¹H NMR (400 MHZ, DMSO-d₆) δ 12.48 (s, 1H), 6.70 (d, 1H), 4.31 (s, 1H), 3.97-3.69 (m, 2H), 2.93 (s, 3H), 2.80 (s, 3H), 1.38 (s, 9H).

Compound (9-4): N²-(tert-butoxycarbonyl)-N⁴-ethyl-D-asparagine: LCMS (ESI) Calcd. for C₁₁H₂₀N₂O₅:259.8, found: [M+H]⁺=260.8. ¹H NMR (400 MHZ, DMSO-d₆) δ 12.52 (s, 1H), 7.89-7.76 (m, 1H), 6.89 (d, 1H), 4.26 (q, 1H), 3.10-2.98 (m, 2H), 2.50-2.35 (m, 2H), 1.37 (s, 9H), 0.99 (t, 3H).

Compound (11-4): (R)-2-((tert-butoxycarbonyl)amino)-4-oxo-4-(piperidin-1-yl)butanoic acid: LCMS (ESI) Calcd. for C₁₄H₂₄N₂O₅:300.3, found: [M+H]⁺=301.3.

Compound (23-4): (R)-2-((tert-butoxycarbonyl)amino)-5-morpholino-5-oxopentanoic acid: 1H NMR (400 MHZ, DMSO-D₆) δ 12.5 (br s, 1H), 7.08 (d, 1H), 3.93-3.88 (m, 1H), 3.55-3.51 (m, 4H), 3.41 (d, 4H), 2.40-2.28 (m, 2H), 1.99-1.88 (m, 1H), 1.80-1.73 (m, 1H), 1.38 (s, 9H).

Compound (45-4): 47-4: (S)-2-((tert-butoxycarbonyl)amino)-4-morpholino-4-oxobutanoic acid: LCMS (ESI) Calcd. for C₁₃H₂₂N₂O₆:300.2, found: [M+H]⁺=301.2.

Characterization of Compounds Prepared from General Procedure D

Compound (4-3): Tert-butyl ((R)-1-(((R)-3-methyl-1-((3aS,4S,6S,7aR)-3a,5,5-trimethylhexahydro-4,6-methanobenzo[d][1,3,2]dioxaborol-2-yl)butyl)amino)-4-morpholino-1,4-dioxobutan-2-yl)carbamate: LCMS (ESI) Calcd. for C₂₈H₄₈BN₃O₇: 549.2, found: [M+H]⁺=550.2.

Compound (5-3): ((R)-4-morpholino-1,4-dioxo-1-(((R)-3-phenyl-1-(4,4,5,5-tetramethyl-1,3,2-dioxaborolan-2-yl)propyl)amino)butan-2-yl)carbamate: LCMS (ESI) Calcd. for C₂₈H₄₄BN₃O₇: 545.4, found: [M−H]⁻=544.4.

Compound (6-3): Tert-butyl ((R)-4-morpholino-1,4-dioxo-1-(((R)-1-((3aS,4S,6S,7aR)-3a,5,5-trimethylhexa hydro-4,6-methanobenzo[d][1,3,2]dioxaborol-2-yl)butyl)amino)butan-2-yl)carbamate: LCMS (ESI) Calcd. for C₂₇H₄₆BN₃O₇: 535.5, found: [M−H]⁻=534.5.

Compound (7-3): Tert-butyl ((R)-1-(((R)-3-methyl-1-((3aS,4S,6S,7aR)-3a,5,5-trimethylhexahydro-4,6-methanobenzo[d][1,3,2]dioxaborol-2-yl)butyl)amino)-4-morpholino-1,4-dioxobutan-2-yl)carbamate: LCMS (ESI) Calcd. for C₂₈H₄₈BN₃O₇: 549, found: [M−H]⁺: 548.

Compound (8-6): Tert-butyl ((R)-4-(dimethylamino)-1,4-dioxo-1-(((R)-4-phenyl-1-(4,4,5,5-tetramethyl-1,3,2-dioxaborolan-2-yl)butyl)amino)butan-2-yl)carbamate: LCMS (ESI) Calcd. C₂₇H₄₄BN₃O₆: 517.1, found: [M+H]⁺=518.1.

Compound (9-6): Tert-butyl ((R)-4-(ethylamino)-1,4-dioxo-1-(((R)-4-phenyl-1-(4,4,5,5-tetramethyl-1,3,2-dioxaborolan-2-yl)butyl)amino)butan-2-yl)carbamate: LCMS (ESI) Calcd. C₂₇H₄₄BN₃O₆: 517.1, found: [M+H]: 518.1.

Compound (11-6): Tert-butyl ((R)-1,4-dioxo-1-(((R)-4-phenyl-1-(4,4,5,5-tetramethyl-1,3,2-dioxaborolan-2-yl)butyl)amino)-4-(piperidin-1-yl)butan-2-yl)carbamate: LCMS (ESI) Calcd. for C₃₀H₄₈BN₃O₆: 557.0, found: [M+H]⁺=558.0.

Compound (23-6): Tert-butyl ((R)-5-morpholino-1,5-dioxo-1-(((R)-4-phenyl-1-(4,4,5,5-tetramethyl-1,3,2-dioxaborolan-2-yl)butyl)amino) pentan-2-yl)carbamate: LCMS (ESI) Calcd. for C₃₀H₄₈BN₃O₇: 573, found: [M−H]⁺=572.

Compound (31-3): Tert-butyl ((R)-4-morpholino-1,4-dioxo-1-(((R)-1-(4,4,5,5-tetramethyl-1,3,2-dioxaborolan-2-yl)pentyl)amino)butan-2-yl)carbamate: LCMS (ESI) Calcd. for C₂₄H₄₄BN₃O₇: 497.1, found: [M+H]⁺=498.1.

Compound (32-3): Tert-butyl ((R)-4-morpholino-1,4-dioxo-1-(((R)-1-(4,4,5,5-tetramethyl-1,3,2-dioxaborolan-2-yl)propyl)amino)butan-2-yl)carbamate: LCMS (ESI) Calcd. for C₂₂H₄₀BN₃O₇: 469.4, found: [M−H]⁻=468.4.

Compound (38-3): Tert-butyl ((R)-1-(((R)-2-(benzyloxy)-1-((3aS,4S,6S,7aR)-3a,5,5-trimethylhexahydro-4,6-methanobenzo[d][1,3,2]dioxaborol-2-yl)ethyl)amino)-3-methoxy-1-oxopropan-2-yl)carbamate: LCMS (ESI) Calcd. for C₂₈H₄₃BN₂O₇: 530.0, found: [M+H]⁺=531.0.

Compound (39-3): Tert-butyl ((R)-1-(((R)-2-(benzyloxy)-1-((3aS,4S,6S,7aR)-3a,5,5-trimethylhexahydro-4,6-methanobenzo[d][1,3,2]dioxaborol-2-yl)ethyl)amino)-1-oxopentan-2-yl)carbamate: LCMS (ESI) Calcd. for C₂₉H₄₅BN₂O₆: 528.0, found: [M+H]⁺=529.0.

Compound (40-3): Tert-butyl ((R)-4-morpholino-1,4-dioxo-1-(((S)-4-phenyl-1-(4,4,5,5-tetramethyl-1,3,2-dioxaborolan-2-yl)butyl)amino)butan-2-yl)carbamate: LCMS (ESI) Calcd. for C₂₉H₄₆BN₃O₇: 559.1, found: [M+H]⁺: 560.1.

Compound (45-6): Tert-butyl ((S)-4-morpholino-1,4-dioxo-1-(((R)-4-phenyl-1-(4,4,5,5-tetramethyl-1,3,2-dioxaborolan-2-yl)butyl)amino)butan-2-yl)carbamate: LCMS (ESI) Calcd. for C₂₉H₄₆BN₃O₇: 557.4, found: [M+H]⁺=558.4.

Compound (46-3): Tert-butyl ((S)-4-morpholino-1,4-dioxo-1-(((S)-4-phenyl-1-(4,4,5,5-tetramethyl-1,3,2-dioxaborolan-2-yl)butyl)amino)butan-2-yl)carbamate: LCMS (ESI) Calcd. for C₂₉H₄₆BN₃O₇: 559.5, found: [M−H]⁺=558.5.

Characterization of Compounds Prepared from General Procedure E

Compound (4-4): (R)-2-amino-N—((R)-3-methyl-1-((3aS,4S,6S,7aR)-3a,5,5-trimethylhexahydro-4,6-methanobenzo[d][1,3,2]dioxaborol-2-yl)butyl)-4-morpholino-4-oxobutanamide hydrochloride: LCMS (ESI) Calcd. for C₂₃H₄₀BN₃O₅: 449.2, found: [M+H]⁺=450.2.

Compound (5-4): (R)-2-amino-4-morpholino-4-oxo-N—((R)-3-phenyl-1-(4,4,5,5-tetramethyl-1,3,2-dioxaborolan-2-yl)propyl)butanamide hydrochloride: LCMS (ESI) Calcd. for C₂₃H₃₆BN₃O₅: 445.4, found: [M−H]⁻=444.4.

Compound (6-4): (R)-2-amino-4-morpholino-4-oxo-N—((R)-1-((3aS,4S,6S,7aR)-3a,5,5-trimethylhexahydro-4,6-methanobenzo[d][1,3,2]dioxaborol-2-yl)butyl)butanamide hydrochloride: LCMS (ESI) Calcd. for C₂₂H₃₈BN₃O₅: 435.3, found: [M−H]⁻=434.3.

Compound (7-4): (R)-2-amino-N—((R)-3-methyl-1-((3aS,4S,6S,7aR)-3a,5,5-trimethylhexahydro-4,6-methanobenzo[d][1,3,2]dioxaborol-2-yl)butyl)-4-morpholino-4-oxobutanamide: LCMS (ESI) Calcd. for C₂₃H₄₀BN₃O₅: 449.6, found: [M−H]⁺=448.6.

Compound (R)-2-amino-N⁴,N⁴-dimethyl-N¹—((R)-4-phenyl-1-(4,4,5,5-tetramethyl-1,3,2-dioxaborolan-2-yl)butyl) succinamide hydrochloride: The product was used directly in the next step without further purification or characterization.

Compound (9-7): (R)-2-amino-N⁴-ethyl-N¹—((R)-4-phenyl-1-(4,4,5,5-tetramethyl-1,3,2-dioxaborolan-2-yl)butyl) succinamide hydrochloride: The product was used directly in next step without purification or characterization.

Compound (11-7): (R)-2-amino-4-oxo-N—((R)-4-phenyl-1-(4,4,5,5-tetramethyl-1,3,2-dioxaborolan-2-yl)butyl)-4-(piperidin-1-yl)butanamide hydrochloride: The product was used directly in next step without purification or characterization.

Compound (23-7): (R)-2-amino-5-morpholino-5-oxo-N—((R)-4-phenyl-1-(4,4,5,5-tetramethyl-1,3,2-dioxaborolan-2-yl)butyl) pentanamide: LCMS (ESI) Calcd. for C₂₅H₄₀BN₃O₅: 473, found: [M−H]⁺=472.

Compound (31-4): (R)-2-amino-4-morpholino-4-oxo-N—((R)-1-(4,4,5,5-tetramethyl-1,3,2-dioxaborolan-2-yl)pentyl)butanamide hydrochloride: LCMS (ESI) Calcd. for C₁₉H₃₆BN₃O₅: 397.4, found: [M−H]⁻=396.4.

Compound (32-4): (R)-2-amino-4-morpholino-4-oxo-N—((R)-1-(4,4,5,5-tetramethyl-1,3,2-dioxaborolan-2-yl)propyl)butanamide hydrochloride: LCMS (ESI) Calcd. for C₁₇H₃₂BN₃O₅: 369.3, found: [M+H]⁺=370.3

Compound (38-4): (R)-2-amino-N—((R)-2-(benzyloxy)-1-((3aS,4S,6S,7aR)-3a,5,5-trimethylhexahydro-4,6-methanobenzo[d][1,3,2]dioxaborol-2-yl)ethyl)-3-methoxypropanamide hydrochloride: LCMS (ESI) Calcd. for C₂₃H₃₆BClN₂O₅: 429.9, found: [M+H]⁺=430.9.

Compound (39-4): (R)-2-amino-N—((R)-2-(benzyloxy)-1-((3aS,4S,6S,7aR)-3a,5,5-trimethylhexahydro-4,6-methanobenzo[d][1,3,2]dioxaborol-2-yl)ethyl) pentanamide hydrochloride: LCMS (ESI) Calcd. for C₂₄H₃₈BClN₂O₄: 428.9, found: [M+H]⁺=429.9.

Compound (40-4): (R)-2-amino-4-morpholino-4-oxo-N—((S)-4-phenyl-1-(4,4,5,5-tetramethyl-1,3,2-dioxaborolan-2-yl)butyl)butanamide hydrochloride: The product was directly used in the next step without purification or characterization.

Compound (45-7): (S)-2-amino-4-morpholino-4-oxo-N—((R)-4-phenyl-1-(4,4,5,5-tetramethyl-1,3,2-dioxaborolan-2-yl)butyl)butanamide hydrochloride: LCMS (ESI) Calcd. for C₂₄H₃₈BN₃O₅: 457.4, found: [M+H]⁺=458.4.

Compound (46-4): (S)-2-amino-4-morpholino-4-oxo-N—((S)-4-phenyl-1-(4,4,5,5-tetramethyl-1,3,2-dioxaborolan-2-yl)butyl)butanamide hydrochloride: LCMS (ESI) Calcd. for C₂₄H₃₈BN₃O₅: 457.4, found: [M+H]⁺=458.4.

Characterization of Compounds Prepared from General Procedure F

Compound 4: ((R)-3-methyl-1-((R)-4-morpholino-4-oxo-2-(pyrazine-2-carboxamido)butanamido) butyl)boronic acid: LCMS (ESI) Calcd. for C₁₈H₂₈BN₅O₆: 421.3, found: [M−H]⁻=420.3. ¹H NMR (400 MHZ, MeOD) δ 9.25 (s, 1H), 8.80 (d, 1H), 8.69 (s, 1H), 5.30 (t, 1H), 3.68-3.61 (m, 4H), 3.55-3.51 (m, 4H), 3.28-3.27 (m, 1H), 3.02-2.97 (m, 1H), 2.70 (t, 1H), 1.67-1.66 (m, 1H), 1.35 (t, 2H), 0.80-0.87 (m, 6H).

Compound 5: ((R)-1-((R)-4-morpholino-4-oxo-2-(pyrazine-2-carboxamido)butanamido)-3-phenyl propyl)boronic acid: LCMS (ESI) Calcd. for C₂₂H₂₈BN₅O₆: 469.3, found: [M−H]⁻=468.3. ¹H NMR (400 MHZ, MeOD) δ 9.27 (d, 1H), 8.81 (d, 1H), 8.70-8.69 (m, 1H), 7.22-7.16 (m, 4H), 7.09 (t, 1H), 5.31 (t, 1H), 3.65 (t, 2H), 3.63-3.6 (m, 2H), 3.57-3.51 (m, 5H), 3.04-2.98 (m, 1H), 2.63 (t, 3H), 1.84-1.71 (m, 2H).

Compound 6: ((R)-1-((R)-4-morpholino-4-oxo-2-(pyrazine-2-carboxamido)butanamido)pentyl) boronic acid: LCMS (ESI) Calcd. for C₁₈H₂₈BN₅O₆: 421.2, found: [M−H]⁻=420.2. ¹H NMR (400 MHz, MeOD) δ 9.26 (s, 1H), 8.80 (d, 1H), 8.69 (s, 1H), 5.30 (d, 1H), 3.68-3.65 (m, 2H), 3.64-3.62 (m, 2H), 3.54-3.47 (m, 4H), 3.27 (s, 1H), 2.99 (dd, 1H), 2.57 (t, 1H), 1.51-1.48 (m, 1H), 1.46-1.44 (m, 1H), 1.31-1.28 (m, 4H), 0.88 (s, 3H).

Compound 7: ((R)-1-((R)-2-(2,4-dimethyloxazole-5-carboxamido)-4-morpholino-4-oxobutanamido)-3-methylbutyl)boronic acid: LCMS (ESI) Calcd. for C₁₉H₃₁BN₄O₇: 438, found: [M−H]⁺=437. ¹H NMR (400 MHZ, MeOD) δ 5.20 (t, 1H), 3.68-3.66 (m, 2H), 3.62 (d, 2H), 3.55-3.51 (m, 4H), 3.17-3.11 (m, 1H), 3.01-2.96 (m, 1H), 2.71 (t, 1H), 2.48 (s, 3H), 2.39 (s, 3H), 1.98 (br s, 1H), 1.70-1.63 (m, 1H), 1.35 (t, 2H), 0.90 (d, 6H).

Compound 8: ((R)-1-((R)-4-(dimethylamino)-4-oxo-2-(pyrazine-2-carboxamido)butanamido)-4-phenylbutyl)boronic acid: LCMS (ESI) Calcd. for C₂₁H₂₈BN₅O₅: 441.2, found: [M−H]⁺=440.2. ¹H NMR (400 MHZ, Methanol-d₄) δ 9.24 (s, 1H), 8.80 (d, 1H), 8.71-8.65 (m, 1H), 7.24-7.05 (m, 5H), 5.26 (s, 1H), 3.05 (s, 3H), 2.90 (s, 3H), 2.58 (d, 3H), 1.77-1.41 (m, 6H).

Compound 9: ((R)-1-((R)-4-(ethylamino)-4-oxo-2-(pyrazine-2-carboxamido)butanamido)-4-phenylbutyl)boronic acid: LCMS (ESI) Calcd. for C₂₁H₂₈BN₅O₅: 441.1, found: [M−H]⁺=440.1. ¹H NMR (400 MHZ, Methanol-d₄) δ 9.23 (s, 1H), 8.80 (d, 1H), 8.72-8.66 (m, 1H), 7.24-7.05 (m, 5H), 5.23-5.12 (m, 1H), 3.23-3.06 (m, 2H), 2.93 (dd, 1H), 2.83 (dd, 1H), 2.63-2.54 (m, 3H), 1.81-1.36 (m, 4H), 1.07 (t, 3H).

Compound 11: ((R)-1-((R)-4-oxo-4-(piperidin-1-yl)-2-(pyrazine-2-carboxamido)butanamido)-4-phenylbutyl)boronic acid: LCMS (ESI) Calcd. for C₂₄H₃₂BN₅O₅: 481.2, found: [M−H]⁺=480.2. ¹H NMR (400 MHZ, MeOD) δ 9.24 (s, 1H), 8.79 (d, 1H), 8.68 (s, 1H), 7.21-7.08 (m, 5H), 5.27 (t, 1H), 3.51-3.45 (m, 4H), 2.99-2.98 (m, 2H), 2.60-2.56 (m, 3H), 1.65-1.48 (m, 10H).

Compound 19: (R)-1-((R)-2-acetamido-4-morpholino-4-oxobutanamido)-4-phenylbutyl)boronic acid: LCMS (ESI) Calcd. for C₂₀H₃₀BN₃O₆: 419.3, found: [M−H]⁺=418.3. ¹H NMR (400 MHZ, MeOD) δ 7.24-7.10 (m, 5H), 5.00 (t, 1H), 3.66-3.59 (m, 4H), 3.52-3.48 (m, 4H), 3.0-2.86 (m, 2H), 2.64-2.57 (m, 3H), 1.97 (s, 3H), 1.71-1.47 (m, 4H).

Compound 20: ((R)-1-((R)-2-(morpholine-4-carboxamido)-4-morpholino-4-oxobutanamido)-4-phenylbutyl)boronic acid: LCMS (ESI) Calcd. for C₂₃H₃₅BN₄O₇: 490.4, found: [M−H]⁺=489.4. ¹H NMR (400 MHZ, MeOD) δ 7.22-7.11 (m, 5H), 4.93 (t, 1H), 3.65-3.59 (m, 8H), 3.51-3.48 (m, 4H), 3.37-3.33 (m, 4H), 2.99-2.91 (m, 2H), 2.62-2.59 (m, 3H), 1.75-1.40 (m, 4H).

Compound 23: ((R)-1-((R)-5-morpholino-5-oxo-2-(pyrazine-2-carboxamido) pentanamido)-4-phenylbutyl)boronic acid: LCMS (ESI) Calcd. for C₂₄H₃₂BN₅O₆: 497, found: [M−H]⁺=496. ¹H NMR (400 MHZ, MeOD) δ 9.21 (s, 1H), 8.80 (d, 1H), 8.70 (t, 1H), 7.22-7.19 (m, 2H), 7.16-7.10 (m, 3H), 3.60-3.54 (m, 6H), 3.47-3.45 (m, 2H), 2.65-2.53 (m, 5H), 2.34-2.28 (m, 1H), 2.24-2.17 (m, 1H), 1.69-1.62 (m, 2H) 1.59-1.49 (m, 3H).

Compound 31: ((R)-1-((R)-4-morpholino-4-oxo-2-(pyrazine-2-carboxamido)butanamido)butyl) boronic acid: LCMS (ESI) Calcd. for C₁₇H₂₆BN₅O₆: 407.1, found: [M−H]⁻=406.1. ¹H NMR (400 MHz, MeOD) δ 9.26 (d, 1H), 8.80 (d, 1H), 8.70-8.69 (m, 1H), 5.29 (t, 1H), 3.68-3.61 (m, 4H), 3.58-3.49 (m, 4H), 3.32-3.27 (m, 1H), 2.99 (dd, 1H), 2.59 (t, 1H), 1.51-1.28 (m, 4H), 0.90 (t, 3H).

Compound 32: ((R)-1-((R)-4-morpholino-4-oxo-2-(pyrazine-2-carboxamido)butanamido)propyl) boronic acid: LCMS (ESI) Calcd. for C₁₆H₂₄BN₅O₆: 393.2, found: [M−H]⁻=392.2. ¹H NMR (400 MHz, MeOD) δ 9.26 (d, 1H), 8.80 (d, 1H), 8.69 (t, 1H), 5.30 (t, 1H), 3.63-3.61 (m, 2H), 3.58 (d, 2H), 3.55-3.51 (m, 4H), 3.02-2.97 (dd, 1H), 2.50 (t, 1H), 1.59-1.52 (m, 1H), 1.50-1.48 (m, 1H), 1.28 (s, 1H), 0.92 (t, 3H).

Compound 33: (R)-1-((R)-2-(3,3-dimethylureido)-4-morpholino-4-oxobutanamido)-4-phenylbutyl) boronic acid: LCMS (ESI) Calcd. for C₂₁H₃₃BN₄O₆: 448.2, found: [M−H]⁺=447.2. ¹H NMR (400 MHz, MeOD) δ 7.24-7.11 (m, 5H), 4.92 (t, 1H), 3.65-3.59 (m, 4H), 3.51-3.48 (m, 4H), 3.03-3.01 (m, 1H), 2.91-2.86 (m, 7H), 2.62-2.56 (m, 3H), 1.70-1.48 (m, 4H).

Compound 38: ((R)-2-(benzyloxy)-1-((R)-3-methoxy-2-(pyrazine-2-carboxamido)propanamido) ethyl)boronic acid: LCMS (ESI) Calcd. for C₁₈H₂₃BN₄O₆: 401.9, found: [M−H]⁺=400.9. ¹H NMR (400 MHZ, MeOD) δ 9.24 (s, 1H), 8.80 (d, 1H), 8.70 (d, 1H), 7.34-7.23 (m, 5H), 5.06 (t, 1H), 4.49 (d, 2H), 3.90-3.86 (m, 1H), 3.81-3.77 (m, 1H), 3.59-3.56 (m, 1H), 3.46-3.43 (m, 1H), 3.39 (br s, 3H), 2.95 (d, 1H).

Compound 39: ((R)-2-(benzyloxy)-1-((R)-2-(pyrazine-2-carboxamido) pentanamido)ethyl)boronic acid: LCMS (ESI) Calcd. for C₁₉H₂₅BN₄O₅: 400.4, found: [M−H]⁺=399.4. ¹H NMR (400 MHZ, MeOD) δ 9.23 (s, 1H), 8.80 (d, 1H), 8.70 (s, 1H), 7.35-7.23 (m, 5H), 4.88 (t, 1H), 4.49 (br s, 2H), 3.60-3.56 (m, 1H), 3.42 (t, 1H), 2.93-2.89 (m, 1H), 1.94-1.90 (m, 2H), 1.50-1.43 (br s, 2H), 0.98 (t, 3H).

Compound 40: ((S)-1-((R)-4-morpholino-4-oxo-2-(pyrazine-2-carboxamido)butanamido)-4-phenylbutyl)boronic acid: LCMS (ESI) Calcd. for C₂₃H₃₀BN₅O₆: 483.4, found: [M−H]⁺=482.4. ¹H NMR (400 MHZ, MeOD) δ 9.25 (s, 1H), 8.79 (d, 1H), 8.68 (s, 1H), 7.21-7.07 (m, 5H), 5.28 (t, 1H), 3.66 (t, 2H), 3.61-3.47 (m, 6H), 2.98-2.93 (m, 1H), 2.65 (t, 1H), 2.58-2.57 (m, 2H), 1.65-1.39 (m, 5H).

Compound 41: ((R)-1-((R)-2-(cyclohexanecarboxamido)-4-morpholino-4-oxobutanamido)-4-phenylbutyl)boronic acid: LCMS (ESI) Calcd. for C₂₅H₃₈BN₃O₆: 487.3, found: [M−H]⁺=486.3. ¹H NMR (400 MHZ, MeOD) δ 7.24-7.10 (m, 5H), 4.97 (br s, 1H), 3.65-3.59 (m, 4H), 3.50-3.48 (m, 4H), 2.92-2.90 (m, 2H), 2.61-2.59 (m, 3H), 2.19 (t, 1H), 1.78-1.67 (m, 7H), 1.46-1.28 (m, 7H).

Compound 42: ((R)-1-((R)-4-morpholino-4-oxo-2-(tetrahydro-2H-pyran-4-carboxamido) butanamido)-4-phenylbutyl)boronic acid: LCMS (ESI) Calcd. for C₂₄H₃₆BN₃O₇: 489.2, found: [M−H]⁺=488.2. ¹H NMR (400 MHZ, MeOD) δ 7.24-7.12 (m, 5H), 4.97 (t, 1H), 3.95-3.92 (m, 2H), 3.67-3.59 (m, 4H), 3.51-3.37 (m, 6H), 2.92 (d, 2H), 2.60-2.45 (m, 4H), 1.72-1.47 (m, 8H).

Compound 43: ((R)-1-((R)-4-morpholino-4-oxo-2-(phenylsulfonamido)butanamido)-4-phenylbutyl)boronic acid: LCMS (ESI) Calcd. for C₂₄H₃₂BN₃O₇S: 517.3, found: [M−H]⁺=516.3. ¹H NMR (400 MHZ, MeOD) δ 7.87-7.85 (m, 2H), 7.60-7.51 (m, 3H), 7.26-7.13 (m, 5H), 4.45 (t, 1H), 3.58-3.53 (m, 4H), 3.43-3.33 (m, 4H), 2.76-2.69 (m, 2H), 2.61-2.56 (m, 3H), 1.61-1.59 (m, 2H), 1.50-1.20 (m, 2H).

Compound 44: ((1R)-1-((2R)-4-morpholino-4-oxo-2-(tetrahydro-2H-pyran-2-carboxamido) butanamido)-4-phenylbutyl)boronic acid: LCMS (ESI) Calcd. for C₂₄H₃₆BN₃O₇: 489.5, found: [M−H]⁺=488.5. ¹H NMR (400 MHZ, MeOD) δ 7.23-7.12 (m, 5H), 5.05 (br s, 1H), 4.05 (d, 1H), 3.78-3.88 (m, 1H), 3.66-3.59 (m, 4H), 3.51-3.48 (m, 6H), 3.28-3.05 (m, 2H), 2.94-2.84 (m, 1H), 2.63-2.58 (m, 3H), 1.97-1.88 (m.3H) 1.67-1.50 (m, 10H).

Compound 45: ((R)-1-((S)-4-morpholino-4-oxo-2-(pyrazine-2-carboxamido)butanamido)-4-phenylbutyl)boronic acid: LCMS (ESI) Calcd. for C₂₃H₃₀BN₅O₆: 483.5, found: [M−H]⁺=482.5. ¹H NMR (400 MHZ, MeOD) δ 9.25 (s, 1H), 8.79 (d, 1H), 8.68 (s, 1H), 7.21-7.07 (m, 5H), 5.28 (t, 1H), 3.66 (t, 2H), 3.61-3.47 (m, 6H), 2.98-2.93 (m, 1H), 2.64 (t, 1H), 2.58-2.55 (m, 2H), 1.65-1.28 (m, 5H).

Compound 46: ((S)-1-((S)-4-morpholino-4-oxo-2-(pyrazine-2-carboxamido)butanamido)-4-phenylbutyl)boronic acid: LCMS (ESI) Calcd. for C₂₃H₃₀BN₅O₆: 483.3, found: [M−H]⁺=482.3. ¹H NMR (400 MHZ, MeOD) δ 9.25 (s, 1H), 8.79 (d, 1H), 8.68 (s, 1H), 7.19-7.09 (m, 5H), 5.28 (t, 1H), 3.66-3.61 (m, 4H), 3.59-3.51 (m, 4H), 3.01-3.00 (m, 1H), 2.60-2.56 (m, 4H), 1.65-1.63 (m, 4H).

Characterization of Compounds Prepared from General Procedure G

Compound 21: ((R)-1-((R)-2-((tert-butoxycarbonyl)amino)-4-morpholino-4-oxobutanamido)-4-phenylbutyl)boronic acid: LCMS (ESI) Calcd. for C₂₃H₃₆BN₃O₇: 477.5, found: [M−H]⁺=476.5. ¹H NMR (400 MHZ, MeOD) δ 7.26-7.13 (m, 5H), 4.74 (t, 1H), 3.72-3.60 (m, 4H), 3.52-2.49 (m, 4H), 2.92 (t, 2H), 2.65-2.55 (m, 3H), 1.71-1.50 (m, 4H), 1.45 (s, 9H).

### Example 36-Biological/Biochemical Evaluation

### General Protocol for In Vitro Analysis of Compounds:

The inhibitory activity of the compounds of the present invention against LONP1, 20S proteasome and other proteases are determined by assays known to persons of ordinary skill in the art (see, e.g., Fishovitz, J. et al. “Active-Site-Directed Chemical Tools for Profiling Mitochondrial Lon Protease” ACS Chem. Biol. 6, 781-788 (2011)).

In this Example, LONP1 (NM_004793.4) activity was measured by a FRET-based assay for protease activity using a fluorogenic peptide DabcylYRGIT(2Abu)SGRQK(5-FAM) (Cambridge Research Biochemicals) as substrate. LONP1 activity is followed by an increase in fluorescence signal due to the degradation of the peptide. Inhibition of LONP1 protease activity by an inhibitor compound of the disclosure elicits a decrease in the fluorescent signal.

The assay is performed in a 384-well plate (Greiner, cat. #781076) using the following reagents and conditions: substrate (3 μM) was incubated for 1 hour at 37° C. in the presence of LONP1 (15 nM as monomer), 25 mM Tris pH 8.0, 10 mM MgCl₂, 0.03 mg/mL BSA, 0.5 mM DTT, 0.0003% Tween-20, 10 mM NaCl, 0.06 mM ATP and 0.5 mM EGTA in a 15 μL final volume. The LONP1-containing mix (10 μL) was incubated with the test compound for 15 min at 37° C. before adding the peptide-containing mix (5 μL). Solutions were dispensed using a small cassette-Multidrop Combi (Thermo Scientific). Fluorescence was measured using a PheraStar plate reader (BMG Labtech) FI-FRET EX 485 nm Em 520 nm.

The IC₅₀ values for binding to LONP1 are summarized in Table 2 below. Each value is based on an average of a minimum of two repeats.

TABLE 2
IC50 assay data for compounds of the disclosure binding to LONP1;
A: <0.05 μM; B: 0.05-0.5 μM; C: >5 μM

| | Activity IC₅₀ |
|---|---|
| Compound | (μM) |
| 1 | A |
| 2 | A |
| 3 | A |
| 4 | A |
| 5 | A |
| 6 | A |
| 7 | A |
| 8 | A |
| 9 | A |
| 10 | A |
| 11 | A |
| 12 | A |
| 13 | A |
| 14 | A |
| 17 | A |
| 19 | A |
| 20 | A |
| 21 | A |
| 22 | B |
| 23 | A |
| 27 | A |
| 29 | A |
| 31 | A |
| 32 | A |
| 33 | A |
| 35 | A |
| 37 | A |
| 38 | A |
| 39 | A |
| 40 | B |
| 41 | A |
| 42 | A |
| 43 | A |
| 44 | A |
| 45 | A |
| 46 | A |
| 47 | C |
| 48 | A |
| 50 | A |
| 51 | B |
| 52 | B |

### Cell Viability Assay:

### Materials and Kits:

Cell Proliferation Kit I (MTT), Merck, Cat #11465007001

DMEM GlutaMax, Thermo Fisher Scientific, Cat #31966021—for expansion and assay

DMEM GlutaMax, low glucose, Thermo Fisher Scientific, Cat #21885025—for expansion for cell viability assay

FBS, Gibco, Cat #A3840402

### Assay Procedure:

One day before treatment, 3,000-5,000/mL of 143b cells are placed in aliquots of 100 μL per well in flat bottom ThermoFisher 96 well plate. The starting seeding number is optimised in relation to the batch of cells and medium. The assay lasts for 8 days from seeding to MTT assay, and so the seeding number must be selected to avoid over-confluency at the last day of the assay.

On day 0, 100 μl of medium (Cat #21885025) is transferred to compound/DMSO plate, then the compound/DMSO-containing medium is transferred to the plates with pre-seeded cells.

Incubate for 7 days at 37° C., 5% CO₂ incubator.

On day 7, the medium is discarded. 100 μl of MTT labelling reagent mixed 1:10 in culture medium (Cat #21885025) is added and incubated for 4 hours at 37° C., 5% CO₂ incubator. 100 μl of MTT solubilization solution is added, mixed well and incubated overnight at 37° C.

Absorbance is measured at 570 nm on a plate reader.

### Compound Plate Setup:

The compounds are dispensed in a 96-well Greiner plate (cat no. 651201).

Volume of each compound solution: 200 nL

Final conc of DMSO: 0.1% in all wells

Starting concentration: 10 mM (final concentration on assay plate: 10 μM). In total, 8 doses and three replicates per dose per compound.

Dilution factor: 3.162

The compounds are dissolved in DMSO and dispensed according to the concentration titrations and experimental design (indicated above).

Two plates of the same compounds are dispensed and the remaining plates are retained as a backup.

Compounds can be dispensed in an Echo dispenser and sealed immediately so that they are not exposed to air and contamination. The protocol is performed under the LAF bench.

On the first day of treatment (Day 0), compound plates are opened under the LAF bench. 100 μl of assay medium (Cat #21885025) is added to each well and 100.2 μl of medium+compound/DMSO is transferred to the assay plates containing pre-seeded cells.

1. A compound of structural Formula 1: 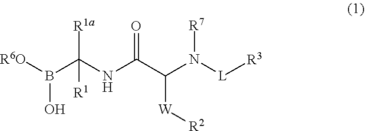
or a pharmaceutically acceptable salt, solvate, stereoisomer or mixture of stereoisomers, tautomer, isotopic form, pharmaceutically active metabolite thereof, or combinations thereof, wherein:
R¹ is selected from the group consisting of: deuterium, C1-C4 alkyl, C1-C4 alkoxyl, C1-C4 oxoalkyl, C1-C5 alkyl-alkoxyl, wherein each alkyl, oxoalkyl or alkoxyl is optionally substituted with C3-C6 cycloalkyl, phenyl, phenoxy, or a 5- or 6-membered heteroaryl, wherein said phenyl, phenoxy, or heteroaryl are each optionally substituted with one or more substituent selected from deuterium, halogen, hydroxyl, CN, CO2H, CO2R¹², CONR¹²R¹³, NR¹²R¹³, SR¹²SO2NR¹²R¹³, C1-C4 alkyl, C1-C4 haloalkyl, C1-C4 alkoxy, phenyl, or a 5- or 6-membered heteroaryl;
R^{1a} is selected from hydrogen, deuterium or C1-C2 alkyl;
W is C1-C4 alkylene, optionally substituted with one or more of deuterium, halogen, hydroxyl, oxo, CN, methyl or ethyl;
R² is selected from the group consisting of: hydrogen, deuterium, R⁴, —OR⁴, —C(O)R⁴, —S—R⁴, —SO—R⁴, —SO₂—R⁴, —SO₂—NR⁵R¹¹,
L is C(O), C(O)O, C(O)NR⁸, S(O)₂, or a bond;
R³ is C₁-C₄ alkyl optionally substituted with one or more substituents each independently selected from the group consisting of deuterium, halogen, cyano, hydroxyl, C₁-C₄ alkoxyl, C₁-C₄ alkylthiol or phenyl; or
R³ is saturated or unsaturated cycloalkyl or saturated or unsaturated heterocycloalkyl having one or more heteroatoms selected from N, O and S, wherein the cycloalkyl or heterocycloalkyl is optionally substituted with one or more substituents selected from deuterium, halogen, cyano, hydroxyl, oxo, C₁-C₄ alkoxyl, or C₁-C₄ alkyl that is optionally substituted with one to three substituents selected from deuterium, halogen, cyano, hydroxyl, C₁-C₄ alkylthiol or C₁-C₄ alkoxyl; or
R³ is aryl or heteroaryl having one or more heteroatoms selected from N, O and S, wherein aryl or heteroaryl is optionally substituted with one or more substituents selected from deuterium, halogen, cyano, hydroxyl, OR, CO2H, CO2R¹², CONR¹²R¹³, NR¹²R¹³, SR¹², SO2NR¹²R¹³, C₁-C₄ alkoxyl, or C₁-C₄ alkyl that is optionally substituted with one to three substituents selected from deuterium, halogen, cyano, hydroxyl, or C₁-C₄ alkoxyl;
R⁴ is hydrogen, deuterium, C₁-C₄ alkyl or C₁-C₄ alkoxyl optionally substituted with one or more substituents each independently selected from the group consisting of deuterium, halogen, cyano, hydroxyl, C₁-C₄ alkoxyl, C₁-C₄ alkylthiol, C₁-C₅ alkyl-alkoxyl or phenyl; or
R⁴ is saturated or unsaturated cycloalkyl or saturated or unsaturated heterocycloalkyl having one or more heteroatoms selected from N, O and S, wherein the cycloalkyl or heterocycloalkyl is optionally substituted with one or more substituents selected from deuterium, halogen, cyano, hydroxyl, C₁-C₄ alkoxyl, or C₁-C₄ alkyl that is optionally substituted with one to three substituents selected from deuterium, halogen, cyano, hydroxyl, or C₁-C₄ alkoxyl, C₁-C₄ alkylthiol and C₁-C₅ alkyl-alkoxyl; or
R⁴ is aryl or heteroaryl having one or more heteroatoms selected from N, O and S, wherein aryl, or heteroaryl is optionally substituted with one or more substituents selected from deuterium, halogen, cyano, hydroxyl, C₁-C₄ alkoxyl, or C₁-C₄ alkyl that is optionally substituted with one to three substituents selected from deuterium, halogen, cyano, hydroxyl, or C₁-C₄ alkoxyl, C₁-C₄ alkylthiol and C₁-C₅ alkyl-alkoxyl; or
R⁴ is NR⁹R¹⁰;
R⁵ and R¹¹ are each independently selected from hydrogen, deuterium or C1-C5 alkyl optionally substituted with one to three halogen; or
R⁵ and R¹¹ together with the N to which they are attached form a 5 or 6 membered saturated or unsaturated heterocyclic ring optionally having one or more additional heteroatoms selected from N, O and S, wherein the 5 or 6 membered heterocyclic ring is optionally substituted with one or more substituent selected from deuterium, halogen, hydroxyl, CN, C1-C4 alkyl, C1-C4 haloalkyl or C1-C4 alkoxy;
R⁶ is hydrogen, or R⁶ and R¹, together with the boron atom to which —OR⁶ is attached form a 5-membered heteroalkyl ring.
R⁷ is selected from hydrogen, deuterium, or C1-C4 alkyl optionally substituted with one or more substituents each independently selected from the group consisting of deuterium, halogen, hydroxyl, cyano, methoxyl, methylthiol and phenyl;
R⁸ is hydrogen or C₁-C₄ alkyl optionally substituted with one or more of deuterium, halogen, hydroxyl and phenyl, wherein phenyl is optionally substituted with one or more substituent selected from deuterium, halogen, hydroxyl and C₁-C₂ alkyl;
R⁹ and R¹⁰ are each independently selected from hydrogen, deuterium or C₁-C₆ alkyl that is optionally substituted with one to three substituents selected from deuterium, halogen, cyano, or C₁-C₄ alkoxyl; or
R⁹ and R¹⁰ together with the N to which they are attached form a 5 or 6 membered saturated or unsaturated heterocyclic ring optionally having one or more additional heteroatoms selected from N, O and S, wherein the 5 or 6 membered heterocyclic ring is optionally substituted with one or more substituent selected from deuterium, halogen, hydroxyl, CN, C1-C4 alkyl, C1-C4 haloalkyl or C1-C4 alkoxy; and
R¹² and R¹³ are each independently selected from hydrogen, deuterium, C1-C4 alkyl; C1-C4 haloalkyl, C1-C5 alkyl-alkoxyl, C3-C7 cycloalkyl, or R¹² and R¹³ together with the N to which they are attached form 3 to 7 membered heterocyclic ring optionally having one or more additional heteroatoms selected from N, O and S, wherein the C3-C7 cycloalkyl or 3 to 7 membered heterocyclic ring is optionally substituted with one or more substituent selected from deuterium, halogen, hydroxyl, oxo, CN, C1-C4 alkyl, C1-C4 haloalkyl or C1-C4 alkoxyl.

2. The compound according to claim 1, wherein R¹ is selected from C1-C4 alkyl, C1-C4 alkoxyl, C1-C4 alkyl-alkoxyl, each optionally substituted with phenyl or a 5- or 6-membered heteroaryl.

3. The compound according to claim 1, wherein R^{1a} is hydrogen or methyl.

4. The compound according to claim 1, wherein W is C1-C4 alkylene optionally substituted with one or more of hydroxyl or oxo.

5. The compound according to claim 1, wherein R² is selected from R⁴, OR⁴, C(O)R⁴, SO—R⁴, SO₂—R⁴ or SO₂—NR⁵R¹¹.

6. The compound according to claim 1, wherein R⁴ is C₁-C₄ alkyl, an amino optionally substituted with one or more C₁-C₄ alkyl, an optionally substituted 5- or 6-membered heterocyclic ring having one or more heteroatoms, an optionally substituted 5- or 6-membered heteroaromatic ring having one or more heteroatoms, or an optionally substituted 5- or 6-membered aromatic ring;

7. The compound according to claim 1, wherein R⁵ and R¹¹ are each independently selected from hydrogen, or C₁-C₄ alkyl optionally substituted with one to three fluoro.

8. The compound according to claim 1, wherein R⁶ is hydrogen.

9. The compound according to claim 1, wherein R⁷ is selected from hydrogen, F, Cl, hydroxyl, methyl, ethyl, CF₃, OMe, phenyl-(CH₂)— or phenyl-(CH₂)₂—.

10. The compound according to claim 1, wherein R⁷ is selected from hydrogen or methyl.

11. The compound according to claim 1, wherein R⁷ is hydrogen.

12. The compound according to claim 1, wherein R⁸ is selected from hydrogen, or C₁-C₄ alkyl optionally substituted with one to three fluoro.

13. The compound according to claim 1, wherein R⁹ and R¹⁰ are each independently selected from hydrogen or C₁-C₄ alkyl that is optionally substituted with one to three substituents selected from halogen, cyano, or C₁-C₂ alkoxy.

14. The compound according to claim 1, wherein R⁹ and R¹⁰ are each independently selected from hydrogen or C₁-C₂ alkyl that is optionally substituted with one to three substituents selected from fluoro or chloro.

15. The compound according to claim 1, wherein R⁹ and R¹⁰ are each methyl.

16. The compound according to claim 1, wherein R¹² and R¹³ are each independently selected from hydrogen, deuterium, C1-C2 alkyl; C1-C2 haloalkyl, C1-C2 alkyl-alkoxyl or C3-C7 cycloalkyl, wherein C3-C7 cycloalkyl is optionally substituted with one or more substituent selected from deuterium, F, Cl, hydroxyl, oxo, CN, C1-C2 alkyl, C1-C2 haloalkyl or C1-C2 alkoxyl.

17. The compound according to claim 1, wherein R¹² and R¹³ together with the N to which they are attached form 3 to 7 membered heterocyclic ring optionally having one or two additional heteroatoms selected from N, O and S, which is optionally substituted with one or more substituent selected from deuterium, F, Cl, hydroxyl, oxo, CN, C1-C2 alkyl, C1-C2 haloalkyl or C1-C2 alkoxyl.

18. The according to claim 1, having the structural Formula 2: 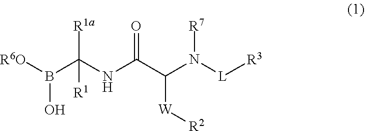
wherein:
R¹ is C₁-C₄ alkyl, C₁-C₅ alkoxy, C₁-C₄ alkyl alkoxy, each optionally substituted with phenyl or heteroaryl;
W is C₁-C₄ alkylene;
R² is C(O)R⁴, OR⁴, R⁴, SO₂—NR⁵R¹¹, SO₂—R⁴, SO—R⁴;
L is C(O), C(O)O, C(O)NR⁸, S(O)₂, or a bond;
R³ is C₁-C₄ alkyl optionally substituted with one or more substituents each independently selected from the groups consisting of fluoro, chloro, cyano, or methoxyl, cycloalkyl, heterocyclyl having one or more heteroatoms, aryl, cycloaryl, or heteroaryl having one or more heteroatoms, which is optionally substituted with one or more fluoro, chloro, cyano, methoxyl, or C1-C4 alkyl that is optionally substituted with one to three fluoro, chloro, cyano, or methoxyl;
R⁴ is C₁-C₄ alkyl, an amino optionally substituted with one or more C₁-C₄ alkyl, an optionally substituted 5- or 6-membered heterocyclic ring having one or more heteroatoms, an optionally substituted 5- or 6-membered heteroaromatic ring having one or more heteroatoms, or an optionally substituted 5- or 6-membered aromatic ring; and
R⁵, R⁸ and R¹¹ are each independently selected from hydrogen or C₁-C₄ alkyl optionally substituted with one to three fluoro.

19. The compound according to claim 1, wherein R¹ is selected from methyl, ethyl, n-propyl, i-propyl, n-butyl or tert-butyl, each optionally substituted with a phenyl ring.

20. The compound according to claim 1, wherein R¹ is selected from methyl, n-propyl, n-butyl or tert-butyl.

21. The compound according to claim 1, wherein R¹ is selected from phenyl-(CH₂)₂— or phenyl-(CH₂)₃—.

22. The compound according to claim 1, wherein R¹ is selected from tert-butyl or phenyl-(CH₂)₃—.

23. The compound according to claim 1, wherein W is C1-C3 alkylene optionally substituted with oxo.

24. The compound according to claim 1, wherein W is propylene.

25. The compound according to claim 1, wherein W is C1-C2 alkylene optionally substituted with oxo.

26. The compound according to claim 25, wherein W is 2-oxoethylene.

27. The compound according to claim 1, wherein W is C1-C2 alkylene, optionally substituted with one or more of deuterium, halogen, hydroxyl, CN, methyl or ethyl.

28. The compound according to claim 1, wherein W is selected from methylene or ethylene, wherein the methylene or ethylene is optionally substituted with one to three substituents selected from deuterium, fluoro, chloro, hydroxyl or methyl.

29. The compound according to claim 1, wherein W is methylene or ethylene.

30. The compound according to claim 1, wherein W is methylene.

31. The compound according to claim 1, wherein R² is C(O)R⁴, OR⁴ or R⁴.

32. The compound according to claim 1, wherein R² is SO₂—NR⁵R¹¹, SO₂—R⁴ or SO—R⁴.

33. The compound according to claim 1, wherein R⁴ is a 5 or 6 membered heterocyclic ring having one or more heteroatoms selected from N, O and S, wherein the heterocyclic ring is optionally substituted with one or more substituent selected from deuterium, halogen, hydroxyl, CN, C1-C4 alkyl, C1-C4 haloalkyl or C1-C4 alkoxy.

34. The compound according to claim 1, wherein R⁴ is a 5 or 6 membered heterocyclic ring having one or two heteroatoms selected from N, O and S, wherein the heterocyclic ring is optionally substituted with one or more substituent selected from deuterium, halogen, hydroxyl, CN, methyl, ethyl, C1-C2 haloalkyl or C1-C2 alkoxy.

35. The compound according to claim 1, wherein R⁴ is a 5 or 6 membered heterocyclic ring having one heteroatom selected from N, O and S, wherein the heterocyclic ring is optionally substituted with one or more substituent selected from F, Cl, hydroxyl, methyl, ethyl, wherein the methyl and ethyl are optionally substituted with one or more halogen or deuterium.

36. The compound according to claim 1, wherein R⁴ is a 5 or 6 membered heterocyclic ring having two heteroatoms selected from N, O and S, wherein the heterocyclic ring is optionally substituted with one or more substituent selected from F, Cl, hydroxyl, methyl, ethyl, wherein the methyl and ethyl are optionally substituted with one or more halogen or deuterium.

37. The compound according to claim 1, wherein R⁴ is a 9 or 10 membered bicyclic heterocyclic ring having one or more heteroatoms selected from N, O and S, wherein the heterocyclic ring is optionally substituted with one or more substituent selected from deuterium, halogen, hydroxyl, CN, C1-C4 alkyl, C1-C4 haloalkyl or C1-C4 alkoxy.

38. The compound according to claim 1, wherein R⁴ is a 9 or 10 membered heterocyclic ring having one or two heteroatoms selected from N, O and S, wherein the heterocyclic ring is optionally substituted with one or more substituent selected from deuterium, halogen, hydroxyl, CN, methyl, ethyl, C1-C2 haloalkyl or C1-C2 alkoxy.

39. The compound according to claim 1, wherein R⁴ is a 9 or 10 membered heterocyclic ring having one heteroatom selected from N, O and S, wherein the heterocyclic ring is optionally substituted with one or more substituent selected from F, Cl, hydroxyl, methyl, ethyl, wherein the methyl and ethyl are optionally substituted with one or more halogen or deuterium.

40. The compound according to claim 1, wherein R⁴ is a 9 or 10 membered heterocyclic ring having two heteroatoms selected from N, O and S, wherein the heterocyclic ring is optionally substituted with one or more substituent selected from F, Cl, hydroxyl, methyl, ethyl, wherein the methyl and ethyl are optionally substituted with one or more halogen or deuterium.

41. The compound according to claim 33, wherein the heteroatom or heteroatoms are selected from: (i) N; (ii) N and O; (iii) N and S; or (iv) O and S.

42. The compound according to claim 1, wherein R⁴ is a heterocyclic ring selected from optionally substituted: tetrahydrofuranyl, furanyl, pyrrolidinyl, pyrrolyl, thiophenyl, imidazolyl, pyrazolyl, oxazolyl, isooxazolyl, thiazolyl, isothiazolyl, oxadiazolyl, pyridinyl, piperidinyl, pyridazinyl, piperazinyl, pyrimidinyl, pyrazinyl, tetrahydropyranyl, pyranyl, dioxanyl, morpholinyl, azepanyl, oxepanyl, oxazepanyl, pyrrolizidinyl, indolyl, isoindolyl, indolizinyl, benzimidazolyl, purinyl, quinolinyl, isoquinolinyl, quinazolinyl, or pteridinyl; and wherein the heterocyclic ring is joined to W via a carbon atom or via a heteroatom.

43. The compound according to claim 1, wherein R⁴ is a heterocyclic ring selected from: imidazolyl, pyrazolyl, oxazolyl, thiazolyl or indolyl.

44. The compound according to claim 1, wherein R⁴ is a heterocyclic ring selected from: 2-imidazolyl, 3-pyrazolyl, 2-oxazolyl, 5-oxazolyl, 2-thiazolyl or 3-indolyl.

45. The compound according to claim 1, wherein R⁴ is substituted with methyl, and wherein methyl may be attached to a carbon atom or to a heteroatom.

46. The compound according to claim 1, wherein R⁴ is a heterocyclic ring selected from: 1-methyl-2-imidazolyl, 1-methyl-3-pyrazolyl, 2-oxazolyl, 5-oxazolyl, 2-thiazolyl or 3-indolyl.

47. The compound according to claim 1, wherein R⁴ is a heterocyclic ring selected from: 1-methyl-2-imidazolyl, 1-methyl-3-pyrazolyl.

48. The compound according to claim 1, wherein R² is R⁴.

49. The compound according to claim 1, wherein R⁸ is selected from hydrogen, F, Cl, hydroxyl, methyl, ethyl, CF₃, or OMe.

50. The compound according to claim 1, wherein R⁸ is selected from hydrogen or methyl.

51. The compound according to claim 1, wherein L is selected from C(O), C(O)O, C(O) NH, C(O)N(CH₃), SO₂.

52. The compound according to claim 1, wherein L is selected from C(O), C(O)O and C(O) NH.

53. The compound according to claim 1, wherein L is C(O).

54. The compound according to claim 1, wherein R³ is C₁-C₄ alkyl, a 5- or 6-membered heteroaryl, C6 aryl, a 5- or 6-membered heterocycloalkyl or C6 cycloalkyl, and wherein R³ is optionally substituted.

55. The compound according to claim 1, wherein R³ is methyl, ethyl, n-propyl, i-propyl, n-butyl or tert-butyl, each optionally substituted with a phenyl ring.

56. The compound according to claim 1, wherein R³ is selected from methyl, i-propyl and tert-butyl.

57. The compound according to claim 1, wherein R³ is selected from phenyl, phenyl-(CH₂)— and phenyl-(CH₂)₂—, wherein the phenyl group is optionally substituted.

58. The compound according to claim 1, wherein R³ is selected from an aryl, heteroaryl, cycloalkyl or heterocycloalkyl selected from tetrahydropyranyl, pyrazinyl, tetrahydropyrrolyl, tetrahydrofuranyl, tetrahydropyranyl, cyclohexanyl, oxazolyl and morpholinyl, wherein said aryl, heteroaryl, cycloalkyl or heterocycloalkyl is optionally substituted.

59. The compound according to claim 1, wherein R³ is selected from n-tetrahydropyrrolyl, morpholinyl and pyrazinyl.

60. The compound according to claim 57, wherein said substituent is selected from one to three of halogen, hydroxyl and C1-C2 alkyl.

61. The compound according to claim 57, wherein said substituent is selected from one or two of Cl, hydroxyl and methyl.

62. The compound according to claim 57, wherein R³ is selected from 2-chlorophenyl, 3-chlorophenyl, 2,5-dichlorophenyl, 2,4-dimethyloxazolyl, and 3-hydroxy n-tetrahydropyrrolyl.

63. The compound according to claim 1, wherein R^{1a} is hydrogen.

64. The compound according to claim 1, wherein R⁵ and R¹¹ are each independently selected from hydrogen or C1-C2 alkyl.

65. The compound according to claim 1, wherein R⁵ and R¹¹ are hydrogen.

66. The compound according to claim 1, wherein R⁸ is selected from hydrogen or C1-C2 alkyl.

67. The compound according to claim 1, wherein R⁸ is hydrogen.

68. The compound according to claim 1, wherein halogen is selected from fluoro or chloro.

69. The compound according to claim 1, wherein halogen is chloro.

70. The compound according to claim 1, which is selected from any one of Compounds 3, 3a, 3b, 39 51 and 52.

71. The compound according to claim 1, wherein the compound is an inhibitor of LONP1.

72. A pharmaceutical composition comprising one or more compounds according to claim 1 or pharmaceutically acceptable salt, solvate, stereoisomer or mixture of stereoisomers, tautomer, isotopic form, or pharmaceutically active metabolite thereof, or combinations thereof, and one or more pharmaceutically acceptable carrier.

73. A pharmaceutical composition comprising a compound according to Formula 1, 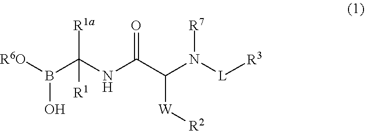
or a pharmaceutically acceptable salt, solvate, stereoisomer or mixture of stereoisomers, tautomer, isotopic form, or pharmaceutically active metabolite thereof, or combinations thereof, and one or more pharmaceutically acceptable carrier, wherein:
R¹ is selected from the group consisting of: deuterium, C1-C4 alkyl, C1-C4 alkoxyl, C₁-C₄ oxoalkyl, C1-C5 alkyl-alkoxyl, wherein each alkyl, oxoalkyl or alkoxyl is optionally substituted with C3-C6 cycloalkyl, phenyl, phenoxy, or a 5- or 6-membered heteroaryl, wherein said phenyl, phenoxy, or heteroaryl are each optionally substituted with one or more substituent selected from deuterium, halogen, hydroxyl, CN, CO2H, CO2R¹², CONR¹²R¹³, NR¹²R¹³, SR¹²SO2NR¹²R¹³, C1-C4 alkyl, C1-C4 haloalkyl, C1-C4 alkoxy, phenyl, or a 5- or 6-membered heteroaryl;
R^{1a} is selected from hydrogen, deuterium or C1-C2 alkyl;
W is C1-C4 alkylene, optionally substituted with one or more of deuterium, halogen, hydroxyl, oxo, CN, methyl or ethyl;
R² is selected from the group consisting of: hydrogen, deuterium, R⁴, —OR⁴, —C(O)R⁴, —S—R⁴, —SO—R⁴, —SO₂—R⁴, —SO₂—NR⁵R¹¹,
L is C(O), C(O)O, C(O)NR⁸, S(O)₂, or a bond;
R³ is C₁-C₄ alkyl optionally substituted with one or more substituents each independently selected from the group consisting of deuterium, halogen, cyano, hydroxyl, C₁-C₄ alkoxyl, C₁-C₄ alkylthiol or phenyl; or
R³ is saturated or unsaturated cycloalkyl or saturated or unsaturated heterocycloalkyl having one or more heteroatoms selected from N, O and S, wherein the cycloalkyl or heterocycloalkyl is optionally substituted with one or more substituents selected from deuterium, halogen, cyano, hydroxyl, oxo, C₁-C₄ alkoxyl, or C₁-C₄ alkyl that is optionally substituted with one to three substituents selected from deuterium, halogen, cyano, hydroxyl, C₁-C₄ alkylthiol or C₁-C₄ alkoxyl; or
R³ is aryl or heteroaryl having one or more heteroatoms selected from N, O and S, wherein aryl or heteroaryl is optionally substituted with one or more substituents selected from deuterium, halogen, cyano, hydroxyl, OR, CO2H, CO2R¹², CONR¹²R¹³, NR 12R¹³, SR¹²SO2NR¹²R¹³, C₁-C₄ alkoxyl, or C₁-C₄ alkyl that is optionally substituted with one to three substituents selected from deuterium, halogen, cyano, hydroxyl, or C₁-C₄ alkoxyl;
R⁴ is hydrogen, deuterium, C₁-C₄ alkyl or C₁-C₄ alkoxyl optionally substituted with one or more substituents each independently selected from the group consisting of deuterium, halogen, cyano, hydroxyl, C₁-C₄ alkoxyl, C₁-C₄ alkylthiol, C₁-C₅ alkyl-alkoxyl or phenyl; or
R⁴ is saturated or unsaturated cycloalkyl or saturated or unsaturated heterocycloalkyl having one or more heteroatoms selected from N, O and S, wherein the cycloalkyl or heterocycloalkyl is optionally substituted with one or more substituents selected from deuterium, halogen, cyano, hydroxyl, C₁-C₄ alkoxyl, or C₁-C₄ alkyl that is optionally substituted with one to three substituents selected from deuterium, halogen, cyano, hydroxyl, or C₁-C₄ alkoxyl, C₁-C₄ alkylthiol and C₁-C₅ alkyl-alkoxyl; or
R⁴ is aryl or heteroaryl having one or more heteroatoms selected from N, O and S, wherein aryl, or heteroaryl is optionally substituted with one or more substituents selected from deuterium, halogen, cyano, hydroxyl, C₁-C₄ alkoxyl, or C₁-C₄ alkyl that is optionally substituted with one to three substituents selected from deuterium, halogen, cyano, hydroxyl, or C₁-C₄ alkoxyl, C₁-C₄ alkylthiol and C₁-C₅ alkyl-alkoxyl; or
R⁴ is NR⁹R¹⁰;
R⁵ and R¹¹ are each independently selected from hydrogen, deuterium or C1-C5 alkyl optionally substituted with one to three halogen; or
R⁵ and R¹¹ together with the N to which they are attached form a 5 or 6 membered saturated or unsaturated heterocyclic ring optionally having one or more additional heteroatoms selected from N, O and S, wherein the 5 or 6 membered heterocyclic ring is optionally substituted with one or more substituent selected from deuterium, halogen, hydroxyl, CN, C1-C4 alkyl, C1-C4 haloalkyl or C1-C4 alkoxy;
R⁶ is hydrogen, or R⁶ and R¹, together with the boron atom to which —OR⁶ is attached form a 5-membered heteroalkyl ring.
R⁷ is selected from hydrogen, deuterium, or C₁-C₄ alkyl optionally substituted with one or more substituents each independently selected from the group consisting of deuterium, halogen, hydroxyl, cyano, methoxyl, methylthiol and phenyl;
R⁸ is hydrogen or C₁-C₄ alkyl optionally substituted with one or more of deuterium, halogen, hydroxyl and phenyl, wherein phenyl is optionally substituted with one or more substituent selected from deuterium, halogen, hydroxyl and C₁-C₂ alkyl;
R⁹ and R¹⁰ are each independently selected from hydrogen, deuterium or C₁-C₆ alkyl that is optionally substituted with one to three substituents selected from deuterium, halogen, cyano, or C₁-C₄ alkoxyl; or
R⁹ and R¹⁰ together with the N to which they are attached form a 5 or 6 membered saturated or unsaturated heterocyclic ring optionally having one or more additional heteroatoms selected from N, O and S, wherein the 5 or 6 membered heterocyclic ring is optionally substituted with one or more substituent selected from deuterium, halogen, hydroxyl, CN, C1-C4 alkyl, C1-C4 haloalkyl or C1-C4 alkoxy; and
R¹² and R¹³ are each independently selected from hydrogen, deuterium, C1-C4 alkyl; C1-C4 haloalkyl, C1-C5 alkyl-alkoxyl, C3-C7 cycloalkyl, or R¹² and R¹³ together with the N to which they are attached form 3 to 7 membered heterocyclic ring optionally having one or more additional heteroatoms selected from N, O and S, wherein the C3-C7 cycloalkyl or 3 to 7 membered heterocyclic ring is optionally substituted with one or more substituent selected from deuterium, halogen, hydroxyl, oxo, CN, C1-C4 alkyl, C1-C4 haloalkyl or C1-C4 alkoxyl.

74. The pharmaceutical composition of claim 73, wherein the compound of Formula 1 is defined according to claim 2.

75. The compound according to claim 1, or the for use in the treatment of a disease or disorder.

76. The compound for use according to claim 75, wherein the disease or disorder is characterized by mitochondrial dysfunction, such as mitochondrial disorders, including a neurodegenerative disorder, a metabolic disorder and a disease associated with the aging process.

77. The compound for use according to claim 75, wherein the disease or disorder is an oncologic disease or disorder, such as a cancer and/or a proliferative disease or disorder.

78. The compound for use according to claim 77, wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma colon cancer, colorectal cancer, cancer of connective tissue, esophageal cancer, embryonal carcinoma, fibrosarcoma, glioblastomas, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), liposarcoma, liver cancer, lung cancer, lymphoid cancers (e.g., Hodgkin's and non-Hodgkin's lymphomas, mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML).

79. The compound for use according to claim 75, wherein the use comprises administering the compound orally; topically; by inhalation; by intranasal administration; or systemically by intravenous, intraperitoneal, subcutaneous, or intramuscular injection.

80. The compound for use according to claim 75, wherein the use comprises administering to a subject one or more compounds according to claim 1, optionally in combination with one or more additional therapeutic agent.

81. The compound for use according to claim 80, wherein the administering comprises administering the one or more compounds according to claim 1 simultaneously, sequentially or separately from the one or more additional therapeutic agent.

82. A method for treating or preventing a disease or disorder in a subject where inhibition of LONP1 may be beneficial, wherein said method comprises administering to the subject one or more compounds according to claim 1.

83. The method according to claim 82, wherein the disease or disorder is characterized by mitochondrial dysfunction, such as mitochondrial disorders, including a neurodegenerative disorder, a metabolic disorder and a disease associated with the aging process.

84. The method according to claim 82, wherein the disease or disorder is an oncologic disease or disorder, such as a cancer and/or a proliferative disease or disorder.

85. The method according to claim 86, wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma colon cancer, colorectal cancer, cancer of connective tissue, esophageal cancer, embryonal carcinoma, fibrosarcoma, glioblastomas, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), liposarcoma, liver cancer, lung cancer, lymphoid cancers (e.g., Hodgkin's and non-Hodgkin's lymphomas, mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML).

86. The method according to claim 82, wherein one or more compounds according to claim 1 is administered in combination with one or more additional therapeutic agent.

87. The method according to claim 86, wherein the administering comprises administering the one or more compounds according to claim 1 simultaneously, sequentially or separately from the one or more additional therapeutic agent.
